(19) United States
(12) Patent Application Publication
(10) Pub. No.: US 2025/0333520 A1
(43) Pub. Date: Oct. 30, 2025

(54) COMPOSITIONS AND METHODS RELATED TO IL2 RECEPTOR BINDING

(71) Applicant: Synthekine, Inc., Menlo Park, CA (US)

(72) Inventors: Robert Kastelein, Menlo Park, CA (US); Rene De Waal Malefyt, Menlo Park, CA (US); Deepti Rokkam, Menlo Park, CA (US); Patrick Lupardus, Menlo Park, CA (US); Sandro Vivona, Menlo Park, CA (US)

(21) Appl. No.: 18/260,709

(22) PCT Filed: Jan. 11, 2022
(86) PCT No.: PCT/US2022/012055

§ 371 Date: Jul. 7, 2023

Related U.S. Application Data
(63) Continuation-in-part of application No. PCT/US21/44853, filed on Aug. 5, 2021.
(60) Provisional application No. 63/135,884, filed on Jan. 11, 2021.
(60) Provisional application No. 63/136,095, filed on Jan. 11, 2021.

(51) Int. Cl. C07K 16/28; A61K 39/00

(52) U.S. Cl. CPC C07K 16/2866; A61K 2039/505; C07K 2317/40; C07K 2317/569; C07K 2317/92

(57) ABSTRACT

Provided herein am IL2R binding proteins that bind to IL2Rβ and IL2Rγ and comprise an anti-IL2Rβ VHH antibody and an anti-IL2Rγ VHH antibody.

### CROSS-REFERENCE TO RELATED PATENT APPLICATIONS

The present patent application is a 371 national stage application of PCT/US2022/012055, filed Jan. 11, 2022, which claims priority to U.S. Provisional Application No. 63/136,095, filed Jan. 11, 2021, U.S. Provisional Application No. 63/135,884, filed Jan. 11, 2021, and PCT Application No. PCT/US2021/044853, filed Aug. 5, 2021, the disclosure of each of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Feb. 25, 2022, is named 106249-1293186-007300PC_SL.txt and is 900,711 bytes in size.

### BACKGROUND OF THE DISCLOSURE

Interleukin 2 (IL2) is a pluripotent cytokine produced primarily by activated CD4 T cells that is involved in producing a normal immune response. IL2 exerts a wide spectrum of effects on the immune system and plays important roles in regulating both immune activation, suppression and homeostasis. IL2 promotes proliferation and expansion of activated T lymphocytes, potentiates B cell growth, and activates monocytes and natural killer cells. The amino acid sequence of human IL2 is found in Genbank under accession locator NP 000577.2.

As an immune system stimulator, IL2 has found use in the treatment of cancer and chronic viral infections. However, the effects of IL2 have also been associated with mediation of autoimmunity and transplant rejection. IL2 therapy, especially at high doses, has been associated with significant toxicity in human subjects. Consequently, a therapeutic goal is to maintain desired actions of IL2 while minimizing associated autoimmune or immunosuppressive responses. Because of its roles in immune regulation and disease, the search for new IL2 analogs and variants remains an active area of research.

IL2 exerts its effect on mammalian immune cells through interaction with three different cell surface proteins: (1) CD25 (also referred to as the IL2 receptor alpha, IL2Rα, or p55), CD122 (also referred to as the IL2 receptor beta, IL2Rβ, IL15Rβ, or p70-75), and CD132 (also referred to as the IL2 receptor gamma, IL2Rγ, or common gamma chain as it is a component of other multimeric receptors in this family).

CD25 is a 55 kD polypeptide that is constituitively expressed in Treg cells and inducibly expressed on other T cells in response to activation (e.g., by CD3). hIL2 binds to hCD25 with a Kd of approximately 10⁻⁸ M. CD25 is also referred to in the literature as the “low affinity” IL2 receptor. The human CD25 is expressed as a 272 amino acid pre-protein comprising a 21 amino acid signal sequence which is post-translationally removed to render a 251 amino acid mature protein. Amino acids 22-240 (amino acids 1-219 of the mature protein) correspond to the extracellular domain. Amino acids 241-259 (amino acids 220-238 of the mature protein) correspond to transmembrane domain. Amino acids 260-272 (amino acids 239-251 of the mature protein) correspond to intracellular domain. The intracellular domain of CD25 is comparatively small (13 amino acids) and has not been associated with any independent signaling activity. The IL2/CD25 complex has not been observed to produce a detectable intracellular signaling response. Human CD25 nucleic acid and protein sequences may be found as Genbank accession numbers NM_000417 and NP_0004Q8 respectively.

CD122 is a single pass type I transmembrane protein. The human CD122 (hCD122) is expressed as a 551 amino acid protein, the first 26 amino acids comprising a signal sequence which is post-translationally cleaved in the mature 525 amino acid protein. Amino acids 27 240 (amino acids 1-214 of the mature protein) correspond to the extracellular domain, amino acids 241-265 (amino acids 225-239 of the mature protein) correspond to the transmembrane domain and amino acids 266-551 (amino acids 240-525 of the mature protein) correspond to the intracellular domain. As used herein, the term CD122 includes naturally occurring variants of the CD122 protein including the S57F and D365E (as numbered in accordance with the mature hCD122 protein). hCD122 is referenced at UniProtKB database as entry P14784. Human CD122 nucleic acid and protein sequences may be found as Genbank accession numbers NM 000878 and NP_000869 respectively.

CD132 is a type 1 cytokine receptor and is shared by the receptor complexes for IL4, IL7, IL9, IL15, and IL21, hence the reference to the “common” gamma chain. Human CD132 (hCD132) is expressed as a 369 amino acid pre-protein comprising a 22 amino acid N-terminal signal sequence. Amino acids 23-262 (amino acids 1-240 of the mature protein) correspond to the extracellular domain, amino acids 263-283 (amino acids 241-262 of the mature protein) correspond to the 21 amino acid transmembrane domain, and amino acids 284-369 (amino acids 262-347 of the mature protein) correspond to the intracellular domain. hCD132 is referenced at UniProtKB database as entry P31785. Human CD132 nucleic acid and protein sequences may be found as Genbank accession numbers: NM 000206 and NP_000197 respectively.

The IL2 receptor proteins combine to produce two additional IL2 receptor complexes: (a) an “intermediate affinity” IL2 receptor comprising CD122 and CD132 (also referred to as IL2Rβγ) and (b) a “high affinity” IL2 receptor complex comprising the CD25, CD122, and CD132 proteins (also referred to as IL2Rαβγ). hIL2 possesses a Kd of approximately 10⁻⁹ M with respect to the intermediate affinity CD122/CD132 (IL2βγ) receptor complex. The intermediate affinity CD122/CD132 (IL2βγ) receptor complex is predominantly expressed on resting T-cells and NK cells. hIL2 possesses a Kd of approximately 10⁻¹¹ M with respect to the high IL2 affinity receptor complex. Most cells, such as resting T cells, demonstrate low responsiveness to IL2 since they only express the CD122 and CD132 which have comparatively low affinity for IL2 relative to the CD25/CD122/CD132 high affinity receptor complex. The high affinity receptor complex is predominantly identified on activated lymphocytes which inducibly express CD25 and Treg cells that express CD25 constituitively.

### SUMMARY OF THE DISCLOSURE

In one aspect, provided herein is an IL2 receptor (IL2R) binding protein that specifically binds to IL2Rβ and IL2Rγ, comprising an anti-IL2Rβ VHH antibody and an anti-IL2Rγ VHH antibody. In some embodiments, the IL2R binding molecule comprises a single-domain antibody (sdAb) that specifically binds to IL2Rβ (an IL2Rb sdAb) and a sdAb that specifically binds to IL2Rγ (an anti-IL2Rγ sdAb).

In some embodiments, the anti-IL2Rβ VHH antibody comprises a complementarity determining region 1 (CDR1) comprising an amino acid sequence of SEQ ID NO: 1 or SEQ ID NO: 410, a CDR2 comprising an amino acid sequence of SEQ ID NO: 2, and CDR3 a comprising an amino acid sequence of SEQ ID NO: 3; and the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
wherein each CDR independently comprises 0, 1, 2, or 3 amino acid changes relative to the sequence.

In some embodiments, the anti-IL2Rβ VHH antibody comprises a complementarity determining region 1 (CDR1) comprising an amino acid sequence of SEQ ID NO: 5 or SEQ ID NO: 411, a CDR2 comprising an amino acid sequence of SEQ ID NO: 6, and CDR3 a comprising an amino acid sequence of SEQ ID NO: 7; and the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   wherein each CDR independently comprises 0, 1, 2, or 3 amino acid changes relative to the sequence.

In some embodiments, the anti-IL2Rβ VHH antibody comprises a complementarity determining region 1 (CDR1) comprising an amino acid sequence of SEQ ID NO: 9 or SEQ ID NO: 412, a CDR2 comprising an amino acid sequence of SEQ ID NO: 10, and CDR3 a comprising an amino acid sequence of SEQ ID NO: 11; and the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   wherein each CDR independently comprises 0, 1, 2, or 3 amino acid changes relative to the sequence.

In some embodiments, the anti-IL2Rβ VHH antibody comprises a complementarity determining region 1 (CDR1) comprising an amino acid sequence of SEQ ID NO: 13 or SEQ ID NO: 413, a CDR2 comprising an amino acid sequence of SEQ ID NO: 14, and CDR3 a comprising an amino acid sequence of SEQ ID NO: 15; and the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   wherein each CDR independently comprises 0, 1, 2, or 3 amino acid changes relative to the sequence.

In some embodiments, the anti-IL2Rβ VHH antibody comprises a complementarity determining region 1 (CDR1) comprising an amino acid sequence of SEQ ID NO: 17 or SEQ ID NO: 414, a CDR2 comprising an amino acid sequence of SEQ ID NO: 18, and CDR3 a comprising an amino acid sequence of SEQ ID NO: 19; and the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   wherein each CDR independently comprises 0, 1, 2, or 3 amino acid changes relative to the sequence.

In some embodiments, the anti-IL2Rβ VHH antibody comprises a complementarity determining region 1 (CDR1) comprising an amino acid sequence of SEQ ID NO: 21 or SEQ ID NO: 415, a CDR2 comprising an amino acid sequence of SEQ ID NO: 122, and CDR3 a comprising an amino acid sequence of SEQ ID NO: 23; and the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   wherein each CDR independently comprises 0, 1, 2, or 3 amino acid changes relative to the sequence.

In some embodiments, the anti-IL2Rβ VHH antibody comprises a complementarity determining region 1 (CDR1) comprising an amino acid sequence of SEQ ID NO: 25 or SEQ ID NO: 416, a CDR2 comprising an amino acid sequence of SEQ ID NO: 26, and CDR3 a comprising an amino acid sequence of SEQ ID NO: 27; and the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   wherein each CDR independently comprises 0, 1, 2, or 3 amino acid changes relative to the sequence.

In some embodiments, the anti-IL2Rβ VHH antibody comprises a complementarity determining region 1 (CDR1) comprising an amino acid sequence of SEQ ID NO: 29 or SEQ ID NO: 417, a CDR2 comprising an amino acid sequence of SEQ ID NO: 30, and CDR3 a comprising an amino acid sequence of SEQ ID NO: 31; and the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   wherein each CDR independently comprises 0, 1, 2, or 3 amino acid changes relative to the sequence.

In some embodiments, the anti-IL2Rβ VHH antibody comprises a complementarity determining region 1 (CDR1) comprising an amino acid sequence of SEQ ID NO: 33 or SEQ ID NO: 418, a CDR2 comprising an amino acid sequence of SEQ ID NO: 34, and CDR3 a comprising an amino acid sequence of SEQ ID NO: 35; and the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   wherein each CDR independently comprises 0, 1, 2, or 3 amino acid changes relative to the sequence.

In some embodiments, the anti-IL2Rβ VHH antibody comprises a complementarity determining region 1 (CDR1) comprising an amino acid sequence of SEQ ID NO: 37 or SEQ ID NO: 419, a CDR2 comprising an amino acid sequence of SEQ ID NO: 38, and CDR3 a comprising an amino acid sequence of SEQ ID NO: 39; and the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   wherein each CDR independently comprises 0, 1, 2, or 3 amino acid changes relative to the sequence.

In some embodiments, the anti-IL2Rβ VHH antibody comprises:
(1) a complementarity determining region 1 (CDR1) having a sequence of any one of SEQ ID NOS: 1, 5, 9, 13, 17, 21, 25, 29, 33, and 37, 410, 411, 412, 413, 414, 415, 416, 417, 418, and 419;
(2) a CDR2 having a sequence of any one of SEQ ID NOS: 2, 6, 10, 14, 18, 22, 26, 30, 34, and 38; and
(3) a CDR3 having a sequence of any one of SEQ ID NOS: 3, 7, 11, 15, 19, 23, 27, 31, 35, and 39.

In some embodiments, the anti-IL2Rβ VHH antibody comprises CDR1, CDR2, and CDR3 sequences of an anti-IL2Rβ VHH antibody selected from the group consisting of DR214, DR217, DR583, DR584, DR585, DR586, DR587, DR588, DR589, and DR590.

In some embodiments the anti-IL2Rβ VHH antibody comprises a sequence having at least 90% identity to a sequence of any one of DR214 (SEQ ID NO: 4), DR217 (SEQ ID NO: 8), DR583 (SEQ ID NO: 12), DR584 (SEQ ID NO: 16), DR585 (SEQ ID NO: 20), DR586 (SEQ ID NO: 24), DR587 (SEQ ID NO: 28), DR588 (SEQ ID NO: 32), DR589 (SEQ ID NO: 36), and DR590 (SEQ ID NO: 40).

In some embodiments, the anti-IL2Rγ VHH antibody comprises:
(1) a complementarity determining region 1 (CDR1) having a sequence of any one of SEQ ID NOS: 41, 45, 49, 53, 57, and 61, 420, 421, 422, 423, 424, and 425;
(2) a CDR2 having a sequence of any one of SEQ ID NOS: 42, 46, 50, 54, 58, and 62; and
(3) a CDR3 having a sequence of any one of SEQ ID NOS: 43, 47, 51, 55, 59, and 63.

In some embodiments, the anti-IL2Rγ VHH antibody comprises CDR1, CDR2, and CDR3 sequences of an anti-IL2Rγ VHH antibody selected from the group consisting of DR229, DR230, DR231, DR232, DR233, and DR234.

In some embodiments, the anti-IL2Rγ VHH antibody comprises a sequence having at least 90% identity to a sequence of any one of DR229 (SEQ ID NO: 44), DR230 (SEQ ID NO: 48), DR231 (SEQ ID NO: 52), DR232 (SEQ ID NO: 56), DR233 (SEQ ID NO: 60), and DR234 (SEQ ID NO: 64).

In some embodiments, the anti-IL2Rβ VHH antibody comprises a CDR1 comprising an amino acid sequence of SEQ ID NO: 29 or SEQ ID NO: 417, a CDR2 comprising an amino acid sequence of SEQ ID NO: 30, and CDR3 a comprising an amino acid sequence of SEQ ID NO: 31; and the anti-IL2Rγ VHH antibody comprises a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51. In some embodiments the anti-IL2Rβ VHH antibody comprises a sequence having at least 90% identity to SEQ ID NO: 32. In some embodiments, the anti-IL2Rγ VHH antibody comprises a sequence having at least 90% identity to SEQ ID NO: 52. In some embodiments, the IL2R binding molecule comprises a sequence having at least 90% identity to SEQ ID NO: 76, optionally without the HHHHHH (SEQ ID NO: 127) sequence. In some embodiments, the IL2R binding molecule comprises a sequence having at least 90% identity to SEQ ID NO: 274, optionally without the HHHHHH (SEQ ID NO: 127) sequence. {DR736}

In some embodiments, the anti-IL2Rβ VHH antibody is at the N-terminus and the anti-IL2Rγ VHH antibody is at the C-terminus. In some embodiments, the binding protein comprises a sequence having at least 90% identity to a sequence of any one of SEQ ID NOS: 65-80.

In some embodiments, the anti-IL2Rγ VHH antibody is at the N-terminus and the anti-IL2Rβ VHH antibody is at the C-terminus. In some embodiments, the binding protein comprises a sequence having at least 90% identity to a sequence of any one of SEQ ID NOS: 81-106.

In some embodiments, the anti-IL2Rβ VHH antibody and the anti-IL2Rγ VHH antibody are joined by a peptide linker. In some embodiments, the peptide linker comprises between 1 and 50 amino acids.

In some embodiments, the binding protein comprises a sequence with at least 90% (e.g., 90%, 91%, 92%, 93%, 94%, 95%, 96%, 97%, 98%, 99%, or 100%) identity to a sequence of any one of SEQ ID NOS: 65-80, SEQ ID NOS: 81-106, or SEQ ID NOS: 170-289, optionally without a HHHHHHHHH (SEQ ID NO: 127) sequence.

In some embodiments, the binding protein is conjugated to an Fc polypeptide or an Fc domain.

In some embodiments, the Fc polypeptide or the Fc domain is from an IgG1, IgG2, IgG3 or IgG4.

In some embodiments, the binding protein is PEGylated.

In another aspect, the disclosure provides an IL2R binding protein that specifically binds to IL2Rβ and IL2Rγ, comprising an anti-IL2Rβ VHH antibody and an anti-IL2Rγ VHH antibody, wherein the IL2Rβ/IL2Rγ binding protein is linked to a Fc polypeptide or a Fc domain from an IgG1, IgG2, IgG3 or IgG4.

In another aspect, described herein is a heterodimeric IL2Rβ binding protein/IL2Rγ binding protein pair, the heterodimeric IL2Rβ binding protein/IL2Rγ binding protein pair comprising a first polypeptide of the formula #1:

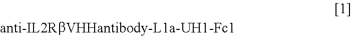

and a second polypeptide of the formula #2:

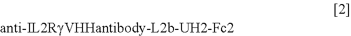

wherein:
L1 and L2 are GSA linkers and a and b are independently selected from 0 (absent) or 1 (present);
UH1 and UH2 are each an upper hinge domain of human immunoglobulin independently selected from the group consisting of the IgG1, IgG2, IgG3 and IgG4 upper hinge, optionally comprising the amino acid substitution C220S (EU numbering);
Fc1 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc2, and
FC2 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc1, and
wherein the polypeptide of formula 1 and the polypeptide of formula 2 are linked by at least one interchain disulfide bond. In some embodiments:
   (A) the anti-IL2Rβ VHH antibody comprises a complementarity determining region 1 (CDR1) comprising an amino acid sequence of SEQ ID NO: 1 or SEQ ID NO: 410, a CDR2 comprising an amino acid sequence of SEQ ID NO: 2, and CDR3 a comprising an amino acid sequence of SEQ ID NO: 3; and wherein the anti-IL2Rγ VHH antibody comprises:
   i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
   ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
   iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
   iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
   v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
   vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   (B) the anti-IL2Rβ VHH antibody comprises a complementarity determining region 1 (CDR1) comprising an amino acid sequence of SEQ ID NO: 5 or SEQ ID NO: 411, a CDR2 comprising an amino acid sequence of SEQ ID NO: 6, and CDR3 a comprising an amino acid sequence of SEQ ID NO: 7; and wherein the anti-IL2Rγ VHH antibody comprises:
   i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
   ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
   iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
   iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
   v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
   vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   (C) the anti-IL2Rβ VHH antibody comprises a complementarity determining region 1 (CDR1) comprising an amino acid sequence of SEQ ID NO: 9 or SEQ ID NO: 412, a CDR2 comprising an amino acid sequence of SEQ ID NO: 10, and CDR3 a comprising an amino acid sequence of SEQ ID NO: 11; and wherein the anti-IL2Rγ VHH antibody comprises:
   i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
   ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
   iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
   iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
   v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
   vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   (D) the anti-IL2Rβ VHH antibody comprises a complementarity determining region 1 (CDR1) comprising an amino acid sequence of SEQ ID NO: 13 or SEQ ID NO: 413, a CDR2 comprising an amino acid sequence of SEQ ID NO: 14, and CDR3 a comprising an amino acid sequence of SEQ ID NO: 15; and wherein the anti-IL2Rγ VHH antibody comprises:
   i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
   ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
   iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
   iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
   v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
   vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   (E) the anti-IL2Rβ VHH antibody comprises a complementarity determining region 1 (CDR1) comprising an amino acid sequence of SEQ ID NO: 17 or SEQ ID NO: 414, a CDR2 comprising an amino acid sequence of SEQ ID NO: 18, and CDR3 a comprising an amino acid sequence of SEQ ID NO: 19; and wherein the anti-IL2Rγ VHH antibody comprises:
   i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
   ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
   iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
   iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
   v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
   vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   (F) the anti-IL2Rβ VHH antibody comprises a complementarity determining region 1 (CDR1) comprising an amino acid sequence of SEQ ID NO: 21 or SEQ ID NO: 415, a CDR2 comprising an amino acid sequence of SEQ ID NO: 122, and CDR3 a comprising an amino acid sequence of SEQ ID NO: 23; and wherein the anti-IL2Rγ VHH antibody comprises:
   i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
   ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
   iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
   iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
   v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
   vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   (G) the anti-IL2Rβ VHH antibody comprises a complementarity determining region 1 (CDR1) comprising an amino acid sequence of SEQ ID NO: 25 or SEQ ID NO: 416, a CDR2 comprising an amino acid sequence of SEQ ID NO: 26, and CDR3 a comprising an amino acid sequence of SEQ ID NO: 27; and wherein the anti-IL2Rγ VHH antibody comprises:
   i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
   ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
   iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
   iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
   v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
   vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   (H) the anti-IL2Rβ VHH antibody comprises a complementarity determining region 1 (CDR1) comprising an amino acid sequence of SEQ ID NO: 29 or SEQ ID NO: 417, a CDR2 comprising an amino acid sequence of SEQ ID NO: 30, and CDR3 a comprising an amino acid sequence of SEQ ID NO: 31; and wherein the anti-IL2Rγ VHH antibody comprises:
   i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
   ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
   iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
   iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
   v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
   vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   (I) the anti-IL2Rβ VHH antibody comprises a complementarity determining region 1 (CDR1) comprising an amino acid sequence of SEQ ID NO: 33 or SEQ ID NO: 418, a CDR2 comprising an amino acid sequence of SEQ ID NO: 34, and CDR3 a comprising an amino acid sequence of SEQ ID NO: 35; and wherein the anti-IL2Rγ VHH antibody comprises:
   i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
   ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
   iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
   iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
   v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
   vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   (J) the anti-IL2Rβ VHH antibody comprises a complementarity determining region 1 (CDR1) comprising an amino acid sequence of SEQ ID NO: 37 or SEQ ID NO: 419, a CDR2 comprising an amino acid sequence of SEQ ID NO: 38, and CDR3 a comprising an amino acid sequence of SEQ ID NO: 39; and wherein the anti-IL2Rγ VHH antibody comprises:
   i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
   ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
   iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
   iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
   v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59;
   vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
   wherein each CDR independently comprises 0, 1, 2, or 3 amino acid changes relative to the sequence.

In some embodiments, the anti-IL2Rβ VHH antibody comprises:
a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR1 in a row of Table 30 or Table 31; a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR2 in a row of Table 30 or Table 31; and
a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR3 listed in Table 30 or Table 31; and the anti-IL2Rγ VHH antibody comprises:
a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR1 listed in Table 32 or Table 33; a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR2 listed in Table 32 or Table 33; and a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR3 listed in Table 32 or Table 33.

In some embodiments, the anti-IL2Rβ VHH antibody comprises an amino acid sequence having at least 90% (e.g., 91%, 92%, 93%, 94%, 95%, 96%, 97%, 98%, 99%, or 100%) sequence identity) to a sequence in a row of Table 34 or Table 35.

In some embodiments, the anti-IL2Rγ VHH antibody comprises an amino acid sequence having at least 90% (e.g., 91%, 92%, 93%, 94%, 95%, 96%, 97%, 98%, 99%, or 100%) sequence identity) to a sequence in a row of Table 36 or Table 37.

In another aspect, the disclosure provides an isolated nucleic acid encoding an IL2R binding protein, or a heterodimeric IL2Rβ binding protein/IL2Rγ binding protein pair as described herein.

In another aspect, the disclosure provides an expression vector comprising the nucleic acid described herein.

In another aspect, the disclosure provides an isolated host cell comprising the vector comprising the nucleic acid described herein.

In another aspect, the disclosure provides a pharmaceutical composition comprising the IL2R binding protein or a heterodimeric IL2Rβ binding protein/IL2Rγ binding protein pair described herein and a pharmaceutically acceptable carrier.

In another aspect, the disclosure provides a method of treating a neoplastic disease in a subject in need thereof, comprising administering to the subject a therapeutically effective amount of an IL2R binding protein or a heterodimeric IL2Rβ binding protein/IL2Rγ binding protein pair described herein, or a pharmaceutical composition comprising (i) the IL2R binding protein or (ii) the heterodimeric IL2Rβ binding protein/IL2Rγ binding protein pair described herein and a pharmaceutically acceptable carrier.

In some embodiments of this aspect, the method further comprises the administration of a supplementary agent to the subject. In some embodiments the supplementary agent is selected from the group consisting of a chemotherapeutic agent, an a therapeutic antibody, an immune checkpoint modulator, a TIL, a CAR-T cell, and a physical method. In some embodiments the therapeutic antibody is an antibody that binds to at least one tumor antigen selected from the group consisting of HER2, nectin-4, CD79, CTLA4, CD22, CCR4, IL23p19, PDL1, IL17a, CD38, SLAMF7, CD20, CD30, CD33, CD52, EpCam, CEA, fpA33, TAG-72, CAIX, PSMA, PSA, folate binding protein, GD2, GD3, IL6, GM2, Ley, VEGF, VEGFR, VEGFR2, PDGFR, EGFR, ERBB2, ERBB3, MET, IGF1R, EPHA3, TRAIL R1, TRAIL R2, RANKL RAP, tenascin, integrin V 3, and integrin 4 1.

In some embodiments the neoplastic disease disorder is selected from the group consisting of: adenomas, fibromas, hemangiomas, hyperplasia, atypia, metaplasia, dysplasia, carcinomas, leukemias, breast cancers, sarcomas, leukemias, lymphomas, genitourinary cancers, ovarian cancers, urethral cancers, bladder cancers, prostate cancers, gastrointestinal cancers, colon cancers, esophageal cancers, stomach cancers, lung cancers; myelomas; pancreatic cancers; liver cancers; kidney cancers; endocrine cancers; skin cancers; gliomas, neuroblastomas, astrocytomas, myelodysplastic disorders; cervical carcinoma-in-situ; intestinal polyposes; oral leukoplakias; histiocytoses, hyperprofroliferative scars including keloid scars, respiratory system carcinomas, gastrointestinal system carcinomas, genitourinary system carcinomas, testicular carcinomas, breast carcinomas, prostatic carcinomas, endocrine system carcinomas, melanomas, adenocarcinomas, myeloproliferative neoplasms, myeloid and lymphoid disorders with eosinophilia, myeloproliferative/myelodysplastic neoplasms, myelodysplastic syndromes, acute myeloid leukemia and related precursor neoplasms, and acute leukemia of ambiguous lineage, promyeloid leukemia (APML), acute myelogenous leukemia (AML) and chronic myelogenous leukemia (CML), precursor lymphoid neoplasms, mature B-cell neoplasms, mature T-cell neoplasms, Hodgkin's Lymphoma, and immunodeficiency-associated lymphoproliferative disorders, lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). erythroblastic leukemia and acute megakaryoblastic leukemia, malignant lymphomas including, but are not limited to, non-Hodgkins lymphoma and variants thereof, peripheral T cell lymphomas, adult T-cell leukemia/lymphoma (ATL), cutaneous T cell lymphoma (CTCL), large granular lymphocytic leukemia (LGF), Hodgkin's disease and Reed-Stemberg disease.

In another aspect, the disclosure provides a method of treating an autoimmune or inflammatory disease, disorder, or condition or a viral infection in a subject in need thereof, comprising administering to the subject a therapeutically effective amount of an IL2R binding protein, or a heterodimeric IL2Rβ binding protein/IL2Rγ binding protein pair described herein or a pharmaceutical composition comprising (i) the IL2R binding protein or (ii) the heterodimeric IL2Rβ binding protein/IL2Rγ binding protein pair described herein and a pharmaceutically acceptable carrier.

In some embodiments of this aspect, the method further comprises administering one or more supplementary agents selected from the group consisting of a corticosteroid, a Janus kinase inhibitor, a calcineurin inhibitor, a mTor inhibitor, an IMDH inhibitor, a biologic, a vaccine, and a therapeutic antibody. In certain embodiments the therapeutic antibody is an antibody that binds a protein selected from the group consisting of BLyS, CD11a, CD20, CD25, CD3, CD52, IgEIL12/IL23, IL17a, IL1β, IL4Rα, IL5, IL6R, integrin-α4β7, RANKL, TNFα, VEGF-A, and VLA-4.

In some embodiments of this aspect, the disease, disorder, or condition is selected from viral infections, heliobacter pylori infection, HTLV, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome, juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, SEA Syndrome, psoriasis, psoriatic arthritis, dermatitis (eczema), exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid, urticaria, prokeratosis, rheumatoid arthritis; seborrheic dermatitis, solar dermatitis; seborrheic keratosis, senile keratosis, actinic keratosis, photo-induced keratosis, keratosis follicularis; acne vulgaris; keloids; nevi; warts including verruca, condyloma or condyloma acuminatum, and human papilloma viral (HPV) infections.

In another aspect, the disclosure provides a method to selectively induce proliferation of a first cell type over a second cell type, comprising contacting a population of cells comprising both the first and second cell types with an IL2 binding protein, or an heterodimeric IL2Rβ binding protein/IL2Rγ binding protein pair desribed herein, thereby selectively inducing proliferation in one or more of the first cell type over one or more of the second cell type.

In some embodiments the first cell type is T cells and the second cell type is NK cells. In some embodiments the first cell type is NK cells and the second cell type is T cells. In some embodiments, the number of cells of the first cell type is at least 1.2 (e.g., at least 1.4, 1.6, 1.8, 2, 2.2, 2.4, 2.6, 2.8, 3, 3.2, 3.4, 3.6, 3.8, 4, 6, 8, 10, 12, 14, 16, 18, or 20) fold more than the number of cells of the second cell type.

### BRIEF DESCRIPTION OF THE FIGURES

FIG. 1 of the attached drawings provides a schematic representation of one embodiment of the binding molecule of the present disclosure comprising a first single domain antibody (1) and a second single domain antibody (3) and a linker (2) depicted as interacting with a cell membrane (10) associated heterodimeric receptor comprising a first receptor subunit comprising an extracellular domain (4), and transmembrane domain (5) and an intracellular domain (6) nteraction of a binding molecule and a second first receptor subunit comprising an extracellular domain (7), and transmembrane domain (8) and an intracellular domain (9) wherein the intracellular domain of the first receptor (6) and the intracellular domain of the second receptor (9) on of a binding molecule are within a proximal distance (11).

FIG. 2 of the attached drawings provides a schematic representation of two illustrative configurations of binding molecules of the present disclosure. Panel A provides a schematic representation of an illustrative binding molecule comprising a first single domain antibody (1) and a second single domain antibody (3) and a linker (2). Panel B provides a schematic representation of a binding molecule comprising two polypeptide chains, the first polypeptide chain comprising (from amino to carboxy) a first single domain antibody (1), a linker sequence (2), a second single domain antibody (3), an IgG hinge sequence (12) and an Fc knob domain (13) and a second polypeptide comprising an Fc hole (14) wherein the first and second polypeptides are in stable association via the interaction of the knob-into-hole Fc domain.

FIG. 3 of the attached drawings provides a schematic representations of two illustrative configurations of binding molecules of the present disclosure. Panel A provides a schematic representation of an illustrative binding molecule construct comprising two binding molecules each attached to a subunit of a knob-into-hole Fc domain, the construct comprising two polypeptide chains, the first polypeptide chain comprising, from amino to carboxy, a first single domain antibody (1), a linker (2) and a second single domain antibody (3), a IgG hinge sequence (12) and a Fc knob subunit (13) and a second polypeptide chain comprising, from amino to carboxy, a first single domain antibody (1), a linker (2) and a second single domain antibody (3), a IgG hinge sequence (12) and a Fc hole subunit (14) wherein the first and second polypeptides are in stable association via the interaction of the knob-into-hole Fc domain. Panel B provides schematic representation of a an alternative arrangement of a binding molecule construct comprising two polypeptides a first polypeptide chain comprising, from amino to carboxy, a first single domain antibody (1), a linker (2) and a second single domain antibody (3), an IgG hinge sequence (12) and a Fc knob subunit (13) and a second polypeptide chain comprising, from amino to carboxy, a first second domain antibody (3), a linker (2) and a first single domain antibody (1), a IgG hinge sequence (12) and a Fc hole subunit (14), wherein the first and second polypeptides are in stable association via the interaction of the knob-into-hole Fc domain.

FIG. 4, Panel A provides alternative schematic representations of configurations of the binding molecules of the present disclosure where one single domain antibody is attached to each subunit of a knob-into-hole Fc domain comprising two polypeptides, the first polypeptide comprising from amino to carboxy, a first single domain antibody (1), an IgG hinge sequence (12) and a Fc knob subunit (13), the second polypeptide comprising from amino to carboxy, a second single domain antibody (3), an IgG hinge sequence (12) and a Fc hole subunit (14), wherein the first and second single domain antibodies are in stable associate via the interaction of the knob-into-hole Fc domain.

FIG. 4, Panel B provides a schematic representations of a binding molecule where the binding domains are single domain antibodies associated via transition metal coordinate covalent complex. As illustrated, the binding molecules comprises two polypeptide subunits: the first subunit comprising a first single domain antibody (1) is attached via a first linker (15) to a first chelating peptide (17) and the second subunit comprising a second single domain antibody (3) is attached via a second linker (16) to a second chelating peptide (18), wherein the first chelating peptide (17) and second chelating peptide (18) form a coordinate covalent complex with a single transition metal ion (“M”). The transition metal ion may be in a kinetically labile or kinetically inert oxidation state.

FIG. 5 provides data with respect to IL2R binding molecules of the present disclosure on the induction of IFN gamma in NK cells measured by luminescent. This data illustrates that varying the sdAb components may provide substantial variations in activity significantly greater than wt IL2 in some instances.

FIG. 6 of the attached drawings provides data from the evaluation of T cells outgrown on PBMCs isolated from two separate donors. The data shows that the IL2R binding molecules enable selective T cell proliferation activity with respect to NK cells, even though the NK cells express the IL2Rb/g receptors. This demonstrates that variation in receptor binding affinity can be used to modulate the activity of the IL2R binding molecules in selective cell types.

FIGS. 7A-7D show different configurations of one or two IL2Rβ/IL2Rγ binding proteins conjugated to an Fc domain.

FIG. 8 shows the dose response of anti-IL2Rβ/γ VHH2s on NK cell proliferation.

FIG. 9 shows the effect on CD45 expression in CD8 and CD4 cells obtained from blood and spleen cell populations in mice in response to the anti-IL2Rβ/γ VHH2 DR638 on as evaluated by FACS (left) and, at right, the percent of CD45+CD4+ cells and CD45+CD8+ cells in both blood and spleen populations in obtained from mice in response to the administration of various test articles as more fully described in Example 21.

FIG. 10 illustrates the expression levels of various phenotypic markers on CD8+NK1.1 T cells in blood (upper row) and spleen cell (lower row) populations obtained from mice in response to the administration of various test articles as more fully described in Example 21.

FIG. 11 illustrates the expression levels of various phenotypic markers on CD8+ T cells in blood (upper row) and spleen cell (lower row) populations obtained from mice in response to the administration of various test articles as more fully described in Example 21.

FIG. 12 illustrates the expression levels of various phenotypic markers on CD4+ T cells in blood (upper row) and spleen cell (lower row) populations obtained from mice in response to the administration of various test articles as more fully described in Example 21.

FIG. 13 illustrates the expression levels of various phenotypic markers on CD4+CD25+ Tregs cells in blood (upper row) and spleen cell (lower row) populations obtained from mice in response to the administration of various test articles as more fully described in Example 21.

FIG. 14 illustrates the expression levels of various phenotypic markers on CD8+CD25+ Tregs cells in blood (upper row) and spleen cell (lower row) populations obtained from mice in response to the administration of various test articles as more fully described in Example 21.

FIG. 15 illustrates the expression levels of various phenotypic markers on CD8+Cd25 negative T cells in blood (upper row) and spleen cell (lower row) populations obtained from mice in response to the administration of various test articles as more fully described in Example 21.

FIG. 16 is a graphical representation of the survival of of hIL2Rβ/hIL2Rγ dtg and WT BL/6 mice following administration of anti-IL2Rβ/γ V_{H}H² DR638peg, DR736peg or Neoleukin. as more fully described in Example 21.

### DETAILED DESCRIPTION OF THE DISCLOSURE

### I. Introduction

The present disclosure provides compositions useful in the pairing of cellular receptors to generate desirable effects useful in treatment of diseases. In general, binding proteins are provided that comprise a first domain that binds to IL2Rβ and a second domain that binds to IL2Rγ, such that upon contacting with a cell expressing IL2Rβ and IL2Rγ, the binding protein causes the functional association of IL2Rβ and IL2Rγ, thereby resulting in functional dimerization of the receptors and downstream signaling.

Several advantages flow from the binding proteins described herein. The natral ligand of IL2R, IL2, causes IL2Rβ and IL2Rγ to come into proximity (i.e., by their simultaneous binding of IL2). However, when IL2 is used as a therapeutic in mammalian, particularly human, subjects, it may also trigger a number of adverse and undesirable effects by a variety of mechanisms including the presence of IL2Rβ and IL2Rγ on other cell types and the binding to IL2Rβ and IL2Rγ on the other cell types may result in undesirable effects and/or undesired signaling on cells expressing IL2Rβ and IL2Rγ. The present disclosure is directed to methods and compositions that modulate the multiple effects of IL2Rβ and IL2Rγ binding so that desired therapeutic signaling occurs, particularly in a desired cellular or tissue subtype, while minimizing undesired activity and/or intracellular signaling.

In some embodiments, the binding proteins described herein are designed such that the binding proteins provide the maximal desired IL2 intracellular signaling from binding to IL2Rβ and IL2Rγ on the desired cell types, while providing significantly less IL2 signaling on other undesired cell types. This can be achieved, for example, by selection of binding proteins having differing affinities or causing different Eₘₐₓ for IL2Rβ and IL2Rγ as compared to the affinity of IL2 for IL2Rβ and IL2Rγ. Because different cell types respond to the binding of ligands to its cognate receptor with different sensitivity, by modulating the affinity of the dimeric ligand (or its individual binding moieties) for the IL2 receptor relative to wild-type IL2 binding facilitates the stimulation of desired activities while reducing undesired activities on non-target cells. To measure downstream signaling activity, a number of methods are available. For example, in some embodiments, one can measure JAK/STAT signaling by the presence of phosphorylated receptors and/or phosphorylated STATs. In other embodiments, the expression of one or more downstream genes, whose expression levels can be effected by the level of downstream signalinging caused by the binding protein, can also be measured.

### II. Definitions

As used herein, the term “antibody” refers collectively to: (a) glycosylated and non-glycosylated immunoglobulins (including but not limited to mammalian immunoglobulin classes IgG1, IgG2, IgG3 and IgG4) that specifically binds to target molecule and (b) immunoglobulin derivatives including but not limited to IgG(1-4)deltaCx2, F(ab′)₂, Fab, ScFv, V_{H}, V_{L}, tetrabodies, triabodies, diabodies, dsFv, F(ab′)₃, scFv-Fc and (scFv)₂ that competes with the immunoglobulin from which it was derived for binding to the target molecule. The term antibody is not restricted to immunoglobulins derived from any particular mammalian species and includes murine, human, equine, and camelids antibodies (e.g., human antibodies).

The term antibody also includes so called “single-domain antibodies” or “sdAbs,” as well as “heavy chain antibodies” or “V_{H}Hs,” which are further defined herein. V_{H}Hs can be obtained from immunization of camelids (including camels, llamas, and alpacas (see, e.g., Hamers-Casterman, et al. (1993) Nature 363:446-448) or by screening libraries (e.g., phage libraries) constructed in V_{H}H frameworks. Antibodies having a given specificity may also be derived from non-mammalian sources such as V_{H}Hs obtained from immunization of cartilaginous fishes including, but not limited to, sharks. The term “antibody” encompasses antibodies isolatable from natural sources or from animals following immunization with an antigen and as well as engineered antibodies including monoclonal antibodies, bispecific antibodies, trispecific, chimeric antibodies, humanized antibodies, human antibodies, CDR-grafted, veneered, or deimmunized (e.g., to remove T-cell epitopes) antibodies. The term “human antibody” includes antibodies obtained from human beings as well as antibodies obtained from transgenic mammals comprising human immunoglobulin genes such that, upon stimulation with an antigen the transgenic animal produces antibodies comprising amino acid sequences characteristic of antibodies produced by human beings.

The term antibody includes both the parent antibody and its derivatives such as affinity matured, veneered, CDR grafted, humanized, camelized (in the case of V_{H}Hs), or binding molecules comprising binding domains of antibodies (e.g., CDRs) in non-immunoglobulin scaffolds.

The term “antibody” should not be construed as limited to any particular means of synthesis and includes naturally occurring antibodies isolatable from natural sources and as well as engineered antibodies molecules that are prepared by “recombinant” means including antibodies isolated from transgenic animals that are transgenic for human immunoglobulin genes or a hybridoma prepared therefrom, antibodies isolated from a host cell transformed with a nucleic acid construct that results in expression of an antibody, antibodies isolated from a combinatorial antibody library including phage display libraries. In one embodiment, an “antibody” is a mammalian immunoglobulin. In some embodiments, the antibody is a “full length antibody” comprising variable and constant domains providing binding and effector functions.

The term antibody includes antibody conjugates comprising modifications to prolong duration of action such as fusion proteins or conjugation to polymers (e.g., PEGylated).

As used herein, the term “binding protein” refers to a protein that can bind to one or more cell surface receptors or domains or subunits thereof. In some embodiments, a binding protein specifically binds to two different receptors (or domains or subunits thereof) such that the receptors (or domains or subunits) are maintained in proximity to each other such that the receptors (or domains or subunits), including domains thereof (e.g., intracellular domains) interact with each other and result in downstream signaling.

As used herein, the term “CDR” or “complementarity determining region” is intended to mean the non-contiguous antigen combining sites found within the variable region of both heavy and light chain immunoglobulin polypeptides. CDRs have been described by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. In the context of the present disclosure, the numbering of the CDR positions is provided according to Kabat numbering conventions. The term “Chothia Numbering” as used herein is recognized in the arts and refers to a system of numbering amino acid residues based on the location of the structural loop regions (Chothia et al. 1986, Science 233:755-758; Chothia & Lesk 1987, JMB 196:901-917; Chothia et al. 1992, JMB 227:799-817). For purposes of the present disclosure, unless otherwise specifically identified, the positioning of CDRs 2 and 3 in the variable region of an antibody follows Kabat numbering, or simply “Kabat.” The positioning of CDR1 in the variable region of an antibody can follow Kabat numbering unless indicated as determined by a hybrid of Kabat and Chothia numbering schemes.

As used herein, the term “conservative amino acid substitution” refers to an amino acid replacement that changes a given amino acid to a different amino acid with similar biochemical properties (e.g., charge, hydrophobicity, and size). For example, the amino acids in each of the following groups can be considered as conservative amino acids of each other. (1) hydrophobic amino acids: alanine, isoleucine, leucine, tryptophan, phenylalanine, valine, proline, and glycine; (2) polar amino acids: glutamine, asparagine, histidine, serine, threonine, tyrosine, methionine, and cysteine; (3) basic amino acids: lysine and arginine; and (4) acidic amino acids: aspartic acid and glutamic acid.

As used herein, the term “downstream signaling” refers to the cellular signaling process that is caused by the interaction of two or more cell surface receptors that are brought into proximity of each other.

As used herein, the term “linker” refers to a linkage between two elements, e.g., protein domains. A linker can be a covalent bond or a peptide linker. The term “bond” refers to a chemical bond, e.g., an amide bond or a disulfide bond, or any kind of bond created from a chemical reaction, e.g., chemical conjugation. The term “peptide linker” refers to an amino acid or polyeptide that may be employed to link two protein domains to provide space and/or flexibility between the two protein domains.

As used herein, the term “multimerization” refers to two or more cell surface receptors, or domains or subunits thereof, being brought in close proximity to each other such that the receptors, or domains or subunits thereof, can interact with each other and cause downstream signaling.

As used herein, the terms “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

As used herein, the term “neoplastic disease” refers to disorders or conditions in a subject arising from cellular hyper-proliferation or unregulated (or dysregulated) cell replication. The term neoplastic disease refers to disorders arising from the presence of neoplasms in the subject. Neoplasms may be classified as: (1) benign (2) pre-malignant (or “pre-cancerous”); and (3) malignant (or “cancerous”). The term “neoplastic disease” includes neoplastic-related diseases, disorders and conditions referring to conditions that are associated, directly or indirectly, with neoplastic disease, and includes, e.g., angiogenesis and precancerous conditions such as dysplasia.

As used herein, the terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

As used herein, the term “percent (%) sequence identity” used in the context of nucleic acids or polypeptides, refers to a sequence that has at least 50% sequence identity with a reference sequence. Alternatively, percent sequence identity can be any integer from 50% to 100%. In some embodiments, a sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined with BLAST using standard parameters, as described below.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Nat. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁻⁵, and most preferably less than about 10⁻²⁰.

As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences; fusion proteins with or without N-terminus methionine residues; fusion proteins with immunologically tagged proteins; fusion proteins of immunologically active proteins (e.g., antigenic diphtheria or tetanus toxin fragments) and the like.

As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed due to genetic, experiential or environmental factors to having a particular disease, disorder or condition. In certain instances, the terms “prevent”, “preventing”, “prevention” are also used to refer to the slowing of the progression of a disease, disorder or condition from a present its state to a more deleterious state.

As used herein, the term “single-domain antibody” or “sdAb” refers to an antibody having a single monomeric variable antibody domain. A sdAb is able to bind selectively to a specific antigen. A VHH antibody, further defined below, is an example of a sdAb.

As used herein, the term “specifically bind” refers to the degree of selectivity or affinity for which one molecule binds to another. In the context of binding pairs (e.g., a binding protein described herein/receptor, a ligand/receptor, antibody/antigen, antibody/ligand, antibody/receptor binding pairs), a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample.

In a particular embodiment, a VHH in a bispecific V_{H}H² binding protein described herein binds to a receptor (e.g., the first receptor or the second receptor of the natural or non-natural receptor pairs) if the equilibrium dissociation constant between the V_{H}H and the receptor is greater than about 10⁶ M, alternatively greater than about 10⁸ M, alternatively greater than about 10¹⁰ M, alternatively greater than about 10¹¹ M, alternatively greater than about 10¹⁰ M, greater than about 10¹² M as determined by, e.g., Scatchard analysis (Munsen, et al. 1980 Analyt. Biochem. 107:220-239). Specific binding may be assessed using techniques known in the art including but not limited to competition ELISA, BIACORE® assays and/or KINEXA® assays.

As used herein, the term “subject”, “recipient”, “individual”, or “patient”, refers to any mammalian subject for whom diagnosis, treatment, or therapy is desired, particularly humans. These terms can also be used interchangeably herein. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

As used herein the term “T-cell” or “T cell” is used in its conventional sense to refer to a lymphocytes that differentiates in the thymus, possess specific cell-surface antigen receptors, and include some that control the initiation or suppression of cell-mediated and humoral immunity and others that lyse antigen-bearing cells. In some embodiments the T cell includes without limitation naive CD8⁺ T cells, cytotoxic CD8⁺ T cells, naive CD4⁺ T cells, helper T cells, e.g. T_{H}1, T_{H}2, T_{H}9, T_{H}11, T_{H}22, T_{FH}; regulatory T cells, e.g. T_{R}1, Tregs, inducible Tregs; memory T cells, e.g. central memory T cells, effector memory T cells, NKT cells, tumor infiltrating lymphocytes (TILs) and engineered variants of such T-cells including but not limited to CAR-T cells, recombinantly modified TILs and TCR engineered cells.

As used herein, the term “therapeutically effective amount” as used herein in reference to the administration of an agent to a subject, either alone or as part of a pharmaceutical composition or treatment regimen, in a single dose or as part of a series of doses in an amount capable of having any detectable, positive effect on any symptom, aspect, or characteristic of a disease, disorder or condition when administered to the subject. The therapeutically effective amount can be ascertained by measuring relevant physiological effects, and it may be adjusted in connection with a dosing regimen and in response to diagnostic analysis of the subject's condition, and the like. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, biomarkers (such as inflammatory cytokines, IFN-7, granzyme, and the like), reduction in serum tumor markers, improvement in Response Evaluation Criteria In Solid Tumors (RECIST), improvement in Immune-Related Response Criteria (irRC), increase in duration of survival, extended duration of progression free survival, extension of the time to progression, increased time to treatment failure, extended duration of event free survival, extension of time to next treatment, improvement objective response rate, improvement in the duration of response, reduction of tumor burden, complete response, partial response, stable disease, and the like that that are relied upon by clinicians in the field for the assessment of an improvement in the condition of the subject in response to administration of an agent. As used herein the terms “Complete Response (CR),” “Partial Response (PR)” “Stable Disease (SD)” and “Progressive Disease (PD)” with respect to target lesions and the terms “Complete Response (CR),” “Incomplete Response/Stable Disease (SD)” and Progressive Disease (PD) with respect to non-target lesions are understood to be as defined in the RECIST criteria. As used herein the terms “immune-related Complete Response (irCR),” “immune-related Partial Response (irPR),” “immune-related Progressive Disease (irPD)” and “immune-related Stable Disease (irSD)” as as defined in accordance with the Immune-Related Response Criteria (irRC). As used herein, the term “Immune-Related Response Criteria (irRC)” refers to a system for evaluation of response to immunotherapies as described in Wolchok, et al. (2009) Guidelines for the Evaluation of Immune Therapy Activity in Solid Tumors: Immune-Related Response Criteria, Clinical Cancer Research 15(23): 7412-7420. A therapeutically effective amount may be adjusted over a course of treatment of a subject in connection with the dosing regimen and/or evaluation of the subject's condition and variations in the foregoing factors. In one embodiment, a therapeutically effective amount is an amount of an agent when used alone or in combination with another agent does not result in non-reversible serious adverse events in the course of administration to a mammalian subject.

The terms “treat”, “treating”, treatment” and the like refer to a course of action (such as administering a binding protein described herein, or a pharmaceutical composition comprising same) initiated with respect to a subject after a disease, disorder or condition, or a symptom thereof, has been diagnosed, observed, or the like in the subject so as to eliminate, reduce, suppress, mitigate, or ameliorate, either temporarily or permanently, at least one of the underlying causes of such disease, disorder, or condition afflicting a subject, or at least one of the symptoms associated with such disease, disorder, or condition. The treatment includes a course of action taken with respect to a subject suffering from a disease where the course of action results in the inhibition (e.g., arrests the development of the disease, disorder or condition or ameliorates one or more symptoms associated therewith) of the disease in the subject.

As used herein, the term “V_{H}H” is a type of sdAb that has a single monomeric heavy chain variable antibody domain. Such antibodies can be found in or produced from Camelid mammals (e.g., camels, llamas) which are naturally devoid of light chains.

As used herein, the term “V_{H}H²” refers to two V_{H}Hs that are joined together by way of a linker (e.g., a covalent bond or a peptide linker). A “bispecific V_{H}H²” refers to a V_{H}H² that has a first V_{H}H binding to a first receptor, or domain or subunit thereof, and a second V_{H}H binding to a second receptor, or domain or subunit thereof.

The terms “IL2Rβ,” “IL2Rb” and “IL2Rbeta” are used interchangeably. The terms “IL2Rγ,” “IL2Rg” and IL2Rgamma” are used interchangeably.

### III. Compositions and Methods

The disclosure describes IL2R binding proteins that bind to IL2Rβ and IL2Rγ or domains thereof. The various IL2R binding proteins can be screened for binding to IL2Rβ and IL2Rγ or domains thereof and for signal transduction in therapeutically relevant cell types.

The IL2R binding proteins described herein can specifically bind to IL2Rβ and IL2Rγ and can comprise an anti-IL2Rβ V_{H}H antibody and an anti-IL2Rγ V_{H}H antibody. The IL2R binding proteins described herein are also referred to as anti-IL2Rβ/γ V_{H}H². The IL2R binding protein can cause the multimerization of IL2Rβ and IL2Rγ and downstream signaling.

### Anti-IL2Rβ V_{H}H Antibody

In some embodiments, the present disclosure provides polypeptides comprising any of the anti-IL2Rβ V_{H}H antibodies described herein, e.g., a polypeptide comprising an anti-IL2Rβ V_{H}H comprising a CDR1, a CDR2, and a CDR3 selected from Table 1 below. In certain embodiments, the present disclosure provides a polypeptide comprising a set of CDR1, CDR2, and CDR3 (e.g., CDR1, CDR2, and CDR3 described in the same row) selected from a row of Table 1 below. In certain embodiments, the present disclosure provides a polypeptide comprising a sequence having at least 90% (e.g., 90%, 91%, 92%, 93%, 94%, 95%, 96%, 97%, 98%, 99%, or 100%) identity to a sequence of an anti-IL2Rβ V_{H}H antibody selected from Table 1 below. In some embodiments, a polypeptide provided by the present disclosure can comprise a dimer or multimer of two or more of anti-IL2Rβ V_{H}H antibodies as described in Table 1, in which the anti-IL2Rβ V_{H}H antibodies can be the same or different.

In some embodiments, the present disclosure provides an anti-IL2Rβ V_{H}H antibody, which may be incorporated into a multivalent binding protein as descried herein, comprising one or more of the CDR1s, CDR2s, CDR2s or V_{H}H amino acid sequences as listed in Table 1 below. In some embodiments, the anti-IL2Rβ V_{H}H antibody can comprise: (1) a CDR1 having a sequence of any one of SEQ ID NOS: 1, 5, 9, 13, 17, 21, 25, 29, 33, 37, or 410-419 or a variant thereof that has a sequence having one, two, or three amino acid substitutions relative to a sequence of any one of SEQ ID NOS: 1, 5, 9, 13, 17, 21, 25, 29, 33, 37, or 410-419; (2) a CDR2 having a sequence of any one of SEQ ID NOS: 2, 6, 10, 14, 18, 22, 26, 30, 34, and 38 or a variant thereof that has a sequence having one, two, or three amino acid substitutions relative to a sequence of any one of SEQ ID NOS: 2, 6, 10, 14, 18, 22, 26, 30, 34, and 38; (3) a CDR3 having a sequence of any one of SEQ ID NOS: 3, 7, 11, 15, 19, 23, 27, 31, 35, and 39 or a variant thereof that has a sequence having one, two, or three amino acid substitutions relative to a sequence of any one of SEQ ID NOS: 3, 7, 11, 15, 19, 23, 27, 31, 35, and 39. In some embodiments, an anti-IL2Rβ V_{H}H antibody may be modified for extended half-life (e.g., Fc conjugation, PEGylation) either alone or in the context of a multivalent binding protein as described herein. In some embodiments the moiety providing half-life extension (e.g., PEG, Fc polypeptide, or Fc domain) is conjugated, optionally via a linker, to the N-terminus of the antibody, the C-terminus of the antibody, or an internal amino acid residue (particularly via conjugation to the side chains of lysine or cysteine residues).

In some embodiments, the anti-IL2Rβ V_{H}H antibody can comprise a set of CDR1, CDR2, and CDR3 (e.g., CDR1, CDR2, and CDR3 described in the same row) selected from a row of Table 1 below. In each set of CDR1, CDR2, and CDR3, (1) the CDR1 can have the indicated sequence in the set or a variant thereof that has a sequence having one, two, or three amino acid substitutions relative to the indicated sequence; (2) the CDR2 can have the indicated sequence in the set or a variant thereof that has a sequence having one, two, or three amino acid substitutions relative to the indicated sequence; (3) the CDR3 can have the indicated sequence in the set or a variant thereof that has a sequence having one, two, or three amino acid substitutions relative to the indicated sequence.

Further, an anti-IL2Rβ V_{H}H antibody can comprise CDR1, CDR2, and CDR3 having the sequences of SEQ ID NOS: 1-3 or 410, 2 and 3. Further, an anti-IL2Rβ V_{H}H antibody can comprise CDR1, CDR2, and CDR3 having the sequences of SEQ ID NOS: 5-7 or 411, 6 and 7. Further, an anti-IL2Rβ V_{H}H antibody can comprise CDR1, CDR2, and CDR3 having the sequences of SEQ ID NOS: 9-11 or 412, 10 and 11. Further, an anti-IL2Rβ V_{H}H antibody can comprise CDR1, CDR2, and CDR3 having the sequences of SEQ ID NOS: 13-15 or 413, 14 and 15. Further, an anti-IL2Rβ V_{H}H antibody can comprise CDR1, CDR2, and CDR3 having the sequences of SEQ ID NOS: 17-19 or 414, 18 and 19. Further, an anti-IL2Rβ V_{H}H antibody can comprise CDR1, CDR2, and CDR3 having the sequences of SEQ ID NOS: 21-23 or 415, 22, and 23. Further, an anti-IL2Rβ V_{H}H antibody can comprise CDR1, CDR2, and CDR3 having the sequences of SEQ ID NOS: 25-27 or 416, 26, and 27. Further, an anti-IL2Rβ V_{H}H antibody can comprise CDR1, CDR2, and CDR3 having the sequences of SEQ ID NOS: 29-31 or 417, 30, and 31. Further, an anti-IL2Rβ V_{H}H antibody can comprise CDR1, CDR2, and CDR3 having the sequences of SEQ ID NOS: 33-35 or 418, 34, and 35. Further, an anti-IL2Rβ V_{H}H antibody can comprise CDR1, CDR2, and CDR3 having the sequences of SEQ ID NOS: 37-39 or 419, 38, and 39.

Further, an anti-IL2Rβ V_{H}H antibody can comprise CDR1, CDR2, and CDR3 having the sequences of SEQ ID NOS: 1-3, or 410, 2 and 3, respectively, and at least 90% (e.g., 90%, 91%, 92%, 93%, 94%, 95%, 96%, 97%, 98%, 99%, or 100%) identity to the sequence of SEQ ID NO: 4. Further, an anti-IL2Rβ V_{H}H antibody can comprise CDR1, CDR2, and CDR3 having the sequences of SEQ ID NOS: 5-7, or 411, 6 and 7, respectively, and at least 90% (e.g., 90%, 91%, 92%, 93%, 94%, 95%, 96%, 97%, 98%, 99%, or 100%) identity to the sequence of SEQ ID NO: 8. Further, an anti-IL2Rβ V_{H}H antibody can comprise CDR1, CDR2, and CDR3 having the sequences of SEQ ID NOS: 9-11, or 412, 10 and 11, respectively, and at least 90% (e.g., 90%, 91%, 92%, 93%, 94%, 95%, 96%, 97%, 98%, 99%, or 100%) identity to the sequence of SEQ ID NO: 12. Further, an anti-IL2Rβ V_{H}H antibody can comprise CDR1, CDR2, and CDR3 having the sequences of SEQ ID NOS: 13-15, or 413, 14 and 15, respectively, and at least 90% (e.g., 90%, 91%, 92%, 93%, 94%, 95%, 96%, 97%, 98%, 99%, or 100%) identity to the sequence of SEQ ID NO: 16. Further, an anti-IL2Rβ V_{H}H antibody can comprise CDR1, CDR2, and CDR3 having the sequences of SEQ ID NOS: 17-19, or 414, 18 and 19, respectively, and at least 90% (e.g., 90%, 91%, 92%, 93%, 94%, 95%, 96%, 97%, 98%, 99%, or 100%) identity to the sequence of SEQ ID NO: 20. Further, an anti-IL2Rβ V_{H}H antibody can comprise CDR1, CDR2, and CDR3 having the sequences of SEQ ID NOS: 21-23, or 415, 22, and 23, respectively, and at least 90% (e.g., 90%, 91%, 92%, 93%, 94%, 95%, 96%, 97%, 98%, 99%, or 100%) identity to the sequence of SEQ ID NO: 24. Further, an anti-IL2Rβ V_{H}H antibody can comprise CDR1, CDR2, and CDR3 having the sequences of SEQ ID NOS: 25-27, or 416, 26, and 27, respectively, and at least 90% (e.g., 90%, 91%, 92%, 93%, 94%, 95%, 96%, 97%, 98%, 99%, or 100%) identity to the sequence of SEQ ID NO: 28. Further, an anti-IL2Rβ V_{H}H antibody can comprise CDR1, CDR2, and CDR3 having the sequences of SEQ ID NOS: 29-31, or 417, 30, and 31, respectively, and at least 90% (e.g., 90%, 91%, 92%, 93%, 94%, 95%, 96%, 97%, 98%, 99%, or 100%) identity to the sequence of SEQ ID NO: 32.

Further, an anti-IL2Rβ V_{H}H antibody can comprise CDR1, CDR2, and CDR3 having the sequences of SEQ ID NOS: 33-35, or 418, 34, and 35, respectively, and at least 90% (e.g., 90%, 91%, 92%, 93%, 94%, 95%, 96%, 97%, 98%, 99%, or 100%) identity to the sequence of SEQ ID NO: 36. Further, an anti-IL2Rβ V_{H}H antibody can comprise CDR1, CDR2, and CDR3 having the sequences of SEQ ID NOS: 37-39, or 419, 38, and 39, respectively, and at least 90% (e.g., 90%, 91%, 92%, 93%, 94%, 95%, 96%, 97%, 98%, 99%, or 100%) identity to the sequence of SEQ ID NO: 40.

TABLE 1
Anti-IL2Rβ V_{H}H antibody sequences

| | | CDR1 | | | |
|---|---|---|---|---|---|
| V_{H}H | CDR1 | (Chothia | | | |
| Ab. | (Kabat) | /Kabat) | CDR2 | CDR3 | V_{H}H |
| DR214 | TQDMS | TQDMS | SILTPN | VDERCE | QVQLLESGGGLVQPGGSLRLSCAAS |
| | (SEQ ID | (SEQ ID | GSTYYA | AEDQID | GVRISTQDMSWVRQAPGKGLEWVSS |
| | NO: | NO: | DSVKG | Y | ILTPNGSTYYADSVKGRFTISRDNS |
| | 1) | 410) | (SEQ ID | (SEQ ID | KNTLYLQMNSLRAEDTAVYYCAGVD |
| | | | NO: | NO: | ERCEAEDQIDYWGQGTLVTVSS |
| | | | 2) | 3) | (SEQ ID NO: 4) |
| DR217 | SEDMS | SEDMS | TIASDD | FDAQDA | QVQLLESGGGLVQPGGSLRLSCAAS |
| | (SEQ ID | (SEQ ID | GSTYYA | AIEY | GDMIISEDMSWVRQAPGKGLEWVST |
| | NO: | NO: | DSVKG | (SEQ ID | IASDDGSTYYADSVKGRFTISRDNS |
| | 5) | 411) | (SEQ ID | NO: | KNTLYLQMNSLRAEDTAVYYCAGFD |
| | | | NO: | 7) | AQDAAIEYWGQGTLVTVSS |
| | | | 6) | | (SEQ ID NO: 8) |
| DR583 | TSDMS | YTYDTS | DIDSGD | IDSYWK | QVQLQESGGGSVQAGGSLRLSCVGS |
| | (SEQ ID | DMS | WAAYAD | WGKLNN | GYTYDTSDMSWYRQAPGKEREFVSD |
| | NO: | (SEQ ID | AVKG | F | IDSGDWAAYADAVKGRFTISRDNAK |
| | 9) | NO: | (SEQ ID | (SEQ ID | KTVYLQMNSLEPEDTAMYYCKASYW |
| | | 412) | NO: | NO: | KWGKLNNFWGPGTQVTVSS |
| | | | 10) | 11) | (SEQ ID NO: |
| | | | | | 12) |
| DR584 | NYGMS | FRFSNY | YINGDG | WSLSAA | GLSRDGQVQLQESGGGLVQPGGSLK |
| | (SEQ ID | GMS | SRTHYA | S | LSCAASGFRFSNYGMSWVRQAPGEG |
| | NO: | (SEQ ID | DSVKG | (SEQ ID | LEWVSYINGDGSRTHYADSVKGRFT |
| | 13) | NO: | (SEQ ID | NO: | ISRDNAKNTLYLQLNSLKTEDTAMY |
| | | 413) | NO: | 15) | YCEKGLSRDGWSLSAASRGQGTQVT |
| | | | 14) | | VSS |
| | | | | | (SEQ ID NO: 16) |
| DR585 | FNYMG | YTTYSF | VIYTGG | DDQRFA | NYMGWFRQAPGKEREGVAVIYTGGG |
| | (SEQ ID | NYMG | GSTLYA | SPLYAY | STLYADQVQLQESGGGSVQTGGSLR |
| | NO: | (SEQ ID | DSVKG | FGY | LSCAVSGYTTYSFSVKGRFTISQDN |
| | 17) | NO: | (SEQ ID | (SEQ ID | AKNTVYLQMNSLKPEDTAMYYCAAD |
| | | 414) | NO: | NO: | DQRFASPLYAYFGYWGQGTQVTVSS |
| | | | 18) | 19) | (SEQ ID NO: 20) |
| DR586 | NYWIF | FTFSNY | TSNTGG | GRCARS | QVQLQESGGGLVQPGGSLRLSCVAS |
| | (SEQ ID | WIF | DTTKYA | G | GFTFSNYWIFWVRQAAGKGLEWLST |
| | NO: | (SEQ ID | DSVKG | (SEQ ID | SNTGGDTTKYADSVKGRFTISRDSA |
| | 21) | NO: | (SEQ ID | NO: | KNTEYLQMNSLKPEDTAVYYCETGR |
| | | 415) | NO: | 23) | CARSGGYQGTQVTVSS |
| | | | 22) | | (SEQ ID NO: 24) |
| DR587 | IRCMG | DTKSIR | AIDREG | QNMCRV | QVQLQESGGGSVQVGGSLRLSCATS |
| | (SEQ ID | CMG | FATYAD | VRGAMT | GDTKSIRCMGWFRQTPGKEREGIAA |
| | NO: | (SEQ ID | SVYD | GVDY | IDREGFATYADSVYDRFTIAQDNAQ |
| | 25) | NO: | (SEQ ID | (SEQ ID | NTLYLEMNALKPEDTAMYYCAAQNM |
| | | 416) | NO: | NO: | CRVVRGAMTGVDYWGKGTQVTVSS |
| | | | 26) | 27) | (SEQ ID NO: 28) |
| DR588 | SYYCMG | YTYSSY | AIDSDG | SYEVVY | DCYPSGYCMGWFRQAPGKEREGVAA |
| | (SEQ ID | YCMG | STSYAD | GQDYSE | IDSDGSTSYADSVQVQLQESGGGSV |
| | NO: | (SEQ ID | SVKG | Q ID | QVGGSLKLSCAASGYTYSSYKGRFT |
| | 29) | NO: | (SEQ ID | NO: | ISQDDAKNTLYLQMNSLKPEDTAMY |
| | | 417:) | NO: | 31) | YCAASYEVVDCYPSGYGQDYWGKGT |
| | | | 30) | | QVTVSS |
| | | | | | (SEQ ID NO: 32) |
| DR589 | RYCMA | YTASRY | AIHPGG | GSLWVP | CMAWFRQAPGKEREGVAAIHPGGGT |
| | (SEQ ID | CMA | GTTYYA | FGDRCA | TYYADSQVQLQESGGGSVQAGGSLR |
| | NO: | (SEQ ID | DSVKG | ANY | LSCAASEYTASRYVKGRFSISQDSA |
| | 33) | NO: | (SEQ ID | (SEQ ID | DNTLYLQMNSLKPEDTAMYYCAAGS |
| | | 418) | NO: | NO: | LWVPFGDRCAANYWGQGTQVTVSS |
| | | | 34) | 35) | (SEQ ID NO: 36) |
| DR590 | RIHMT | YEYCRI | SIGSDG | EYLYGL | QVQLQESGGGSVQAGGSLRLSCAAS |
| | (SEQ ID | HMT | RKTYAN | GCPDGS | GYEYCRIHMTWYRQGPGKEREFVSS |
| | NO: | (SEQ ID | SVTG | AY | IGSDGRKTYANSVTGRFTISRDNAN |
| | 37) | NO: | (SEQ ID | (SEQ ID | HTVYLQMNSLSPEDTAMYYCKTEYL |
| | | 419) | NO: | NO: | YGLGCPDGSAYWGQGTQVTVSS |
| | | | 38) | 39) | (SEQ ID NO: 40) |

### Anti-IL2Rγ V_{H}H Antibody

In some embodiments, the present disclosure provides polypeptides comprising any of the anti-IL2Rγ V_{H}H antibodies described herein, e.g., a polypeptide comprising an anti-IL2Rγ V_{H}H comprising a CDR1, a CDR2, and a CDR3 selected from Table 2 below. In certain embodiments, the present disclosure provides a polypeptide comprising a set of CDR1, CDR2, and CDR3 (e.g., CDR1, CDR2, and CDR3 described in the same row) selected from a row of Table 2 below. In certain embodiments, the present disclosure provides a polypeptide comprising a sequence having at least 90% (e.g., 90%, 91%, 92%, 93%, 94%, 95%, 96%, 97%, 98%, 99%, or 100%) identity to a sequence of an anti-IL2Rγ V_{H}H antibody selected from Table 2 below. In some embodiments, a polypeptide provided by the present disclosure can comprise a dimer or multimer of two or more of anti-IL2Rγ V_{H}H antibodies as described in Table 2, in which the anti-IL2Rγ V_{H}H antibodies can be the same or different.

In some embodiments, the present disclosure provides an anti-IL2Rγ V_{H}H antibody, which may be incorporated into a multivalent binding protein as described herein, comprising one or more of CDR1 s, CD2s, CDR3s or V_{H}H amino acid sequences as listed in Table 2 below. In some embodiments, the anti-IL2Rγ V_{H}H antibody can comprise: (1) a CDR1 having a sequence of any one of SEQ ID NOS: 41, 45, 49, 53, 57, 61, or 420-425, or a variant thereof that has a sequence having one, two, or three amino acid substitutions relative to a sequence of any one of SEQ ID NOS: 41, 45, 49, 53, 57, 61, or 420-425; (2) a CDR2 having a sequence of any one of SEQ ID NOS: 42, 46, 50, 54, 58, and 62 or a variant thereof that has a sequence having one, two, or three amino acid substitutions relative to a sequence of any one of SEQ ID NOS: 42, 46, 50, 54, 58, and 62; (3) a CDR3 having a sequence of any one of SEQ ID NOS: 43, 47, 51, 55, 59, and 63 or a variant thereof that has a sequence having one, two, or three amino acid substitutions relative to a sequence of any one of SEQ ID NOS: 43, 47, 51, 55, 59, and 63. In some embodiments, an anti-IL2Rγ V_{H}H may be modified for extended half-life (e.g., Fc conjugation, PEGylation) either alone or in the context of a multivalent binding protein as described herein. In some embodiments the moiety providing half-life extension (e.g., PEG, Fc polypeptide, Fc domain) is conjugated, optionally via a linker, to the N-terminus of the antibody, the C-terminus of the antibody, or an internal amino acid residue (particularly via conjugation to the side chains of lysine or cysteine residues).

In some embodiments, the anti-IL2Rγ V_{H}H antibody can comprise a set of CDR1, CDR2, and CDR3 (e.g., CDR1, CDR2, and CDR3 described in the same row) selected from a row of Table 2 below. In each set of CDR1, CDR2, and CDR3, (1) the CDR1 can have the indicated sequence in the set or a variant thereof that has a sequence having one, two, or three amino acid substitutions relative to the indicated sequence; (2) the CDR2 can have the indicated sequence in the set or a variant thereof that has a sequence having one, two, or three amino acid substitutions relative to the indicated sequence; (3) the CDR3 can have the indicated sequence in the set or a variant thereof that has a sequence having one, two, or three amino acid substitutions relative to the indicated sequence.

Further, an anti-IL2Rγ V_{H}H antibody can comprise CDR1, CDR2, and CDR3 having the sequences of SEQ ID NOS: 41-43 or 420, 42, and 43. Further, an anti-IL2Rγ V_{H}H antibody can comprise CDR1, CDR2, and CDR3 having the sequences of SEQ ID NOS: 45-47 or 421, 46, and 47. Further, an anti-IL2Rγ V_{H}H antibody can comprise CDR1, CDR2, and CDR3 having the sequences of SEQ ID NOS: 49-51 or 422, 50, and 51. Further, an anti-IL2Rγ V_{H}H antibody can comprise CDR1, CDR2, and CDR3 having the sequences of SEQ ID NOS: 53-55 or 423, 54, and 55. Further, an anti-IL2Rγ V_{H}H antibody can comprise CDR1, CDR2, and CDR3 having the sequences of SEQ ID NOS: 57-59 or 424, 58, and 59. Further, an anti-IL2Rγ V_{H}H antibody can comprise CDR1, CDR2, and CDR3 having the sequences of SEQ ID NOS: 61-63 or 425, 62, and 63.

Further, an anti-IL2Rγ V_{H}H antibody can comprise CDR1, CDR2, and CDR3 having the sequences of SEQ ID NOS: 41-43, respectively, and at least 90% (e.g., 90%, 91%, 92%, 93%, 94%, 95%, 96%, 97%, 98%, 99%, or 100%) identity to the sequence of SEQ ID NO: 44. Further, an anti-IL2Rγ V_{H}H antibody can comprise CDR1, CDR2, and CDR3 having the sequences of SEQ ID NOS: 45-47, respectively, and at least 90% (e.g., 90%, 91%, 92%, 93%, 94%, 95%, 96%, 97%, 98%, 99%, or 100%) identity to the sequence of SEQ ID NO: 48. Further, an anti-IL2Rγ V_{H}H antibody can comprise CDR1, CDR2, and CDR3 having the sequences of SEQ ID NOS: 49-51, respectively, and at least 90% (e.g., 90%, 91%, 92%, 93%, 94%, 95%, 96%, 97%, 98%, 99%, or 100%) identity to the sequence of SEQ ID NO: 52. Further, an anti-IL2Rγ V_{H}H antibody can comprise CDR1, CDR2, and CDR3 having the sequences of SEQ ID NOS: 53-55, respectively, and at least 90% (e.g., 90%, 91%, 92%, 93%, 94%, 95%, 96%, 97%, 98%, 99%, or 100%) identity to the sequence of SEQ ID NO: 56. Further, an anti-IL2Rγ V_{H}H antibody can comprise CDR1, CDR2, and CDR3 having the sequences of SEQ ID NOS: 57-59, respectively, and at least 90% (e.g., 90%, 91%, 92%, 93%, 94%, 95%, 96%, 97%, 98%, 99%, or 100%) identity to the sequence of SEQ ID NO: 60. Further, an anti-IL2Rγ V_{H}H antibody can comprise CDR1, CDR2, and CDR3 having the sequences of SEQ ID NOS: 61-63, respectively, and at least 90% (e.g., 90%, 91%, 92%, 93%, 94%, 95%, 96%, 97%, 98%, 99%, or 100%) identity to the sequence of SEQ ID NO: 64.

TABLE 2
Anti-IL2Rγ V_{H}H antibody sequences

| | | CDR1 | | | |
|---|---|---|---|---|---|
| V_{H}H | CDR1 | (Chothia/ | | | |
| Ab. | (Kabat) | Kabat) | CDR2 | CDR3 | V_{H}H |
| DR229 | SYPMT | FSFSSYP | TIASD | GYGDG | QVQLQES |
| | (SEQ ID | MT | GGSTA | TPA | GGGLVQP |
| | NO: 41) | (SEQ ID | YAASV | (SEQ ID | GGSLRLS |
| | | NO: 420) | EG | NO: 43) | CTASGFS |
| | | | (SEQ ID | | FSSYPMT |
| | | | NO: 42) | | WARQAPG |
| | | | | | KGLEWVS |
| | | | | | TIASDGG |
| | | | | | STAYAAS |
| | | | | | VEGRFTI |
| | | | | | SRDNAKS |
| | | | | | TLYLQLN |
| | | | | | SLKTEDT |
| | | | | | AMYYCTK |
| | | | | | GYGDGTP |
| | | | | | APGQGTQ |
| | | | | | VTVSS |
| | | | | | (SEQ ID |
| | | | | | NO: 44) |
| DR230 | SAHMS | FTFSSAH | SIYSG | NRLHY | YSDDDSM |
| | (SEQ ID | MS | GGTFY | L | SWVRQAP |
| | NO: 45) | (SEQ ID | ADSVK | (SEQ ID | GKGREWI |
| | | NO: 421) | G | NO: 47) | ASIYSGG |
| | | | (SEQ ID | | GTFYADS |
| | | | NO: 46) | | VKGQVQL |
| | | | | | QESGGGL |
| | | | | | VQPGGSL |
| | | | | | RLSCAAS |
| | | | | | GFTFSSA |
| | | | | | HRFTISR |
| | | | | | DNAKNTL |
| | | | | | YLQLNSL |
| | | | | | KAEDTAM |
| | | | | | YYCATNR |
| | | | | | LHYYSDD |
| | | | | | DSLRGQG |
| | | | | | TQVTVSS |
| | | | | | (SEQ ID |
| | | | | | NO: 48) |
| DR231 | DREMN | FTFDDRE | TISSD | DFMIA | QVQLQES |
| | (SEQ ID | MN | GSTYY | IQAPG | GGGSVQA |
| | NO: 49) | (SEQ ID | ADSVK | AGC | GGSLRLS |
| | | NO: 422) | G | (SEQ ID | CTASGFT |
| | | | (SEQ ID | NO: 51) | FDDREMN |
| | | | NO: 50) | | WYRQAPG |
| | | | | | NECELVS |
| | | | | | TISSDGS |
| | | | | | TYYADSV |
| | | | | | KGRFTIS |
| | | | | | QDNAKNT |
| | | | | | VYLQMDS |
| | | | | | VKPEDTA |
| | | | | | VYYCAAD |
| | | | | | FMIAIQA |
| | | | | | PGAGCWG |
| | | | | | QGTQVTV |
| | | | | | SS |
| | | | | | (SEQ ID |
| | | | | | NO: 52) |
| DR232 | CMG | YTSCMG | TIYTR | GGYSW | WFRQAPG |
| | (SEQ ID | (SEQ ID | GRSIY | SAGCE | KEREAVA |
| | NO: 53) | NO: 423) | YADSV | FNY | TIYTRGR |
| | | | KG | (SEQ ID | SIYYADS |
| | | | (SEQ ID | NO: 55) | VKGRFTI |
| | | | NO: 54) | | SQDNAKN |
| | | | | | TLYLQMN |
| | | | | | SLKPEDI |
| | | | | | AMYSCAA |
| | | | | | GQVQLQE |
| | | | | | SGGGSVQ |
| | | | | | AGGSLRL |
| | | | | | SCVASGY |
| | | | | | TSCMGGY |
| | | | | | SWSAGCE |
| | | | | | FNYWGQG |
| | | | | | TQVTVSS |
| | | | | | (SEQ ID |
| | | | | | NO: 56) |
| DR233 | DSDMG | FTFDDSD | TISSD | EPRGY | QVQLQES |
| | (SEQ ID | MG | GSTYY | YSNYG | GGGSVQA |
| | NO: 57) | (SEQ ID | ADSVK | GRREC | GGSLRLS |
| | | NO: 424) | G | NY | CTASGFT |
| | | | (SEQ ID | (SEQ ID | FDDSDMG |
| | | | NO: 58) | NO: 59) | WYRQAPG |
| | | | | | NECELVS |
| | | | | | TISSDGS |
| | | | | | TYYADSV |
| | | | | | KGRFTIS |
| | | | | | QDNAKNT |
| | | | | | VYLQMNS |
| | | | | | LKPEDTA |
| | | | | | VYYCAAE |
| | | | | | PRGYYSN |
| | | | | | YGGRREC |
| | | | | | NYWGQGT |
| | | | | | QVTVSS |
| | | | | | (SEQ ID |
| | | | | | NO: 60) |
| DR234 | SYCMG | YTFSSYC | ALGGG | AWVAC | QVQLQES |
| | (SEQ ID | MG | STYYA | LEFGG | GGGSVQA |
| | NO: 61) | (SEQ ID | DSVKG | SWYDL | GGSLRLS |
| | | NO: 425) | (SEQ ID | ARYKH | CVASGYT |
| | | | NO: 62) | (SEQ ID | FSSYCMG |
| | | | | NO: 63) | WFRQAPG |
| | | | | | KEREGVA |
| | | | | | ALGGGST |
| | | | | | YYADSVK |
| | | | | | GRFTISQ |
| | | | | | DNAKNTL |
| | | | | | YLQMNSL |
| | | | | | KPEDTAM |
| | | | | | YYCAAAW |
| | | | | | VACLEFG |
| | | | | | GSWYDLA |
| | | | | | RYKHWGQ |
| | | | | | GTQVTVS |
| | | | | | S |
| | | | | | (SEQ ID |
| | | | | | NO: 64) |

Anti-IL2Rβ/γ V_{H}H²

An IL2R binding protein described herein can comprise an anti-IL2Rβ V_{H}H antibody selected from Table 1 and an anti-IL2Rγ V_{H}H antibody selected from Table 2. In some embodiments, the N-terminal V_{H}H of the IL2R binding molecule is an anti-IL2Rβ V_{H}H antibody and the C-terminal V_{H}H of the IL2R binding protein is an anti-IL2Rγ V_{H}H antibody, optionally a linker can be used between the two V_{H}H antibodies. In some embodiments, the N-terminal V_{H}H of the IL2R binding protein is an anti-IL2Rγ V_{H}H antibody and the C-terminal V_{H}H of the IL2R binding protein is an anti-IL2Rβ V_{H}H antibody, optionally a linker can be used between the two V_{H}H antibodies. Examples of linkers (e.g., GGGS (SEQ ID NO: 107)) that can be used to fuse the anti-IL2Rβ V_{H}H antibody and the anti-IL2Rγ V_{H}H antibody are described in detail further herein. In some embodiments, the IL2R binding protein may be operably linked to a metal chelating peptide. Chelating peptides include but are not limited to the Ala-Ser-His-His-His-His-His-His (“ASH6”, SEQ ID NO: 126) or the His-His-His-His-His-His (“H6”, SEQ ID NO: 127) purification handle to facilitate purification of the binding protein by chelating peptide immobilized metal affinity chromatography (“CP-IMAC, as described in U.S. Pat. No. 4,569,794).

Table 3 below further illustrates examples of IL2R binding proteins described herein that comprise an anti-IL2Rβ V_{H}H antibody at the N-terminus and an anti-IL2Rγ V_{H}H antibody at the C-terminus. In some embodiments, an IL2R binding protein comprises the V_{H}H sequence of DR214, the V_{H}H sequence of DR233, and has at least 90% (e.g., 90%, 91%, 92%, 93%, 94%, 95%, 96%, 97%, 98%, 99%, or 100%) identity to the sequence of SEQ ID NO: 65, optionally without the terminal HHHHHHHHH (SEQ ID NO: 127). In some embodiments, an IL2R binding protein comprises the V_{H}H sequence of DR217, the V_{H}H sequence of DR232, and has at least 90% (e.g., 90%, 91%, 92%, 93%, 94%, 95%, 96%, 97%, 98%, 99%, or 100%) identity to the sequence of SEQ ID NO: 66, optionally without the terminal HHHHHH (SEQ ID NO: 127). In some embodiments, an IL2R binding protein comprises the V_{H}H sequence of DR584, the V_{H}H sequence of DR233, and has at least 90% (e.g., 90%, 91%, 92%, 93%, 94%, 95%, 96%, 97%, 98%, 99%, or 100%) identity to the sequence of SEQ ID NO: 67, optionally without the terminal HHHHHH (SEQ ID NO: 127). In some embodiments, an IL2R binding protein comprises the V_{H}H sequence of DR585, the V_{H}H sequence of DR229, and has at least 90% (e.g., 90%, 91%, 92%, 93%, 94%, 95%, 96%, 97%, 98%, 99%, or 100%) identity to the sequence of SEQ ID NO: 68, optionally without the terminal HHHHHHHHH (SEQ ID NO: 127). In some embodiments, an IL2R binding protein comprises the V_{H}H sequence of DR585, the V_{H}H sequence of DR230, and has at least 90% (e.g., 90%, 91%, 92%, 93%, 94%, 95%, 96%, 97%, 98%, 99%, or 100%) identity to the sequence of SEQ ID NO: 69, optionally without the terminal HHHHHH (SEQ ID NO: 127). In some embodiments, an IL2R binding protein comprises the V_{H}H sequence of DR585, the V_{H}H sequence of DR231, and has at 90% (e.g., 90%, 91%, 92%, 93%, 94%, 95%, 96%, 97%, 98%, 99%, or 100%) identity to the sequence of SEQ ID NO: 70, optionally without the terminal HHHHHH (SEQ ID NO: 127). In some embodiments, an IL2R binding protein comprises the V_{H}H sequence of DR585, the V_{H}H sequence of DR232, and has at least 90% (e.g., 90%, 91%, 92%, 93%, 94%, 95%, 96%, 97%, 98%, 99%, or 100%) identity to the sequence of SEQ ID NO: 71, optionally without the terminal HHHHHH (SEQ ID NO: 127). In some embodiments, an IL2R binding protein comprises the V_{H}H sequence of DR585, the V_{H}H sequence of DR233, and has at least 90% (e.g., 90%, 91%, 92%, 93%, 94%, 95%, 96%, 97%, 98%, 99%, or 100%) identity to the sequence of SEQ ID NO: 72, optionally without the terminal HHHHHHHHH (SEQ ID NO: 127). In some embodiments, an IL2R binding protein comprises the V_{H}H sequence of DR585, the V_{H}H sequence of DR234, and has at least 90% (e.g., 90%, 91%, 92%, 93%, 94%, 95%, 96%, 97%, 98%, 99%, or 100%) identity to the sequence of SEQ ID NO: 73, optionally without the terminal HHHHHH (SEQ ID NO: 127). In some embodiments, an IL2R binding protein comprises the V_{H}H sequence of DR586, the V_{H}H sequence of DR229, and has at least 90% (e.g., 90%, 91%, 92%, 93%, 94%, 95%, 96%, 97%, 98%, 99%, or 100%) identity to the sequence of SEQ ID NO: 74, optionally without the terminal HHHHHHHHH (SEQ ID NO: 127). In some embodiments, an IL2R binding protein comprises the V_{H}H sequence of DR586, the V_{H}H sequence of DR231, and has at least 90% (e.g., 90%, 91%, 92%, 93%, 94%, 95%, 96%, 97%, 98%, 99%, or 100%) identity to the sequence of SEQ ID NO: 75, optionally without the terminal HHHHHH (SEQ ID NO: 127). In some embodiments, an IL2R binding protein comprises the V_{H}H sequence of DR588, the V_{H}H sequence of DR231, and has at least 90% (e.g., 90%, 91%, 92%, 93%, 94%, 95%, 96%, 97%, 98%, 99%, or 100%) identity to the sequence of SEQ ID NO: 76, optionally without the terminal HHHHHHHHH (SEQ ID NO: 127). In some embodiments, an IL2R binding protein comprises the V_{H}H sequence of DR589, the V_{H}H sequence of DR229, and has at least 90% (e.g., 90%, 91%, 92%, 93%, 94%, 95%, 96%, 97%, 98%, 99%, or 100%) identity to the sequence of SEQ ID NO: 77, optionally without the terminal HHHHHH (SEQ ID NO: 127). In some embodiments, an IL2R binding protein comprises the V_{H}H sequence of DR589, the V_{H}H sequence of DR230, and has at least 90% (e.g., 90%, 91%, 92%, 93%, 94%, 95%, 96%, 97%, 98%, 99%, or 100%) identity to the sequence of SEQ ID NO: 78, optionally without the terminal HHHHHHHHH (SEQ ID NO: 127). In some embodiments, an IL2R binding protein comprises the V_{H}H sequence of DR589, the V_{H}H sequence of DR233, and has at least 90% (e.g., 90%, 91%, 92%, 93%, 94%, 95%, 96%, 97%, 98%, 99%, or 100%) identity to the sequence of SEQ ID NO: 79, optionally without the terminal HHHHHH (SEQ ID NO: 127). In some embodiments, an IL2R binding protein comprises the V_{H}H sequence of DR590, the V_{H}H sequence of DR230, and has at least 90% (e.g., 90%, 91%, 92%, 93%, 94%, 95%, 96%, 97%, 98%, 99%, or 100%) identity to the sequence of SEQ ID NO: 80, optionally without the terminal HHHHHH (SEQ ID NO: 127).

TABLE 3
Anti-IL2Rβ/γ V_{H}H² (anti-IL2Rβ V_{H}H-linker-anti-IL2Rγ V_{H}H)

| Anti- | N- | C- | |
|---|---|---|---|
| IL2Rβ/γ | terminal | terminal | |
| V_{H}H² | V_{H}H | V_{H}H | Anti-IL2Rβ/γ V_{H}H² |
| DR696 | DR214 | DR233 | QVQLLESGGGLVQPGGSLRLSCAASGVRIS |
| | | | TQDMSWVRQAPGKGLEWVSSILTPNGSTYY |
| | | | ADSVKGRFTISRDNSKNTLYLQMNSLRAED |
| | | | TAVYYCAGVDERCEAEDQIDYWGQGTLVTV |
| | | | SSGGGSQVQLQESGGGSVQAGGSLRLSCTA |
| | | | SGFTFDDSDMGWYRQAPGNECELVSTISSD |
| | | | GSTYYADSVKGRFTISQDNAKNTVYLQMNS |
| | | | LKPEDTAVYYCAAEPRGYYSNYGGRRECNY |
| | | | WGQGTQVTVSSASHHHHHH |
| | | | (SEQ ID NO: 65) |
| DR701 | DR217 | DR232 | QVQLLESGGGLVQPGGSLRLSCAASGDMII |
| | | | SEDMSWVRQAPGKGLEWVSTIASDDGSTYY |
| | | | ADSVKGRFTISRDNSKNTLYLQMNSLRAED |
| | | | TAVYYCAGFDAQDAAIEYWGQGTLVTVSSG |
| | | | GGSQVQLQESGGGSVQAGGSLRLSCVASGY |
| | | | TSCMGWFRQAPGKEREAVATIYTRGRSIYY |
| | | | ADSVKGRFTISQDNAKNTLYLQMNSLKPED |
| | | | IAMYSCAAGGYSWSAGCEFNYWGQGTQVTV |
| | | | SSASHHHHHH |
| | | | (SEQ ID NO: 66) |
| DR714 | DR584 | DR233 | QVQLQESGGGLVQPGGSLKLSCAASGFRFS |
| | | | NYGMSWVRQAPGEGLEWVSYINGDGSRTHY |
| | | | ADSVKGRFTISRDNAKNTLYLQLNSLKTED |
| | | | TAMYYCEKGLSRDGWSLSAASRGQGTQVTV |
| | | | SSGGGSQVQLQESGGGSVQAGGSLRLSCTA |
| | | | SGFTFDDSDMGWYRQAPGNECELVSTISSD |
| | | | GSTYYADSVKGRFTISQDNAKNTVYLQMNS |
| | | | LKPEDTAVYYCAAEPRGYYSNYGGRRECNY |
| | | | WGQGTQVTVSSASHHHHHH |
| | | | (SEQ ID NO: 67) |
| DR716 | DR585 | DR229 | QVQLQESGGGSVQTGGSLRLSCAVSGYTTY |
| | | | SFNYMGWFRQAPGKEREGVAVIYTGGGSTL |
| | | | YADSVKGRFTISQDNAKNTVYLQMNSLKPE |
| | | | DTAMYYCAADDQRFASPLYAYFGYWGQGTQ |
| | | | VTVSSGGGSQVQLQESGGGLVQPGGSLRLS |
| | | | CTASGFSFSSYPMTWARQAPGKGLEWVSTI |
| | | | ASDGGSTAYAASVEGRFTISRDNAKSTLYL |
| | | | QLNSLKTEDTAMYYCTKGYGDGTPAPGQGT |
| | | | QVTVSSASHHHHHH |
| | | | (SEQ ID NO: 68) |
| DR717 | DR585 | DR230 | QVQLQESGGGSVQTGGSLRLSCAVSGYTTY |
| | | | SFNYMGWFRQAPGKEREGVAVIYTGGGSTL |
| | | | YADSVKGRFTISQDNAKNTVYLQMNSLKPE |
| | | | DTAMYYCAADDQRFASPLYAYFGYWGQGTQ |
| | | | VTVSSGGGSQVQLQESGGGLVQPGGSLRLS |
| | | | CAASGFTFSSAHMSWVRQAPGKGREWIASI |
| | | | YSGGGTFYADSVKGRFTISRDNAKNTLYLQ |
| | | | LNSLKAEDTAMYYCATNRLHYYSDDDSLRG |
| | | | QGTQVTVSSASHHHHHH |
| | | | (SEQ ID NO: 69) |
| DR718 | DR585 | DR231 | QVQLQESGGGSVQTGGSLRLSCAVSGYTTY |
| | | | SFNYMGWFRQAPGKEREGVAVIYTGGGSTL |
| | | | YADSVKGRFTISQDNAKNTVYLQMNSLKPE |
| | | | DTAMYYCAADDQRFASPLYAYFGYWGQGTQ |
| | | | VTVSSGGGSQVQLQESGGGSVQAGGSLRLS |
| | | | CTASGFTFDDREMNWYRQAPGNECELVSTI |
| | | | SSDGSTYYADSVKGRFTISQDNAKNTVYLQ |
| | | | MDSVKPEDTAVYYCAADFMIAIQAPGAGCW |
| | | | GQGTQVTVSSASHHHHHH |
| | | | (SEQ ID NO: 70) |
| DR719 | DR585 | DR232 | QVQLQESGGGSVQTGGSLRLSCAVSGYTTY |
| | | | SFNYMGWFRQAPGKEREGVAVIYTGGGSTL |
| | | | YADSVKGRFTISQDNAKNTVYLQMNSLKPE |
| | | | DTAMYYCAADDQRFASPLYAYFGYWGQGTQ |
| | | | VTVSSGGGSQVQLQESGGGSVQAGGSLRLS |
| | | | CVASGYTSCMGWFRQAPGKEREAVATIYTR |
| | | | GRSIYYADSVKGRFTISQDNAKNTLYLQMN |
| | | | SLKPEDIAMYSCAAGGYSWSAGCEFNYWGQ |
| | | | GTQVTVSSASHHHHHH |
| | | | (SEQ ID NO: 71) |
| DR720 | DR585 | DR233 | QVQLQESGGGSVQTGGSLRLSCAVSGYTTY |
| | | | SFNYMGWFRQAPGKEREGVAVIYTGGGSTL |
| | | | YADSVKGRFTISQDNAKNTVYLQMNSLKPE |
| | | | DTAMYYCAADDQRFASPLYAYFGYWGQGTQ |
| | | | VTVSSGGGSQVQLQESGGGSVQAGGSLRLS |
| | | | CTASGFTFDDSDMGWYRQAPGNECELVSTI |
| | | | SSDGSTYYADSVKGRFTISQDNAKNTVYLQ |
| | | | MNSLKPEDTAVYYCAAEPRGYYSNYGGRRE |
| | | | CNYWGQGTQVTVSSASHHHHHH |
| | | | (SEQ ID NO: 72) |
| DR721 | DR585 | DR234 | QVQLQESGGGSVQTGGSLRLSCAVSGYTTY |
| | | | SFNYMGWFRQAPGKEREGVAVIYTGGGSTL |
| | | | YADSVKGRFTISQDNAKNTVYLQMNSLKPE |
| | | | DTAMYYCAADDQRFASPLYAYFGYWGQGTQ |
| | | | VTVSSGGGSQVQLQESGGGSVQAGGSLRLS |
| | | | CVASGYTFSSYCMGWFRQAPGKEREGVAAL |
| | | | GGGSTYYADSVKGRFTISQDNAKNTLYLQM |
| | | | NSLKPEDTAMYYCAAAWVACLEFGGSWYDL |
| | | | ARYKHWGQGTQVTVSSASHHHHHH |
| | | | (SEQ ID NO: 73) |
| DR722 | DR586 | DR229 | QVQLQESGGGLVQPGGSLRLSCVASGFTFS |
| | | | NYWIFWVRQAAGKGLEWLSTSNTGGDTTKY |
| | | | ADSVKGRFTISRDSAKNTEYLQMNSLKPED |
| | | | TAVYYCETGRCARSGGYQGTQVTVSSGGGS |
| | | | QVQLQESGGGLVQPGGSLRLSCTASGFSFS |
| | | | SYPMTWARQAPGKGLEWVSTIASDGGSTAY |
| | | | AASVEGRFTISRDNAKSTLYLQLNSLKTED |
| | | | TAMYYCTKGYGDGTPAPGQGTQVTVSSASH |
| | | | HHHHH |
| | | | (SEQ ID NO: 74) |
| DR724 | DR586 | DR231 | QVQLQESGGGLVQPGGSLRLSCVASGFTFS |
| | | | NYWIFWVRQAAGKGLEWLSTSNTGGDTTKY |
| | | | ADSVKGRFTISRDSAKNTEYLQMNSLKPED |
| | | | TAVYYCETGRCARSGGYQGTQVTVSSGGGS |
| | | | QVQLQESGGGSVQAGGSLRLSCTASGFTFD |
| | | | DREMNWYRQAPGNECELVSTISSDGSTYYA |
| | | | DSVKGRFTISQDNAKNTVYLQMDSVKPEDT |
| | | | AVYYCAADFMIAIQAPGAGCWGQGTQVTVS |
| | | | SASHHHHHH |
| | | | (SEQ ID |
| | | | NO: 75) |
| DR736 | DR588 | DR231 | QVQLQESGGGSVQVGGSLKLSCAASGYTYS |
| | | | SYYCMGWFRQAPGKEREGVAAIDSDGSTSY |
| | | | ADSVKGRFTISQDDAKNTLYLQMNSLKPED |
| | | | TAMYYCAASYEVVDCYPSGYGQDYWGKGTQ |
| | | | VTVSSGGGSQVQLQESGGGSVQAGGSLRLS |
| | | | CTASGFTFDDREMNWYRQAPGNECELVSTI |
| | | | SSDGSTYYADSVKGRFTISQDNAKNTVYLQ |
| | | | MDSVKPEDTAVYYCAADFMIAIQAPGAGCW |
| | | | GQGTQVTVSSASHHHHHH |
| | | | (SEQ ID NO: 76) |
| DR740 | DR589 | DR229 | QVQLQESGGGSVQAGGSLRLSCAASEYTAS |
| | | | RYCMAWFRQAPGKEREGVAAIHPGGGTTYY |
| | | | ADSVKGRFSISQDSADNTLYLQMNSLKPED |
| | | | TAMYYCAAGSLWVPFGDRCAANYWGQGTQV |
| | | | TVSSGGGSQVQLQESGGGLVQPGGSLRLSC |
| | | | TASGFSFSSYPMTWARQAPGKGLEWVSTIA |
| | | | SDGGSTAYAASVEGRFTISRDNAKSTLYLQ |
| | | | LNSLKTEDTAMYYCTKGYGDGTPAPGQGTQ |
| | | | VTVSSASHHHHHH |
| | | | (SEQ ID NO: 77) |
| DR741 | DR589 | DR230 | QVQLQESGGGSVQAGGSLRLSCAASEYTAS |
| | | | RYCMAWFRQAPGKEREGVAAIHPGGGTTYY |
| | | | ADSVKGRFSISQDSADNTLYLQMNSLKPED |
| | | | TAMYYCAAGSLWVPFGDRCAANYWGQGTQV |
| | | | TVSSGGGSQVQLQESGGGLVQPGGSLRLSC |
| | | | AASGFTFSSAHMSWVRQAPGKGREWIASIY |
| | | | SGGGTFYADSVKGRFTISRDNAKNTLYLQL |
| | | | NSLKAEDTAMYYCATNRLHYYSDDDSLRGQ |
| | | | GTQVTVSSASHHHHHH |
| | | | (SEQ ID NO: 78) |
| DR744 | DR589 | DR233 | QVQLQESGGGSVQAGGSLRLSCAASEYTAS |
| | | | RYCMAWFRQAPGKEREGVAAIHPGGGTTYY |
| | | | ADSVKGRFSISQDSADNTLYLQMNSLKPED |
| | | | TAMYYCAAGSLWVPFGDRCAANYWGQGTQV |
| | | | TVSSGGGSQVQLQESGGGSVQAGGSLRLSC |
| | | | TASGFTFDDSDMGWYRQAPGNECELVSTIS |
| | | | SDGSTYYADSVKGRFTISQDNAKNTVYLQM |
| | | | NSLKPEDTAVYYCAAEPRGYYSNYGGRREC |
| | | | NYWGQGTQVTVSSASHHHHHH |
| | | | (SEQ ID NO: 79) |
| DR747 | DR590 | DR230 | QVQLQESGGGSVQAGGSLRLSCAASGYEYC |
| | | | RIHMTWYRQGPGKEREFVSSIGSDGRKTYA |
| | | | NSVTGRFTISRDNANHTVYLQMNSLSPEDT |
| | | | AMYYCKTEYLYGLGCPDGSAYWGQGTQVTV |
| | | | SSGGGSQVQLQESGGGLVQPGGSLRLSCAA |
| | | | SGFTFSSAHMSWVRQAPGKGREWIASIYSG |
| | | | GGTFYADSVKGRFTISRDNAKNTLYLQLNS |
| | | | LKAEDTAMYYCATNRLHYYSDDDSLRGQGT |
| | | | QVTVSSASHHHHHH |
| | | | (SEQ ID NO: 80) |

Table 4 below further illustrates examples of IL2R binding proteins described herein that comprise anti-IL2Rγ V_{H}H antibody at the N-terminus and anti-IL2Rβ V_{H}H antibody at the C-terminus.

In some embodiments, an IL2R binding protein comprises the V_{H}H sequence of DR229, the V_{H}H sequence of DR583, and has at least 90% (e.g., 90%, 91%, 92%, 93%, 94%, 95%, 96%, 97%, 98%, 99%, or 100%) identity to the sequence of SEQ ID NO: 81, optionally without the terminal HHHHHH (SEQ ID NO: 127). In some embodiments, an IL2R binding protein comprises the V_{H}H sequence of DR229, the V_{H}H sequence of DR584, and has at least 90% (e.g., 90%, 91%, 92%, 93%, 94%, 95%, 96%, 97%, 98%, 99%, or 100%) identity to the sequence of SEQ ID NO: 82, optionally without the terminal HHHHHHHHH (SEQ ID NO: 127). In some embodiments, an IL2R binding protein comprises the V_{H}H sequence of DR229, the V_{H}H sequence of DR585, and has at least 90% (e.g., 90%, 91%, 92%, 93%, 94%, 95%, 96%, 97%, 98%, 99%, or 100%) identity to the sequence of SEQ ID NO: 83, optionally without the terminal HHHHHH (SEQ ID NO: 127). In some embodiments, an IL2R binding protein comprises the V_{H}H sequence of DR229, the V_{H}H sequence of DR587, and has at least 90% (e.g., 90%, 91%, 92%, 93%, 94%, 95%, 96%, 97%, 98%, 99%, or 100%) identity to the sequence of SEQ ID NO: 84, optionally without the terminal HHHHHHHHH (SEQ ID NO: 127). In some embodiments, an IL2R binding protein comprises the V_{H}H sequence of DR229, the V_{H}H sequence of DR588, and has at least 90% (e.g., 90%, 91%, 92%, 93%, 94%, 95%, 96%, 97%, 98%, 99%, or 100%) identity to the sequence of SEQ ID NO: 85, optionally without the terminal HHHHHH (SEQ ID NO: 127). In some embodiments, an IL2R binding protein comprises the V_{H}H sequence of DR230, the V_{H}H sequence of DR214, and has at least 90% (e.g., 90%, 91%, 92%, 93%, 94%, 95%, 96%, 97%, 98%, 99%, or 100%) identity to the sequence of SEQ ID NO: 86, optionally without the terminal HHHHHHHHH (SEQ ID NO: 127). In some embodiments, an IL2R binding protein comprises the V_{H}H sequence of DR230, the V_{H}H sequence of DR217, and has at least 90% (e.g., 90%, 91%, 92%, 93%, 94%, 95%, 96%, 97%, 98%, 99%, or 100%) identity to the sequence of SEQ ID NO: 87, optionally without the terminal HHHHHH (SEQ ID NO: 127). In some embodiments, an IL2R binding protein comprises the V_{H}H sequence of DR230, the V_{H}H sequence of DR583, and has at least 90% (e.g., 90%, 91%, 92%, 93%, 94%, 95%, 96%, 97%, 98%, 99%, or 100%) identity to the sequence of SEQ ID NO: 88, optionally without the terminal HHHHHHHHH (SEQ ID NO: 127). In some embodiments, an IL2R binding protein comprises the V_{H}H sequence of DR230, the V_{H}H sequence of DR584, and has at least 90% (e.g., 90%, 91%, 92%, 93%, 94%, 95%, 96%, 97%, 98%, 99%, or 100%) identity to the sequence of SEQ ID NO: 89, optionally without the terminal HHHHHH (SEQ ID NO: 127). In some embodiments, an IL2R binding protein comprises the V_{H}H sequence of DR230, the V_{H}H sequence of DR586, and has at least 90% (e.g., 90%, 91%, 92%, 93%, 94%, 95%, 96%, 97%, 98%, 99%, or 100%) identity to the sequence of SEQ ID NO: 90, optionally without the terminal HHHHHHHHH (SEQ ID NO: 127). In some embodiments, an IL2R binding protein comprises the V_{H}H sequence of DR230, the V_{H}H sequence of DR587, and has at least 90% (e.g., 90%, 91%, 92%, 93%, 94%, 95%, 96%, 97%, 98%, 99%, or 100%) identity to the sequence of SEQ ID NO: 91, optionally without the terminal HHHHHH (SEQ ID NO: 127). In some embodiments, an IL2R binding protein comprises the V_{H}H sequence of DR231, the V_{H}H sequence of DR214, and has at least 90% (e.g., 90%, 91%, 92%, 93%, 94%, 95%, 96%, 97%, 98%, 99%, or 100%) identity to the sequence of SEQ ID NO: 92, optionally without the terminal HHHHHHHHH (SEQ ID NO: 127). In some embodiments, an IL2R binding protein comprises the V_{H}H sequence of DR231, the V_{H}H sequence of DR584, and has at least 90% (e.g., 90%, 91%, 92%, 93%, 94%, 95%, 96%, 97%, 98%, 99%, or 100%) identity to the sequence of SEQ ID NO: 93, optionally without the terminal HHHHHH (SEQ ID NO: 127). In some embodiments, an IL2R binding protein comprises the V_{H}H sequence of DR231, the V_{H}H sequence of DR585, and has at least 90% (e.g., 90%, 91%, 92%, 93%, 94%, 95%, 96%, 97%, 98%, 99%, or 100%) identity to the sequence of SEQ ID NO: 94, optionally without the terminal HHHHHHHHH (SEQ ID NO: 127). In some embodiments, an IL2R binding protein comprises the V_{H}H sequence of DR232, the V_{H}H sequence of DR590, and has at least 90% (e.g., 90%, 91%, 92%, 93%, 94%, 95%, 96%, 97%, 98%, 99%, or 100%) identity to the sequence of SEQ ID NO: 95, optionally without the terminal HHHHHH (SEQ ID NO: 127). In some embodiments, an IL2R binding protein comprises the V_{H}H sequence of DR233, the V_{H}H sequence of DR214, and has at least 90% (e.g., 90%, 91%, 92%, 93%, 94%, 95%, 96%, 97%, 98%, 99%, or 100%) identity to the sequence of SEQ ID NO: 96, optionally without the terminal HHHHHHHHH (SEQ ID NO: 127).

In some embodiments, an IL2R binding protein comprises the V_{H}H sequence of DR233, the V_{H}H sequence of DR217, and has at least 90% (e.g., 90%, 91%, 92%, 93%, 94%, 95%, 96%, 97%, 98%, 99%, or 100%) identity to the sequence of SEQ ID NO: 97, optionally without the terminal HHHHHH (SEQ ID NO: 127). In some embodiments, an IL2R binding protein comprises the V_{H}H sequence of DR233, the V_{H}H sequence of DR583, and has at least 90% (e.g., 90%, 91%, 92%, 93%, 94%, 95%, 96%, 97%, 98%, 99%, or 100%) identity to the sequence of SEQ ID NO: 98, optionally without the terminal HHHHHHHHH (SEQ ID NO: 127). In some embodiments, an IL2R binding protein comprises the V_{H}H sequence of DR233, the V_{H}H sequence of DR584, and has at least 90% (e.g., 90%, 91%, 92%, 93%, 94%, 95%, 96%, 97%, 98%, 99%, or 100%) identity to the sequence of SEQ ID NO: 99, optionally without the terminal HHHHHH (SEQ ID NO: 127). In some embodiments, an IL2R binding protein comprises the V_{H}H sequence of DR233, the V_{H}H sequence of DR585, and has at least 90% (e.g., 90%, 91%, 92%, 93%, 94%, 95%, 96%, 97%, 98%, 99%, or 100%) identity to the sequence of SEQ ID NO: 100, optionally without the terminal HHHHHH (SEQ ID NO: 127). In some embodiments, an IL2R binding protein comprises the V_{H}H sequence of DR233, the V_{H}H sequence of DR587, and has at least 90% (e.g., 90%, 91%, 92%, 93%, 94%, 95%, 96%, 97%, 98%, 99%, or 100%) identity to the sequence of SEQ ID NO: 101, optionally without the terminal HHHHHH (SEQ ID NO: 127). In some embodiments, an IL2R binding protein comprises the V_{H}H sequence of DR233, the V_{H}H sequence of DR588, and has at least 90% (e.g., 90%, 91%, 92%, 93%, 94%, 95%, 96%, 97%, 98%, 99%, or 100%) identity to the sequence of SEQ ID NO: 102, optionally without the terminal HHHHHH (SEQ ID NO: 127). In some embodiments, an IL2R binding protein comprises the V_{H}H sequence of DR233, the V_{H}H sequence of DR589, and has at least 90% (e.g., 90%, 91%, 92%, 93%, 94%, 95%, 96%, 97%, 98%, 99%, or 100%) identity to the sequence of SEQ ID NO: 103, optionally without the terminal HHHHHH (SEQ ID NO: 127). In some embodiments, an IL2R binding protein comprises the V_{H}H sequence of DR233, the V_{H}H sequence of DR590, and has at least 90% (e.g., 90%, 91%, 92%, 93%, 94%, 95%, 96%, 97%, 98%, 99%, or 100%) identity to the sequence of SEQ ID NO: 104, optionally without the terminal HHHHHH (SEQ ID NO: 127). In some embodiments, an IL2R binding protein comprises the V_{H}H sequence of DR234, the V_{H}H sequence of DR214, and has at least 90% (e.g., 90%, 91%, 92%, 93%, 94%, 95%, 96%, 97%, 98%, 99%, or 100%) identity to the sequence of SEQ ID NO: 105, optionally without the terminal HHHHHH (SEQ ID NO: 127). In some embodiments, an IL2R binding protein comprises the V_{H}H sequence of DR234, the V_{H}H sequence of DR587, and has at least 90% (e.g., 90%, 91%, 92%, 93%, 94%, 95%, 96%, 97%, 98%, 99%, or 100%) identity to the sequence of SEQ ID NO: 106, optionally without the terminal HHHHHH (SEQ ID NO: 127).

TABLE 4
Anti-IL2Rβ/γ V_{H}H² (anti-IL2Rγ V_{H}H-linker-anti-IL2Rβ V_{H}H)

| Anti- | N- | C- | |
|---|---|---|---|
| IL2Rβ/γ | terminal | terminal | |
| V_{H}H² | V_{H}H | V_{H}H | Anti-IL2Rβ/γ V_{H}H² |
| DR634 | DR229 | DR583 | QVQLQESGGGLVQPGGSLRLSCTASGFSFSSYPMTWA |
| | | | RQAPGKGLEWVSTIASDGGSTAYAASVEGRFTISRDN |
| | | | AKSTLYLQLNSLKTEDTAMYYCTKGYGDGTPAPGQG |
| | | | TQVTVSSGGGSQVQLQESGGGSVQAGGSLRLSCVGSG |
| | | | YTYDTSDMSWYRQAPGKEREFVSDIDSGDWAAYADA |
| | | | VKGRFTISRDNAKKTVYLQMNSLEPEDTAMYYCKAS |
| | | | YWKWGKLNNFWGPGTQVTVSSASHHHHHH |
| | | | (SEQ ID NO: 81) |
| DR635 | DR229 | DR584 | QVQLQESGGGLVQPGGSLRLSCTASGFSFSSYPMTWA |
| | | | RQAPGKGLEWVSTIASDGGSTAYAASVEGRFTISRDN |
| | | | AKSTLYLQLNSLKTEDTAMYYCTKGYGDGTPAPGQG |
| | | | TQVTVSSGGGSQVQLQESGGGLVQPGGSLKLSCAASG |
| | | | FRFSNYGMSWVRQAPGEGLEWVSYINGDGSRTHYAD |
| | | | SVKGRFTISRDNAKNTLYLQLNSLKTEDTAMYYCEKG |
| | | | LSRDGWSLSAASRGQGTQVTVSSASHHHHHH |
| | | | (SEQ ID NO: 82) |
| DR636 | DR229 | DR585 | QVQLQESGGGLVQPGGSLRLSCTASGFSFSSYPMTWA |
| | | | RQAPGKGLEWVSTIASDGGSTAYAASVEGRFTISRDN |
| | | | AKSTLYLQLNSLKTEDTAMYYCTKGYGDGTPAPGQG |
| | | | TQVTVSSGGGSQVQLQESGGGSVQTGGSLRLSCAVSG |
| | | | YTTYSFNYMGWFRQAPGKEREGVAVIYTGGGSTLYA |
| | | | DSVKGRFTISQDNAKNTVYLQMNSLKPEDTAMYYCA |
| | | | ADDQRFASPLYAYFGYWGQGTQVTVSSASHHHHHH |
| | | | (SEQ ID NO: 83) |
| DR638 | DR229 | DR587 | QVQLQESGGGLVQPGGSLRLSCTASGFSFSSYPMTWA |
| | | | RQAPGKGLEWVSTIASDGGSTAYAASVEGRFTISRDN |
| | | | AKSTLYLQLNSLKTEDTAMYYCTKGYGDGTPAPGQG |
| | | | TQVTVSSGGGSQVQLQESGGGSVQVGGSLRLSCATSG |
| | | | DTKSIRCMGWFRQTPGKEREGIAAIDREGFATYADSV |
| | | | YDRFTIAQDNAQNTLYLEMNALKPEDTAMYYCAAQN |
| | | | MCRVVRGAMTGVDYWGKGTQVTVSSASHHHHHH |
| | | | (SEQ ID NO: 84) |
| DR639 | DR229 | DR588 | QVQLQESGGGLVQPGGSLRLSCTASGFSFSSYPMTWA |
| | | | RQAPGKGLEWVSTIASDGGSTAYAASVEGRFTISRDN |
| | | | AKSTLYLQLNSLKTEDTAMYYCTKGYGDGTPAPGQG |
| | | | TQVTVSSGGGSQVQLQESGGGSVQVGGSLKLSCAASG |
| | | | YTYSSYYCMGWFRQAPGKEREGVAAIDSDGSTSYADS |
| | | | VKGRFTISQDDAKNTLYLQMNSLKPEDTAMYYCAAS |
| | | | YEVVDCYPSGYGQDYWGKGTQVTVSSASHHHHHH |
| | | | (SEQ ID NO: 85) |
| DR642 | DR230 | DR214 | QVQLQESGGGLVQPGGSLRLSCAASGFTFSSAHMSWV |
| | | | RQAPGKGREWIASIYSGGGTFYADSVKGRFTISRDNAK |
| | | | NTLYLQLNSLKAEDTAMYYCATNRLHYYSDDDSLRG |
| | | | QGTQVTVSSGGGSQVQLLESGGGLVQPGGSLRLSCAA |
| | | | SGVRISTQDMSWVRQAPGKGLEWVSSILTPNGSTYYA |
| | | | DSVKGRFTISRDNSKNTLYLQMNSLRAEDTAVYYCAG |
| | | | VDERCEAEDQIDYWGQGTLVTVSSASHHHHHH |
| | | | (SEQ ID NO: 86) |
| DR643 | DR230 | DR217 | QVQLQESGGGLVQPGGSLRLSCAASGFTFSSAHMSWV |
| | | | RQAPGKGREWIASIYSGGGTFYADSVKGRFTISRDNAK |
| | | | NTLYLQLNSLKAEDTAMYYCATNRLHYYSDDDSLRG |
| | | | QGTQVTVSSGGGSQVQLLESGGGLVQPGGSLRLSCAA |
| | | | SGDMIISEDMSWVRQAPGKGLEWVSTIASDDGSTYYA |
| | | | DSVKGRFTISRDNSKNTLYLQMNSLRAEDTAVYYCAG |
| | | | FDAQDAAIEYWGQGTLVTVSSASHHHHHH |
| | | | (SEQ ID NO: 87) |
| DR644 | DR230 | DR583 | QVQLQESGGGLVQPGGSLRLSCAASGFTFSSAHMSWV |
| | | | RQAPGKGREWIASIYSGGGTFYADSVKGRFTISRDNAK |
| | | | NTLYLQLNSLKAEDTAMYYCATNRLHYYSDDDSLRG |
| | | | QGTQVTVSSGGGSQVQLQESGGGSVQAGGSLRLSCVG |
| | | | SGYTYDTSDMSWYRQAPGKEREFVSDIDSGDWAAYA |
| | | | DAVKGRFTISRDNAKKTVYLQMNSLEPEDTAMYYCK |
| | | | ASYWKWGKLNNFWGPGTQVTVSSASHHHHHH |
| | | | (SEQ ID NO: 88) |
| DR645 | DR230 | DR584 | QVQLQESGGGLVQPGGSLRLSCAASGFTFSSAHMSWV |
| | | | RQAPGKGREWIASIYSGGGTFYADSVKGRFTISRDNAK |
| | | | NTLYLQLNSLKAEDTAMYYCATNRLHYYSDDDSLRG |
| | | | QGTQVTVSSGGGSQVQLQESGGGLVQPGGSLKLSCAA |
| | | | SGFRFSNYGMSWVRQAPGEGLEWVSYINGDGSRTHY |
| | | | ADSVKGRFTISRDNAKNTLYLQLNSLKTEDTAMYYCE |
| | | | KGLSRDGWSLSAASRGQGTQVTVSSASHHHHHH |
| | | | (SEQ ID NO: 89) |
| DR647 | DR230 | DR586 | QVQLQESGGGLVQPGGSLRLSCAASGFTFSSAHMSWV |
| | | | RQAPGKGREWIASIYSGGGTFYADSVKGRFTISRDNAK |
| | | | NTLYLQLNSLKAEDTAMYYCATNRLHYYSDDDSLRG |
| | | | QGTQVTVSSGGGSQVQLQESGGGLVQPGGSLRLSCVA |
| | | | SGFTFSNYWIFWVRQAAGKGLEWLSTSNTGGDTTKY |
| | | | ADSVKGRFTISRDSAKNTEYLQMNSLKPEDTAVYYCE |
| | | | TGRCARSGGYQGTQVTVSSASHHHHHH |
| | | | (SEQ ID NO: 90) |
| DR648 | DR230 | DR587 | QVQLQESGGGLVQPGGSLRLSCAASGFTFSSAHMSWV |
| | | | RQAPGKGREWIASIYSGGGTFYADSVKGRFTISRDNAK |
| | | | NTLYLQLNSLKAEDTAMYYCATNRLHYYSDDDSLRG |
| | | | QGTQVTVSSGGGSQVQLQESGGGSVQVGGSLRLSCAT |
| | | | SGDTKSIRCMGWFRQTPGKEREGIAAIDREGFATYADS |
| | | | VYDRFTIAQDNAQNTLYLEMNALKPEDTAMYYCAAQ |
| | | | NMCRVVRGAMTGVDYWGKGTQVTVSSASHHHHHH |
| | | | (SEQ ID NO: 91) |
| DR652 | DR231 | DR214 | QVQLQESGGGSVQAGGSLRLSCTASGFTFDDREMNW |
| | | | YRQAPGNECELVSTISSDGSTYYADSVKGRFTISQDNA |
| | | | KNTVYLQMDSVKPEDTAVYYCAADFMIAIQAPGAGC |
| | | | WGQGTQVTVSSGGGSQVQLLESGGGLVQPGGSLRLSC |
| | | | AASGVRISTQDMSWVRQAPGKGLEWVSSILTPNGSTY |
| | | | YADSVKGRFTISRDNSKNTLYLQMNSLRAEDTAVYYC |
| | | | AGVDERCEAEDQIDYWGQGTLVTVSSASHHHHHH |
| | | | (SEQ ID NO: 92) |
| DR655 | DR231 | DR584 | QVQLQESGGGSVQAGGSLRLSCTASGFTFDDREMNW |
| | | | YRQAPGNECELVSTISSDGSTYYADSVKGRFTISQDNA |
| | | | KNTVYLQMDSVKPEDTAVYYCAADFMIAIQAPGAGC |
| | | | WGQGTQVTVSSGGGSQVQLQESGGGLVQPGGSLKLS |
| | | | CAASGFRFSNYGMSWVRQAPGEGLEWVSYINGDGSR |
| | | | THYADSVKGRFTISRDNAKNTLYLQLNSLKTEDTAMY |
| | | | YCEKGLSRDGWSLSAASRGQGTQVTVSSASHHHHHH |
| | | | (SEQ ID NO: 93) |
| DR656 | DR231 | DR585 | QVQLQESGGGSVQAGGSLRLSCTASGFTFDDREMNW |
| | | | YRQAPGNECELVSTISSDGSTYYADSVKGRFTISQDNA |
| | | | KNTVYLQMDSVKPEDTAVYYCAADFMIAIQAPGAGC |
| | | | WGQGTQVTVSSGGGSQVQLQESGGGSVQTGGSLRLS |
| | | | CAVSGYTTYSFNYMGWFRQAPGKEREGVAVIYTGGG |
| | | | STLYADSVKGRFTISQDNAKNTVYLQMNSLKPEDTAM |
| | | | YYCAADDQRFASPLYAYFGYWGQGTQVTVSSASHHH |
| | | | HHH (SEQ ID NO: 94) |
| DR671 | DR232 | DR590 | QVQLQESGGGSVQAGGSLRLSCVASGYTSCMGWFRQ |
| | | | APGKEREAVATIYTRGRSIYYADSVKGRFTISQDNAKN |
| | | | TLYLQMNSLKPEDIAMYSCAAGGYSWSAGCEFNYWG |
| | | | QGTQVTVSSGGGSQVQLQESGGGSVQAGGSLRLSCAA |
| | | | SGYEYCRIHMTWYRQGPGKEREFVSSIGSDGRKTYAN |
| | | | SVTGRFTISRDNANHTVYLQMNSLSPEDTAMYYCKTE |
| | | | YLYGLGCPDGSAYWGQGTQVTVSSASHHHHHH |
| | | | (SEQ ID NO: 95) |
| DR672 | DR233 | DR214 | QVQLQESGGGSVQAGGSLRLSCTASGFTFDDSDMGW |
| | | | YRQAPGNECELVSTISSDGSTYYADSVKGRFTISQDNA |
| | | | KNTVYLQMNSLKPEDTAVYYCAAEPRGYYSNYGGRR |
| | | | ECNYWGQGTQVTVSSGGGSQVQLLESGGGLVQPGGS |
| | | | LRLSCAASGVRISTQDMSWVRQAPGKGLEWVSSILTP |
| | | | NGSTYYADSVKGRFTISRDNSKNTLYLQMNSLRAEDT |
| | | | AVYYCAGVDERCEAEDQIDYWGQGTLVTVSSASHHH |
| | | | HHH (SEQ ID NO: 96) |
| DR673 | DR233 | DR217 | QVQLQESGGGSVQAGGSLRLSCTASGFTFDDSDMGW |
| | | | YRQAPGNECELVSTISSDGSTYYADSVKGRFTISQDNA |
| | | | KNTVYLQMNSLKPEDTAVYYCAAEPRGYYSNYGGRR |
| | | | ECNYWGQGTQVTVSSGGGSQVQLLESGGGLVQPGGS |
| | | | LRLSCAASGDMIISEDMSWVRQAPGKGLEWVSTIASD |
| | | | DGSTYYADSVKGRFTISRDNSKNTLYLQMNSLRAEDT |
| | | | AVYYCAGFDAQDAAIEYWGQGTLVTVSSASHHHHHH |
| | | | (SEQ ID NO: 97) |
| DR674 | DR233 | DR583 | QVQLQESGGGSVQAGGSLRLSCTASGFTFDDSDMGW |
| | | | YRQAPGNECELVSTISSDGSTYYADSVKGRFTISQDNA |
| | | | KNTVYLQMNSLKPEDTAVYYCAAEPRGYYSNYGGRR |
| | | | ECNYWGQGTQVTVSSGGGSQVQLQESGGGSVQAGGS |
| | | | LRLSCVGSGYTYDTSDMSWYRQAPGKEREFVSDIDSG |
| | | | DWAAYADAVKGRFTISRDNAKKTVYLQMNSLEPEDT |
| | | | AMYYCKASYWKWGKLNNFWGPGTQVTVSSASHHHH |
| | | | HH (SEQ ID NO: 98) |
| DR675 | DR233 | DR584 | QVQLQESGGGSVQAGGSLRLSCTASGFTFDDSDMGW |
| | | | YRQAPGNECELVSTISSDGSTYYADSVKGRFTISQDNA |
| | | | KNTVYLQMNSLKPEDTAVYYCAAEPRGYYSNYGGRR |
| | | | ECNYWGQGTQVTVSSGGGSQVQLQESGGGLVQPGGS |
| | | | LKLSCAASGFRFSNYGMSWVRQAPGEGLEWVSYING |
| | | | DGSRTHYADSVKGRFTISRDNAKNTLYLQLNSLKTED |
| | | | TAMYYCEKGLSRDGWSLSAASRGQGTQVTVSSASHH |
| | | | HHHH (SEQ ID NO: 99) |
| DR676 | DR233 | DR585 | QVQLQESGGGSVQAGGSLRLSCTASGFTFDDSDMGW |
| | | | YRQAPGNECELVSTISSDGSTYYADSVKGRFTISQDNA |
| | | | KNTVYLQMNSLKPEDTAVYYCAAEPRGYYSNYGGRR |
| | | | ECNYWGQGTQVTVSSGGGSQVQLQESGGGSVQTGGS |
| | | | LRLSCAVSGYTTYSFNYMGWFRQAPGKEREGVAVIYT |
| | | | GGGSTLYADSVKGRFTISQDNAKNTVYLQMNSLKPED |
| | | | TAMYYCAADDQRFASPLYAYFGYWGQGTQVTVSSAS |
| | | | HHHHHH (SEQ ID NO: 100) |
| DR678 | DR233 | DR587 | QVQLQESGGGSVQAGGSLRLSCTASGFTFDDSDMGW |
| | | | YRQAPGNECELVSTISSDGSTYYADSVKGRFTISQDNA |
| | | | KNTVYLQMNSLKPEDTAVYYCAAEPRGYYSNYGGRR |
| | | | ECNYWGQGTQVTVSSGGGSQVQLQESGGGSVQVGGS |
| | | | LRLSCATSGDTKSIRCMGWFRQTPGKEREGIAAIDREG |
| | | | FATYADSVYDRFTIAQDNAQNTLYLEMNALKPEDTA |
| | | | MYYCAAQNMCRVVRGAMTGVDYWGKGTQVTVSSA |
| | | | SHHHHHH (SEQ ID NO: 101) |
| DR679 | DR233 | DR588 | QVQLQESGGGSVQAGGSLRLSCTASGFTFDDSDMGW |
| | | | YRQAPGNECELVSTISSDGSTYYADSVKGRFTISQDNA |
| | | | KNTVYLQMNSLKPEDTAVYYCAAEPRGYYSNYGGRR |
| | | | ECNYWGQGTQVTVSSGGGSQVQLQESGGGSVQVGGS |
| | | | LKLSCAASGYTYSSYYCMGWFRQAPGKEREGVAAIDS |
| | | | DGSTSYADSVKGRFTISQDDAKNTLYLQMNSLKPEDT |
| | | | AMYYCAASYEVVDCYPSGYGQDYWGKGTQVTVSSA |
| | | | SHHHHHH (SEQ ID NO: 102) |
| DR680 | DR233 | DR589 | QVQLQESGGGSVQAGGSLRLSCTASGFTFDDSDMGW |
| | | | YRQAPGNECELVSTISSDGSTYYADSVKGRFTISQDNA |
| | | | KNTVYLQMNSLKPEDTAVYYCAAEPRGYYSNYGGRR |
| | | | ECNYWGQGTQVTVSSGGGSQVQLQESGGGSVQAGGS |
| | | | LRLSCAASEYTASRYCMAWFRQAPGKEREGVAAIHPG |
| | | | GGTTYYADSVKGRFSISQDSADNTLYLQMNSLKPEDT |
| | | | AMYYCAAGSLWVPFGDRCAANYWGQGTQVTVSSAS |
| | | | HHHHHH (SEQ ID NO: 103) |
| DR681 | DR233 | DR590 | QVQLQESGGGSVQAGGSLRLSCTASGFTFDDSDMGW |
| | | | YRQAPGNECELVSTISSDGSTYYADSVKGRFTISQDNA |
| | | | KNTVYLQMNSLKPEDTAVYYCAAEPRGYYSNYGGRR |
| | | | ECNYWGQGTQVTVSSGGGSQVQLQESGGGSVQAGGS |
| | | | LRLSCAASGYEYCRIHMTWYRQGPGKEREFVSSIGSD |
| | | | GRKTYANSVTGRFTISRDNANHTVYLQMNSLSPEDTA |
| | | | MYYCKTEYLYGLGCPDGSAYWGQGTQVTVSSASHHH |
| | | | HHH (SEQ ID NO: 104) |
| DR682 | DR234 | DR214 | QVQLQESGGGSVQAGGSLRLSCVASGYTFSSYCMGW |
| | | | FRQAPGKEREGVAALGGGSTYYADSVKGRFTISQDNA |
| | | | KNTLYLQMNSLKPEDTAMYYCAAAWVACLEFGGSW |
| | | | YDLARYKHWGQGTQVTVSSGGGSQVQLLESGGGLVQ |
| | | | PGGSLRLSCAASGVRISTQDMSWVRQAPGKGLEWVSS |
| | | | ILTPNGSTYYADSVKGRFTISRDNSKNTLYLQMNSLRA |
| | | | EDTAVYYCAGVDERCEAEDQIDYWGQGTLVTVSSAS |
| | | | HHHHHH (SEQ ID NO: 105) |
| DR688 | DR234 | DR587 | QVQLQESGGGSVQAGGSLRLSCVASGYTFSSYCMGW |
| | | | FRQAPGKEREGVAALGGGSTYYADSVKGRFTISQDNA |
| | | | KNTLYLQMNSLKPEDTAMYYCAAAWVACLEFGGSW |
| | | | YDLARYKHWGQGTQVTVSSGGGSQVQLQESGGGSVQ |
| | | | VGGSLRLSCATSGDTKSIRCMGWFRQTPGKEREGIAAI |
| | | | DREGFATYADSVYDRFTIAQDNAQNTLYLEMNALKPE |
| | | | DTAMYYCAAQNMCRVVRGAMTGVDYWGKGTQVTV |
| | | | SSASHHHHHH (SEQ ID NO: 106) |

As shown in the illustrative examples of IL2R binding proteins of Table 3 and Table 4, the IL2R binding protein sequences listed therein contain GGGS (SEQ ID NO: 107) as a linker. In some embodiments, the GGGS (SEQ ID NO: 107) can be replaced by other linkers as described further herein. Furthermore, the IL2R binding protein sequences shown in Table 3 and Table 4 may be operably linked to a chelating peptide such as the “ASH6” (SEQ ID NO: 126) metal chelating peptide which may be used to facilitate purification via metal affinity chromatography. In some embodiments, this purification handle can be removed or replaced by other purification handles (e.g., H6 (SEQ ID NO: 127)).

Further, in each of SEQ ID NOS: 170-289 below, each title of the sequence follows the formula “anti-IL2Rβ/IL2Rγ V_{H}H² (V_{H}H antibody at the N-terminus—V_{H}H antibody at the C-terminus).” For example, “DR632(DR229-DR214)” refers to the anti-IL2Rβ/IL2Rγ V_{H}H² binding protein with DR229 V_{H}H at the N-terminus and DR214 V_{H}H antibody at the C-terminus. In each of SEQ ID NOS: 170-289 below, the linker is in bold, and each of CDR1, CDR2, CDR3 of the N-terminal V_{H}H antibody and CDR1, CDR2, CDR3 of the C-terminal V_{H}H antibody is underlined, respectively. An IL2Rβ/IL2Rγ binding protein described herein can comprise the V_{H}H sequence of the N-terminal V_{H}H antibody, the V_{H}H sequence of the C-terminal V_{H}H antibody, and has at least 90% (e.g., 90%, 91%, 92%, 93%, 94%, 95%, 96%, 97%, 98%, 99%, or 100%) identity to a sequence of any one of SEQ ID NOS: 170-289, optionally without the terminal HHHHHH (SEQ ID NO: 127). Moreover, the GGGS (SEQ ID NO: 107) in each of SEQ ID NOS: 170-289 below can be replaced by other linkers as described further herein. The purification handle “ASH₆” (SEQ ID NO: 126) at the end of each of SEQ ID NOS: 170-289 can be removed or replaced by other purification handles (e.g., H₆ (SEQ ID NO: 127)).

> SEQ ID NO: 170; DR632(DR229-DR214)
QVQLQESGGGLVQPGGSLRLSCTASGFSFSSYPMTWARQAPGKGLEWVS
TIASDGGSTAYAASVEGRFTISRDNAKSTLYLQLNSLKTEDTAMYYCTK
GYGDGTPAPGQGTQVTVSSGGGSQVQLLESGGGLVQPGGSLRLSCAASG
VRISTQDMSWVRQAPGKGLEWVSSILTPNGSTYYADSVKGRFTISRDNS
KNTLYLQMNSLRAEDTAVYYCAGVDERCEAEDQIDYWGQGTLVTVSSAS
HHHHHH
> SEQ ID NO: 171; DR633(DR229-DR217)
QVQLQESGGGLVQPGGSLRLSCTASGFSFSSYPMTWARQAPGKGLEWVS
TIASDGGSTAYAASVEGRFTISRDNAKSTLYLQLNSLKTEDTAMYYCTK
GYGDGTPAPGQGTQVTVSSGGGSQVQLLESGGGLVQPGGSLRLSCAASG
DMIISEDMSWVRQAPGKGLEWVSTIASDDGSTYYADSVKGRFTISRDNS
KNTLYLQMNSLRAEDTAVYYCAGFDAQDAAIEYWGQGTLVTVSSASHHH
HHH
> SEQ ID NO: 172; DR634(DR229-DR583)
QVQLQESGGGLVQPGGSLRLSCTASGFSFSSYPMTWARQAPGKGLEWVS
TIASDGGSTAYAASVEGRFTISRDNAKSTLYLQLNSLKTEDTAMYYCTK
GYGDGTPAPGQGTQVTVSSGGGSQVQLQESGGGSVQAGGSLRLSCVGSG
YTYDTSDMSWYRQAPGKEREFVSDIDSGDWAAYADAVKGRFTISRDNAK
KTVYLQMNSLEPEDTAMYYCKASYWKWGKLNNFWGPGTQVTVSSASHHH
HHH
> SEQ ID NO: 173; DR635(DR229-DR584)
QVQLQESGGGLVQPGGSLRLSCTASGFSFSSYPMTWARQAPGKGLEWVS
TIASDGGSTAYAASVEGRFTISRDNAKSTLYLQLNSLKTEDTAMYYCTK
GYGDGTPAPGQGTQVTVSSGGGSQVQLQESGGGLVQPGGSLKLSCAASG
FRFSNYGMSWVRQAPGEGLEWVSYINGDGSRTHYADSVKGRFTISRDNA
KNTLYLQLNSLKTEDTAMYYCEKGLSRDGWSLSAASRGQGTQVTVSSAS
HHHHHH
> SEQ ID NO: 174; DR636(DR229-DR585)
QVQLQESGGGLVQPGGSLRLSCTASGFSFSSYPMTWARQAPGKGLEWVS
TIASDGGSTAYAASVEGRFTISRDNAKSTLYLQLNSLKTEDTAMYYCTK
GYGDGTPAPGQGTQVTVSSGGGSQVQLQESGGGSVQTGGSLRLSCAVSG
YTTYSFNYMGWFRQAPGKEREGVAVIYTGGGSTLYADSVKGRFTISQDN
AKNTVYLQMNSLKPEDTAMYYCAADDQRFASPLYAYFGYWGQGTQVTVS
SASHHHHHH
> SEQ ID NO: 175; DR637(DR229-DR586)
QVQLQESGGGLVQPGGSLRLSCTASGFSFSSYPMTWARQAPGKGLEWVS
TIASDGGSTAYAASVEGRFTISRDNAKSTLYLQLNSLKTEDTAMYYCTK
GYGDGTPAPGQGTQVTVSSGGGSQVQLQESGGGLVQPGGSLRLSCVASG
FTFSNYWIFWVRQAAGKGLEWLSTSNTGGDTTKYADSVKGRFTISRDSA
KNTEYLQMNSLKPEDTAVYYCETGRCARSGGYQGTQVTVSSASHHHHHH
> SEQ ID NO: 176; DR638(DR229-DR587)
QVQLQESGGGLVQPGGSLRLSCTASGFSFSSYPMTWARQAPGKGLEWVS
TIASDGGSTAYAASVEGRFTISRDNAKSTLYLQLNSLKTEDTAMYYCTK
GYGDGTPAPGQGTQVTVSSGGGSQVQLQESGGGSVQVGGSLRLSCATSG
DTKSIRCMGWFRQTPGKEREGIAAIDREGFATYADSVYDRFTIAQDNAQ
NTLYLEMNALKPEDTAMYYCAAQNMCRVVRGAMTGVDYWGKGTQVTVSS
ASHHHHHH
> SEQ ID NO: 177; DR639(DR229-DR588)
QVQLQESGGGLVQPGGSLRLSCTASGFSFSSYPMTWARQAPGKGLEWVS
TIASDGGSTAYAASVEGRFTISRDNAKSTLYLQLNSLKTEDTAMYYCTK
GYGDGTPAPGQGTQVTVSSGGGSQVQLQESGGGSVQVGGSLKLSCAASG
YTYSSYYCMGWFRQAPGKEREGVAAIDSDGSTSYADSVKGRFTISQDDA
KNTLYLQMNSLKPEDTAMYYCAASYEVVDCYPSGYGQDYWGKGTQVTVS
SASHHHHHH
> SEQ ID NO: 178; DR640(DR229-DR589)
QVQLQESGGGLVQPGGSLRLSCTASGFSFSSYPMTWARQAPGKGLEWVS
TIASDGGSTAYAASVEGRFTISRDNAKSTLYLQLNSLKTEDTAMYYCTK
GYGDGTPAPGQGTQVTVSSGGGSQVQLQESGGGSVQAGGSLRLSCAASE
YTASRYCMAWFRQAPGKEREGVAAIHPGGGTTYYADSVKGRFSISQDSA
DNTLYLQMNSLKPEDTAMYYCAAGSLWVPFGDRCAANYWGQGTQVTVSS
ASHHHHHH
> SEQ ID NO: 179; DR641(DR229-DR590)
QVQLQESGGGLVQPGGSLRLSCTASGFSFSSYPMTWARQAPGKGLEWVS
TIASDGGSTAYAASVEGRFTISRDNAKSTLYLQLNSLKTEDTAMYYCTK
GYGDGTPAPGQGTQVTVSSGGGSQVQLQESGGGSVQAGGSLRLSCAASG
YEYCRIHMTWYRQGPGKEREFVSSIGSDGRKTYANSVTGRFTISRDNAN
HTVYLQMNSLSPEDTAMYYCKTEYLYGLGCPDGSAYWGQGTQVTVSSAS
HHHHHH
> SEQ ID NO: 180; DR642(DR230-DR214)
QVQLQESGGGLVQPGGSLRLSCAASGFTFSSAHMSWVRQAPGKGREWIA
SIYSGGGTFYADSVKGRFTISRDNAKNTLYLQLNSLKAEDTAMYYCATN
RLHYYSDDDSLRGQGTQVTVSSGGGSQVQLLESGGGLVQPGGSLRLSCA
ASGVRISTQDMSWVRQAPGKGLEWVSSILTPNGSTYYADSVKGRFTISR
DNSKNTLYLQMNSLRAEDTAVYYCAGVDERCEAEDQIDYWGQGTLVTVS
SASHHHHHH
> SEQ ID NO: 181; DR643(DR230-DR217)
QVQLQESGGGLVQPGGSLRLSCAASGFTFSSAHMSWVRQAPGKGREWIA
SIYSGGGTFYADSVKGRFTISRDNAKNTLYLQLNSLKAEDTAMYYCATN
RLHYYSDDDSLRGQGTQVTVSSGGGSQVQLLESGGGLVQPGGSLRLSCA
ASGDMIISEDMSWVRQAPGKGLEWVSTIASDDGSTYYADSVKGRFTISR
DNSKNTLYLQMNSLRAEDTAVYYCAGFDAQDAAIEYWGQGTLVTVSSAS
HHHHHH
> SEQ ID NO: 182; DR644(DR230-DR583)
QVQLQESGGGLVQPGGSLRLSCAASGFTFSSAHMSWVRQAPGKGREWIA
SIYSGGGTFYADSVKGRFTISRDNAKNTLYLQLNSLKAEDTAMYYCATN
RLHYYSDDDSLRGQGTQVTVSSGGGSQVQLQESGGGSVQAGGSLRLSCV
GSGYTYDTSDMSWYRQAPGKEREFVSDIDSGDWAAYADAVKGRFTISRD
NAKKTVYLQMNSLEPEDTAMYYCKASYWKWGKLNNFWGPGTQVTVSSAS
HHHHHH
> SEQ ID NO: 183; DR645(DR230-DR584)
QVQLQESGGGLVQPGGSLRLSCAASGFTFSSAHMSWVRQAPGKGREWIA
SIYSGGGTFYADSVKGRFTISRDNAKNTLYLQLNSLKAEDTAMYYCATN
RLHYYSDDDSLRGQGTQVTVSSGGGSQVQLQESGGGLVQPGGSLKLSCA
ASGFRFSNYGMSWVRQAPGEGLEWVSYINGDGSRTHYADSVKGRFTISR
DNAKNTLYLQLNSLKTEDTAMYYCEKGLSRDGWSLSAASRGQGTQVTVS
SASHHHHHH
> SEQ ID NO: 184; DR646(DR230-DR585)
QVQLQESGGGLVQPGGSLRLSCAASGFTFSSAHMSWVRQAPGKGREWIA
SIYSGGGTFYADSVKGRFTISRDNAKNTLYLQLNSLKAEDTAMYYCATN
RLHYYSDDDSLRGQGTQVTVSSGGGSQVQLQESGGGSVQTGGSLRLSCA
VSGYTTYSFNYMGWFRQAPGKEREGVAVIYTGGGSTLYADSVKGRFTIS
QDNAKNTVYLQMNSLKPEDTAMYYCAADDQRFASPLYAYFGYWGQGTQV
TVSSASHHHHHH
> SEQ ID NO: 185; DR647(DR230-DR586)
QVQLQESGGGLVQPGGSLRLSCAASGFTFSSAHMSWVRQAPGKGREWIA
SIYSGGGTFYADSVKGRFTISRDNAKNTLYLQLNSLKAEDTAMYYCATN
RLHYYSDDDSLRGQGTQVTVSSGGGSQVQLQESGGGLVQPGGSLRLSCV
ASGFTFSNYWIFWVRQAAGKGLEWLSTSNTGGDTTKYADSVKGRFTISR
DSAKNTEYLQMNSLKPEDTAVYYCETGRCARSGGYQGTQVTVSSASHHH
HHH
> SEQ ID NO: 186; DR648(DR230-DR587)
QVQLQESGGGLVQPGGSLRLSCAASGFTFSSAHMSWVRQAPGKGREWIA
SIYSGGGTFYADSVKGRFTISRDNAKNTLYLQLNSLKAEDTAMYYCATN
RLHYYSDDDSLRGQGTQVTVSSGGGSQVQLQESGGGSVQVGGSLRLSCA
TSGDTKSIRCMGWFRQTPGKEREGIAAIDREGFATYADSVYDRFTIAQD
NAQNTLYLEMNALKPEDTAMYYCAAQNMCRVVRGAMTGVDYWGKGTQVT
VSSASHHHHHH
> SEQ ID NO: 187; DR649(DR230-DR588)
QVQLQESGGGLVQPGGSLRLSCAASGFTFSSAHMSWVRQAPGKGREWIA
SIYSGGGTFYADSVKGRFTISRDNAKNTLYLQLNSLKAEDTAMYYCATN
RLHYYSDDDSLRGQGTQVTVSSGGGSQVQLQESGGGSVQVGGSLKLSCA
ASGYTYSSYYCMGWFRQAPGKEREGVAAIDSDGSTSYADSVKGRFTISQ
DDAKNTLYLQMNSLKPEDTAMYYCAASYEVVDCYPSGYGQDYWGKGTQV
TVSSASHHHHHH
> SEQ ID NO: 188; DR650(DR230-DR589)
QVQLQESGGGLVQPGGSLRLSCAASGFTFSSAHMSWVRQAPGKGREWIA
SIYSGGGTFYADSVKGRFTISRDNAKNTLYLQLNSLKAEDTAMYYCATN
RLHYYSDDDSLRGQGTQVTVSSGGGSQVQLQESGGGSVQAGGSLRLSCA
ASEYTASRYCMAWFRQAPGKEREGVAAIHPGGGTTYYADSVKGRFSISQ
DSADNTLYLQMNSLKPEDTAMYYCAAGSLWVPFGDRCAANYWGQGTQVT
VSSASHHHHHH
> SEQ ID NO: 189; DR651(DR230-DR590)
QVQLQESGGGLVQPGGSLRLSCAASGFTFSSAHMSWVRQAPGKGREWIA
SIYSGGGTFYADSVKGRFTISRDNAKNTLYLQLNSLKAEDTAMYYCATN
RLHYYSDDDSLRGQGTQVTVSSGGGSQVQLQESGGGSVQAGGSLRLSCA
ASGYEYCRIHMTWYRQGPGKEREFVSSIGSDGRKTYANSVTGRFTISRD
NANHTVYLQMNSLSPEDTAMYYCKTEYLYGLGCPDGSAYWGQGTQVTVS
SASHHHHHH
> SEQ ID NO: 190; DR652(DR231-DR214)
QVQLQESGGGSVQAGGSLRLSCTASGFTFDDREMNWYRQAPGNECELVS
TISSDGSTYYADSVKGRFTISQDNAKNTVYLQMDSVKPEDTAVYYCAAD
FMIAIQAPGAGCWGQGTQVTVSSGGGSQVQLLESGGGLVQPGGSLRLSC
AASGVRISTQDMSWVRQAPGKGLEWVSSILTPNGSTYYADSVKGRFTIS
RDNSKNTLYLQMNSLRAEDTAVYYCAGVDERCEAEDQIDYWGQGTLVTV
SSASHHHHHH
> SEQ ID NO: 191; DR653(DR231-DR217)
QVQLQESGGGSVQAGGSLRLSCTASGFTFDDREMNWYRQAPGNECELVS
TISSDGSTYYADSVKGRFTISQDNAKNTVYLQMDSVKPEDTAVYYCAAD
FMIAIQAPGAGCWGQGTQVTVSSGGGSQVQLLESGGGLVQPGGSLRLSC
AASGDMIISEDMSWVRQAPGKGLEWVSTIASDDGSTYYADSVKGRFTIS
RDNSKNTLYLQMNSLRAEDTAVYYCAGFDAQDAAIEYWGQGTLVTVSSA
SHHHHHH
> SEQ ID NO: 192; DR654(DR231-DR583)
QVQLQESGGGSVQAGGSLRLSCTASGFTFDDREMNWYRQAPGNECELVS
TISSDGSTYYADSVKGRFTISQDNAKNTVYLQMDSVKPEDTAVYYCAAD
FMIAIQAPGAGCWGQGTQVTVSSGGGSQVQLQESGGGSVQAGGSLRLSC
VGSGYTYDTSDMSWYRQAPGKEREFVSDIDSGDWAAYADAVKGRFTISR
DNAKKTVYLQMNSLEPEDTAMYYCKASYWKWGKLNNFWGPGTQVTVSSA
SHHHHHH
> SEQ ID NO: 193; DR655(DR231-DR584)
QVQLQESGGGSVQAGGSLRLSCTASGFTFDDREMNWYRQAPGNECELVS
TISSDGSTYYADSVKGRFTISQDNAKNTVYLQMDSVKPEDTAVYYCAAD
FMIAIQAPGAGCWGQGTQVTVSSGGGSQVQLQESGGGLVQPGGSLKLSC
AASGFRFSNYGMSWVRQAPGEGLEWVSYINGDGSRTHYADSVKGRFTIS
RDNAKNTLYLQLNSLKTEDTAMYYCEKGLSRDGWSLSAASRGQGTQVTV
SSASHHHHHH
> SEQ ID NO: 194; DR656(DR231-DR585)
QVQLQESGGGSVQAGGSLRLSCTASGFTFDDREMNWYRQAPGNECELVS
TISSDGSTYYADSVKGRFTISQDNAKNTVYLQMDSVKPEDTAVYYCAAD
FMIAIQAPGAGCWGQGTQVTVSSGGGSQVQLQESGGGSVQTGGSLRLSC
AVSGYTTYSFNYMGWFRQAPGKEREGVAVIYTGGGSTLYADSVKGRFTI
SQDNAKNTVYLQMNSLKPEDTAMYYCAADDQRFASPLYAYFGYWGQGTQ
VTVSSASHHHHHH
> SEQ ID NO: 195; DR657(DR231-DR586)
QVQLQESGGGSVQAGGSLRLSCTASGFTFDDREMNWYRQAPGNECELVS
TISSDGSTYYADSVKGRFTISQDNAKNTVYLQMDSVKPEDTAVYYCAAD
FMIAIQAPGAGCWGQGTQVTVSSGGGSQVQLQESGGGLVQPGGSLRLSC
VASGFTFSNYWIFWVRQAAGKGLEWLSTSNTGGDTTKYADSVKGRFTIS
RDSAKNTEYLQMNSLKPEDTAVYYCETGRCARSGGYQGTQVTVSSASHH
HHHH
> SEQ ID NO: 196; DR658(DR231-DR587)
QVQLQESGGGSVQAGGSLRLSCTASGFTFDDREMNWYRQAPGNECELVS
TISSDGSTYYADSVKGRFTISQDNAKNTVYLQMDSVKPEDTAVYYCAAD
FMIAIQAPGAGCWGQGTQVTVSSGGGSQVQLQESGGGSVQVGGSLRLSC
ATSGDTKSIRCMGWFRQTPGKEREGIAAIDREGFATYADSVYDRFTIAQ
DNAQNTLYLEMNALKPEDTAMYYCAAQNMCRVVRGAMTGVDYWGKGTQV
TVSSASHHHHHH
> SEQ ID NO: 197; DR659(DR231-DR588)
QVQLQESGGGSVQAGGSLRLSCTASGFTFDDREMNWYRQAPGNECELVS
TISSDGSTYYADSVKGRFTISQDNAKNTVYLQMDSVKPEDTAVYYCAAD
FMIAIQAPGAGCWGQGTQVTVSSGGGSQVQLQESGGGSVQVGGSLKLSC
AASGYTYSSYYCMGWFRQAPGKEREGVAAIDSDGSTSYADSVKGRFTIS
QDDAKNTLYLQMNSLKPEDTAMYYCAASYEVVDCYPSGYGQDYWGKGTQ
VTVSSASHHHHHH
> SEQ ID NO: 198; DR660(DR231-DR589)
QVQLQESGGGSVQAGGSLRLSCTASGFTFDDREMNWYRQAPGNECELVS
TISSDGSTYYADSVKGRFTISQDNAKNTVYLQMDSVKPEDTAVYYCAAD
FMIAIQAPGAGCWGQGTQVTVSSGGGSQVQLQESGGGSVQAGGSLRLSC
AASEYTASRYCMAWFRQAPGKEREGVAAIHPGGGTTYYADSVKGRFSIS
QDSADNTLYLQMNSLKPEDTAMYYCAAGSLWVPFGDRCAANYWGQGTQV
TVSSASHHHHHH
> SEQ ID NO: 199; DR661(DR231-DR590)
QVQLQESGGGSVQAGGSLRLSCTASGFTFDDREMNWYRQAPGNECELVS
TISSDGSTYYADSVKGRFTISQDNAKNTVYLQMDSVKPEDTAVYYCAAD
FMIAIQAPGAGCWGQGTQVTVSSGGGSQVQLQESGGGSVQAGGSLRLSC
AASGYEYCRIHMTWYRQGPGKEREFVSSIGSDGRKTYANSVTGRFTISR
DNANHTVYLQMNSLSPEDTAMYYCKTEYLYGLGCPDGSAYWGQGTQVTV
SSASHHHHHH
> SEQ ID NO: 200; DR662(DR232-DR214)
QVQLQESGGGSVQAGGSLRLSCVASGYTSCMGWFRQAPGKEREAVATIY
TRGRSIYYADSVKGRFTISQDNAKNTLYLQMNSLKPEDIAMYSCAAGGY
SWSAGCEFNYWGQGTQVTVSSGGGSQVQLLESGGGLVQPGGSLRLSCAA
SGVRISTQDMSWVRQAPGKGLEWVSSILTPNGSTYYADSVKGRFTISRD
NSKNTLYLQMNSLRAEDTAVYYCAGVDERCEAEDQIDYWGQGTLVTVSS
ASHHHHHH
> SEQ ID NO: 201; DR663(DR232-DR217)
QVQLQESGGGSVQAGGSLRLSCVASGYTSCMGWFRQAPGKEREAVATIY
TRGRSIYYADSVKGRFTISQDNAKNTLYLQMNSLKPEDIAMYSCAAGGY
SWSAGCEFNYWGQGTQVTVSSGGGSQVQLLESGGGLVQPGGSLRLSCAA
SGDMIISEDMSWVRQAPGKGLEWVSTIASDDGSTYYADSVKGRFTISRD
NSKNTLYLQMNSLRAEDTAVYYCAGFDAQDAAIEYWGQGTLVTVSSASH
HHHHH
> SEQ ID NO: 202; DR664(DR232-DR583)
QVQLQESGGGSVQAGGSLRLSCVASGYTSCMGWFRQAPGKEREAVATIY
TRGRSIYYADSVKGRFTISQDNAKNTLYLQMNSLKPEDIAMYSCAAGGY
SWSAGCEFNYWGQGTQVTVSSGGGSQVQLQESGGGSVQAGGSLRLSCVG
SGYTYDTSDMSWYRQAPGKEREFVSDIDSGDWAAYADAVKGRFTISRDN
AKKTVYLQMNSLEPEDTAMYYCKASYWKWGKLNNFWGPGTQVTVSSASH
HHHHH
> SEQ ID NO: 203; DR665(DR232-DR584)
QVQLQESGGGSVQAGGSLRLSCVASGYTSCMGWFRQAPGKEREAVATIY
TRGRSIYYADSVKGRFTISQDNAKNTLYLQMNSLKPEDIAMYSCAAGGY
SWSAGCEFNYWGQGTQVTVSSGGGSQVQLQESGGGLVQPGGSLKLSCAA
SGFRFSNYGMSWVRQAPGEGLEWVSYINGDGSRTHYADSVKGRFTISRD
NAKNTLYLQLNSLKTEDTAMYYCEKGLSRDGWSLSAASRGQGTQVTVSS
ASHHHHHH
> SEQ ID NO: 204; DR666(DR232-DR585)
QVQLQESGGGSVQAGGSLRLSCVASGYTSCMGWFRQAPGKEREAVATIY
TRGRSIYYADSVKGRFTISQDNAKNTLYLQMNSLKPEDIAMYSCAAGGY
SWSAGCEFNYWGQGTQVTVSSGGGSQVQLQESGGGSVQTGGSLRLSCAV
SGYTTYSFNYMGWFRQAPGKEREGVAVIYTGGGSTLYADSVKGRFTISQ
DNAKNTVYLQMNSLKPEDTAMYYCAADDQRFASPLYAYFGYWGQGTQVT
VSSASHHHHHH
> SEQ ID NQ: 205; DR667(DR232-DR586)
QVQLQESGGGSVQAGGSLRLSCVASGYTSCMGWFRQAPGKEREAVATIY
TRGRSIYYADSVKGRFTISQDNAKNTLYLQMNSLKPEDIAMYSCAAGGY
SWSAGCEFNYWGQGTQVTVSSGGGSQVQLQESGGGLVQPGGSLRLSCVA
SGFTFSNYWIFWVRQAAGKGLEWLSTSNTGGDTTKYADSVKGRFTISRD
SAKNTEYLQMNSLKPEDTAVYYCETGRCARSGGYQGTQVTVSSASHHHH
HH
> SEQ ID NO: 206; DR668(DR232-DR587)
QVQLQESGGGSVQAGGSLRLSCVASGYTSCMGWFRQAPGKEREAVATIY
TRGRSIYYADSVKGRFTISQDNAKNTLYLQMNSLKPEDIAMYSCAAGGY
SWSAGCEFNYWGQGTQVTVSSGGGSQVQLQESGGGSVQVGGSLRLSCAT
SGDTKSIRCMGWFRQTPGKEREGIAAIDREGFATYADSVYDRFTIAQDN
AQNTLYLEMNALKPEDTAMYYCAAQNMCRVVRGAMTGVDYWGKGTQVTV
SSASHHHHHH
> SEQ ID NO: 207; DR669(DR232-DR588)
QVQLQESGGGSVQAGGSLRLSCVASGYTSCMGWFRQAPGKEREAVATIY
TRGRSIYYADSVKGRFTISQDNAKNTLYLQMNSLKPEDIAMYSCAAGGY
SWSAGCEFNYWGQGTQVTVSSGGGSQVQLQESGGGSVQVGGSLKLSCAA
SGYTYSSYYCMGWFRQAPGKEREGVAAIDSDGSTSYADSVKGRFTISQD
DAKNTLYLQMNSLKPEDTAMYYCAASYEVVDCYPSGYGQDYWGKGTQVT
VSSASHHHHHH
> SEQ ID NO: 208; DR670(DR232-DR589)
QVQLQESGGGSVQAGGSLRLSCVASGYTSCMGWFRQAPGKEREAVATIY
TRGRSIYYADSVKGRFTISQDNAKNTLYLQMNSLKPEDIAMYSCAAGGY
SWSAGCEFNYWGQGTQVTVSSGGGSQVQLQESGGGSVQAGGSLRLSCAA
SEYTASRYCMAWFRQAPGKEREGVAAIHPGGGTTYYADSVKGRFSISQD
SADNTLYLQMNSLKPEDTAMYYCAAGSLWVPFGDRCAANYWGQGTQVTV
SSASHHHHHH
> SEQ ID NO: 209; DR671(DR232-DR590)
QVQLQESGGGSVQAGGSLRLSCVASGYTSCMGWFRQAPGKEREAVATIY
TRGRSIYYADSVKGRFTISQDNAKNTLYLQMNSLKPEDIAMYSCAAGGY
SWSAGCEFNYWGQGTQVTVSSGGGSQVQLQESGGGSVQAGGSLRLSCAA
SGYEYCRIHMTWYRQGPGKEREFVSSIGSDGRKTYANSVTGRFTISRDN
ANHTVYLQMNSLSPEDTAMYYCKTEYLYGLGCPDGSAYWGQGTQVTVSS
ASHHHHHH
> SEQ ID NO: 210; DR672(DR233-DR214)
QVQLQESGGGSVQAGGSLRLSCTASGFTFDDSDMGWYRQAPGNECELVS
TISSDGSTYYADSVKGRFTISQDNAKNTVYLQMNSLKPEDTAVYYCAAE
PRGYYSNYGGRRECNYWGQGTQVTVSSGGGSQVQLLESGGGLVQPGGSL
RLSCAASGVRISTQDMSWVRQAPGKGLEWVSSILTPNGSTYYADSVKGR
FTISRDNSKNTLYLQMNSLRAEDTAVYYCAGVDERCEAEDQIDYWGQGT
LVTVSSASHHHHHH
> SEQ ID NO: 211; DR673(DR233-DR217)
QVQLQESGGGSVQAGGSLRLSCTASGFTFDDSDMGWYRQAPGNECELVS
TISSDGSTYYADSVKGRFTISQDNAKNTVYLQMNSLKPEDTAVYYCAAE
PRGYYSNYGGRRECNYWGQGTQVTVSSGGGSQVQLLESGGGLVQPGGSL
RLSCAASGDMIISEDMSWVRQAPGKGLEWVSTIASDDGSTYYADSVKGR
FTISRDNSKNTLYLQMNSLRAEDTAVYYCAGFDAQDAAIEYWGQGTLVT
VSSASHHHHHH
> SEQ ID NO: 212; DR674(DR233-DR583)
QVQLQESGGGSVQAGGSLRLSCTASGFTFDDSDMGWYRQAPGNECELVS
TISSDGSTYYADSVKGRFTISQDNAKNTVYLQMNSLKPEDTAVYYCAAE
PRGYYSNYGGRRECNYWGQGTQVTVSSGGGSQVQLQESGGGSVQAGGSL
RLSCVGSGYTYDTSDMSWYRQAPGKEREFVSDIDSGDWAAYADAVKGRF
TISRDNAKKTVYLQMNSLEPEDTAMYYCKASYWKWGKLNNFWGPGTQVT
VSSASHHHHHH
> SEQ ID NO: 213; DR675(DR233-DR584)
QVQLQESGGGSVQAGGSLRLSCTASGFTFDDSDMGWYRQAPGNECELVS
TISSDGSTYYADSVKGRFTISQDNAKNTVYLQMNSLKPEDTAVYYCAAE
PRGYYSNYGGRRECNYWGQGTQVTVSSGGGSQVQLQESGGGLVQPGGSL
KLSCAASGFRFSNYGMSWVRQAPGEGLEWVSYINGDGSRTHYADSVKGR
FTISRDNAKNTLYLQLNSLKTEDTAMYYCEKGLSRDGWSLSAASRGQGT
QVTVSSASHHHHHH
> SEQ ID NO: 214; DR676(DR233-DR585)
QVQLQESGGGSVQAGGSLRLSCTASGFTFDDSDMGWYRQAPGNECELVS
TISSDGSTYYADSVKGRFTISQDNAKNTVYLQMNSLKPEDTAVYYCAAE
PRGYYSNYGGRRECNYWGQGTQVTVSSGGGSQVQLQESGGGSVQTGGSL
RLSCAVSGYTTYSFNYMGWFRQAPGKEREGVAVIYTGGGSTLYADSVKG
RFTISQDNAKNTVYLQMNSLKPEDTAMYYCAADDQRFASPLYAYFGYWG
QGTQVTVSSASHHHHHH
> SEQ ID NO: 215; DR677(DR233-DR586)
QVQLQESGGGSVQAGGSLRLSCTASGFTFDDSDMGWYRQAPGNECELVS
TISSDGSTYYADSVKGRFTISQDNAKNTVYLQMNSLKPEDTAVYYCAAE
PRGYYSNYGGRRECNYWGQGTQVTVSSGGGSQVQLQESGGGLVQPGGSL
RLSCVASGFTFSNYWIFWVRQAAGKGLEWLSTSNTGGDTTKYADSVKGR
FTISRDSAKNTEYLQMNSLKPEDTAVYYCETGRCARSGGYQGTQVTVSS
ASHHHHHH
> SEQ ID NO: 216; DR678(DR233-DR587)
QVQLQESGGGSVQAGGSLRLSCTASGFTFDDSDMGWYRQAPGNECELVS
TISSDGSTYYADSVKGRFTISQDNAKNTVYLQMNSLKPEDTAVYYCAAE
PRGYYSNYGGRRECNYWGQGTQVTVSSGGGSQVQLQESGGGSVQVGGSL
RLSCATSGDTKSIRCMGWFRQTPGKEREGIAAIDREGFATYADSVYDRF
TIAQDNAQNTLYLEMNALKPEDTAMYYCAAQNMCRVVRGAMTGVDYWGK
GTQVTVSSASHHHHHH
> SEQ ID NO: 217; DR679(DR233-DR588)
QVQLQESGGGSVQAGGSLRLSCTASGFTFDDSDMGWYRQAPGNECELVS
TISSDGSTYYADSVKGRFTISQDNAKNTVYLQMNSLKPEDTAVYYCAAE
PRGYYSNYGGRRECNYWGQGTQVTVSSGGGSQVQLQESGGGSVQVGGSL
KLSCAASGYTYSSYYCMGWFRQAPGKEREGVAAIDSDGSTSYADSVKGR
FTISQDDAKNTLYLQMNSLKPEDTAMYYCAASYEVVDCYPSGYGQDYWG
KGTQVTVSSASHHHHHH
> SEQ ID NO: 218; DR680(DR233-DR589)
QVQLQESGGGSVQAGGSLRLSCTASGFTFDDSDMGWYRQAPGNECELVS
TISSDGSTYYADSVKGRFTISQDNAKNTVYLQMNSLKPEDTAVYYCAAE
PRGYYSNYGGRRECNYWGQGTQVTVSSGGGSQVQLQESGGGSVQAGGSL
RLSCAASEYTASRYCMAWFRQAPGKEREGVAAIHPGGGTTYYADSVKGR
FSISQDSADNTLYLQMNSLKPEDTAMYYCAAGSLWVPFGDRCAANYWGQ
GTQVTVSSASHHHHHH
> SEQ ID NO: 219; DR681(DR233-DR590)
QVQLQESGGGSVQAGGSLRLSCTASGFTFDDSDMGWYRQAPGNECELVS
TISSDGSTYYADSVKGRFTISQDNAKNTVYLQMNSLKPEDTAVYYCAAE
PRGYYSNYGGRRECNYWGQGTQVTVSSGGGSQVQLQESGGGSVQAGGSL
RLSCAASGYEYCRIHMTWYRQGPGKEREFVSSIGSDGRKTYANSVTGRF
TISRDNANHTVYLQMNSLSPEDTAMYYCKTEYLYGLGCPDGSAYWGQGT
QVTVSSASHHHHHH
> SEQ ID NO: 220; DR682(DR234-DR214)
QVQLQESGGGSVQAGGSLRLSCVASGYTFSSYCMGWFRQAPGKEREGVA
ALGGGSTYYADSVKGRFTISQDNAKNTLYLQMNSLKPEDTAMYYCAAAW
VACLEFGGSWYDLARYKHWGQGTQVTVSSGGGSQVQLLESGGGLVQPGG
SLRLSCAASGVRISTQDMSWVRQAPGKGLEWVSSILTPNGSTYYADSVK
GRFTISRDNSKNTLYLQMNSLRAEDTAVYYCAGVDERCEAEDQIDYWGQ
GTLVTVSSASHHHHHH
> SEQ ID NO: 221; DR683(DR234-DR217)
QVQLQESGGGSVQAGGSLRLSCVASGYTFSSYCMGWFRQAPGKEREGVA
ALGGGSTYYADSVKGRFTISQDNAKNTLYLQMNSLKPEDTAMYYCAAAW
VACLEFGGSWYDLARYKHWGQGTQVTVSSGGGSQVQLLESGGGLVQPGG
SLRLSCAASGDMIISEDMSWVRQAPGKGLEWVSTIASDDGSTYYADSVK
GRFTISRDNSKNTLYLQMNSLRAEDTAVYYCAGFDAQDAAIEYWGQGTL
VTVSSASHHHHHH
> SEQ ID NO: 222; DR684(DR234-DR583)
QVQLQESGGGSVQAGGSLRLSCVASGYTFSSYCMGWFRQAPGKEREGVA
ALGGGSTYYADSVKGRFTISQDNAKNTLYLQMNSLKPEDTAMYYCAAAW
VACLEFGGSWYDLARYKHWGQGTQVTVSSGGGSQVQLQESGGGSVQAGG
SLRLSCVGSGYTYDTSDMSWYRQAPGKEREFVSDIDSGDWAAYADAVKG
RFTISRDNAKKTVYLQMNSLEPEDTAMYYCKASYWKWGKLNNFWGPGTQ
VTVSSASHHHHHH
> SEQ ID NO: 223; DR685(DR234-DR584)
QVQLQESGGGSVQAGGSLRLSCVASGYTFSSYCMGWFRQAPGKEREGVA
ALGGGSTYYADSVKGRFTISQDNAKNTLYLQMNSLKPEDTAMYYCAAAW
VACLEFGGSWYDLARYKHWGQGTQVTVSSGGGSQVQLQESGGGLVQPGG
SLKLSCAASGFRFSNYGMSWVRQAPGEGLEWVSYINGDGSRTHYADSVK
GRFTISRDNAKNTLYLQLNSLKTEDTAMYYCEKGLSRDGWSLSAASRGQ
GTQVTVSSASHHHHHH
> SEQ ID NO: 224; DR686(DR234-DR585)
QVQLQESGGGSVQAGGSLRLSCVASGYTFSSYCMGWFRQAPGKEREGVA
ALGGGSTYYADSVKGRFTISQDNAKNTLYLQMNSLKPEDTAMYYCAAAW
VACLEFGGSWYDLARYKHWGQGTQVTVSSGGGSQVQLQESGGGSVQTGG
SLRLSCAVSGYTTYSFNYMGWFRQAPGKEREGVAVIYTGGGSTLYADSV
KGRFTISQDNAKNTVYLQMNSLKPEDTAMYYCAADDQRFASPLYAYFGY
WGQGTQVTVSSASHHHHHH
> SEQ ID NO: 225; DR687(DR234-DR586)
QVQLQESGGGSVQAGGSLRLSCVASGYTFSSYCMGWFRQAPGKEREGVA
ALGGGSTYYADSVKGRFTISQDNAKNTLYLQMNSLKPEDTAMYYCAAAW
VACLEFGGSWYDLARYKHWGQGTQVTVSSGGGSQVQLQESGGGLVQPGG
SLRLSCVASGFTFSNYWIFWVRQAAGKGLEWLSTSNTGGDTTKYADSVK
GRFTISRDSAKNTEYLQMNSLKPEDTAVYYCETGRCARSGGYQGTQVTV
SSASHHHHHH
> SEQ ID NO: 226; DR688(DR234-DR587)
QVQLQESGGGSVQAGGSLRLSCVASGYTFSSYCMGWFRQAPGKEREGVA
ALGGGSTYYADSVKGRFTISQDNAKNTLYLQMNSLKPEDTAMYYCAAAW
VACLEFGGSWYDLARYKHWGQGTQVTVSSGGGSQVQLQESGGGSVQVGG
SLRLSCATSGDTKSIRCMGWFRQTPGKEREGIAAIDREGFATYADSVYD
RFTIAQDNAQNTLYLEMNALKPEDTAMYYCAAQNMCRVVRGAMTGVDYW
GKGTQVTVSSASHHHHHH
> SEQ ID NO: 227; DR689(DR234-DR588)
QVQLQESGGGSVQAGGSLRLSCVASGYTFSSYCMGWFRQAPGKEREGVA
ALGGGSTYYADSVKGRFTISQDNAKNTLYLQMNSLKPEDTAMYYCAAAW
VACLEFGGSWYDLARYKHWGQGTQVTVSSGGGSQVQLQESGGGSVQVGG
SLKLSCAASGYTYSSYYCMGWFRQAPGKEREGVAAIDSDGSTSYADSVK
GRFTISQDDAKNTLYLQMNSLKPEDTAMYYCAASYEVVDCYPSGYGQDY
WGKGTQVTVSSASHHHHHH
> SEQ ID NO: 228; DR690(DR234-DR589)
QVQLQESGGGSVQAGGSLRLSCVASGYTFSSYCMGWFRQAPGKEREGVA
ALGGGSTYYADSVKGRFTISQDNAKNTLYLQMNSLKPEDTAMYYCAAAW
VACLEFGGSWYDLARYKHWGQGTQVTVSSGGGSQVQLQESGGGSVQAGG
SLRLSCAASEYTASRYCMAWFRQAPGKEREGVAAIHPGGGTTYYADSVK
GRFSISQDSADNTLYLQMNSLKPEDTAMYYCAAGSLWVPFGDRCAANYW
GQGTQVTVSSASHHHHHH
> SEQ ID NO: 229; DR691(DR234-DR590)
QVQLQESGGGSVQAGGSLRLSCVASGYTFSSYCMGWFRQAPGKEREGVA
ALGGGSTYYADSVKGRFTISQDNAKNTLYLQMNSLKPEDTAMYYCAAAW
VACLEFGGSWYDLARYKHWGQGTQVTVSSGGGSQVQLQESGGGSVQAGG
SLRLSCAASGYEYCRIHMTWYRQGPGKEREFVSSIGSDGRKTYANSVTG
RFTISRDNANHTVYLQMNSLSPEDTAMYYCKTEYLYGLGCPDGSAYWGQ
GTQVTVSSASHHHHHH
> SEQ ID NO: 230; DR692(DR214-DR229)
QVQLLESGGGLVQPGGSLRLSCAASGVRISTQDMSWVRQAPGKGLEWVS
SILTPNGSTYYADSVKGRFTISRDNSKNTLYLQMNSLRAEDTAVYYCAG
VDERCEAEDQIDYWGQGTLVTVSSGGGSQVQLQESGGGLVQPGGSLRLS
CTASGFSFSSYPMTWARQAPGKGLEWVSTIASDGGSTAYAASVEGRFTI
SRDNAKSTLYLQLNSLKTEDTAMYYCTKGYGDGTPAPGQGTQVTVSSAS
HHHHHH
> SEQ ID NO: 231; DR693(DR214-DR230)
QVQLLESGGGLVQPGGSLRLSCAASGVRISTQDMSWVRQAPGKGLEWVS
SILTPNGSTYYADSVKGRFTISRDNSKNTLYLQMNSLRAEDTAVYYCAG
VDERCEAEDQIDYWGQGTLVTVSSGGGSQVQLQESGGGLVQPGGSLRLS
CAASGFTFSSAHMSWVRQAPGKGREWIASIYSGGGTFYADSVKGRFTIS
RDNAKNTLYLQLNSLKAEDTAMYYCATNRLHYYSDDDSLRGQGTQVTVS
SASHHHHHH
> SEQ ID NO: 232; DR694(DR214-DR231)
QVQLLESGGGLVQPGGSLRLSCAASGVRISTQDMSWVRQAPGKGLEWVS
SILTPNGSTYYADSVKGRFTISRDNSKNTLYLQMNSLRAEDTAVYYCAG
VDERCEAEDQIDYWGQGTLVTVSSGGGSQVQLQESGGGSVQAGGSLRLS
CTASGFTFDDREMNWYRQAPGNECELVSTISSDGSTYYADSVKGRFTIS
QDNAKNTVYLQMDSVKPEDTAVYYCAADFMIAIQAPGAGCWGQGTQVTV
SSASHHHHHH
> SEQ ID NO: 233; DR695(DR214-DR232)
QVQLLESGGGLVQPGGSLRLSCAASGVRISTQDMSWVRQAPGKGLEWVS
SILTPNGSTYYADSVKGRFTISRDNSKNTLYLQMNSLRAEDTAVYYCAG
VDERCEAEDQIDYWGQGTLVTVSSGGGSQVQLQESGGGSVQAGGSLRLS
CVASGYTSCMGWFRQAPGKEREAVATIYTRGRSIYYADSVKGRFTISQD
NAKNTLYLQMNSLKPEDIAMYSCAAGGYSWSAGCEFNYWGQGTQVTVSS
ASHHHHHH
> SEQ ID NO: 234; DR696(DR214-DR233)
QVQLLESGGGLVQPGGSLRLSCAASGVRISTQDMSWVRQAPGKGLEWVS
SILTPNGSTYYADSVKGRFTISRDNSKNTLYLQMNSLRAEDTAVYYCAG
VDERCEAEDQIDYWGQGTLVTVSSGGGSQVQLQESGGGSVQAGGSLRLS
CTASGFTFDDSDMGWYRQAPGNECELVSTISSDGSTYYADSVKGRFTIS
QDNAKNTVYLQMNSLKPEDTAVYYCAAEPRGYYSNYGGRRECNYWGQGT
QVTVSSASHHHHHH
> SEQ ID NO: 235; DR697(DR214-DR234)
QVQLLESGGGLVQPGGSLRLSCAASGVRISTQDMSWVRQAPGKGLEWVS
SILTPNGSTYYADSVKGRFTISRDNSKNTLYLQMNSLRAEDTAVYYCAG
VDERCEAEDQIDYWGQGTLVTVSSGGGSQVQLQESGGGSVQAGGSLRLS
CVASGYTFSSYCMGWFRQAPGKEREGVAALGGGSTYYADSVKGRFTISQ
DNAKNTLYLQMNSLKPEDTAMYYCAAAWVACLEFGGSWYDLARYKHWGQ
GTQVTVSSASHHHHHH
> SEQ ID NO: 236; DR698(DR217-DR229)
QVQLLESGGGLVQPGGSLRLSCAASGDMIISEDMSWVRQAPGKGLEWVS
TIASDDGSTYYADSVKGRFTISRDNSKNTLYLQMNSLRAEDTAVYYCAG
FDAQDAAIEYWGQGTLVTVSSGGGSQVQLQESGGGLVQPGGSLRLSCTA
SGFSFSSYPMTWARQAPGKGLEWVSTIASDGGSTAYAASVEGRFTISRD
NAKSTLYLQLNSLKTEDTAMYYCTKGYGDGTPAPGQGTQVTVSSASHHH
HHH
> SEQ ID NO: 237; DR699(DR217-DR230)
QVQLLESGGGLVQPGGSLRLSCAASGDMIISEDMSWVRQAPGKGLEWVS
TIASDDGSTYYADSVKGRFTISRDNSKNTLYLQMNSLRAEDTAVYYCAG
FDAQDAAIEYWGQGTLVTVSSGGGSQVQLQESGGGLVQPGGSLRLSCAA
SGFTFSSAHMSWVRQAPGKGREWIASIYSGGGTFYADSVKGRFTISRDN
AKNTLYLQLNSLKAEDTAMYYCATNRLHYYSDDDSLRGQGTQVTVSSAS
HHHHHH
> SEQ ID NO: 238; DR700(DR217-DR231)
QVQLLESGGGLVQPGGSLRLSCAASGDMIISEDMSWVRQAPGKGLEWVS
TIASDDGSTYYADSVKGRFTISRDNSKNTLYLQMNSLRAEDTAVYYCAG
FDAQDAAIEYWGQGTLVTVSSGGGSQVQLQESGGGSVQAGGSLRLSCTA
SGFTFDDREMNWYRQAPGNECELVSTISSDGSTYYADSVKGRFTISQDN
AKNTVYLQMDSVKPEDTAVYYCAADFMIAIQAPGAGCWGQGTQVTVSSA
SHHHHHH
> SEQ ID NO: 239; DR701(DR217-DR232)
QVQLLESGGGLVQPGGSLRLSCAASGDMIISEDMSWVRQAPGKGLEWVS
TIASDDGSTYYADSVKGRFTISRDNSKNTLYLQMNSLRAEDTAVYYCAG
FDAQDAAIEYWGQGTLVTVSSGGGSQVQLQESGGGSVQAGGSLRLSCVA
SGYTSCMGWFRQAPGKEREAVATIYTRGRSIYYADSVKGRFTISQDNAK
NTLYLQMNSLKPEDIAMYSCAAGGYSWSAGCEFNYWGQGTQVTVSSASH
HHHHH
> SEQ ID NO: 240; DR702(DR217-DR233)
QVQLLESGGGLVQPGGSLRLSCAASGDMIISEDMSWVRQAPGKGLEWVS
TIASDDGSTYYADSVKGRFTISRDNSKNTLYLQMNSLRAEDTAVYYCAG
FDAQDAAIEYWGQGTLVTVSSGGGSQVQLQESGGGSVQAGGSLRLSCTA
SGFTFDDSDMGWYRQAPGNECELVSTISSDGSTYYADSVKGRFTISQDN
AKNTVYLQMNSLKPEDTAVYYCAAEPRGYYSNYGGRRECNYWGQGTQVT
VSSASHHHHHH
> SEQ ID NO: 241; DR703(DR217-DR234)
QVQLLESGGGLVQPGGSLRLSCAASGDMIISEDMSWVRQAPGKGLEWVS
TIASDDGSTYYADSVKGRFTISRDNSKNTLYLQMNSLRAEDTAVYYCAG
FDAQDAAIEYWGQGTLVTVSSGGGSQVQLQESGGGSVQAGGSLRLSCVA
SGYTFSSYCMGWFRQAPGKEREGVAALGGGSTYYADSVKGRFTISQDNA
KNTLYLQMNSLKPEDTAMYYCAAAWVACLEFGGSWYDLARYKHWGQGTQ
VTVSSASHHHHHH
> SEQ ID NO: 242; DR704(DR583-DR229)
QVQLQESGGGSVQAGGSLRLSCVGSGYTYDTSDMSWYRQAPGKEREFVS
DIDSGDWAAYADAVKGRFTISRDNAKKTVYLQMNSLEPEDTAMYYCKAS
YWKWGKLNNFWGPGTQVTVSSGGGSQVQLQESGGGLVQPGGSLRLSCTA
SGFSFSSYPMTWARQAPGKGLEWVSTIASDGGSTAYAASVEGRFTISRD
NAKSTLYLQLNSLKTEDTAMYYCTKGYGDGTPAPGQGTQVTVSSASHHH
HHH
> SEQ ID NO: 243; DR705(DR583-DR230)
QVQLQESGGGSVQAGGSLRLSCVGSGYTYDTSDMSWYRQAPGKEREFVS
DIDSGDWAAYADAVKGRFTISRDNAKKTVYLQMNSLEPEDTAMYYCKAS
YWKWGKLNNFWGPGTQVTVSSGGGSQVQLQESGGGLVQPGGSLRLSCAA
SGFTFSSAHMSWVRQAPGKGREWIASIYSGGGTFYADSVKGRFTISRDN
AKNTLYLQLNSLKAEDTAMYYCATNRLHYYSDDDSLRGQGTQVTVSSAS
HHHHHH
> SEQ ID NO: 244; DR706(DR583-DR231)
QVQLQESGGGSVQAGGSLRLSCVGSGYTYDTSDMSWYRQAPGKEREFVS
DIDSGDWAAYADAVKGRFTISRDNAKKTVYLQMNSLEPEDTAMYYCKAS
YWKWGKLNNFWGPGTQVTVSSGGGSQVQLQESGGGSVQAGGSLRLSCTA
SGFTFDDREMNWYRQAPGNECELVSTISSDGSTYYADSVKGRFTISQDN
AKNTVYLQMDSVKPEDTAVYYCAADFMIAIQAPGAGCWGQGTQVTVSSA
SHHHHHH
> SEQ ID NO: 245; DR707(DR583-DR232)
QVQLQESGGGSVQAGGSLRLSCVGSGYTYDTSDMSWYRQAPGKEREFVS
DIDSGDWAAYADAVKGRFTISRDNAKKTVYLQMNSLEPEDTAMYYCKAS
YWKWGKLNNFWGPGTQVTVSSGGGSQVQLQESGGGSVQAGGSLRLSCVA
SGYTSCMGWFRQAPGKEREAVATIYTRGRSIYYADSVKGRFTISQDNAK
NTLYLQMNSLKPEDIAMYSCAAGGYSWSAGCEFNYWGQGTQVTVSSASH
HHHHH
> SEQ ID NO: 246; DR708(DR583-DR233)
QVQLQESGGGSVQAGGSLRLSCVGSGYTYDTSDMSWYRQAPGKEREFVS
DIDSGDWAAYADAVKGRFTISRDNAKKTVYLQMNSLEPEDTAMYYCKAS
YWKWGKLNNFWGPGTQVTVSSGGGSQVQLQESGGGSVQAGGSLRLSCTA
SGFTFDDSDMGWYRQAPGNECELVSTISSDGSTYYADSVKGRFTISQDN
AKNTVYLQMNSLKPEDTAVYYCAAEPRGYYSNYGGRRECNYWGQGTQVT
VSSASHHHHHH
> SEQ ID NO: 247; DR709(DR583-DR234)
QVQLQESGGGSVQAGGSLRLSCVGSGYTYDTSDMSWYRQAPGKEREFVS
DIDSGDWAAYADAVKGRFTISRDNAKKTVYLQMNSLEPEDTAMYYCKAS
YWKWGKLNNFWGPGTQVTVSSGGGSQVQLQESGGGSVQAGGSLRLSCVA
SGYTFSSYCMGWFRQAPGKEREGVAALGGGSTYYADSVKGRFTISQDNA
KNTLYLQMNSLKPEDTAMYYCAAAWVACLEFGGSWYDLARYKHWGQGTQ
VTVSSASHHHHHH
> SEQ ID NO: 248; DR710(DR584-DR229)
QVQLQESGGGLVQPGGSLKLSCAASGFRFSNYGMSWVRQAPGEGLEWVS
YINGDGSRTHYADSVKGRFTISRDNAKNTLYLQLNSLKTEDTAMYYCEK
GLSRDGWSLSAASRGQGTQVTVSSGGGSQVQLQESGGGLVQPGGSLRLS
CTASGFSFSSYPMTWARQAPGKGLEWVSTIASDGGSTAYAASVEGRFTI
SRDNAKSTLYLQLNSLKTEDTAMYYCTKGYGDGTPAPGQGTQVTVSSAS
HHHHHH
> SEQ ID NO: 249; DR711(DR584-DR230)
QVQLQESGGGLVQPGGSLKLSCAASGFRFSNYGMSWVRQAPGEGLEWVS
YINGDGSRTHYADSVKGRFTISRDNAKNTLYLQLNSLKTEDTAMYYCEK
GLSRDGWSLSAASRGQGTQVTVSSGGGSQVQLQESGGGLVQPGGSLRLS
CAASGFTFSSAHMSWVRQAPGKGREWIASIYSGGGTFYADSVKGRFTIS
RDNAKNTLYLQLNSLKAEDTAMYYCATNRLHYYSDDDSLRGQGTQVTVS
SASHHHHHH
> SEQ ID NO: 250; DR712(DR584-DR231)
QVQLQESGGGLVQPGGSLKLSCAASGFRFSNYGMSWVRQAPGEGLEWVS
YINGDGSRTHYADSVKGRFTISRDNAKNTLYLQLNSLKTEDTAMYYCEK
GLSRDGWSLSAASRGQGTQVTVSSGGGSQVQLQESGGGSVQAGGSLRLS
CTASGFTFDDREMNWYRQAPGNECELVSTISSDGSTYYADSVKGRFTIS
QDNAKNTVYLQMDSVKPEDTAVYYCAADFMIAIQAPGAGCWGQGTQVTV
SSASHHHHHH
> SEQ ID NO: 251; DR713(DR584-DR232)
QVQLQESGGGLVQPGGSLKLSCAASGFRFSNYGMSWVRQAPGEGLEWVS
YINGDGSRTHYADSVKGRFTISRDNAKNTLYLQLNSLKTEDTAMYYCEK
GLSRDGWSLSAASRGQGTQVTVSSGGGSQVQLQESGGGSVQAGGSLRLS
CVASGYTSCMGWFRQAPGKEREAVATIYTRGRSIYYADSVKGRFTISQD
NAKNTLYLQMNSLKPEDIAMYSCAAGGYSWSAGCEFNYWGQGTQVTVSS
ASHHHHHH
> SEQ ID NO: 252; DR714(DR584-DR233)
QVQLQESGGGLVQPGGSLKLSCAASGFRFSNYGMSWVRQAPGEGLEWVS
YINGDGSRTHYADSVKGRFTISRDNAKNTLYLQLNSLKTEDTAMYYCEK
GLSRDGWSLSAASRGQGTQVTVSSGGGSQVQLQESGGGSVQAGGSLRLS
CTASGFTFDDSDMGWYRQAPGNECELVSTISSDGSTYYADSVKGRFTIS
QDNAKNTVYLQMNSLKPEDTAVYYCAAEPRGYYSNYGGRRECNYWGQGT
QVTVSSASHHHHHH
> SEQ ID NO: 253; DR715(DR584-DR234)
QVQLQESGGGLVQPGGSLKLSCAASGFRFSNYGMSWVRQAPGEGLEWVS
YINGDGSRTHYADSVKGRFTISRDNAKNTLYLQLNSLKTEDTAMYYCEK
GLSRDGWSLSAASRGQGTQVTVSSGGGSQVQLQESGGGSVQAGGSLRLS
CVASGYTFSSYCMGWFRQAPGKEREGVAALGGGSTYYADSVKGRFTISQ
DNAKNTLYLQMNSLKPEDTAMYYCAAAWVACLEFGGSWYDLARYKHWGQ
GTQVTVSSASHHHHHH
> SEQ ID NO: 254 DR716(DR585-DR229)
QVQLQESGGGSVQTGGSLRLSCAVSGYTTYSFNYMGWFRQAPGKEREGV
AVIYTGGGSTLYADSVKGRFTISQDNAKNTVYLQMNSLKPEDTAMYYCA
ADDQRFASPLYAYFGYWGQGTQVTVSSGGGSQVQLQESGGGLVQPGGSL
RLSCTASGFSFSSYPMTWARQAPGKGLEWVSTIASDGGSTAYAASVEGR
FTISRDNAKSTLYLQLNSLKTEDTAMYYCTKGYGDGTPAPGQGTQVTVS
SASHHHHHH
> SEQ ID NO: 255; DR717(DR585-DR230)
QVQLQESGGGSVQTGGSLRLSCAVSGYTTYSFNYMGWFRQAPGKEREGV
AVIYTGGGSTLYADSVKGRFTISQDNAKNTVYLQMNSLKPEDTAMYYCA
ADDQRFASPLYAYFGYWGQGTQVTVSSGGGSQVQLQESGGGLVQPGGSL
RLSCAASGFTFSSAHMSWVRQAPGKGREWIASIYSGGGTFYADSVKGRF
TISRDNAKNTLYLQLNSLKAEDTAMYYCATNRLHYYSDDDSLRGQGTQV
TVSSASHHHHHH
> SEQ ID NO: 256; DR718(DR585-DR231)
QVQLQESGGGSVQTGGSLRLSCAVSGYTTYSFNYMGWFRQAPGKEREGV
AVIYTGGGSTLYADSVKGRFTISQDNAKNTVYLQMNSLKPEDTAMYYCA
ADDQRFASPLYAYFGYWGQGTQVTVSSGGGSQVQLQESGGGSVQAGGSL
RLSCTASGFTFDDREMNWYRQAPGNECELVSTISSDGSTYYADSVKGRF
TISQDNAKNTVYLQMDSVKPEDTAVYYCAADFMIAIQAPGAGCWGQGTQ
VTVSSASHHHHHH
> SEQ ID NO: 257; DR719(DR585-DR232)
QVQLQESGGGSVQTGGSLRLSCAVSGYTTYSFNYMGWFRQAPGKEREGV
AVIYTGGGSTLYADSVKGRFTISQDNAKNTVYLQMNSLKPEDTAMYYCA
ADDQRFASPLYAYFGYWGQGTQVTVSSGGGSQVQLQESGGGSVQAGGSL
RLSCVASGYTSCMGWFRQAPGKEREAVATIYTRGRSIYYADSVKGRFTI
SQDNAKNTLYLQMNSLKPEDIAMYSCAAGGYSWSAGCEFNYWGQGTQVT
VSSASHHHHHH
> SEQ ID NO: 258; DR720(DR585-DR233)
QVQLQESGGGSVQTGGSLRLSCAVSGYTTYSFNYMGWFRQAPGKEREGV
AVIYTGGGSTLYADSVKGRFTISQDNAKNTVYLQMNSLKPEDTAMYYCA
ADDQRFASPLYAYFGYWGQGTQVTVSSGGGSQVQLQESGGGSVQAGGSL
RLSCTASGFTFDDSDMGWYRQAPGNECELVSTISSDGSTYYADSVKGRF
TISQDNAKNTVYLQMNSLKPEDTAVYYCAAEPRGYYSNYGGRRECNYWG
QGTQVTVSSASHHHHHH
> SEQ ID NO: 259; DR721(DR585-DR234)
QVQLQESGGGSVQTGGSLRLSCAVSGYTTYSFNYMGWFRQAPGKEREGV
AVIYTGGGSTLYADSVKGRFTISQDNAKNTVYLQMNSLKPEDTAMYYCA
ADDQRFASPLYAYFGYWGQGTQVTVSSGGGSQVQLQESGGGSVQAGGSL
RLSCVASGYTFSSYCMGWFRQAPGKEREGVAALGGGSTYYADSVKGRFT
ISQDNAKNTLYLQMNSLKPEDTAMYYCAAAWVACLEFGGSWYDLARYKH
WGQGTQVTVSSASHHHHHH
> SEQ ID NO: 260; DR722(DR586-DR229)
QVQLQESGGGLVQPGGSLRLSCVASGFTFSNYWIFWVRQAAGKGLEWLS
TSNTGGDTTKYADSVKGRFTISRDSAKNTEYLQMNSLKPEDTAVYYCET
GRCARSGGYQGTQVTVSSGGGSQVQLQESGGGLVQPGGSLRLSCTASGF
SFSSYPMTWARQAPGKGLEWVSTIASDGGSTAYAASVEGRFTISRDNAK
STLYLQLNSLKTEDTAMYYCTKGYGDGTPAPGQGTQVTVSSASHHHHHH
> SEQ ID NO: 261; DR723(DR586-DR230)
QVQLQESGGGLVQPGGSLRLSCVASGFTFSNYWIFWVRQAAGKGLEWLS
TSNTGGDTTKYADSVKGRFTISRDSAKNTEYLQMNSLKPEDTAVYYCET
GRCARSGGYQGTQVTVSSGGGSQVQLQESGGGLVQPGGSLRLSCAASGF
TFSSAHMSWVRQAPGKGREWIASIYSGGGTFYADSVKGRFTISRDNAKN
TLYLQLNSLKAEDTAMYYCATNRLHYYSDDDSLRGQGTQVTVSSASHHH
HHH
> SEQ ID NO: 262; DR724(DR586-DR231)
QVQLQESGGGLVQPGGSLRLSCVASGFTFSNYWIFWVRQAAGKGLEWLS
TSNTGGDTTKYADSVKGRFTISRDSAKNTEYLQMNSLKPEDTAVYYCET
GRCARSGGYQGTQVTVSSGGGSQVQLQESGGGSVQAGGSLRLSCTASGF
TFDDREMNWYRQAPGNECELVSTISSDGSTYYADSVKGRFTISQDNAKN
TVYLQMDSVKPEDTAVYYCAADFMIAIQAPGAGCWGQGTQVTVSSASHH
HHHH
> SEQ ID NO: 263; DR725(DR586-DR232)
QVQLQESGGGLVQPGGSLRLSCVASGFTFSNYWIFWVRQAAGKGLEWLS
TSNTGGDTTKYADSVKGRFTISRDSAKNTEYLQMNSLKPEDTAVYYCET
GRCARSGGYQGTQVTVSSGGGSQVQLQESGGGSVQAGGSLRLSCVASGY
TSCMGWFRQAPGKEREAVATIYTRGRSIYYADSVKGRFTISQDNAKNTL
YLQMNSLKPEDIAMYSCAAGGYSWSAGCEFNYWGQGTQVTVSSASHHHH
HH
> SEQ ID NO: 264; DR726(DR586-DR233)
QVQLQESGGGLVQPGGSLRLSCVASGFTFSNYWIFWVRQAAGKGLEWLS
TSNTGGDTTKYADSVKGRFTISRDSAKNTEYLQMNSLKPEDTAVYYCET
GRCARSGGYQGTQVTVSSGGGSQVQLQESGGGSVQAGGSLRLSCTASGF
TFDDSDMGWYRQAPGNECELVSTISSDGSTYYADSVKGRFTISQDNAKN
TVYLQMNSLKPEDTAVYYCAAEPRGYYSNYGGRRECNYWGQGTQVTVSS
ASHHHHHH
> SEQ ID NO: 265; DR727(DR586-DR234)
QVQLQESGGGLVQPGGSLRLSCVASGFTFSNYWIFWVRQAAGKGLEWLS
TSNTGGDTTKYADSVKGRFTISRDSAKNTEYLQMNSLKPEDTAVYYCET
GRCARSGGYQGTQVTVSSGGGSQVQLQESGGGSVQAGGSLRLSCVASGY
TFSSYCMGWFRQAPGKEREGVAALGGGSTYYADSVKGRFTISQDNAKNT
LYLQMNSLKPEDTAMYYCAAAWVACLEFGGSWYDLARYKHWGQGTQVTV
SSASHHHHHH
> SEQ ID NO: 266; DR728(DR587-DR229)
QVQLQESGGGSVQVGGSLRLSCATSGDTKSIRCMGWFRQTPGKEREGIA
AIDREGFATYADSVYDRFTIAQDNAQNTLYLEMNALKPEDTAMYYCAAQ
NMCRVVRGAMTGVDYWGKGTQVTVSSGGGSQVQLQESGGGLVQPGGSLR
LSCTASGFSFSSYPMTWARQAPGKGLEWVSTIASDGGSTAYAASVEGRF
TISRDNAKSTLYLQLNSLKTEDTAMYYCTKGYGDGTPAPGQGTQVTVSS
ASHHHHHH
> SEQ ID NO: 267; DR729(DR587-DR230)
QVQLQESGGGSVQVGGSLRLSCATSGDTKSIRCMGWFRQTPGKEREGIA
AIDREGFATYADSVYDRFTIAQDNAQNTLYLEMNALKPEDTAMYYCAAQ
NMCRVVRGAMTGVDYWGKGTQVTVSSGGGSQVQLQESGGGLVQPGGSLR
LSCAASGFTFSSAHMSWVRQAPGKGREWIASIYSGGGTFYADSVKGRFT
ISRDNAKNTLYLQLNSLKAEDTAMYYCATNRLHYYSDDDSLRGQGTQVT
VSSASHHHHHH
> SEQ ID NO: 268; DR730(DR587-DR231)
QVQLQESGGGSVQVGGSLRLSCATSGDTKSIRCMGWFRQTPGKEREGIA
AIDREGFATYADSVYDRFTIAQDNAQNTLYLEMNALKPEDTAMYYCAAQ
NMCRVVRGAMTGVDYWGKGTQVTVSSGGGSQVQLQESGGGSVQAGGSLR
LSCTASGFTFDDREMNWYRQAPGNECELVSTISSDGSTYYADSVKGRFT
ISQDNAKNTVYLQMDSVKPEDTAVYYCAADFMIAIQAPGAGCWGQGTQV
TVSSASHHHHHH
> SEQ ID NO: 269; DR731(DR587-DR232)
QVQLQESGGGSVQVGGSLRLSCATSGDTKSIRCMGWFRQTPGKEREGIA
AIDREGFATYADSVYDRFTIAQDNAQNTLYLEMNALKPEDTAMYYCAAQ
NMCRVVRGAMTGVDYWGKGTQVTVSSGGGSQVQLQESGGGSVQAGGSLR
LSCVASGYTSCMGWFRQAPGKEREAVATIYTRGRSIYYADSVKGRFTIS
QDNAKNTLYLQMNSLKPEDIAMYSCAAGGYSWSAGCEFNYWGQGTQVTV
SSASHHHHHH
> SEQ ID NO: 270; DR732(DR587-DR233)
QVQLQESGGGSVQVGGSLRLSCATSGDTKSIRCMGWFRQTPGKEREGIA
AIDREGFATYADSVYDRFTIAQDNAQNTLYLEMNALKPEDTAMYYCAAQ
NMCRVVRGAMTGVDYWGKGTQVTVSSGGGSQVQLQESGGGSVQAGGSLR
LSCTASGFTFDDSDMGWYRQAPGNECELVSTISSDGSTYYADSVKGRFT
ISQDNAKNTVYLQMNSLKPEDTAVYYCAAEPRGYYSNYGGRRECNYWGQ
GTQVTVSSASHHHHHH
> SEQ ID NO: 271; DR733(DR587-DR234)
QVQLQESGGGSVQVGGSLRLSCATSGDTKSIRCMGWFRQTPGKEREGIA
AIDREGFATYADSVYDRFTIAQDNAQNTLYLEMNALKPEDTAMYYCAAQ
NMCRVVRGAMTGVDYWGKGTQVTVSSGGGSQVQLQESGGGSVQAGGSLR
LSCVASGYTFSSYCMGWFRQAPGKEREGVAALGGGSTYYADSVKGRFTI
SQDNAKNTLYLQMNSLKPEDTAMYYCAAAWVACLEFGGSWYDLARYKHW
GQGTQVTVSSASHHHHHH
> SEQ ID NO: 272; DR734(DR588-DR229)
QVQLQESGGGSVQVGGSLKLSCAASGYTYSSYYCMGWFRQAPGKEREGV
AAIDSDGSTSYADSVKGRFTISQDDAKNTLYLQMNSLKPEDTAMYYCAA
SYEVVDCYPSGYGQDYWGKGTQVTVSSGGGSQVQLQESGGGLVQPGGSL
RLSCTASGFSFSSYPMTWARQAPGKGLEWVSTIASDGGSTAYAASVEGR
FTISRDNAKSTLYLQLNSLKTEDTAMYYCTKGYGDGTPAPGQGTQVTVS
SASHHHHHH
> SEQ ID NO: 273; DR735(DR588-DR230)
QVQLQESGGGSVQVGGSLKLSCAASGYTYSSYYCMGWFRQAPGKEREGV
AAIDSDGSTSYADSVKGRFTISQDDAKNTLYLQMNSLKPEDTAMYYCAA
SYEVVDCYPSGYGQDYWGKGTQVTVSSGGGSQVQLQESGGGLVQPGGSL
RLSCAASGFTFSSAHMSWVRQAPGKGREWIASIYSGGGTFYADSVKGRF
TISRDNAKNTLYLQLNSLKAEDTAMYYCATNRLHYYSDDDSLRGQGTQV
TVSSASHHHHHH
> SEQ ID NO: 274; DR736(DR588-DR231)
QVQLQESGGGSVQVGGSLKLSCAASGYTYSSYYCMGWFRQAPGKEREGV
AAIDSDGSTSYADSVKGRFTISQDDAKNTLYLQMNSLKPEDTAMYYCAA
SYEVVDCYPSGYGQDYWGKGTQVTVSSGGGSQVQLQESGGGSVQAGGSL
RLSCTASGFTFDDREMNWYRQAPGNECELVSTISSDGSTYYADSVKGRF
TISQDNAKNTVYLQMDSVKPEDTAVYYCAADFMIAIQAPGAGCWGQGTQ
VTVSSASHHHHHH
> SEQ ID NO: 275; DR737(DR588-DR232)
QVQLQESGGGSVQVGGSLKLSCAASGYTYSSYYCMGWFRQAPGKEREGV
AAIDSDGSTSYADSVKGRFTISQDDAKNTLYLQMNSLKPEDTAMYYCAA
SYEVVDCYPSGYGQDYWGKGTQVTVSSGGGSQVQLQESGGGSVQAGGSL
RLSCVASGYTSCMGWFRQAPGKEREAVATIYTRGRSIYYADSVKGRFTI
SQDNAKNTLYLQMNSLKPEDIAMYSCAAGGYSWSAGCEFNYWGQGTQVT
VSSASHHHHHH
> SEQ ID NO: 276; DR738(DR588-DR233)
QVQLQESGGGSVQVGGSLKLSCAASGYTYSSYYCMGWFRQAPGKEREGV
AAIDSDGSTSYADSVKGRFTISQDDAKNTLYLQMNSLKPEDTAMYYCAA
SYEVVDCYPSGYGQDYWGKGTQVTVSSGGGSQVQLQESGGGSVQAGGSL
RLSCTASGFTFDDSDMGWYRQAPGNECELVSTISSDGSTYYADSVKGRF
TISQDNAKNTVYLQMNSLKPEDTAVYYCAAEPRGYYSNYGGRRECNYWG
QGTQVTVSSASHHHHHH
> SEQ ID NO: 277; DR739(DR588-DR234)
QVQLQESGGGSVQVGGSLKLSCAASGYTYSSYYCMGWFRQAPGKEREGV
AAIDSDGSTSYADSVKGRFTISQDDAKNTLYLQMNSLKPEDTAMYYCAA
SYEVVDCYPSGYGQDYWGKGTQVTVSSGGGSQVQLQESGGGSVQAGGSL
RLSCVASGYTFSSYCMGWFRQAPGKEREGVAALGGGSTYYADSVKGRFT
ISQDNAKNTLYLQMNSLKPEDTAMYYCAAAWVACLEFGGSWYDLARYKH
WGQGTQVTVSSASHHHHHH
> SEQ ID NO: 278; DR740(DR589-DR229)
QVQLQESGGGSVQAGGSLRLSCAASEYTASRYCMAWFRQAPGKEREGVA
AIHPGGGTTYYADSVKGRFSISQDSADNTLYLQMNSLKPEDTAMYYCAA
GSLWVPFGDRCAANYWGQGTQVTVSSGGGSQVQLQESGGGLVQPGGSLR
LSCTASGFSFSSYPMTWARQAPGKGLEWVSTIASDGGSTAYAASVEGRF
TISRDNAKSTLYLQLNSLKTEDTAMYYCTKGYGDGTPAPGQGTQVTVSS
ASHHHHHH
> SEQ ID NO: 279; DR741(DR589-DR230)
QVQLQESGGGSVQAGGSLRLSCAASEYTASRYCMAWFRQAPGKEREGVA
AIHPGGGTTYYADSVKGRFSISQDSADNTLYLQMNSLKPEDTAMYYCAA
GSLWVPFGDRCAANYWGQGTQVTVSSGGGSQVQLQESGGGLVQPGGSLR
LSCAASGFTFSSAHMSWVRQAPGKGREWIASIYSGGGTFYADSVKGRFT
ISRDNAKNTLYLQLNSLKAEDTAMYYCATNRLHYYSDDDSLRGQGTQVT
VSSASHHHHHH
> SEQ ID NO: 280; DR742(DR589-DR231)
QVQLQESGGGSVQAGGSLRLSCAASEYTASRYCMAWFRQAPGKEREGVA
AIHPGGGTTYYADSVKGRFSISQDSADNTLYLQMNSLKPEDTAMYYCAA
GSLWVPFGDRCAANYWGQGTQVTVSSGGGSQVQLQESGGGSVQAGGSLR
LSCTASGFTFDDREMNWYRQAPGNECELVSTISSDGSTYYADSVKGRFT
ISQDNAKNTVYLQMDSVKPEDTAVYYCAADFMIAIQAPGAGCWGQGTQV
TVSSASHHHHHH
> SEQ ID NO: 281; DR743(DR589-DR232)
QVQLQESGGGSVQAGGSLRLSCAASEYTASRYCMAWFRQAPGKEREGVA
AIHPGGGTTYYADSVKGRFSISQDSADNTLYLQMNSLKPEDTAMYYCAA
GSLWVPFGDRCAANYWGQGTQVTVSSGGGSQVQLQESGGGSVQAGGSLR
LSCVASGYTSCMGWFRQAPGKEREAVATIYTRGRSIYYADSVKGRFTIS
QDNAKNTLYLQMNSLKPEDIAMYSCAAGGYSWSAGCEFNYWGQGTQVTV
SSASHHHHHH
> SEQ ID NO: 282; DR744(DR589-DR233)
QVQLQESGGGSVQAGGSLRLSCAASEYTASRYCMAWFRQAPGKEREGVA
AIHPGGGTTYYADSVKGRFSISQDSADNTLYLQMNSLKPEDTAMYYCAA
GSLWVPFGDRCAANYWGQGTQVTVSSGGGSQVQLQESGGGSVQAGGSLR
LSCTASGFTFDDSDMGWYRQAPGNECELVSTISSDGSTYYADSVKGRFT
ISQDNAKNTVYLQMNSLKPEDTAVYYCAAEPRGYYSNYGGRRECNYWGQ
GTQVTVSSASHHHHHH
> SEQ ID NO: 283; DR745(DR589-DR234)
QVQLQESGGGSVQAGGSLRLSCAASEYTASRYCMAWFRQAPGKEREGVA
AIHPGGGTTYYADSVKGRFSISQDSADNTLYLQMNSLKPEDTAMYYCAA
GSLWVPFGDRCAANYWGQGTQVTVSSGGGSQVQLQESGGGSVQAGGSLR
LSCVASGYTFSSYCMGWFRQAPGKEREGVAALGGGSTYYADSVKGRFTI
SQDNAKNTLYLQMNSLKPEDTAMYYCAAAWVACLEFGGSWYDLARYKHW
GQGTQVTVSSASHHHHHH
> SEQ ID NO: 284; DR746(DR590-DR229)
QVQLQESGGGSVQAGGSLRLSCAASGYEYCRIHMTWYRQGPGKEREFVS
SIGSDGRKTYANSVTGRFTISRDNANHTVYLQMNSLSPEDTAMYYCKTE
YLYGLGCPDGSAYWGQGTQVTVSSGGGSQVQLQESGGGLVQPGGSLRLS
CTASGFSFSSYPMTWARQAPGKGLEWVSTIASDGGSTAYAASVEGRFTI
SRDNAKSTLYLQLNSLKTEDTAMYYCTKGYGDGTPAPGQGTQVTVSSAS
HHHHHH
> SEQ ID NO: 285; DR747(DR590-DR230)
QVQLQESGGGSVQAGGSLRLSCAASGYEYCRIHMTWYRQGPGKEREFVS
SIGSDGRKTYANSVTGRFTISRDNANHTVYLQMNSLSPEDTAMYYCKTE
YLYGLGCPDGSAYWGQGTQVTVSSGGGSQVQLQESGGGLVQPGGSLRLS
CAASGFTFSSAHMSWVRQAPGKGREWIASIYSGGGTFYADSVKGRFTIS
RDNAKNTLYLQLNSLKAEDTAMYYCATNRLHYYSDDDSLRGQGTQVTVS
SASHHHHHH
> SEQ ID NO: 286; DR748(DR590-DR231)
QVQLQESGGGSVQAGGSLRLSCAASGYEYCRIHMTWYRQGPGKEREFVS
SIGSDGRKTYANSVTGRFTISRDNANHTVYLQMNSLSPEDTAMYYCKTE
YLYGLGCPDGSAYWGQGTQVTVSSGGGSQVQLQESGGGSVQAGGSLRLS
CTASGFTFDDREMNWYRQAPGNECELVSTISSDGSTYYADSVKGRFTIS
QDNAKNTVYLQMDSVKPEDTAVYYCAADFMIAIQAPGAGCWGQGTQVTV
SSASHHHHHH
> SEQ ID NO: 287; DR749(DR590-DR232)
QVQLQESGGGSVQAGGSLRLSCAASGYEYCRIHMTWYRQGPGKEREFVS
SIGSDGRKTYANSVTGRFTISRDNANHTVYLQMNSLSPEDTAMYYCKTE
YLYGLGCPDGSAYWGQGTQVTVSSGGGSQVQLQESGGGSVQAGGSLRLS
CVASGYTSCMGWFRQAPGKEREAVATIYTRGRSIYYADSVKGRFTISQD
NAKNTLYLQMNSLKPEDIAMYSCAAGGYSWSAGCEFNYWGQGTQVTVSS
ASHHHHHH
> SEQ ID NO: 288; DR750(DR590-DR233)
QVQLQESGGGSVQAGGSLRLSCAASGYEYCRIHMTWYRQGPGKEREFVS
SIGSDGRKTYANSVTGRFTISRDNANHTVYLQMNSLSPEDTAMYYCKTE
YLYGLGCPDGSAYWGQGTQVTVSSGGGSQVQLQESGGGSVQAGGSLRLS
CTASGFTFDDSDMGWYRQAPGNECELVSTISSDGSTYYADSVKGRFTIS
QDNAKNTVYLQMNSLKPEDTAVYYCAAEPRGYYSNYGGRRECNYWGQGT
QVTVSSASHHHHHH
> SEQ ID NO: 289; DR751(DR590-DR234)
QVQLQESGGGSVQAGGSLRLSCAASGYEYCRIHMTWYRQGPGKEREFVS
SIGSDGRKTYANSVTGRFTISRDNANHTVYLQMNSLSPEDTAMYYCKTE
YLYGLGCPDGSAYWGQGTQVTVSSGGGSQVQLQESGGGSVQAGGSLRLS
CVASGYTFSSYCMGWFRQAPGKEREGVAALGGGSTYYADSVKGRFTISQ
DNAKNTLYLQMNSLKPEDTAMYYCAAAWVACLEFGGSWYDLARYKHWGQ
GTQVTVSSASHHHHHH

In some embodiments, the binding proteins described herein can include one or more anti-IL2Rβ V_{H}H antibodies. When two or more anti-IL2Rβ V_{H}H antibodies are present, neighboring antibodies can be conjugated to each other by way of a linker. In some embodiments, the binding proteins described herein can include one or more anti-IL2Rγ V_{H}H antibodies. When two or more anti-IL2Rγ V_{H}H antibodies are present, neighboring antibodies can be conjugated to each other by way of a linker.

In some embodiments, the binding proteins described herein can include one or more anti-IL2Rβ V_{H}H antibodies and one or more anti-IL2Rγ V_{H}H antibodies. Neighboring antibodies can be conjugated to each other by way of a linker. In some embodiments, the number of anti-IL2Rβ V_{H}H antibodies and the number of anti-IL2Rγ V_{H}H antibodies in a binding protein are the same. In other embodiments, the number of anti-IL2Rβ V_{H}H antibodies and the number of anti-IL2Rγ V_{H}H antibodies in a binding protein are different.

In some embodiments, a binding protein described herein can be represented by the following formula:

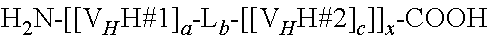

wherein L is a linker, a, b, c are independently selected from 0 or 1, and x is an integer between 1 and 10 (e.g., 1, 2, 3, 4, 5, 6, 7, 8, 9, or 10). In some embodiments, V_{H}H #1 and V_{H}H #2 target the same receptor or subunit thereof. In some embodiments, V_{H}H #1 and V_{H}H #2 target different receptors or subunits thereof. In some embodiments, V_{H}H #1 and V_{H}H #2 can have the same sequence. In other embodiments, V_{H}H #1 and V_{H}H #2 can have different sequences.

In some embodiments, the IL2Rβ/IL2Rγ binding protein is linked to an Fc polypeptide or an Fc domain. In some embodiments, the Fc polypeptide (e.g., subunit of an Fc domain) or an Fc domain is from an IgG1, IgG2, IgG3 or IgG4. In some embodiments, the IL2Rβ/IL2Rγ binding protein is at least 90 percent (e.g., 91%, 92%, 93%, 94%, 95%, 96%, 97%, 98%, 99%, or 100%) identical to any one of SEQ ID NOS: 65-80 (Table 3), 81-106 (Table 4), or 170-289, optionally without the HHHHHH (SEQ ID NO: 127) sequence(s) therein.

### A. “Forward Orientation”

In some embodiments, the IL2R binding molecule of the present disclosure comprises a polypeptide of the structure:

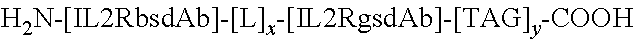

wherein and L is a polypeptide linker of 1-50 amino acids and x=0 or 1, and TAG is a chelating peptide or a subunit of an Fc domain and y=0 or 1.

In some embodiments, a IL2R binding molecule of the foregoing structure comprises a polyptide from amino to carboxy terminus:
(a) an IL2Rβ sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR1 in a row of Table 1;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR2 in a row of Table 1; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR3 in a row of Table 1; or
   (A) a CDR1 comprising an amino acid sequence of SEQ ID NO: 1 or SEQ ID NO: 410, a CDR2 comprising an amino acid sequence of SEQ ID NO: 2, and CDR3 a comprising an amino acid sequence of SEQ ID NO: 3;
   (B) a CDR1 comprising an amino acid sequence of SEQ ID NO: 5 or SEQ ID NO: 411, a CDR2 comprising an amino acid sequence of SEQ ID NO: 6, and CDR3 a comprising an amino acid sequence of SEQ ID NO: 7;
   (C) a CDR1 comprising an amino acid sequence of SEQ ID NO: 9 or SEQ ID NO: 412, a CDR2 comprising an amino acid sequence of SEQ ID NO: 10, and CDR3 a comprising an amino acid sequence of SEQ ID NO: 11;
   (D) a CDR1 comprising an amino acid sequence of SEQ ID NO: 13 or SEQ ID NO: 413, a CDR2 comprising an amino acid sequence of SEQ ID NO: 14, and CDR3 a comprising an amino acid sequence of SEQ ID NO: 15;
   (E) a CDR1 comprising an amino acid sequence of SEQ ID NO: 17 or SEQ ID NO: 414, a CDR2 comprising an amino acid sequence of SEQ ID NO: 18, and CDR3 a comprising an amino acid sequence of SEQ ID NO: 19;
   (F) a CDR1 comprising an amino acid sequence of SEQ ID NO: 21 or SEQ ID NO: 415, a CDR2 comprising an amino acid sequence of SEQ ID NO: 122, and CDR3 a comprising an amino acid sequence of SEQ ID NO: 23;
   (G) a CDR1 comprising an amino acid sequence of SEQ ID NO: 25 or SEQ ID NO: 416, a CDR2 comprising an amino acid sequence of SEQ ID NO: 26, and CDR3 a comprising an amino acid sequence of SEQ ID NO: 27;
   (H) a CDR1 comprising an amino acid sequence of SEQ ID NO: 29 or SEQ ID NO: 417, a CDR2 comprising an amino acid sequence of SEQ ID NO: 30, and CDR3 a comprising an amino acid sequence of SEQ ID NO: 31;
   (I) a CDR1 comprising an amino acid sequence of SEQ ID NO: 33 or SEQ ID NO: 418, a CDR2 comprising an amino acid sequence of SEQ ID NO: 34, and CDR3 a comprising an amino acid sequence of SEQ ID NO: 35; or
   (J) a CDR1 comprising an amino acid sequence of SEQ ID NO: 37 or SEQ ID NO: 419, a CDR2 comprising an amino acid sequence of SEQ ID NO: 38, and CDR3 a comprising an amino acid sequence of SEQ ID NO: 39;
   wherein each CDR independently comprises 0, 1, 2, or 3 amino acid changes relative to the indicated sequence; and
(b) optionally, a polypeptide linker from 1-50 amino acids, alternatively 1-40 amino acids, alternatively 1-30 amino acids, alternatively 1-20 amino acids, alternatively 1-15 amino acids, alternatively 1-10 amino acids, alternatively 1-8 amino acids, alternatively 1-6 amino acids, alternatively 1-4 amino acids; and
(c) an IL2Rγ sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR1 in a row of Table 2;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR2 in a row of Table 2; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR3 in a row of Table 2; or
i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59; or
vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
wherein each CDR independently comprises 0, 1, 2, or 3 amino acid changes relative to the indicated sequence.

In some embodiments, the IL2R binding molecule comprises an IL2Rb sdAb comprising a CDR1, a CDR2, and a CDR3 listed in a row of Table 1, and an IL2Rγ sdAb comprising a CDR1, a CDR2, and a CDR3 as listed in a row of Table 2. In some embodiments, the IL2R binding molecule comprises an IL2Rb sdAb comprising a CDR1, a CDR2, and a CDR3 listed in a row of Table 30, and an IL2Rγ sdAb comprising a CDR1, a CDR2, and a CDR3 as listed in a row of Table 32. In some embodiments, the IL2R binding molecule comprises an IL2Rb sdAb comprising a CDR1, a CDR2, and a CDR3 listed in a row of Table 31, and an IL2Rγ sdAb comprising a CDR1, a CDR2, and a CDR3 as listed in a row of Table 33.

In some embodiments, the IL2Rb sdAb of the IL2R binding molecule comprises a sequence having at least 90% (e.g., 91%, 92%, 93%, 94%, 95%, 96%, 97%, 98%, 99%, or 100%) sequence identity to a sequence listed in a row of Table 34 or Table 35. In some embodiments, the IL2Rγ sdAb of the IL2R binding molecule comprises a sequence having at least 90% (e.g., 91%, 92%, 93%, 94%, 95%, 96%, 97%, 98%, 99%, or 100%) sequence identity to a sequence listed in a row of Table 36 or Table 37.

In some embodiments, the subunit of the Fc domain is from an IgG1, IgG2, IgG3 or IgG4. In some embodiments, the subunit of the Fc domain comprises one or more amino acid substitutions to reduce effector function, for example, the subunit of the Fc domain comprises a set of amino acid substitutions selected from the group consisting of: (a) L234A/L235A/P329A (“LALAPA”); L234A/L235A/P329G (“LALAPG”); L234A/L235E/G237A/A330S/P331 S (“AEASS”); E233P/L234V/L235A/AG237 (PVAdelG); and L234F/L235E/P33IS (“FES”). In some embodiments, the subunit of the Fc domain is modified for multimerization. In some embodiments the subunit of the Fc domain comprises an amino acid substitution at position C220 (EU numbering) of the upper hinge domain to eliminate the sulthydryl side chain. In some embodiments, the substitution at position C220 is C220S (EU numbering) substitution. In some embodiments the subunit of the Fc domain comprises amino acid substitutions in the Fc domain at positions M428 and/or N434 (EU numbering). In some embodiments the amino acid substitutions at positions M428 and/or N434 are M428L and/or N434S. In some embodiments the subunit of the Fc domain comprises amino acid deletions in the Fc domain at positions G446 and/or K447 (EU numbering).

### B. “Reverse Orientation”

In some embodiments, the IL2R binding molecule comprises a polypeptide of the structure:

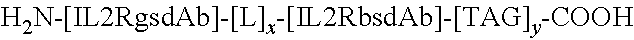

wherein and L is a polypeptide linker of 1-50 amino acids and x=0 or 1, and TAG is a chelating peptide or a subunit of an Fc domain and y=0 or 1.

In some embodiments, a IL2R binding molecule of the foregoing structure comprises a polyptide from amino to carboxy terminus:
(a) an IL2Rg sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR1 in a row of Table 2;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR2 in a row of Table 2; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR3 in a row of Table 2, or
i) a CDR1 comprising an amino acid sequence of SEQ ID NO: 41 or SEQ ID NO: 420, a CDR2 comprising an amino acid sequence of SEQ ID NO: 42, and CDR3 a comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and CDR3 a comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and CDR3 a comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and CDR3 a comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and CDR3 a comprising an amino acid sequence of SEQ ID NO: 59; or
vi) a CDR1 comprising an amino acid sequence of SEQ ID NO: 61 or SEQ ID NO: 425, a CDR2 comprising an amino acid sequence of SEQ ID NO: 62, and CDR3 a comprising an amino acid sequence of SEQ ID NO: 63;
wherein each CDR independently comprises 0, 1, 2, or 3 amino acid changes relative to the indicated sequence, and;
(b) optionally, a polypeptide linker from 1-50 amino acids, alternatively 1-40 amino acids, alternatively 1-30 amino acids, alternatively 1-20 amino acids, alternatively 1 amino acids, alternatively 1-10 amino acids, alternatively 1-8 amino acids, alternatively 1-6 amino acids, alternatively 1-4 amino acids; and
(c) an IL2Rb sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR1 in a row of Table 1.
   CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR2 in a row of Table 1; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR3 in a row of Table 1; or
   (A) a CDR1 comprising an amino acid sequence of SEQ ID NO: 1 or SEQ ID NO: 410, a CDR2 comprising an amino acid sequence of SEQ ID NO: 2, and CDR3 a comprising an amino acid sequence of SEQ ID NO: 3;
   (B) a CDR1 comprising an amino acid sequence of SEQ ID NO: 5 or SEQ ID NO: 411, a CDR2 comprising an amino acid sequence of SEQ ID NO: 6, and CDR3 a comprising an amino acid sequence of SEQ ID NO: 7;
   (C) a CDR1 comprising an amino acid sequence of SEQ ID NO: 9 or SEQ ID NO: 412, a CDR2 comprising an amino acid sequence of SEQ ID NO: 10, and CDR3 a comprising an amino acid sequence of SEQ ID NO: 11;
   (D) a CDR1 comprising an amino acid sequence of SEQ ID NO: 13 or SEQ ID NO: 413, a CDR2 comprising an amino acid sequence of SEQ ID NO: 14, and CDR3 a comprising an amino acid sequence of SEQ ID NO: 15;
   (E) a CDR1 comprising an amino acid sequence of SEQ ID NO: 17 or SEQ ID NO: 414, a CDR2 comprising an amino acid sequence of SEQ ID NO: 18, and CDR3 a comprising an amino acid sequence of SEQ ID NO: 19;
   (F) a CDR1 comprising an amino acid sequence of SEQ ID NO: 21 or SEQ ID NO: 415, a CDR2 comprising an amino acid sequence of SEQ ID NO: 122, and CDR3 a comprising an amino acid sequence of SEQ ID NO: 23;
   (G) a CDR1 comprising an amino acid sequence of SEQ ID NO: 25 or SEQ ID NO: 416, a CDR2 comprising an amino acid sequence of SEQ ID NO: 26, and CDR3 a comprising an amino acid sequence of SEQ ID NO: 27;
   (H) a CDR1 comprising an amino acid sequence of SEQ ID NO: 29 or SEQ ID NO: 417, a CDR2 comprising an amino acid sequence of SEQ ID NO: 30, and CDR3 a comprising an amino acid sequence of SEQ ID NO: 31;
   (I) a CDR1 comprising an amino acid sequence of SEQ ID NO: 33 or SEQ ID NO: 418, a CDR2 comprising an amino acid sequence of SEQ ID NO: 34, and CDR3 a comprising an amino acid sequence of SEQ ID NO: 35; or
   (J) a CDR1 comprising an amino acid sequence of SEQ ID NO: 37 or SEQ ID NO: 419, a CDR2 comprising an amino acid sequence of SEQ ID NO: 38, and CDR3 a comprising an amino acid sequence of SEQ ID NO: 39;
   wherein each CDR independently comprises 0, 1, 2, or 3 amino acid changes relative to the indicated sequence.

In some embodiments, the binding molecule comprises an IL2Rg sdAb comprising a CDR1, a CDR2, and a CDR3 as listed in a row of Table 2, and the IL2Rb sdAb and a CDR1, a CDR2, and a CDR3 as listed in a row of Table 1. In some embodiments, the IL2R binding molecule comprises an IL2Rγ sdAb comprising a CDR1, a CDR2, and a CDR3 as listed in a row of Table 32, and an IL2Rb sdAb comprising a CDR1, a CDR2, and a CDR3 listed in a row of Table 30. In some embodiments, the IL2R binding molecule comprises an IL2Rγ sdAb comprising a CDR1, a CDR2, and a CDR3 as listed in a row of Table 33, and an IL2Rb sdAb comprising a CDR1, a CDR2, and a CDR3 listed in a row of Table 31.

In some embodiments, the IL2Rg sdAb comprises a sequence having at least 90% (e.g., 91%, 92%, 93%, 94%, 95%, 96%, 97%, 98%, 99%, or 100%) sequence identity to a sequence listed in a row of Table 36 or Table 37. In some embodiments, the IL2Rb sdAb comprises a sequence having at least 90% (e.g., 91%, 92%, 93%, 94%, 95%, 96%, 97%, 98%, 99%, or 100%) sequence identity to a sequence listed in a row of Table 34 or Table 35.

In some embodiments, the subunit of the Fc domain is from an IgG1, IgG2, IgG3 or IgG4. In some embodiments, the subunit of the Fc domain comprises one or more amino acid substitutions to reduce effector function, for example, the subunit of the Fc domain comprises a set of amino acid substitutions selected from the group consisting of: (a) L234A/L235A/P329A (“LALAPA”); L234A/L235A/P329G (“LALAPG”); L234A/L235E/G237A/A330S/P331 S (“AEASS”); E233P/L234V/L235A/hG237 (PVAdelG); and L234F/L235E/P331S (“FES”). In some embodiments, the subunit of the Fc domain is modified for multimerization. In some embodiments the subunit of the Fc domain comprises an amino acid substitution at position C220 (EU numbering) of the upper hinge domain to eliminate the sulthydryl side chain. In some embodiments, the substitution at position C220 is C220S (EU numbering) substitution. In some embodiments the subunit of the Fc domain comprises amino acid substitutions in the Fc domain at positions M428 and/or N434 (EU numbering). In some embodiments the amino acid substitutions at positions M428 and/or N434 are M428L and/or N434S. In some embodiments the subunit of the Fc domain comprises amino acid deletions in the Fc domain at positions G446 and/or K447 (EU numbering).

### IV. Single-Domain Antibody and V_{H}H

A single-domain antibody (sdAb) is an antibody containing a single monomeric variable antibody domain. Like a full-length antibody, it is able to bind selectively to a specific antigen. The complementary determining regions (CDRs) of sdAbs are within a single-domain polypeptide. Single-domain antibodies can be engineered from heavy-chain antibodies found in camelids, which are referred to as V_{H}Hs. Cartilaginous fishes also have heavy-chain antibodies (IgNAR, “immunoglobulin new antigen receptor”), from which single-domain antibodies referred to as VNARS can be obtained. The dimeric variable domains from common immunoglobulin G (IgG) from humans or mice can also be split into monomers to make sdAbs. Although most research into sdAbs is currently based on heavy chain variable domains, sdAbs derived from light chains have also been shown to bind specifically to target, see, e.g., Moller et al., J Biol Chem. 285(49):38348-38361, 2010. In some embodiments, a sdAb is composed of a single monomeric light chain variable antibody domain.

A sdAb can be a heavy chain antibody (V_{H}H). A V_{H}H is a type of sdAb that has a single monomeric heavy chain variable antibody domain. Similar to a traditional antibody, a V_{H}H is able to bind selectively to a specific antigen. A binding protein described herein can include two V_{H}Hs (e.g., V_{H}H²) joined together by a linker (e.g., a peptide linker). The binding protein can be a bispecific V_{H}H² that includes a first V_{H}H binding to a first receptor or domain or subunit thereof and a second V_{H}H binding to a second receptor or domain or subunit thereof, in which the two V_{H}Hs are joined by a linker.

An exemplary V_{H}H has a molecular weight of approximately 12-15 kDa which is much smaller than traditional mammalian antibodies (150-160 kDa) composed of two heavy chains and two light chains. V_{H}Hs can be found in or produced from Camelidae mammals (e.g., camels, llamas, dromedary, alpaca, and guanaco) which are naturally devoid of light chains. Descriptions of sdAbs and V_{H}Hs can be found in, e.g., De Greve et al., Curr Opin Biotechnol. 61:96-101, 2019; Ciccarese, et al., Front Genet. 10:997, 2019; Chanier and Chames, Antibodies (Basel) 8(1), 2019; and De Vlieger et al., Antibodies (Basel) 8(1), 2018.

To prepare a binding protein that is a bispecific V_{H}H², in some embodiments, the two V_{H}Hs can be synthesized separately, then joined together by a linker. Alternatively, the bispecific V_{H}H² can be synthesized as a fusion protein. V_{H}Hs having different binding activities and receptor targets can be paired to make a bispecific V_{H}H². The binding proteins can be screened for signal transduction on cells carrying one or both relevant receptors.

### V. Linkers

As previously described, the binding domains of the dimeric binding proteins of the present disclosure may be joined contiguously (e.g., the C-terminal amino acid of the first V_{H}H in the binding protein to the N-terminal amino acid of the second V_{H}H in the binding protein) or the binding domains of the binding protein may optionally be joined via a linker. A linker is a linkage between two elements, e.g., protein domains. In a bispecific V_{H}H² binding protein described herein, a linker is a linkage between the two V_{H}Hs in the binding protein. A linker can be a covalent bond or a peptide linker. In some embodiments, the two V_{H}Hs in a binding protein are joined directly (i.e., via a covalent bond). The length of the linker between two V_{H}Hs in a binding protein can be used to modulate the proximity of the two V_{H}Hs of the binding protein. By varying the length of the linker, the overall size and length of the binding protein can be tailored to bind to specific cell receptors or domains or subunits thereof. For example, if the binding protein is designed to bind to two receptors or domains or subunits thereof that are located close to each other on the same cell, then a short linker can be used. In another example, if the binding protein is designed to bind to two receptors or domains or subunits there of that are located on two different cells, then a long linker can be used.

In some embodiments, the linker is a peptide linker. A peptide linker can include between 1 and 50 amino acids (e.g., between 2 and 50, between 5 and 50, between 10 and 50, between 15 and 50, between 20 and 50, between 25 and 50, between 30 and 50, between 35 and 50, between 40 and 50, between 45 and 50, between 2 and 45, between 2 and 40, between 2 and 35, between 2 and 30, between 2 and 25, between 2 and 20, between 2 and 15, between 2 and 10, between 2 and 5 amino acids). A linker can also be a chemical linker, such as a synthetic polymer, e.g., a polyethylene glycol (PEG) polymer.

In some embodiments, a linker joins the C-terminus of the first V_{H}H in the binding protein to the N-terminus of the second V_{H}H in the binding protein. In other embodiments, a linker joins the C-terminus of the second V_{H}H in the binding protein to the N-terminus of the first V_{H}H in the binding protein.

Suitable peptide linkers are known in the art, and include, for example, peptide linkers containing flexible amino acid residues such as glycine and serine. In certain embodiments, a peptide linker can contain motifs, e.g., multiple or repeating motifs, of GS, GGS, GGGS (SEQ ID NO: 107), GGGGS (SEQ ID NO: 108), GGGGGS (SEQ ID NO: 109), GGSG (SEQ ID NO: 110), or SGGG (SEQ ID NO: 111). In certain embodiments, a peptide linker can contain 2 to 12 amino acids including motifs of GS, e.g., GS, GSGS (SEQ ID NO: 112), GSGSGS (SEQ ID NO: 113), GSGSGSGS (SEQ ID NO: 114), GSGSGSGSGS (SEQ ID NO: 115), or GSGSGSGSGSGS (SEQ ID NO: 116). In certain other embodiments, a peptide linker can contain 3 to 12 amino acids including motifs of GGS, e.g., GGS, GGSGGS (SEQ ID NO: 117), GGSGGSGGS (SEQ ID NO: 118), and GGSGGSGGSGGS (SEQ ID NO: 119). In yet other embodiments, a peptide linker can contain 4 to 20 amino acids including motifs of GGSG (SEQ ID NO: 110), e.g., GGSGGGSG (SEQ ID NO: 120), GGSGGGSGGGSG (SEQ ID NO: 121), GGSGGGSGGGSGGGSG (SEQ ID NO: 122), or GGSGGGSGGGSGGGSGGGSG (SEQ ID NO: 123). In other embodiments, a peptide linker can contain motifs of GGGGS (SEQ ID NO: 108), e.g., GGGGSGGGGS (SEQ ID NO: 124) or GGGGSGGGGSGGGGS (SEQ ID NO: 125).

### VI. Modifications to Extend Duration of Action In Vivo

The binding proteins described herein can be modified to provide for an extended lifetime in vivo and/or extended duration of action in a subject. In some embodiments, the binding protein can be conjugated to carrier molecules to provide desired pharmacological properties such as an extended half-life. In some embodiments, the binding protein can be covalently linked to the Fc domain of IgG, albumin, or other molecules to extend its half-life, e.g., by pegylation, glycosylation, and the like as known in the art.

In some embodiments, the binding protein is conjugated to an Fc polypeptide or an Fc domain (a dimer of two Fc polypeptides), optionally comprising an intervening linker. Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates. The Fc polypeptide or Fc domain useful in the preparation of Fc fusions can be a naturally occurring or synthetic polypeptide that is homologous to an IgG C-terminal domain produced by digestion of IgG with papain. IgG Fc has a molecular weight of approximately 50 kDa. The binding protein described herein can be conjugated to the entire Fc polypeptide or Fc domain, or a smaller portion that retains the ability to extend the circulating half-life of a chimeric polypeptide of which it is a part. In addition, full-length or fragmented Fc polypeptide can be variants of the wild-type molecule. In a typical presentation, each Fc polypeptide in an Fc domain can carry a heterologous polypeptide; the two heterologous polypeptides in the Fc domain being the same or different (e.g., one fused to an anti-IL-2β V_{H}H antibody and the other fused to an anti-IL-2Ry V_{H}H antibody or one or both heterologous polypeptides linked to a anti-IL-2β V_{H}H antibody/anti-IL2Rγ V_{H}H antibody dimer polypeptide).

In some embodiments the present disclosure provides a heterodimeric Fc comprising at least one anti-IL-2β V_{H}H antibody and at least one anti-IL2Rγ V_{H}H antibody, wherein anti-IL-2β V_{H}H antibody/Fc fusion and an anti-IL2Rγ V_{H}H antibody/Fc fusion polypeptides of the heterodimeric Fc are covalently linked via one disulfide bond, optionally two disulfide bonds, optionally three disulfide bonds, or optionally four disulfide bonds. In some embodiments, the anti-IL-2β V_{H}H antibody/FC fusion and an anti-IL2Rγ V_{H}H antibody/Fc fusion polypeptides are covalently linked via a disulfide bond between the sulfhydryl group of amino acid C226 of the lower hinge domain of the anti-IL-2β V_{H}H antibody/Fc fusion and the sulfhydryl group of amino acid C226 of the lower hinge domain of the anti-IL2Rγ V_{H}H antibody/Fc fusion. In some embodiments, the two fusions are covalently linked via a disulfide bond between the sulfhydryl group of amino acid C229 of the lower hinge domain of the anti-IL-2β V_{H}H antibody/Fc fusion and the sulfhydryl group of amino acid C229 of the lower hinge domain of the anti-IL2Rγ V_{H}H antibody/Fc fusion. In some embodiments, a first Fc domain comprises the amino acid substitution S354C, and the second Fc domain comprises the amino acid substitution Y349C. In some embodiments, the heterodimeric Fc comprises a first Fc domain comprising the amino acid substitution S354C and the second Fc domain comprising the amino acid substitution Y349C and wherein the fusions are linked via a disulfide bond between the S354C of the first Fc domain and Y349C of the second Fc domain. In some embodiments, the two polypeptides of the heterodimeric Fc are covalently linked via one or more, optionally two or more optionally three or more disulfide bonds, optionally four or more disulfide bonds between the side chains of the following groups of cystine pairs: (a) C96 of the first Fc fusion and C199 of the second Fc fusion; (b) between C226 of the first Fc fusion and the C226 of the second Fc fusion, (c) between C229 of the first Fc fusion and the C229 of the second Fc fusion; and (d) between S354C of the first Fc fusion comprising a S354C amino acid substitution and Y349C of the second Fc fusion comprising a Y349C amino acid substitution.

In some embodiments the present disclosure provides a heterodimeric Fc wherein either or both of the fusion subunits of the heterodimeric Fc comprise one or more amino acid substitutions to reduce effector function. In some embodiments, the fusion polypeptides comprise a set of amino acid substitutions selected from the group consisting of: (a) L234A/L235A/P329A (“LALAPA”); L234A/L235A/P329G (“LALAPG”); L234A/L235E/G237A/A330S/P331S (“AEASS”); E233P/L234V/L235A/AG237 (PVAde1G); and L234F/L235E/P331S (“FES”).

In some embodiments the present disclosure provides a heterodimeric Fc wherein either or both of the fusion subunits of the heterodimeric Fc comprises an amino acid substitution at position C220 (EU numbering) of the upper hinge domain to eliminate the sulfhydryl side chain. In some embodiments, the substitution at position C220 is C220S (EU numbering) substitution.

In some embodiments the present disclosure provides a heterodimeric Fc wherein either or both of the fusion subunits of the heterodimeric Fc comprises amino acid substitutions in the Fc domain at positions M428 and/or N434 (EU numbering). In some embodiments the amino acid substitutions at positions M428 and/or N434 are M428L and/or N434S.

In some embodiments the present disclosure provides a heterodimeric Fc wherein either or both of the fusion subunits of the heterodimeric Fc comprises amino acid deletions in the Fc domain at positions G446 and/or K447 (EU numbering).

In one embodiment, the Fc domain is an Fc domain that is derived from human IgG4 heavy constant region (UniProt Reference PO1861). The use of hIgG4 as the source of the Fc provides advantages such very low FcgR binding thereby reducing the necessity of mutations immunogenicity or effector function. In some embodiments, when hIgG4 is employed as the source of the Fc domain, the hIgG4 Fc may comprise the amino acid substitution S228P (EU numbering) which is useful to stabilize the Fc dimer. Additionally, or alternatively, the hIgG4 Fc may comprise the amino acid substitution N297G (EU numbering) which reduces FcgR binding. Additionally, or alternatively, the may comprise the a deletion of the C-terminal lysine residue (K447del) (EU numbering) which reduces FcgR binding.

In one embodiment, the present disclosure provides a homodimeric binding protein comprised of two of the same IL2Rb/IL2Rg dimeric binding molecules (H1, DR240-G3S-DR231) each attached via an AS linker to a domain of an hIgG4 Fc (comprising the hIgG4 hinge, CH2 and CH3 domains derived from the human IgG4 heavy constant region UniProt Reference P01861) containing the amino acid substitutions S228P and N297G and the deletion of K447 having the amino acid sequence, the IgG4 Fc/S228P/N297G/K447 del having the sequence:

(SEQ ID NO: 792)
RVESKYGPPCPPCPAPEFLGGPSVFLFPPKPKDTLMISRTPEVTCVVVDV
SQEDPEVQFNWYVDGVEVHNAKTKPREEQFGSTYRVVSVLTVLHQDWLNG
KEYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKSR
WQEGNVFSCSVMHEALHNHYTQKSLSLSLG.

Alternatively, the wild-type human IgG4 Fc (hIgG4 hinge-CH2-CH3) may be employed which has the amino acid sequence:

(SEQ ID NO: 793)
RVESKYGPPCPSCPAPEFLGGPSVFLFPPKPKDTLMISRTPEVTCVVVDV
SQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKSR
WQEGNVFSCSVMHEALHNHYTQKSLSLSLGK.

In some embodiments the present disclosure provides a heterodimeric Fc wherein either or both of the fusion subunits of the heterodimeric Fc are PEGylated. In some embodiments, either or both of the fusion subunits are PEGylated via the sulthydryl side chain of amino acid C220 of the upper hinge.

In some embodiments, the present disclosure provides an expression cassette encoding a heterodimeric Fc comprising a nucleic acid sequence encoding anti-IL-2β V_{H}H antibody/Fc fusion and an anti-IL2Rγ V_{H}H antibody/Fc fusion polypeptides operably linked to one or more heterologous nucleic acid sequences, wherein the nucleic acid sequences encoding the anti-IL-2β V_{H}H antibody/Fc fusion and an anti-IL2Rγ V_{H}H antibody/Fc fusion polypeptides are: (a) under the control a single promoter and (b) are linked via an intervening sequence that facilitates co-expression. In some embodiments wherein the nucleic acid sequences encoding the anti-IL-2β V_{H}H antibody/Fc fusion and an anti-IL2Rγ V_{H}H antibody/Fc fusion polypeptides are linked via an intervening sequence that facilitates co-expression, the nucleic acid sequence encoding the anti-IL-2β V_{H}H antibody/Fc fusion polypeptide is 5′ relative to the nucleic acid sequence encoding the anti-IL2Rγ V_{H}H antibody/Fc fusion polypeptide. In some embodiments wherein the nucleic acid sequences encoding the anti-IL-2β V_{H}H antibody/Fc fusion and an anti-IL2Rγ V_{H}H antibody/Fc fusion polypeptides are linked via an intervening sequence that facilitates co-expression, the nucleic acid sequence encoding the anti-IL2Rγ V_{H}H antibody/Fc fusion polypeptide is 5′ relative to the nucleic acid sequence encoding the anti-IL-2β V_{H}H antibody/Fc fusion polypeptide. In some embodiments, the intervening sequence to facilitate co-expression is an IRES element or a T2A sequence.

In some embodiments, the present disclosure provides an expression cassette encoding a heterodimeric Fc comprising a nucleic acid sequence encoding anti-IL-2β V_{H}H antibody/Fc fusion and an anti-IL2Rγ V_{H}H antibody/Fc fusion polypeptides operably linked to one or more heterologous nucleic acid sequences, wherein the nucleic acid sequences encoding the anti-IL-2β V_{H}H antibody/Fc fusion and an anti-IL2Rγ V_{H}H antibody/Fc fusion polypeptides are: (a) under the control a single promoter and (b) are linked via an intervening sequence that facilitates co-expression in a mammalian cell.

The present disclosure further provides a recombinant vector encoding a heterodimeric Fc, the vector comprising a first expression cassette encoding an anti-IL-2β V_{H}H antibody/Fc fusion polypeptide and a second expression cassette comprising a nucleic acid sequence encoding a anti-IL2Rγ V_{H}H antibody/Fc fusion polypeptide. In some embodiments, the vector is viral vector. In some embodiments, the vector is non-viral vector.

Further provided is a recombinantly modified cell comprising a nucleic acid molecule or vector of the disclosure. In some embodiments, the cell is a prokaryotic cell, such as a bacterial cell. In some embodiments, the cell is a eukaryotic cell, such as a mammalian cell. Also provided is a cell culture comprising at least one recombinantly modified cell of the disclosure, and a culture medium.

In some embodiments, the recombinantly modified cell is transformed with a recombinant vector encoding a heterodimeric Fc, the vector comprising a first expression cassette encoding an anti-IL2Rγ V_{H}H antibody/Fc fusion polypeptide and a second expression cassette comprising a nucleic acid sequence encoding an anti-IL-2β V_{H}H antibody/Fc fusion polypeptide. In some embodiments, the recombinantly modified cell is transformed with a recombinant vector encoding a heterodimeric Fc, the vector comprising a first expression cassette encoding an anti-IL-2β V_{H}H antibody/Fc fusion polypeptide and a second expression cassette comprising a nucleic acid sequence encoding a anti-IL2Rγ V_{H}H antibody/Fc fusion polypeptide.

In some embodiments, the recombinantly modified cell is transformed with a first vector comprising a nucleic acid sequence encoding a anti-IL2Rγ V_{H}H antibody/Fc fusion polypeptide operably linked to one or more expression control sequences and a second vector comprising an expression cassette comprising a nucleic acid sequence encoding a anti-IL-2β V_{H}H antibody/Fc fusion polypeptide operably linked to one or more expression control sequences. In some embodiments, the recombinantly modified cell is transformed with a first vector comprising a nucleic acid sequence encoding a anti-IL-213 V_{H}H antibody/Fc fusion polypeptide operably linked to one or more expression control sequences and a second vector comprising an expression cassette comprising a nucleic acid sequence encoding a anti-IL2Rγ V_{H}H antibody/Fc fusion polypeptide operably linked to one or more expression control sequences. In some embodiments, the cell is a prokaryotic cell, such as a bacterial cell. In some embodiments, the cell is a eukaryotic cell, such as a mammalian cell. Also provided is a cell culture comprising at least one recombinantly modified cell of the disclosure, and a culture medium.

The present disclosure further provides methods for the recombinant production, isolation, purification and characterization of a heterodimeric Fc. Thus, provided herein is a method for producing a heterodimeric Fc of the disclosure. In some embodiments, the method comprises a) providing one or more recombinantly modified cells comprising a nucleic acid molecule or vector comprising a nucleic acid sequence encoding a heterodimeric Fc as disclosed herein; and b) culturing the one or more cells in a culture medium such that the cells produce the heterodimeric Fc encoded by the nucleic acid sequence.

Also provided is a pharmaceutical composition comprising a heterodimeric Fc of the present disclosure. In some embodiments, the pharmaceutical composition comprises a heterodimeric Fc of the present disclosure and a pharmaceutically acceptable carrier. In some embodiments, the pharmaceutical composition comprises a nucleic acid molecule or vector of the disclosure. In some embodiments, the pharmaceutical composition comprises a recombinantly modified cell of the disclosure. In some embodiments, the recombinantly modified cell is a mammalian cell.

The present disclosure provides a heterodimeric Fc, the heterodimeric Fc comprising a first polypeptide of the formula #1:

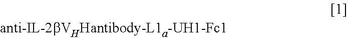

and a second polypeptide of the formula #2:

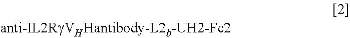

wherein:
L1 and L2 are GSA linkers and a and b are independently selected from 0 (absent) or 1 (present);
UH1 and UH2 are each an upper hinge domain of human immunoglobulin independently selected from the group consisting of the IgG1, IgG2, IgG3 and IgG4 upper hinge, optionally comprising the amino acid substitution C220S (EU numbering);
Fc1 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc2, and
FC2 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc1, and wherein the polypeptide of formula 1 and the polypeptide of formula 2 are linked by at least one interchain disulfide bond.

### Upper Hinge:

The heterodimeric Fcs of the present disclosure are heterodimers comprising polypeptides of the formulae [1] and [2], which each incorporate an upper hinge region of a human immunoglobulin molecule. The term “upper hinge” or “UH” refers to an amino acid sequence corresponding to amino acid residues 216-220 (EU numbering) of a human immunoglobulin molecule. In some embodiments, the upper hinge region is a naturally occurring upper hinge region of a human immunoglobulin selected from the LH regions of human IgG1, human IgG2, human IgG3 and human IgG4 upper hinge domains. In some embodiments, the upper hinge region is the upper hinge region of a human IgG1 immunoglobulin. In some embodiments, the upper hinge region is the upper hinge region of a human IgG1 immunoglobulin comprising the pentameric amino acid sequence: EPKSC (SEQ ID NO: 794).

In some embodiments, the upper hinge region contains an unpaired cysteine residue at position 220 (EU numbering) that typically, in a complete immunoglobulin molecule, binds to a cysteine on a light chain. When only the Fc domain is used comprising the hinge domain, the unpaired cysteine in the hinge domain creates the potential of the formation of improper disulfide bonds. Consequently, in some embodiments the cysteine at position 220 (C220, numbered in accordance with EU numbering) is substituted with an amino acid that does not promote disulfide bonding. In some embodiments, the Fc domain comprises a C220S mutation having the amino acid sequence EPKSS (SEQ ID NO: 795).

### Fc1 and Fc2:

The heterodimeric Fcs of the present disclosure are heterodimers comprising polypeptides of the formulae [1] and [2], which each incorporate an Fc region (Fc1 and Fc2) of a human immunoglobulin molecule modified to promote heterodimerization.

As used herein the term “Fc” and “Fc monomer” are used interchangeably herein to designate the monomeric polypeptide subunit of an Fc dimer. An Fc comprises an amino acid sequence (from amino to carboxy terminal) comprising a lower hinge domain and the CH2 and CH3 domains of a human immunoglobulin molecule. In some embodiments, the Fc monomer is a polypeptide comprising the lower hinge domain and the CH2 and CH3 domains of a human immunoglobulin molecule domains of human IgG1, human IgG2, human IgG3 and human IgG4 hinge domains. The CH2 domain of hIgG1 corresponds to amino acid residues 231-340 (EU numbering) and is provided as SEQ ID NO: 14. The CH3 domain of hIgG1 corresponds to amino acid residues 341-447(EU numbering).

The polypeptides of the formulae [1] and [2] each incorporate a lower hinge region of a human immunoglobulin. As used herein, the term “lower hinge” or “LH” refers to an amino acid sequence corresponding to amino acid residues 221-229 (EU numbering) of a human immunoglobulin molecule. In some embodiments, the lower hinge region is a naturally occurring lower hinge region of a human immunoglobulin selected from the LH regions of IgG1, IgG2, IgG3 and IgG4 lower hinge domains. In some embodiments, the lower hinge region is the lower hinge region of a human IgG1 immunoglobulin. In some embodiments, the lower hinge region is the lower hinge region of a human IgG1 immunoglobulin comprising the decameric amino acid sequence: DKTHTCPPCP (SEQ ID NO: 796).

In some embodiments, Fc1 and Fc2 are derived from a polypeptide corresponding to amino acids 221-447 (EU numbering) of the human IgG1 immunoglobulin having the amino acid sequence (EU numbering indicated:

```
(SEQ ID NO: 797)
              230 240 250 260 270
DKTHTCPPCP APELLGGPSV FLFPPKPKDT LMISRTPEVT CVVVDVSHED
              280 290 300 310 320
PEVKFNWYVD GVEVHNAKTK PREEQYNSTY RVVSVLTVLH QDWLNGKEYK
              330 340 350 360 370
CKVSNKALPA PIEKTISKAK GQPREPQVYT LPPSRDELTK NQVSLTCLVK
              380 390 400 410 420
GFYPSDIAVE WESNGQPENN YKTTPPVLDS DGSFFLYSKL TVDKSRWQQG
              430 440 447
NVFSCSVMHE ALHNHYTQKS LSLSPGK
```

As indicated in above sequence, the C-terminal residue of the wild-type form of the IgG1 Fc domain is a lysine, referred to as K447 in accordance with EU numbering. The K447 is inconsistently removed by the producer cell during recombinant product. As a result, the population of recombinant Fc monomers may be heterogenous in that some fraction of the recombinantly produced Fc monomers will contain K447 and others will not. Such inconsistent proteolytic processing by producer cells may therefore result in a heterogenous population of Fcs. Typically, particularly in the context of human pharmaceutical agents, such heterogeneity of the active pharmaceutical ingredient is to be avoided. Consequently, in addition to modifications to the Fc monomer sequence promote heterodimerization, the present disclosure provides Fc monomers that further comprising a deletion of the C-terminal K447 or a deletion of G446 and K447 and nucleic acid sequences encoding Fc monomers comprising a: (a) a deletion of the lysine residue at position 447 (K447, EU numbering, abbreviated as ΔK447 or des-K447), or (b) deletion of both the glycine at position 456 (G446 EU numbering, abbreviated as des-G446) and K447 (this double deletion of G446 and K447 being referred to herein as des-G446/des-K447 or ΔG446/ΔK447).

### Modifications of Fc Subunits to Promote Heterodimerization

As provided in formulae [1] and [2] above, the Fc1 and Fc2 monomers of the dimeric Fc contain amino acid substitutions that promote heterodimerization between Fc1 and Fc2. A variety of techniques are established for the promotion of heterodimerization of Fc domains. See, e.g. Gillies, et al. United States Patent No. Kim, et al., U.S. patent Ser. No. 11/087,249, issued Aug. 3, 2021. In some embodiments, the modifications to promoter heterodimerization of the Fc1 and Fc2 monomers are the HF-TA mutations and the HA-TF mutations as described in Moore, et al (2011) mAbs 3(6):546-557. The HF-TA method employs the S364H/T394F substitutions on one Fc monomer and the Y349T/F405A substitutions on the complementary Fc monomer. The (HA-TF) method employs the S364H/F405A substitutions on one Fc monomer and the Y349T/T394F substitutions on the complementary Fc monomer. Alternatively, the Fc1 and Fc2 monomers are modified to promote heterodimerization by the ZW1 heterodimerization method which employs the T350V/L351Y/F405A/Y407V substitutions on one Fc monomer and the T350V/T366L/K392L/T394W substitutions on the complementary Fc monomer. Von Kreudenstein, et al (2013) mAbs, 5(5):646-654. Alternatively, the Fc1 and Fc2 monomers are modified to promote heterodimerization by the EW-RVT heterodimerization method which employs the K360E/K409W substitutions on one Fc monomer and the Q347R/D399V/F405T substitutions on the complementary Fc monomer. Choi, et al (2015) Molecular Immunology 65(2):377-83.

In one embodiment, Fc1 and Fc2 are modified to promote heterodimerization by the employment of the “knob-into-hole” (abbreviated KiH) modification as exemplified herein. The KiH modification comprises one or more amino acid substitutions in a first Fc monomer (e.g. Fc1) that create a bulky “knob” domain on a first Fc and one or more amino acid substitutions on a second Fc monomer (e.g. Fc2) that create a complementary pocket or “hole” to receive the “knob” of the first Fc monomer.

A variety of amino acid substitutions have been established for the creation of complementary knob and hole Fc monomers. See, e.g. Ridgway, et al (1996) Protein Engineering 9(7):617-921; Atwell, et al (1997) J. Mol. Biol. 270:26-35; Carter, et al. U.S. Pat. No. 5,807,706 issued Sep. 15, 1998; Carter, et al U.S. Pat. No. 7,695,936 issued Apr. 13, 2010; Zhao et al. “A new approach to produce IgG4-like bispecific antibodies,” Scientific Reports 11: 18630 (2021); Cao et al. “Characterization and Monitoring of a Novel Light-heavy-light Chain Mispair in a Therapeutic Bispecific Antibody,” and Liu et al. “Fc Engineering for Developing Therapeutic Bispecific Antibodies and Novel Scaffolds”. Frontiers in Immunology. 8: 38. doi:10.3389/fimmu.2017.00038 (2017).

In some embodiments, the Fc domain comprises two Fc monomers wherein the CH3 domain of a first Fc monomer wherein the threonine at (EU numbering) position 366 is modified with a bulky residue (e.g. a T366W) create a “knob” and the substitution, and a second Fc monomer comprising one or more substitutions in complementary residues of the CH3 domain of the second Fc monomer to create a pocket or “hole” to receive the bulky residue, for example by amino acid substitutions such as T366S, L368A, and/or Y407V.

In one embodiment, the Fc1 monomer of formula 1 is a “knob” modified Fc monomer comprising the amino acid substitution T366W and the Fc2 monomer of formula 2 is a “hole” modified Fc comprising the set of amino acid substitutions T366S/L368A/Y407V.

Alternatively, the Fc1 monomer of formula 1 is a “hole” modified Fc monomer comprising the set of amino acid substitutions T366S/L368A/Y407V and the Fc2 monomer of formula 2 is a “knob” modified Fc monomer comprising the amino acid substitution T366W.

An example of an engineered Fc heterodimeric pair comprising complementary KiH modifications is provided in the Table below:

TABLE
Amino Acid Substitution Sets of Complementary
IgG1 KiH Heterodimeric Pairs

| Fc | | Amino Acid |
|---|---|---|
| Dimer | Fc | Substitution Set |
| No. | Monomer | (EU Numbering) |
| 1 | Knob | T366W |
| | Hole | T366S/L368A/Y407V |

As noted, the heterodimeric Fcs of the present disclosure are provided as a complementary heterodimeric pair of polypeptides of the formulae [1] and [2] wherein the first and second polypeptide are linked by at least one disulfide bond. In some embodiments, the incorporation of a disulfide bond between the polypeptides of formulae [1] and [2] may be achieved by cysteine substitutions at particular points within the Fc1 and Fc2 domains. In one embodiment, the Fc1 domain of the polypeptide of formula [1] is derived from the Fc domain of hIgG1 comprising an amino acid substitution S354C (EU numbering) and the Fc2 domain of the polypeptide of formula [2] is derived from the Fc domain of hIgG1 comprising an amino acid substitution Y349C (EU numbering) to provide a disulfide bond between the S354C of Fc1 and Y349C of Fc2. Alternatively, the Fc1 domain of the polypeptide of formula [1] is derived from the Fc domain of hIgG1 comprising an amino acid substitution Y349C (EU numbering) and the Fc2 domain of the polypeptide of formula [2] is derived from the Fc domain of hIgG1 comprising an amino acid substitution S354C (EU numbering) to provide a disulfide bond between the S354C of Fc1 and Y349C of Fc2.

Further examples of complementary KiH engineered heterodimeric Fc pairs that may be used in the practice of the present disclosure are provided in the Table below.

TABLE
Knob-into-Hole Fc Dimer Pairs

| Fc Dimer | Monomer | Monomer | UH | UH | Fc Amino Acid Substitutions | Fc |
|---|---|---|---|---|---|---|
| Pair. | Type | SEQ ID | Sequence | SEQ ID | (EU Numbering) | Seq ID |
| 2 | Knob | 19 | wt | 11 | T366W | 17 |
| | Hole | 20 | wt | 11 | T366S/L368A/Y407V | 18 |
| 3 | Knob | 21 | C220S | 12 | T366W | 17 |
| | Hole | 22 | C220S | 12 | T366S/L368A/Y407V | 18 |
| 4 | Knob | 23 | wt | 11 | T366W | 17 |
| | Hole | 24 | wt | 11 | T366S/L368A/Y407V | 18 |
| 5 | Knob | 25 | C220S | 12 | S354C/T366W | 17 |
| | Hole | 26 | C220S | 12 | Y349C/T366S/L368A/Y407V | 18 |

### Additional Fc Modifications

In addition to the modifications to promote heterodimerization of the Fc1 and Fc2 domains, Fc1 and Fc2 may optionally provide additional amino acid modifications that mitigate effector function, or eliminate the glycosylation site at N297 such as N297Q.

### Modifications to Reduce Effector Functions

In some embodiments the amino acid sequence of the Fc1 and/or Fc2 monomers modified to promote heterodimerization may be further modified to reduce effector function. In some embodiments, the Fc domain may be modified to substantially reduce binding to Fc receptors (FcyR and FcR) which reduces or abolishes antibody directed cytotoxicity (ADCC) effector function. Modification of Fc domains to reduce effector function are well known in the art. See, e.g., Wang, et al. (2018) IgG Fc engineering to modulate antibody effector functions, Protein Cell 9(1):63-73. For example, mutation of the lysine residue at position 235 (EU numbering) from leucine (L) to glutamic acid (E) is known to reduce effector function by reducing FcgR and C1q binding. Alegre, et al. (1992) J. Immunology 148:3461-3468.

Additionally, substitution of the two leucine (L) residues at positions 234 and 235 (EU numbering) in the IgG1 hinge region with alanine (A), i.e., L234A and L235A, results in decreased complement dependent cytotoxicity (CDC) and antibody dependent cellular cytotoxicity (ADCC). Hezereh et al., (2001) J. Virol 75(24):12161-68. Furthermore, mutation of the proline at position 329 (EU numbering) to alanine (P329A) or glycine, (P329(3) mitigates effector function and may be combined with the L234A and L235A substitutions. In some embodiments, the Fc domains (Fc1 and Fc2) of the compositions of the present invention may comprises the amino acid substitutions L234A/L235A/P329A (EU numbering) referred to as the “LALAPA” substitutions or L234A/L235A/P329G (EU numbering) referred to as the “LALAPG” substitutions. In some embodiments, the Fc domains (Fc1 and Fc2) of the compositions of the present disclosure may comprises the amino acid substitutions E233P/L234V/L235A/MG237 (referred to in the scientific literature as the PVAdelG mutation).

In some embodiments, the Fc domains (Fc1 and Fc2) of the compositions of the present disclosure are from hIgG4. In such instances where the Fc domains of the heterodimeric IL12 and IL23 muteins are derived from hIgG4, attenuation of effector function may be achieve by introduction of the S228P and/or the L235E mutations (EU numbering).

Examples of paired KiH Fc dimeric constructs that may be incorporated into the Fcs of the present disclosure are provided in the Table below:

TABLE
Amino Acid Substitution Sets of Complementary IgG1 KiH UH/Fc Heterodimeric
Pairs Comprising Mutations to Reduce Effector Function

| Fc | | |
|---|---|---|
| Dimer | UH Fc | Amino Acid Substitution Set |
| No. | Monomer | (EU Numbering) |
| 6 | Knob | L234A/L235A/P329A/T366W/ΔK447 |
| | Hole | L234A/L235A/P329A/T366S/L368A/Y407V/ΔK447 |
| 7 | Knob | C220S/L234A/L235A/P329A/T366W/ΔK447 |
| | Hole | C220S/L234A/L235A/P329A/T366S/L368A/Y407V/ΔK447 |
| 8 | Knob | L234A/L235A/P329A/S354C/T366W/ΔK447 |
| | Hole | L234A/L235A/P329A/Y349C/T366S/L368A/Y407V/ΔK447 |
| 9 | Knob | C220S/L234A/L235A/P329A/S354C/T366W/ΔK447 |
| | Hole | C220S/L234A/L235A/P329A/Y349C/T366S/L368A/Y407V/ΔK447 |
| 10 | Knob | L234A/L235A/P329G/T366W/ΔK447 |
| | Hole | L234A/L235A/P329G/T366S/L368A/Y407V/ΔK447 |
| 11 | Knob | C220S/L234A/L235G/P329A/T366W/ΔK447 |
| | Hole | C220S/L234A/L235G/P329A/T366S/L368A/Y407V/ΔK447 |
| 12 | Knob | L234A/L235A/P329G/S354C/T366W/ΔK447 |
| | Hole | L234A/L235A/P329G/Y349C/T366S/L368A/Y407V/ΔK447 |
| 13 | Knob | C220S/L234A/L235A/P329G/S354C/T366W/ΔK447 |
| | Hole | C220S/L234A/L235A/P329G/Y349C/T366S/L368A/Y407V/ΔK447 |
| 14 | Knob | L234A/L235E/G237A/A330S/P331S/T366W/ΔK447 |
| | Hole | L234A/L235E/G237A/A330S/P331S/T366S/L368A/Y407V/ΔK447 |
| 15 | Knob | C220S L234A/L235E/G237A/A330S/P331S/T366W/ΔK447 |
| | Hole | C220S/L234A/L235E/G237A/A330S/P331S/T366S/L368A/Y407V/ΔK447 |
| 16 | Knob | L234A/L235E/G237A/A330S/P331S/S354C/T366W/ΔK447 |
| | Hole | L234A/L235E/G237A/A330S/P331S/Y349C/T366S/L368A/Y407V/ΔK447 |
| 17 | Knob | C220S/L234A/L235E/G237A/A330S/P331S/S354C/T366W/ΔK447 |
| | Hole | C220S/L234A/L235E/G237A/A330S/P331S/Y349C/T366S/L368A/Y407V/ΔK447 |
| 18 | Knob | L234F/L235E/P331S/T366W/ΔK447 |
| | Hole | L234F/L235E/P331S/T366S/L368A/Y407V/ΔK447 |
| 19 | Knob | C220S/L234F/L235E/P331S/T366W/ΔK447 |
| | Hole | C220S/L234F/L235E/P331S//L368A/Y407V/ΔK447 |
| 20 | Knob | L234F/L235E/P331S/S354C/T366W/ΔK447 |
| | Hole | L234F/L235E/P331S/Y349C/T366S/L368A/Y407V/ΔK447 |
| 21 | Knob | C220S/S/L234F/L235E/P331S/354C/T366W/ΔK447 |
| | Hole | C220S/L234F/L235E/P331S/Y349C/T366S/L368A/Y407V/ΔK447 |

### Sequence Modification to Extend Duration of Action:

In some embodiments the amino acid sequence of the Fc1 and/or Fc2 monomers modified to promote heterodimerization may be further modified to incorporate amino acid substitutions which extend the duration of action of the molecule and prevent clearance. In some embodiments, such modifications to the Fc monomer include the amino acid substitutions M428L and N434S (EU numbering) referred to as the “LS” modification. The LS modification may optionally be combined with amino acid substitutions to reduce effector function and provide for disulfide bonds between Fc1 and Fc2. The table below provides exemplary Fc1 and Fc1 heterodimeric pairs possessing complementary sequence modifications to promote heterodimerization that may be employed in the design of the Fc1 and Fc2 polypeptides of the formulae [1] and [2].

The following Table provides exemplary Fc heterodimeric pairs which may be used in the preparation of Fc1 and Fc2 polypeptides of the heterodimeric Fcs of the present disclosure:

TABLE
Amino Acid Substitution Sets of Complementary IgG1 KiH UH/Fc Heterodimeric Pairs
Comprising Mutations to Reduce Effector Function and LS Halflife Extensions

| Fc | | |
|---|---|---|
| Dimer | UH/Fc | Amino Acid Substitution Set |
| No. | Monomer | (EU Numbering) |
| 22 | Knob | L234A/L235A/P329A/T366W/M428L/N434S/ΔK447 |
| | Hole | L234A/L235A/P329A/T366S/L368A/Y407V/M428L/N434S/ΔK447 |
| 23 | Knob | C220S/L234A/L235A/P329A/T366W/M428L/N434S/ΔK447 |
| | Hole | C220S/L234A/L235A/P329A/T366S/L368A/Y407V/M428L/N434S/ΔK447 |
| 24 | Knob | L234A/L235A/P329A/S354C/T366W/M428L/N434S/ΔK447 |
| | Hole | L234A/L235A/P329A/Y349C/T366S/L368A/Y407V/M428L/N434S/ΔK447 |
| 25 | Knob | C220S/L234A/L235A/P329A/S354C/T366W/M428L/N434S/ΔK447 |
| | Hole | C220S/L234A/L235A/P329A/Y349C/T366S/L368A/Y407V/M428L/N434S/ΔK447 |
| 26 | Knob | L234A/L235A/P329G/T366W/M428L/N434S/ΔK447 |
| | Hole | L234A/L235A/P329G/T366S/L368A/Y407V/M428L/N434S/ΔK447 |
| 27 | Knob | C220S/L234A/L235G/P329A/T366W/M428L/N434S/ΔK447 |
| | Hole | C220S/L234A/L235G/P329A/T366S/L368A/Y407V/M428L/N434S/ΔK447 |
| 28 | Knob | L234A/L235A/P329G/S354C/T366W/M428L/N434S/ΔK447 |
| | Hole | L234A/L235A/P329G/Y349C/T366S/L368A/Y407V/M428L/N434S/ΔK447 |
| 29 | Knob | C220S/L234A/L235A/P329G/S354C/T366W/M428L/N434S/ΔK447 |
| | Hole | C220S/L234A/L235A/P329G/Y349C/T366S/L368A/Y407V/M428L/N434S/ΔK447 |
| 30 | Knob | L234A/L235E/G237A/A330S/P331S/T366W/M428L/N434S/ΔK447 |
| | Hole | L234A/L235E/G237A/A330S/P331S/T366S/L368A/Y407V/M428L/N434S/ΔK447 |
| 31 | Knob | C220S L234A/L235E/G237A/A330S/P331S/T366W/M428L/N434S/ΔK447 |
| | Hole | C220S/L234A/L235E/G237A/A330S/P331S/T366S/L368A/Y407V/M428L/N434S/ΔK447 |
| 32 | Knob | L234A/L235E/G237A/A330S/P331S/S354C/T366W/M428L/N434S/ΔK447 |
| | Hole | L234A/L235E/G237A/A330S/P331S/Y349C/T366S/L368A/Y407V/M428L/N434S/ΔK447 |
| 33 | Knob | C220S/L234A/L235E/G237A/A330S/P331S/S354C/T366W/M428L/N434S/ΔK447 |
| | Hole | C220S/L234A/L235E/G237A/A330S/P331S/Y349C/T366S/L368A/Y407V/M428L/N434S/ΔK447 |
| 34 | Knob | L234F/L235E/P331S/T366W/M428L/N434S/ΔK447 |
| | Hole | L234F/L235E/P331S/T366S/L368A/Y407V/M428L/N434S/ΔK447 |
| 35 | Knob | C220S/L234F/L235E/P331S/T366W/M428L/N434S/ΔK447 |
| | Hole | C220S/L234F/L235E/P331S//L368A/Y407V/M428L/N434S/ΔK447 |
| 36 | Knob | L234F/L235E/P331S/S354C/T366W/M428L/N434S/ΔK447 |
| | Hole | Y L234F/L235E/P331S/349C/T366S/L368A/Y407V/M428L/N434S/ΔK447 |
| 37 | Knob | C220S/S/L234F/L235E/P331S/354C/T366W/M428L/N434S/ΔK447 |
| | Hole | C220S/L234F/L235E/P331S/Y349C/T366S/L368A/Y407V/M428L/N434S/ΔK447 |

In some embodiments, the Fc domains (Fc1 and Fc2) of the compositions of the present disclosure are from hIgG4. In such instances where the Fc domains of the heterodimeric IL12 and IL23 muteins are derived from hIgG4, heterodimerization of the Fc1 and Fc2 domains by the introduction of the mutations K370E, K409W and E357N, D399V, F405T (EU numbering) in the complementary Fc sequences that comprise the heterodimeric Fc domain.

### Modifications to Eliminate Glycosylation Sites

In some embodiments the amino acid sequence of the Fc1 and/or Fc2 monomers modified to promote heterodimerization may be further modified to eliminate N-linked or O-linked glycosylation sites. Aglycosylated variants of Fc domains, particularly of the IgG1 subclass are known to be poor mediators of effector function. Jefferies et al. 1998, Immol. Rev., vol. 163, 50-76). It has been shown that glycosylation at position 297 (EU numbering) contributes to effector function. Edelman, et al (1969) PNAS (USA) 63:78-85. In some embodiments, the Fc domains of the compositions of the present disclosure comprise one or modifications to eliminate N- or O linked glycosylation sites. Examples of modifications at N297 to eliminate glycosylation sites in the Fc domain include the amino acid substitutions N297Q and N297G.

In some embodiments, when the binding protein described herein is to be administered in the format of an Fc domain fusion, particularly in those situations when the polypeptides conjugated to each Fc polypeptide of the Fc domain dimer are different, the Fc domain may be engineered to possess a “knob-into-hole modification.” The knob-into-hole modification is more fully described in Ridgway, et al. (1996) Protein Engineering 9(7):617-621 and U.S. Pat. No. 5,731,168, issued Mar. 24, 1998. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g., tyrosine or tryptophan) creating a projection from the surface (“knob”), and ii) in the CH3 domain of a second heavy chain, an amino acid residue is replaced with an amino acid residue having a smaller side chain (e.g., alanine or threonine), thereby generating a cavity (“hole”) at interface in the second CH3 domain within which the protruding side chain of the first CH3 domain (“knob”) is received by the cavity in the second CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 and Y349 which results in a stabilizing disulfide bridge between the two antibody heavy chains in the Fc region (Carter, et al. (2001) Immunol Methods 248, 7-15). The knob-into-hole format is used to facilitate the expression of a first polypeptide (e.g., a first V_{H}H in a binding protein described herein) on a first Fc polypeptide with a “knob” modification and a second polypeptide (e.g., a second V_{H}H in a binding protein described herein) on the second Fc polypeptide with a “hole” modification to facilitate the expression of heterodimeric polypeptide conjugates.

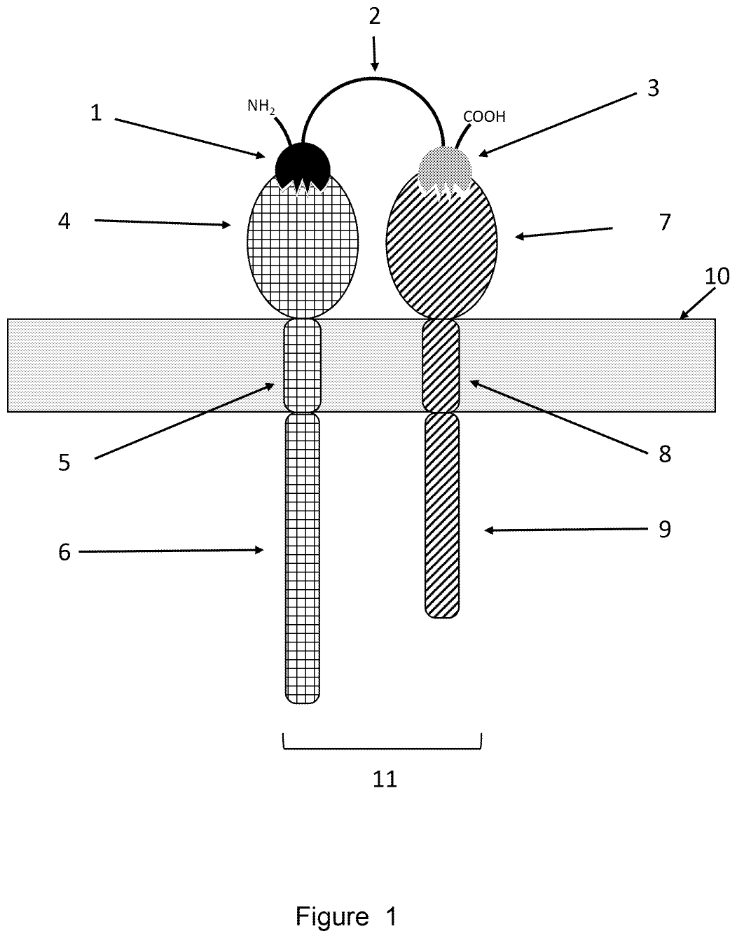

In some embodiments, the binding proteins described herein can have the formats as illustrated in FIGS. 1A-1D. In one example, a first binding protein comprising an anti-IL2Rβ V_{H}H antibody at the N-terminus and an anti-IL2Rγ V_{H}H antibody at the C-terminus (e.g., N-terminus-anti-IL2Rβ V_{H}H antibody-linker-anti-IL2Rγ V_{H}H antibody-C-terminus) can be fused to a first Fc polypeptide, and a second binding protein comprising an anti-IL2Rγ V_{H}H antibody at the N-terminus and an anti-IL2Rβ V_{H}H antibody at the C-terminus (e.g., N-terminus-anti-IL2Rγ V_{H}H antibody-linker-anti-IL2Rβ V_{H}H antibody-C-terminus) can be fused to a second Fc polypeptide (FIG. 1A), in which the first Fc polypeptide and the second Fc polypeptide form an Fc domain. In this example, one Fc polypeptide can comprise T366W as a knob mutation and the other Fc polypeptide can comprise T366S, L368A, Y407V as hole mutations to promoter Fc heterodimer formation.

In another example, two identical binding proteins can each be conjugated to an Fc polypeptide. Two identical binding protein-Fc polypeptide conjugates can then dimerize to form a homodimer (FIG. 1B). In some embodiments, both binding proteins can have an anti-IL2Rβ V_{H}H antibody at the N-terminus and an anti-IL2Rγ V_{H}H antibody at the C-terminus (e.g., N-terminus-anti-IL2Rβ V_{H}H antibody-linker-anti-IL2Rγ V_{H}H antibody-C-terminus). In other embodiments, both binding proteins can have an anti-IL2Rγ V_{H}H antibody at the N-terminus and an anti-IL2Rβ V_{H}H antibody at the C-terminus (e.g., N-terminus-anti-IL2Rγ V_{H}H antibody-linker-anti-IL2Rβ V_{H}H antibody-C-terminus).

In yet another example, a binding protein can be conjugated to one of the two Fc polypeptides in an Fc domain (FIGS. 1C and 1D). In this case, one Fc polypeptide can comprise T366W as a knob mutation and the other Fc polypeptide can comprise T366S, L368A, Y407V as hole mutations to promoter heterodimer formation. The binding protein can have an anti-IL2Rβ V_{H}H antibody at the N-terminus and an anti-IL2Rγ V_{H}H antibody at the C-terminus (e.g., N-terminus-anti-IL2Rβ V_{H}H antibody-linker-anti-IL2Rγ V_{H}H antibody-C-terminus). In other embodiments, the binding protein can have an anti-IL2Rγ V_{H}H antibody at the N-terminus and an anti-IL2Rβ V_{H}H antibody at the C-terminus (e.g., N-terminus-anti-IL2Rγ V_{H}H antibody-linker-anti-IL2Rβ V_{H}H antibody-C-terminus).

In some embodiments, the binding protein can be conjugated to one or more water-soluble polymers, optionally comprising an intervening linker. Examples of water soluble polymers useful in the practice of the present disclosure include polyethylene glycol (PEG), poly-propylene glycol (PPG), polysaccharides (polyvinylpyrrolidone, copolymers of ethylene glycol and propylene glycol, poly(oxyethylated polyol), polyolefinic alcohol), polysaccharides), poly-alpha-hydroxy acid), polyvinyl alcohol (PVA), polyphosphazene, polyoxazolines (POZ), poly(N-acryloylmorpholine), or a combination thereof.

In some embodiments, binding protein can be conjugated to one or more polyethylene glycol molecules or “PEGylated.” Although the method or site of PEG attachment to the binding protein may vary, in certain embodiments the PEGylation does not alter, or only minimally alters, the activity of the binding protein. A variety of technologies are available for site specific incorporation of PEG moieties as reviewed in Dozier, J. K. and Distefano, M. D. (2015) “Site Specific Pegylation of Therapeutic Proteins” International Journal of Molecular Science 16(10):25832-25864.

In some embodiments, selective PEGylation of the binding protein, for example, by the incorporation of non-natural amino acids having side chains to facilitate selective PEG conjugation, may be employed. Specific PEGylation sites can be chosen such that PEGylation of the binding protein does not affect its binding to the target receptors.

In certain embodiments, the increase in half-life is greater than any decrease in biological activity. PEGS suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGS are contemplated by the present disclosure.

A molecular weight of the PEG used in the present disclosure is not restricted to any particular range. The PEG component of the binding protein can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa, or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

The present disclosure also contemplates compositions of conjugates wherein the PEGS have different n values, and thus the various different PEGS are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGS attached, purified free from unmodified protein sequences and from conjugates having other numbers of PEGs attached.

PEGS suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons.

Two widely used first generation activated monomethoxy PEGS (mPEGs) are succinimidyl carbonate PEG (SC-PEG; see, e.g., Zalipsky, et al. (1992) Biotehnol. App. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

Pegylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General PEGylation strategies known in the art can be applied herein.

The PEG can be bound to a binding protein of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the PEGylation of the binding proteins is facilitated by the incorporation of non-natural amino acids bearing unique side chains to facilitate site specific PEGylation. The incorporation of non-natural amino acids into polypeptides to provide functional moieties to achieve site specific PEGylation of such polypeptides is known in the art. See e.g., Ptacin et al., PCT International Application No. PCT/US2018/045257 filed Aug. 3, 2018 and published Feb. 7, 2019 as International Publication Number WO 2019/028419A1.

The PEG conjugated to the polypeptide sequence can be linear or branched. Branched PEG derivatives, “star-PEGs” and multi-armed PEGS are contemplated by the present disclosure. Specific embodiments PEGS useful in the practice of the present disclosure include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g., Sunbright® ME-200AL, NOF), a 20 kDa linear PEG-NHS ester (e.g., Sunbright® ME-20005, Sunbright® ME-200AS, Sunbright® ME-200G5, Sunbright® ME-200H5, NOF), a 20 kDa 2-arm branched PEG-aldehyde the 20 kDA PEG-aldehyde comprising two 10kDA linear PEG molecules (e.g., Sunbright® GL2-200AL3, NOF), a 20 kDa 2-arm branched PEG-NHS ester the 20 kDA PEG-NHS ester comprising two 10kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20kDA linear PEG molecules (e.g., Sunbright® GL2-400AL3), a 40 kDa 2-arm branched PEG-NHS ester the 40 kDA PEG-NHS ester comprising two 20kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

In some embodiments, a linker can used to join the binding protein and the PEG molecule. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids.

Examples of flexible linkers include glycine polymers (G)n, glycine-alanine polymers, alanine-serine polymers, glycine-serine polymers (for example, (GmSo)n (SEQ ID NO: 798), (GSGGS)n (SEQ ID NO: 799), (GmSoGm)n (SEQ ID NO: 800), (GmSoGmSoGm)n (SEQ ID NO: 801), (GSGGSm)n (SEQ ID NO: 802), (GSGSmG)n (SEQ ID NO: 803) and (GGGSm)n (SEQ ID NO: 804), and combinations thereof, where m, n, and o are each independently selected from an integer of at least 1 to 20, e.g., 1-18, 216, 3-14, 4-12, 5-10, 1, 2, 3, 4, 5, 6, 7, 8, 9, or 10), and other flexible linkers. Glycine and glycine-serine polymers are relatively unstructured, and therefore may serve as a neutral tether between components. Examples of flexible linkers are described in Section V.

Additional examples of flexible linkers include glycine polymers (G)n or glycine-serine polymers (e.g., (GS)n (SEQ ID NO: 805), (GSGGS)n (SEQ ID NO: 806), (GGGS)n (SEQ ID NO: 807) and (GGGGS)n (SEQ ID NO: 808), where n=1 to 50, for example, 1, 2, 3, 4, 5, 6, 7, 8, 9, 10, 10-20, 20-30, 30-50). A multimer (e.g., 1, 2, 3, 4, 5, 6, 7, 8, 9, 10, 10-20, 20-30, or 30-50) of these linker sequences may be linked together to provide flexible linkers that may be used to conjugate two molecules. Alternative to a polypeptide linker, the linker can be a chemical linker, e.g., a PEG-aldehyde linker. In some embodiments, the binding protein is acetylated at the N-terminus by enzymatic reaction with N-terminal acetyltransferase and, for example, acetyl CoA. Alternatively, or in addition to N-terminal acetylation, the binding protein can be acetylated at one or more lysine residues, e.g., by enzymatic reaction with a lysine acetyltransferase. See, for example Choudhary et al. (2009) Science 325 (5942):834-840.

In other embodiments, the binding protein can be modified to include an additional polypeptide sequence that functions as an antigenic tag, such as a FLAG sequence. FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the binding protein further comprises a C-terminal c-myc epitope tag.

In some embodiments, the binding protein is expressed as a fusion protein with an albumin molecule (e.g., human serum albumin) which is known in the art to facilitate extended exposure in vivo.

In some embodiment, the binding proteins (including fusion proteins of the binding proteins) of the present disclosure are expressed as a fusion protein with one or more transition metal chelating polypeptide sequences. The incorporation of such a transition metal chelating domain facilitates purification immobilized metal affinity chromatography (IMAC) as described in Smith, et al. U.S. Pat. No. 4,569,794 issued Feb. 11, 1986. Examples of transition metal chelating polypeptides useful in the practice of the present disclosure are described in Smith, et al. supra and Dobeli, et al. U.S. Pat. No. 5,320,663 issued May 10, 1995, the entire teachings of which are hereby incorporated by reference. Particular transition metal chelating polypeptides useful in the practice of the present disclosure are peptides comprising 3-6 contiguous histidine residues (SEQ ID NO: 809) such as a six-histidine peptide (His)₆ (SEQ ID NO: 127) and are frequently referred to in the art as “His-tags.”

The foregoing fusion proteins may be readily produced by recombinant DNA methodology by techniques known in the art by constructing a recombinant vector comprising a nucleic acid sequence comprising a nucleic acid sequence encoding the binding protein in frame with a nucleic acid sequence encoding the fusion partner either at the N-terminus or C-terminus of the binding protein, the sequence optionally further comprising a nucleic acid sequence in frame encoding a linker or spacer polypeptide.

### VII. Pharmaceutical Composition

The binding proteins of the present disclosure may be administered to a subject in a pharmaceutically acceptable dosage form. The preferred formulation depends on the intended mode of administration and therapeutic application. Pharmaceutical dosage forms of the binding proteins described herein comprise physiologically acceptable carriers that are inherently non-toxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of polypeptides include polysaccharides such as sodium carboxymethylcellulose or methylcellulose, polyvinylpyrrolidone, polyacrylates, polyoxyethylene-polyoxypropylene-block polymers, PEG, polymeric amino acids, amino acid copolymers, and lipid aggregates (such as oil droplets or liposomes).

The pharmaceutical compositions may also comprise pharmaceutically-acceptable, non-toxic carriers, excipients, stabilizers, or diluents, which are defined as vehicles commonly used to formulate pharmaceutical compositions for animal or human administration. The diluent is selected so as not to affect the biological activity of the combination. Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g.. Zn-protein complexes); and/or non-ionic surfactants such as TWEEN™, PLURONICS™ or polyethylene glycol (PEG).

Formulations to be used for in vivo administration are typically sterile. Sterilization of the compositions of the present disclosure may readily accomplished by filtration through sterile filtration membranes.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (Langer, Science 249: 1527, 1990 and Hanes, Advanced Drug Delivery Reviews 28: 97-119, 1997). The agents of this disclosure can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Administration of a binding protein described herein may be achieved through any of a variety of art recognized methods including but not limited to the topical, intravascular injection (including intravenous or intraarterial infusion), intradermal injection, subcutaneous injection, intramuscular injection, intraperitoneal injection, intracranial injection, intratumoral injection, intranodal injection, transdermal, transmucosal, iontophoretic delivery, intralymphatic injection (Senti and Kundig (2009) Current Opinions in Allergy and Clinical Immunology 9(6):537-543), intragastric infusion, intraprostatic injection, intravesical infusion (e.g., bladder), respiratory inhalers including nebulizers, intraocular injection, intraabdominal injection, intralesional injection, intraovarian injection, intracerebral infusion or injection, intracerebroventricular injection (ICVI), and the like. In some embodiments, administration includes the administration of the binding protein itself (e.g., parenteral), as well as the administration of a recombinant vector (e.g., viral or non-viral vector) to cause the in situ expression of the binding protein in the subject. Alternatively, a cell, such as a cell isolated from the subject, could also be recombinantly modified to express the binding protein of the present disclosure.

The dosage of the pharmaceutical compositions depends on factors including the route of administration, the disease to be treated, and physical characteristics, e.g., age, weight, general health, of the subject. Typically, the amount of a binding protein contained within a single dose may be an amount that effectively prevents, delays, or treats the disease without inducing significant toxicity. A pharmaceutical composition of the disclosure may include a dosage of a binding protein described herein ranging from 0.01 to 500 mg/kg (e.g., from 0.01 to 450 mg, from 0.01 to 400 mg, from 0.01 to 350 mg, from 0.01 to 300 mg, from 0.01 to 250 mg, from 0.01 to 200 mg, from 0.01 to 150 mg, from 0.01 to 100 mg, from 0.01 to 50 mg, from 0.01 to 10 mg, from 0.01 to 1 mg, from 0.1 to 500 mg/kg, from 1 to 500 mg/kg, from 5 to 500 mg/kg, from 10 to 500 mg/kg, from 50 to 500 mg/kg, from 100 to 500 mg/kg, from 150 to 500 mg/kg, from 200 to 500 mg/kg, from 250 to 500 mg/kg, from 300 to 500 mg/kg, from 350 to 500 mg/kg, from 400 to 500 mg/kg, or from 450 to 500 mg/kg) and, in a more specific embodiment, about 1 to about 100 mg/kg (e.g., about 1 to about 90 mg/kg, about 1 to about 80 mg/kg, about 1 to about 70 mg/kg, about 1 to about 60 mg/kg, about 1 to about 50 mg/kg, about 1 to about 40 mg/kg, about 1 to about 30 mg/kg, about 1 to about 20 mg/kg, about 1 to about 10 mg/kg, about 10 to about 100 mg/kg, about 20 to about 100 mg/kg, about 30 to about 100 mg/kg, about 40 to about 100 mg/kg, about 50 to about 100 mg/kg, about 60 to about 100 mg/kg, about 70 to about 100 mg/kg, about 80 to about 100 mg/kg, or about 90 to about 100 mg/kg). In some embodiments, a pharmaceutical composition of the disclosure may include a dosage of a binding protein described herein ranging from 0.01 to 20 mg/kg (e.g., from 0.01 to 15 mg/kg, from 0.01 to 10 mg/kg, from 0.01 to 8 mg/kg, from 0.01 to 6 mg/kg, from 0.01 to 4 mg/kg, from 0.01 to 2 mg/kg, from 0.01 to 1 mg/kg, from 0.01 to 0.1 mg/kg, from 0.01 to 0.05 mg/kg, from 0.05 to 20 mg/kg, from 0.1 to 20 mg/kg, from 1 to 20 mg/kg, from 2 to 20 mg/kg, from 4 to 20 mg/kg, from 6 to 20 mg/kg, from 8 to 20 mg/kg, from 10 to 20 mg/kg, from 15 to 20 mg/kg). The dosage may be adapted by the physician in accordance with conventional factors such as the extent of the disease and different parameters of the subject.

A pharmaceutical composition containing a binding protein described herein can be administered to a subject in need thereof, for example, one or more times (e.g., 1-10 times or more) daily, weekly, monthly, biannually, annually, or as medically necessary. Dosages may be provided in either a single or multiple dosage regimens. The timing between administrations may decrease as the medical condition improves or increase as the health of the patient declines. A course of therapy may be a single dose or in multiple doses over a period of time. In some embodiments, a single dose is used. In some embodiments, two or more split doses administered over a period of 1, 2, 3, 4, 5, 6, 7, 8, 9, 10, 11, 12, 13, 14, 21, 28, 30, 60, 90, 120 or 180 days are used. Each dose administered in such split dosing protocols may be the same in each administration or may be different. Multi-day dosing protocols over time periods may be provided by the skilled artisan (e.g., physician) monitoring the administration, taking into account the response of the subject to the treatment including adverse effects of the treatment and their modulation as discussed above. In some embodiments, the serum trough concentration of the binding molecule is maintained above a threshold level corresponding to about 0.1 pg/ml, alternatively 0.1 ng/ml, alternatively about 0.5 ng/ml alternatively 1 ng/ml, alternatively 2 ng/ml, for at least 80%, alternatively at least 85%, alternatively at least 90%, alternatively at least 95% of a period of time of at least 24 hours, alternatively 48 hours, alternatively 72 hours, alternatively one week, alternatively 1 month. See, e.g. Mumm, et al. United States Patent Publication US2016/0193300A1 published Jul. 7, 2016.

### VIII. Methods of Use

### Neoplastic Diseases

The present disclosure provides methods of use of binding proteins that bind to IL2Rβ and IL2Rγ in the treatment of subjects suffering from a neoplastic disease disorder or condition by the administration of a therapeutically effective amount of a binding protein (or nucleic acid encoding a binding protein including recombinant vectors encoding the binding protein) as described herein.

The compositions and methods of the present disclosure are useful in the treatment of subject suffering from a neoplastic disease characterized by the presence neoplasms, including benign and malignant neoplasms, and neoplastic disease.

Examples benign neoplasms amenable to treatment using the compositions and methods of the present disclosure include but are not limited to adenomas, fibromas, hemangiomas, and lipomas. Examples of pre-malignant neoplasms amenable to treatment using the compositions and methods of the present disclosure include but are not limited to hyperplasia, atypia, metaplasia, and dysplasia. Examples of malignant neoplasms amenable to treatment using the compositions and methods of the present disclosure include but are not limited to carcinomas (cancers arising from epithelial tissues such as the skin or tissues that line internal organs), leukemias, lymphomas, and sarcomas typically derived from bone fat, muscle, blood vessels or connective tissues). Also included in the term neoplasms are viral induced neoplasms such as warts and EBV induced disease (i.e., infectious mononucleosis), scar formation, hyperproliferative vascular disease including intimal smooth muscle cell hyperplasia, restenosis, and vascular occlusion and the like.

The term “neoplastic disease” includes cancers characterized by solid tumors and non-solid tumors including, but not limited to, breast cancers, sarcomas (including but not limited to osteosarcomas and angiosarcomas and fibrosarcomas), leukemias, lymphomas, genitourinary cancers (including but not limited to ovarian, urethral, bladder, and prostate cancers), gastrointestinal cancers (including but not limited to colon esophageal and stomach cancers), lung cancers, myelomas, pancreatic cancers, liver cancers, kidney cancers, endocrine cancers, skin cancers, and brain or central and peripheral nervous (CNS) system tumors, malignant or benign, including gliomas and neuroblastomas, astrocytomas, myelodysplastic disorders, cervical carcinoma-in-situ, intestinal polyposes, oral leukoplakias, histiocytoses, hyperprofroliferative scars including keloid scars, hemangiomas, hyperproliferative arterial stenosis, psoriasis, inflammatory arthritis, hyperkeratoses, and papulosquamous eruptions including arthritis.

The term “neoplastic disease” includes carcinomas. The term “carcinoma” refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The term neoplastic disease includes adenocarcinomas. An “adenocarcinoma” refers to a carcinoma derived from glandular tissue or in which the tumor cells form recognizable glandular structures.

As used herein, the term “hematopoietic neoplastic disorders” refers to neoplastic diseases involving hyperplastic/neoplastic cells of hematopoietic origin, e.g., arising from myeloid, lymphoid or erythroid lineages, or precursor cells thereof.

Myeloid neoplasms include, but are not limited to, myeloproliferative neoplasms, myeloid and lymphoid disorders with eosinophilia, myeloproliferative/myelodysplastic neoplasms, myelodysplastic syndromes, acute myeloid leukemia and related precursor neoplasms, and acute leukemia of ambiguous lineage. Exemplary myeloid disorders amenable to treatment in accordance with the present disclosure include, but are not limited to, acute promyeloid leukemia (APML), acute myelogenous leukemia (AML), and chronic myelogenous leukemia (CML).

Lymphoid neoplasms include, but are not limited to, precursor lymphoid neoplasms, mature B-cell neoplasms, mature T-cell neoplasms, Hodgkin's Lymphoma, and immunodeficiency-associated lymphoproliferative disorders. Exemplary lymphic disorders amenable to treatment in accordance with the present disclosure include, but are not limited to, acute lymphoblastic leukemia (ALL) which includes B-lineage ALL and T-lineage ALL, chronic lymphocytic leukemia (CLL), prolymphocytic leukemia (PLL), hairy cell leukemia (HLL), and Waldenstrom's macroglobulinemia (WM).

In some instances, the hematopoietic neoplastic disorder arises from poorly differentiated acute leukemias (e.g., erythroblastic leukemia and acute megakaryoblastic leukemia). As used herein, the term “hematopoietic neoplastic disorders” refers malignant lymphomas including, but are not limited to, non-Hodgkins lymphoma and variants thereof, peripheral T cell lymphomas, adult T-cell leukemia/lymphoma (ATL), cutaneous T cell lymphoma (CTCL), large granular lymphocytic leukemia (LGF), Hodgkin's disease, and Reed-Stemberg disease.

The determination of whether a subject is “suffering from a neoplastic disease” refers to a determination made by a physician with respect to a subject based on the available information accepted in the field for the identification of a disease, disorder or condition including but not limited to X-ray, CT-scans, conventional laboratory diagnostic tests (e.g. blood count, etc.), genomic data, protein expression data, immunohistochemistry, that the subject requires or will benefit from treatment.

### Assessing Anti-Neoplastic Efficacy

The determination of efficacy of the methods of the present disclosure in the treatment of cancer is generally associated with the achievement of one or more art recognized parameters such as reduction in lesions particularly reduction of metastatic lesion, reduction in metastatsis, reduction in tumor volume, improvement in ECOG score, and the like. Determining response to treatment can be assessed through the measurement of biomarker that can provide reproducible information useful in any aspect of binding protein therapy, including the existence and extent of a subject's response to such therapy and the existence and extent of untoward effects caused by such therapy. By way of example, but not limitation, biomarkers include enhancement of IFNγ, and upregulation of granzyme A, granzyme B, and perforin; increase in CD8⁺ T-cell number and function; enhancement of IFNγ, an increase in ICOS expression on CD8⁺ T-cells, enhancement of IL10 expressing T_{Reg} cells. The response to treatment may be characterized by improvements in conventional measures of clinical efficacy may be employed such as Complete Response (CR), Partial Response (PR), Stable Disease (SD) and with respect to target lesions, Complete Response (CR),” Incomplete Response/Stable Disease (SD) as defined by RECIST as well as immune-related Complete Response (irCR), immune-related Partial Response (irPR), and immune-related Stable Disease (irSD) as defined Immune-Related Response Criteria (irRC) are considered by those of skill in the art as evidencing efficacy in the treatment of neoplastic disease in mammalian (e.g., human) subjects.

Further embodiments comprise a method or model for determining the optimum amount of an agent(s) in a combination. An optimum amount can be, for example, an amount that achieves an optimal effect in a subject or subject population, or an amount that achieves a therapeutic effect while minimizing or eliminating the adverse effects associated with one or more of the agents. In some embodiments, the methods involving the combination of a binding protein described herein and a supplementary agent which is known to be, or has been determined to be, effective in treating or preventing a disease, disorder or condition described herein (e.g., a cancerous condition) in a subject (e.g., a human) or a subject population, and an amount of one agent is titrated while the amount of the other agent(s) is held constant. By manipulating the amounts of the agent(s) in this manner, a clinician is able to determine the ratio of agents most effective for, for example, treating a particular disease, disorder or condition, or eliminating the adverse effects or reducing the adverse effects such that are acceptable under the circumstances.

Combination Of Binding Proteins with Supplementary Therapeutic Agents

The present disclosure provides the for the use of the binding proteins of the present disclosure in combination with one or more additional active agents (“supplementary agents”). Such further combinations are referred to interchangeably as “supplementary combinations” or “supplementary combination therapy” and those therapeutic agents that are used in combination with binding proteins of the present disclosure are referred to as “supplementary agents.” As used herein, the term “supplementary agents” includes agents that can be administered or introduced separately, for example, formulated separately for separate administration (e.g., as may be provided in a kit) and/or therapies that can be administered or introduced in combination with the binding proteins.

As used herein, the term “in combination with” when used in reference to the administration of multiple agents to a subject refers to the administration of a first agent at least one additional (i.e. second, third, fourth, fifth, etc.) agent to a subject. For purposes of the present invention, one agent (e.g., a binding protein described herein) is considered to be administered in combination with a second agent (e.g. a modulator of an immune checkpoint pathway) if the biological effect resulting from the administration of the first agent persists in the subject at the time of administration of the second agent such that the therapeutic effects of the first agent and second agent overlap. For example, the PD1 immune checkpoint inhibitors (e.g. nivolumab or pembrolizumab) are typically administered by IV infusion every two weeks or every three weeks while the binding proteins of the present disclosure are typically administered more frequently, e.g. daily, BID, or weekly. However, the administration of the first agent (e.g. pembrolizumab) provides a therapeutic effect over an extended time and the administration of the second agent (e.g., a binding protein described herein) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject.

In certain embodiments, the binding protein and the supplementary agent(s) are administered or applied sequentially, e.g., where one agent is administered prior to one or more other agents. In other embodiments, the binding protein and the supplementary agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

### Chemotherapeutic Agents

In some embodiments, the supplementary agent is a chemotherapeutic agent. In some embodiments the supplementary agent is a “cocktail” of multiple chemotherapeutic agents. In some embodiments the chemotherapeutic agent or cocktail is administered in combination with one or more physical methods (e.g., radiation therapy). The term “chemotherapeutic agents” includes but is not limited to alkylating agents such as thiotepa and cyclosphosphamide, alkyl sulfonates such as busulfan, improsulfan and piposulfan, aziridines such as benzodopa, carboquone, meturedopa, and uredopa, ethylenimines and methylamelamines including altretamine, triethylenemelamine, trietylenephosphoramide, triethylenethiophosphaoramide and trimethylolomelamime, nitrogen mustards such as chiorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard, nitrosureas such as carmustine, chlorozotocin, fotemustine, lomustine, nimustine, ranimustine, antibiotics such as aclacinomysins, actinomycin, authramycin, azaserine, bleomycins such as bleomycin A₂, cactinomycin, calicheamicin, carabicin, caminomycin, carzinophilin, chromomycins, dactinomycin, daunorubicin and derivaties such as demethoxy-daunomycin, 11-deoxydaunorubicin, 13-deoxydaunorubicin, detorubicin, 6-diazo-5-oxo-L-norleucine, doxorubicin, epirubicin, esorubicin, idarubicin, marcellomycin, mitomycins such as mitomycin C, N-methyl mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, anti-metabolites such as methotrexate and 5-fluorouracil (5-FU), folic acid analogues such as denopterin, methotrexate, pteropterin, trimetrexate, dideazatetrahydrofolic acid, and folinic acid, purine analogs such as fludarabine, 6-mercaptopurine, thiamiprine, thioguanine, pyrimidine analogs such as ancitabine, azacitidine, 6-azauridine, carmofur, cytarabine, dideoxyuridine, doxifluridine, enocitabine, floxuridine, 5-FU, androgens such as calusterone, dromostanolone propionate, epitiostanol, mepitiostane, testolactone, anti-adrenals such as aminoglutethimide, mitotane, trilostane, folic acid replenisher such as frolinic acid, aceglatone, aldophosphamide glycoside, aminolevulinic acid, amsacrine, bestrabucil, bisantrene, edatraxate, defofamine, demecolcine, diaziquone, elformithine, elliptinium acetate, etoglucid, gallium nitrate, hydroxyurea, lentinan, lonidamine, mitoguazone, mitoxantrone, mopidamol, nitracrine, pentostatin, phenamet, pirarubicin, podophyllinic acid, 2-ethylhydrazide, procarbazine, razoxane, sizofiran, spirogermanium, tenuazonic acid, triaziquone, 2,2′,2″-trichlorotriethylamine, urethan, vindesine, dacarbazine, mannomustine, mitobronitol, mitolactol, pipobroman, gacytosine, arabinoside (Ara-C), cyclophosphamide, thiotepa, taxoids, e.g., paclitaxel, nab-paclitaxel and doxetaxel, chlorambucil, gemcitabine, 6-thioguanine, mercaptopurine, methotrexate, platinum and platinum coordination complexes such as cisplatin, oxaplatin and carboplatin, vinblastine, etoposide (VP-16), ifosfamide, mitomycin C, mitoxantrone, vincristine, vinorelbine, navelbine, novantrone, teniposide, daunomycin, aminopterin, xeloda, ibandronate, CPT11, topoisomerase inhibitors, difluoromethylomithine (DMFO), retinoic acid, esperamicins, capecitabine, taxanes such as paclitaxel, docetaxel, cabazitaxel, carminomycin, adriamycins such as 4′-epiadriamycin, 4-adriamycin-14-benzoate, adriamycin-14-octanoate, adriamycin-14-naphthaleneacetate, cholchicine and pharmaceutically acceptable salts, acids or derivatives of any of the above.

The term “chemotherapeutic agents” also includes anti-hormonal agents that act to regulate or inhibit hormone action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above.

In some embodiments, a supplementary agent is one or more chemical or biological agents identified in the art as useful in the treatment of neoplastic disease, including, but not limited to, a cytokines or cytokine antagonists such as IL12, INFα, or anti-epidermal growth factor receptor, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g.. Gleevec® or Herceptin®) or an immunomodulator to achieve additive or synergistic suppression of tumor growth, non-steroidal anti-inflammatory drugs (NSAIDs), cyclooxygenase-2 (COX-2) inhibitors, steroids, TNF antagonists (e.g.. Remicade® and Enbrel®), interferon-β1a (Avonex®), and interferon-β1b (Betaseron®) as well as combinations of one or more of the foreoing as practied in known chemotherapeutic treatment regimens including but not limited to TAC, FOLFOX, TPC, FEC, ADE, FOLFOX-6, EPOCH, CHOP, CMF, CVP, BEP, OFF, FLOX, CVD, TC, FOLFIRI, PCV, FOLFOXIRI, ICE-V, XELOX, and others that are readily appreciated by the skilled clinician in the art.

In some embodiments, the binding protein is administered in combination with BRAF/MEK inhibitors, kinase inhibitors such as sunitinib, PARP inhibitors such as olaparib, EGFR inhibitors such as osimertinib (Ahn, et al. (2016) J Thorac Oncol 11:S115), IDO inhibitors such as epacadostat, and oncolytic viruses such as talimogene laherparepvec (T-VEC).

Combination with Therapeutic Antibodies

In some embodiments, a “supplementary agent” is a therapeutic antibody (including bi-specific and tri-specific antibodies which bind to one or more tumor associated antigens including but not limited to bispecific T cell engagers (BITEs), dual affinity retargeting (DART) constructs, and trispecific killer engager (TriKE) constructs).

In some embodiments, the therapeutic antibody is an antibody that binds to at least one tumor antigen selected from the group consisting of HER2 (e.g. trastuzumab, pertuzumab, ado-trastuzumab emtansine), nectin-4 (e.g. enfortumab), CD79 (e.g. polatuzumab vedotin), CTLA4 (e.g. ipilumumab), CD22 (e.g. moxetumomab pasudotox), CCR4 (e.g. magamuizumab), IL23p19 (e.g. tildrakizumab), PDL1 (e.g. durvalumab, avelumab, atezolizumab), IL17a (e.g. ixekizumab), CD38 (e.g. daratumumab), SLAMF7 (e.g. elotuzumab), CD20 (e.g. rituximab, tositumomab, ibritumomab and ofatumumab), CD30 (e.g. brentuximab vedotin), CD33 (e.g. gemtuzumab ozogamicin), CD52 (e.g. alemtuzumab), EpCam, CEA, fpA33, TAG-72, CAIX, PSMA, PSA, folate binding protein, GD2 (e.g. dinuntuximab), GD3, IL6 (e.g. silutxumab) GM2, Ley, VEGF (e.g. bevacizumab), VEGFR, VEGFR2 (e.g. ramucirumab), PDGFRα (e.g. olartumumab), EGFR (e.g. cetuximab, panittunurnab and necittuntunab), ERBB2 (e.g. trasturtunab), ERBB3, MET, IGF1R, EPHA3, TRAIL R1, TRAIL R2, RANKL RAP, tenascin, integrin αVβ3, and integrin α4β1.

Examples of antibody therapeutics which are FDA approved and may be used as supplementary agents for use in the treatment of neoplastic disease indication include those provided in Table 5 below.

TABLE 5
FDA Antineoplastic Disease Antibodies and Indications

| Name | Tradename(s) | Target; format | Indication |
|---|---|---|---|
| [fam]-trastuzumab | Enhertu | HER2; Humanized IgG1 ADC | HER2+ breast cancer |
| deruxtecan | | | |
| Enfortumab vedotin | Padcev | Nectin-4; Human IgG1 ADC | Urothelial cancer |
| Polatuzumab vedotin | Polivy | CD79b; Humanized IgG1 ADC | Diffuse large B-cell lymphoma |
| Cemiplimab | Libtayo | PD-1; Human mAb | Cutaneous squamous cell carcinoma |
| Moxetumomab | Lumoxiti | CD22; Murine IgG1 dsFv | Hairy cell leukemia |
| pasudotox | | immunotoxin | |
| Mogamuizumab | Poteligeo | CCR4; Humanized IgG1 | Cutaneous T cell lymphoma |
| Tildrakizumab | Ilumya | IL23p19; Humanized IgG1 | Plaque psoriasis |
| Ibalizumab | Trogarzo | CD4; Humanized IgG4 | HIV infection |
| Durvalumab | IMFINZI | PD-L1; Human IgG1 | Bladder cancer |
| Inotuzumab | BESPONSA | CD22; Humanized IgG4, ADC | Hematological malignancy |
| ozogamicin | | | |
| Avelumab | Bavencio | PD-L1; Human IgG1 | Merkel cell carcinoma |
| Atezolizumab | Tecentriq | PD-L1; Humanized IgG1 | Bladder cancer |
| Olaratumab | Lartruvo | PDGRFα; Human IgG1 | Soft tissue sarcoma |
| Ixekizumab | Taltz | IL17a; Humanized IgG4 | Psoriasis |
| Daratumumab | Darzalex | CD38; Human IgG1 | Multiple myeloma |
| Elotuzumab | Empliciti | SLAMF7; Humanized IgG1 | Multiple myeloma |
| Necitumumab | Portrazza | EGFR; Human IgG1 | Non-small cell lung cancer |
| Dinutuximab | Unituxin | GD2; Chimeric IgG1 | Neuroblastoma |
| Nivolumab | Opdivo | PD1; Human IgG4 | Melanoma, non-small cell lung |
| | | | cancer |
| Blinatumomab | Blincyto | CD19, CD3; Murine bispecific | Acute lymphoblastic leukemia |
| | | tandem scFv | |
| Pembrolizumab | Keytruda | PD1; Humanized IgG4 | Melanoma |
| Ramucirumab | Cyramza | VEGFR2; Human IgG1 | Gastric cancer |
| Siltuximab | Sylvant | IL6; Chimeric IgG1 | Castleman disease |
| Obinutuzumab | Gazyva | CD20; Humanized IgG1; | Chronic lymphocytic leukemia |
| | | Glycoengineered | |
| Ado-trastuzumab | Kadcyla | HER2; Humanized IgG1, ADC | Breast cancer |
| emtansine | | | |
| Pertuzumab | Perjeta | HER2; Humanized IgG1 | Breast Cancer |
| Brentuximab vedotin | Adcetris | CD30; Chimeric IgG1, ADC | Hodgkin lymphoma, systemic |
| | | | anaplastic large cell lymphoma |
| Ipilimumab | Yervoy | CTLA-4; Human IgG1 | Metastatic melanoma |
| Ofatumumab | Arzerra | CD20; Human IgG1 | Chronic lymphocytic leukemia |
| Certolizumab pegol | Cimzia | TNF; Humanized Fab, pegylated | Crohn disease |
| Catumaxomab | Removab | EPCAM/CD3; Rat/mouse | Malignant ascites |
| | | bispecific mAb | |
| Panitumumab | Vectibix | EGFR; Human IgG2 | Colorectal cancer |
| Bevacizumab | Avastin | VEGF; Humanized IgG1 | Colorectal cancer |
| Cetuximab | Erbitux | EGFR; Chimeric IgG1 | Colorectal cancer |
| Tositumomab-I131 | Bexxar | CD20; Murine IgG2a | Non-Hodgkin lymphoma |
| Ibritumomab tiuxetan | Zevalin | CD20; Murine IgG1 | Non-Hodgkin lymphoma |
| Gemtuzumab | Mylotarg | CD33; Humanized IgG4, ADC | Acute myeloid leukemia |
| ozogamicin | | | |
| Trastuzumab | Herceptin | HER2; Humanized IgG1 | Breast cancer |
| Infliximab | Remicade | TNF; Chimeric IgG1 | Crohn disease |
| Rituximab | Mab Thera, Rituxan | CD20; Chimeric IgG1 | Non-Hodgkin lymphoma |
| Edrecolomab | Panorex | EpCAM; Murine IgG2a | Colorectal cancer |

In some embodiments, where the antibody is a bispecific antibody targeting a first and second tumor antigen such as HER2 and HER3 (abbreviated HER2×HER3), FAP×DR-5 bispecific antibodies, CEA×CD3 bispecific antibodies, CD20×CD3 bispecific antibodies, EGFR-EDV-miR16 trispecific antibodies, gp100×CD3 bispecific antibodies, Ny-eso×CD3 bispecific antibodies, EGFR×cMet bispecific antibodies, BCMA×CD3 bispecific antibodies, EGFR-EDV bispecific antibodies, CLEC12A×CD3 bispecific antibodies, HER2×HER3 bispecific antibodies, Lgr5×EGFR bispecific antibodies, PD1×CTLA-4 bispecific antibodies, CD123×CD3 bispecific antibodies, gpA33×CD3 bispecific antibodies, B7-H3×CD3 bispecific antibodies, LAG-3×PD1 bispecific antibodies, DLL4×VEGF bispecific antibodies, Cadherin-P×CD3 bispecific antibodies, BCMA×CD3 bispecific antibodies, DLL4×VEGF bispecific antibodies, CD20×CD3 bispecific antibodies, Ang-2×VEGF-A bispecific antibodies,

CD20×CD3 bispecific antibodies, CD123×CD3 bispecific antibodies, SSTR2×CD3 bispecific antibodies, PD1×CTLA-4 bispecific antibodies, HER2×HER2 bispecific antibodies, GPC3×CD3 bispecific antibodies, PSMA×CD3 bispecific antibodies, LAG-3×PD-L1 bispecific antibodies, CD38×CD3 bispecific antibodies, HER2×CD3 bispecific antibodies, GD2×CD3 bispecific antibodies, and CD33×CD3 bispecific antibodies.

Such therapeutic antibodies may be further conjugated to one or more chemotherapeutic agents (e.g., antibody drug conjugates or ADCs) directly or through a linker, especially acid, base or enzymatically labile linkers.

Combination with Physical Methods

In some embodiments, a supplementary agent is one or more non-pharmacological modalities (e.g., localized radiation therapy or total body radiation therapy or surgery). By way of example, the present disclosure contemplates treatment regimens wherein a radiation phase is preceded or followed by treatment with a treatment regimen comprising a binding protein and one or more supplementary agents. In some embodiments, the present disclosure further contemplates the use of a binding protein in combination with surgery (e.g. tumor resection). In some embodiments, the present disclosure further contemplates the use of a binding protein in combination with bone marrow transplantation, peripheral blood stem cell transplantation or other types of transplantation therapy.

Combination with Immune Checkpoint Modulators:

In some embodiments, a “supplementary agent” is an immune checkpoint modulator for the treatment and/or prevention neoplastic disease in a subject as well as diseases, disorders or conditions associated with neoplastic disease. The term “immune checkpoint pathway” refers to biological response that is triggered by the binding of a first molecule (e.g. a protein such as PD1) that is expressed on an antigen presenting cell (APC) to a second molecule (e.g. a protein such as PDL1) that is expressed on an immune cell (e.g. a T-cell) which modulates the immune response, either through stimulation (e.g. upregulation of T-cell activity) or inhibition (e.g. downregulation of T-cell activity) of the immune response. The molecules that are involved in the formation of the binding pair that modulate the immune response are commonly referred to as “immune checkpoints.” The biological responses modulated by such immune checkpoint pathways are mediated by intracellular signaling pathways that lead to downstream immune effector pathways, such as cell activation, cytokine production, cell migration, cytotoxic factor secretion, and antibody production. Immune checkpoint pathways are commonly triggered by the binding of a first cell surface expressed molecule to a second cell surface molecule associated with the immune checkpoint pathway (e.g. binding of PD1 to PDL1, CTLA4 to CD28, etc.). The activation of immune checkpoint pathways can lead to stimulation or inhibition of the immune response.

An immune checkpoint whose activation results in inhibition or downregulation of the immune response is referred to herein as a “negative immune checkpoint pathway modulator.” The inhibition of the immune response resulting from the activation of a negative immune checkpoint modulator diminishes the ability of the host immune system to recognize foreign antigen such as a tumor-associated antigen. The term negative immune checkpoint pathway includes, but is not limited to, biological pathways modulated by the binding of PD1 to PDL1, PD1 to PDL2, and CTLA4 to CDCD80/86. Examples of such negative immune checkpoint antagonists include but are not limited to antagonists (e.g. antagonist antibodies) that bind T-cell inhibitory receptors including but not limited to PD1 (also referred to as CD279), TIM3 (T-cell membrane protein 3; also known as HAVcr2), BTLA (B and T lymphocyte attenuator; also known as CD272), the VISTA (B7-H5) receptor, LAG3 (lymphocyte activation gene 3; also known as CD233) and CTLA4 (cytotoxic T-lymphocyte associated antigen 4; also known as CD152).

In one embodiment, an immune checkpoint pathway the activation of which results in stimulation of the immune response is referred to herein as a “positive immune checkpoint pathway modulator.” The term positive immune checkpoint pathway modulator includes, but is not limited to, biological pathways modulated by the binding of ICOSL to ICOS(CD278), B7-H6 to NKp30, CD155 to CD96, OX40L to OX40, CD70 to CD27, CD40 to CD40L, and GITRL to GITR. Molecules which agonize positive immune checkpoints (such natural or synthetic ligands for a component of the binding pair that stimulates the immune response) are useful to upregulate the immune response. Examples of such positive immune checkpoint agonists include but are not limited to agonist antibodies that bind T-cell activating receptors such as ICOS (such as JTX-2011, Jounce Therapeutics), OX40 (such as MEDI6383, Medimmune), CD27 (such as varlilumab, Celldex Therapeutics), CD40 (such as dacetuzmumab CP-870,893, Roche, Chi Lob 7/4), HVEM, CD28, CD137 4-1BB, CD226, and GITR (such as MEDI1873, Medimmune; INCAGN 1876, Agenus).

As used herein, the term “immune checkpoint pathway modulator” refers to a molecule that inhibits or stimulates the activity of an immune checkpoint pathway in a biological system including an immunocompetent mammal. An immune checkpoint pathway modulator may exert its effect by binding to an immune checkpoint protein (such as those immune checkpoint proteins expressed on the surface of an antigen presenting cell (APC) such as a cancer cell and/or immune T effector cell) or may exert its effect on upstream and/or downstream reactions in the immune checkpoint pathway. For example, an immune checkpoint pathway modulator may modulate the activity of SHP2, a tyrosine phosphatase that is involved in PD-1 and CTLA-4 signaling. The term “immune checkpoint pathway modulators” encompasses both immune checkpoint pathway modulator(s) capable of down-regulating at least partially the function of an inhibitory immune checkpoint (referred to herein as an “immune checkpoint pathway inhibitor” or “immune checkpoint pathway antagonist”) and immune checkpoint pathway modulator(s) capable of up-regulating at least partially the function of a stimulatory immune checkpoint (referred to herein as an “immune checkpoint pathway effector” or “immune checkpoint pathway agonist.”).

The immune response mediated by immune checkpoint pathways is not limited to T-cell mediated immune response. For example, the KIR receptors of NK cells modulate the immune response to tumor cells mediated by NK cells. Tumor cells express a molecule called HLA-C, which inhibits the KIR receptors of NK cells leading to a dimunition or the anti-tumor immune response. The administration of an agent that antagonizes the binding of HLA-C to the KIR receptor such an anti-KIR3 mab (e.g. lirilumab, BMS) inhibits the ability of HLA-C to bind the NK cell inhibitory receptor (KIR) thereby restoring the ability of NK cells to detect and attack cancer cells. Thus, the immune response mediated by the binding of HLA-C to the KIR receptor is an example a negative immune checkpoint pathway the inhibition of which results in the activation of a of non-T-cell mediated immune response.

In one embodiment, the immune checkpoint pathway modulator is a negative immune checkpoint pathway inhibitor/antagonist. In another embodiment, immune checkpoint pathway modulator employed in combination with the binding protein is a positive immune checkpoint pathway agonist. In another embodiment, immune checkpoint pathway modulator employed in combination with the binding protein is an immune checkpoint pathway antagonist.

The term “negative immune checkpoint pathway inhibitor” refers to an immune checkpoint pathway modulator that interferes with the activation of a negative immune checkpoint pathway resulting in the upregulation or enhancement of the immune response. Exemplary negative immune checkpoint pathway inhibitors include but are not limited to programmed death-1 (PD1) pathway inhibitors, programed death ligand-1 (PDL1) pathway inhibitors, TIM3 pathway inhibitors and anti-cytotoxic T-lymphocyte antigen 4 (CTLA4) pathway inhibitors.

In one embodiment, the immune checkpoint pathway modulator is an antagonist of a negative immune checkpoint pathway that inhibits the binding of PD1 to PDL1 and/or PDL2 (“PD1 pathway inhibitor”). PD1 pathway inhibitors result in the stimulation of a range of favorable immune response such as reversal of T-cell exhaustion, restoration cytokine production, and expansion of antigen-dependent T-cells. PD1 pathway inhibitors have been recognized as effective variety of cancers receiving approval from the USFDA for the treatment of variety of cancers including melanoma, lung cancer, kidney cancer, Hodgkins lymphoma, head and neck cancer, bladder cancer and urothelial cancer.

The term PD1 pathway inhibitors includes monoclonal antibodies that interfere with the binding of PD1 to PDL1 and/or PDL2. Antibody PD1 pathway inhibitors are well known in the art. Examples of commercially available PD1 pathway inhibitors that monoclonal antibodies that interfere with the binding of PD1 to PDL1 and/or PDL2 include nivolumab (Opdivo®, BMS-936558, MDX1106, commercially available from BristolMyers Squibb, Princeton NJ), pembrolizumab (Keytruda® MK-3475, lambrolizumab, commercially available from Merck and Company, Kenilworth NJ), and atezolizumab (Tecentriq®, Genentech/Roche, South San Francisco CA). Additional PD1 pathway inhibitors antibodies are in clinical development including but not limited to durvalumab (MEDI4736, Medimmune/AstraZeneca), pidilizumab (CT-011, CureTech), PDR001 (Novartis), BMS-936559 (MDX1105, BristolMyers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011.

The term PD1 pathway inhibitors are not limited to antagonist antibodies. Non-antibody biologic PD1 pathway inhibitors are also under clinical development including AMP-224, a PD-L2 IgG2a fusion protein, and AMP-514, a PDL2 fusion protein, are under clinical development by Amplimmune and Glaxo SmithKline. Aptamer compounds are also described in the literature useful as PD1 pathway inhibitors (Wang, et al. (2018) 145:125-130.).

The term PD1 pathway inhibitors includes peptidyl PD1 pathway inhibitors such as those described in Sasikumar, et al., U.S. Pat. No. 9,422,339 issued Aug. 23, 2016, and Sasilkumar, et al., U.S. Pat. No. 8,907,053 issued Dec. 9, 2014. CA-170 (AUPM-170, Aurigene/Curis) is reportedly an orally bioavailable small molecule targeting the immune checkpoints PDL1 and VISTA. Pottayil Sasikumar, et al. Oral immune checkpoint antagonists targeting PD-L1/VISTA or PD-L1/Tim3 for cancer therapy. [abstract]. In: Proceedings of the 107th Annual Meeting of the American Association for Cancer Research; 2016 Apr 16-20; New Orleans, LA. Philadelphia (PA): AACR; Cancer Res 2016; 76(14 Suppl): Abstract No. 4861. CA-327 (AUPM-327, Aurigene/Curis) is reportedly an orally available, small molecule that inhibit the immune checkpoints, Programmed Death Ligand-1 (PDL1) and T-cell immunoglobulin and mucin domain containing protein-3 (TIM3).

The term PD1 pathway inhibitors includes small molecule PD1 pathway inhibitors. Examples of small molecule PD1 pathway inhibitors useful in the practice of the present invention are described in the art including Sasikumar, et al., 1,2,4-oxadiazole and thiadiazole compounds as immunomodulators (PCT/IB2016/051266 filed Mar. 7, 2016, published as WO2016142833A1 Sep. 15, 2016) and Sasikumar, et al. 3-substituted-1,2,4-oxadiazole and thiadiazole PCT/IB2016/051343 filed Mar. 9, 2016 and published as WO2016142886A2), BMS-1166 and Chupak L S and Zheng X. Compounds useful as immunomodulators. Bristol-Myers Squibb Co. (2015) WO 2015/034820 A1, EP3041822 B1 granted Aug. 9, 2017; WO2015034820 Al; and Chupak, et al. Compounds useful as immunomodulators. Bristol-Myers Squibb Co. (2015) WO 2015/160641 A2. WO 2015/160641 A2, Chupak, et al. compounds useful as immunomodulators. Bristol-Myers Squibb Co. Sharpe, et al. Modulators of immunoinhibitory receptor PD-1, and methods of use thereof, WO 2011082400 A2 published Jul. 7, 2011; U.S. Pat. No. 7,488,802 (Wyeth) issued Feb. 10, 2009;

In some embodiments, combination of binding proteins described herein and one or more PD1 immune checkpoint modulators are useful in the treatment of neoplastic conditions for which PD1 pathway inhibitors have demonstrated clinical effect in human beings either through FDA approval for treatment of the disease or the demonstration of clinical efficacy in clinical trials including but not limited to melanoma, non-small cell lung cancer, small cell lung cancer, head and neck cancer, renal cell cancer, bladder cancer, ovarian cancer, uterine endometrial cancer, uterine cervical cancer, uterine sarcoma, gastric cancer, esophageal cancer, DNA mismatch repair deficient colon cancer, DNA mismatch repair deficient endometrial cancer, hepatocellular carcinoma, breast cancer, Merkel cell carcinoma, thyroid cancer, Hodgkins lymphoma, follicular lymphoma, diffuse large B-cell lymphoma, mycosisfungoides, peripheral T-cell lymphoma. In some embodiments, the combination of binding proteins and an PD1 immune checkpoint modulator is useful in the treatment of tumors characterized by high levels of expression of PDL1, where the tumor has a tumor mutational burden, where there are high levels of CD8⁺ T-cell in the tumor, an immune activation signature associated with IFNγ and the lack of metastatic disease particularly liver metastasis.

In some embodiments, the binding protein is administered in combination with an antagonist of a negative immune checkpoint pathway that inhibits the binding of CTLA4 to CD28 (“CTLA4 pathway inhibitor”). Examples of CTLA4 pathway inhibitors are well known in the art (See, e.g., U.S. Pat. No. 6,682,736 (Abgenix) issued Jan. 27, 2004; U.S. Pat. No. 6,984,720 (Medarex, Inc.) issued May 29, 2007; U.S. Pat. No. 7,605,238 (medarex, Inc.) issued Oct. 20, 2009)

In some embodiments, the binding protein is administered in combination with an antagonist of a negative immune checkpoint pathway that inhibits the binding of BTLA to HVEM (“BTLA pathway inhibitor”). A number of approaches targeting the BTLA/HVEM pathway using anti-BTLA antibodies and antagonistic HVEM-Ig have been evaluated, and such approaches have suggested promising utility in a number of diseases, disorders and conditions, including transplantation, infection, tumor, and autoimmune disease (See e.g. Wu, et al., (2012) Int. J. Biol. Sci. 8:1420-30).

In some embodiments, the binding protein is administered in combination with an antagonist of a negative immune checkpoint pathway that inhibits the ability TIM3 to binding to TIM3-activating ligands (“TIM3 pathway inhibitor”). Examples of TIM3 pathway inhibitors are known in the art and with representative non-limiting examples described in United States Patent Publication No. PCT/US2016/021005 published Sep. 15, 2016; Lifke, et al. United States Patent Publication No. US 20160257749 A1 published Sep. 8, 2016 (F. Hoffman-LaRoche), Karunsky, U.S. Pat. No. 9,631,026 issued Apr. 27, 2017; Karunsky, Sabatos-Peyton, et al. U.S. Pat. No. 8,841,418 issued Sep. 23, 2014; U.S. Pat. No. 9,605,070; Takayanagi, et al., U.S. Pat. No. 8,552,156 issued Oct. 8, 2013.

In some embodiments, the binding protein is administered in combination with an inhibitor of both LAG3 and PD1 as the blockade of LAG3 and PD1 has been suggested to synergistically reverse energy among tumor-specific CD8⁺ T-cells and virus-specific CD8⁺ T-cells in the setting of chronic infection. IMP321 (ImmuFact) is being evaluated in melanoma, breast cancer, and renal cell carcinoma. See generally Woo et al., (2012) Cancer Res 72:917-27; Goldberg et al., (2011) Curr. Top. Microbiol. Immunol. 344:269-78; Pardoll (2012) Nature Rev. Cancer 12:252-64; Grosso et al., (2007) J. Clin. Invest. 117:3383-392.

In some embodiments, the binding protein is administered in combination with an A2aR inhibitor. A2aR inhibits T-cell responses by stimulating CD4⁺ T-cells towards developing into T_{reg} cells. A2aR is particularly important in tumor immunity because the rate of cell death in tumors from cell turnover is high, and dying cells release adenosine, which is the ligand for A2aR. In addition, deletion of A2aR has been associated with enhanced and sometimes pathological inflammatory responses to infection. Inhibition of A2aR can be effected by the administration of molecules such as antibodies that block adenosine binding or by adenosine analogs. Such agents may be used in combination with the binding proteins for use in the treatment disorders such as cancer and Parkinson's disease.

In some embodiments, the binding protein is administered in combination with an inhibitor of IDO (Indoleamine 2,3-dioxygenase). IDO down-regulates the immune response mediated through oxidation of tryptophan resulting in in inhibition of T-cell activation and induction of T-cell apoptosis, creating an environment in which tumor-specific cytotoxic T lymphocytes are rendered functionally inactive or are no longer able to attack a subject's cancer cells. Indoximod (NewLink Genetics) is an IDO inhibitor being evaluated in metastatic breast cancer.

As previously described, the present invention provides for a method of treatment of neoplastic disease (e.g., cancer) in a mammalian subject by the administration of a binding protein in combination with an agent(s) that modulate at least one immune checkpoint pathway including immune checkpoint pathway modulators that modulate two, three or more immune checkpoint pathways.

In some embodiments the binding protein is administered in combination with an immune checkpoint modulator that is capable of modulating multiple immune checkpoint pathways. Multiple immune checkpoint pathways may be modulated by the administration of multi-functional molecules which are capable of acting as modulators of multiple immune checkpoint pathways. Examples of such multiple immune checkpoint pathway modulators include but are not limited to bi-specific or poly-specific antibodies. Examples of poly-specific antibodies capable of acting as modulators or multiple immune checkpoint pathways are known in the art. For example, United States Patent Publication No. 2013/0156774 describes bispecific and multispecific agents (e.g., antibodies), and methods of their use, for targeting cells that co-express PD1 and TIM3. Moreover, dual blockade of BTLA and PD1 has been shown to enhance antitumor immunity (Pardoll, (April 2012) Nature Rev. Cancer 12:252-64). The present disclosure contemplates the use of binding proteins in combination with immune checkpoint pathway modulators that target multiple immune checkpoint pathways, including but limited to bi-specific antibodies which bind to both PD1 and LAG3. Thus, antitumor immunity can be enhanced at multiple levels, and combinatorial strategies can be generated in view of various mechanistic considerations.

In some embodiments, the binding protein may be administered in combination with two, three, four or more checkpoint pathway modulators. Such combinations may be advantageous in that immune checkpoint pathways may have distinct mechanisms of action, which provides the opportunity to attack the underlying disease, disorder or conditions from multiple distinct therapeutic angles.

It should be noted that therapeutic responses to immune checkpoint pathway inhibitors often manifest themselves much later than responses to traditional chemotherapies such as tyrosine kinase inhibitors. In some instance, it can take six months or more after treatment initiation with immune checkpoint pathway inhibitors before objective indicia of a therapeutic response are observed. Therefore, a determination as to whether treatment with an immune checkpoint pathway inhibitors(s) in combination with a binding protein of the present disclosure must be made over a time-to-progression that is frequently longer than with conventional chemotherapies. The desired response can be any result deemed favorable under the circumstances. In some embodiments, the desired response is prevention of the progression of the disease, disorder or condition, while in other embodiments the desired response is a regression or stabilization of one or more characteristics of the disease, disorder or conditions (e.g., reduction in tumor size). In still other embodiments, the desired response is reduction or elimination of one or more adverse effects associated with one or more agents of the combination.

### Cell Therapy Agents and Methods as Supplementary Agents

In some embodiments, the methods of the disclosure may include the combination of the administration of a binding protein with supplementary agents in the form of cell therapies for the treatment of neoplastic, autoimmune or inflammatory diseases. Examples of cell therapies that are amenable to use in combination with the methods of the present disclosure include but are not limited to engineered T cell products comprising one or more activated CAR-T cells, engineered TCR cells, tumor infiltrating lymphocytes (TILs), engineered Treg cells. As engineered T-cell products are commonly activated ex vivo prior to their administration to the subject and therefore provide upregulated levels of CD25, cell products comprising such activated engineered T cells types are amenable to further support via the administration of a CD25 biased binding protein as described herein.

### CAR-T Cells

In some embodiments of the methods of the present disclosure, the supplementary agent is a “chimeric antigen receptor T-cell” and “CAR-T cell” are used interchangeably to refer to a T-cell that has been recombinantly modified to express a chimeric antigen receptor. As used herein, the terms As used herein, the terms “chimeric antigen receptor” and “CAR” are used interchangeably to refer to a chimeric polypeptide comprising multiple functional domains arranged from amino to carboxy terminus in the sequence: (a) an antigen binding domain (ABD), (b) a transmembrane domain (TD); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface of a cell transformed with an expression vector comprising a nucleic acid sequence encoding the CAR CARs useful in the practice of the present invention are prepared in accordance with principles well known in the art. See e.g., Eshhaar et al. U.S. Pat. No. 7,741,465 B1 issued Jun. 22, 2010; Sadelain, et al (2013) Cancer Discovery 3(4):388-398; Jensen and Riddell (2015) Current Opinions in Immunology 33:9-15; Gross, et al. (1989) PNAS(USA) 86(24):10024-10028; Curran, et al. (2012) J Gene Med 14(6):405-15. Examples of commercially available CAR-T cell products that may be modified to incorporate an orthogonal receptor of the present invention include axicabtagene ciloleucel (marketed as Yescarta® commercially available from Gilead Pharmaceuticals) and tisagenlecleucel (marketed as Kymriah® commercially available from Novartis).

As used herein, the term antigen binding domain (ABD) refers to a polypeptide that specifically binds to an antigen expressed on the surface of a target cell. The ABD may be any polypeptide that specifically binds to one or more cell surface molecules (e.g. tumor antigens) expressed on the surface of a target cell. In some embodiments, the ABD is a polypeptide that specifically binds to a cell surface molecule associated with a tumor cell is selected from the group consisting of GD2, BCMA, CD19, CD33, CD38, CD70, GD2, IL3Rα2, CD19, mesothelin, Her2, EpCam, Mucl, ROR1, CD133, CEA, EGRFRVIII, PSCA, GPC3, Pan-ErbB and FAP. In some embodiments, the ABD is an antibody (as defined hereinabove to include molecules such as one or more V_{H}Hs, scFvs, etc.) that specifically binds to at least one cell surface molecule associated with a tumor cell (i.e. at least one tumor antigen) wherein the cell surface molecule associated with a tumor cell is selected from the group consisting of GD2, BCMA, CD19, CD33, CD38, CD70, GD2, IL3Rα2, CD19, mesothelin, Her2, EpCam, Mucl, ROR1, CD133, CEA, EGRFRVIII, PSCA, GPC3, Pan-ErbB and FAP. Examples of CAR-T cells useful as supplementary agents in the practice of the methods of the present disclosure include but are not limited to CAR-T cells expressing CARs comprising an ABD further comprising at least one of: anti-GD2 antibodies, anti-BCMA antibodies, anti-CD19 antibodies, anti-CD33 antibodies, anti-CD38 antibodies, anti-CD70 antibodies, anti-GD2 antibodies and IL3Rα2 antibodies, anti-CD19 antibodies, anti-mesothelin antibodies, anti-Her2 antibodies, anti-EpCam antibodies, anti-Mucl antibodies, anti-ROR1 antibodies, anti-CD133 antibodies, anti-CEA antibodies, anti-PSMA antibodies, anti-EGRFRVIII antibodies, anti-PSCA antibodies, anti-GPC3 antibodies, anti-Pan-ErbB antibodies, anti-FAP antibodies,

CARs of CAR-T cells useful in the practice of the methods of the present disclosure further comprise a transmembrane domain joining the ABD (or linker, if employed, see discussion of linkers below) to the intracellular cytoplasmic domain of the CAR The transmembrane domain is comprised of any polypeptide sequence which is thermodynamically stable in a eukaryotic cell membrane. The transmembrane spanning domain may be derived from the transmembrane domain of a naturally occurring membrane spanning protein or may be synthetic. In designing synthetic transmembrane domains, amino acids favoring alpha-helical structures are preferred. Transmembrane domains useful in construction of CARs are comprised of approximately 10, 11, 12, 13, 14, 15, 16, 17, 18, 19, 20, 21, 22, 22, 23, or 24 amino acids favoring the formation having an alpha-helical secondary structure. Amino acids having a to favor alpha-helical conformations are well known in the art. See, e.g Pace, et al. (1998) Biophysical Journal 75: 422-427. Amino acids that are particularly favored in alpha helical conformations include methionine, alanine, leucine, glutamate, and lysine. In some embodiments, the CAR transmembrane domain may be derived from the transmembrane domain from type I membrane spanning proteins, such as CD3ζ, CD4, CD8, CD28, etc.

The cytoplasmic domain of the CAR polypeptide comprises one or more intracellular signal domains. In one embodiment, the intracellular signal domains comprise the cytoplasmic sequences of the T-cell receptor (TCR) and co-receptors that initiate signal transduction following antigen receptor engagement and functional derivatives and sub-fragments thereof. A cytoplasmic signaling domain, such as those derived from the T cell receptor zeta-chain, is employed as part of the CAR in order to produce stimulatory signals for T lymphocyte proliferation and effector function following engagement of the chimeric receptor with the target antigen. Examples of cytoplasmic signaling domains include but are not limited to the cytoplasmic domain of CD27, the cytoplasmic domain S of CD28, the cytoplasmic domain of CD137 (also referred to as 4-1BB and TNFRSF9), the cytoplasmic domain of CD278 (also referred to as ICOS), p110α, β, or δ catalytic subunit of PI3 kinase, the human CD3 ζ− chain, cytoplasmic domain of CD134 (also referred to as OX40 and TNFRSF4), FcεR1γ and β chains, MB1 (Igα) chain, B29 (Igβ) chain, etc.), CD3 polypeptides (δ, Δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28.

In some embodiments, the CAR may also provide a co-stimulatory domain. The term “co-stimulatory domain” (“CSD”), refers to a stimulatory domain, typically an endodomain, of a CAR that provides a secondary non-specific activation mechanism through which a primary specific stimulation is propagated. The co-stimulatory domain refers to the portion of the CAR which enhances the proliferation, survival or development of memory cells. Examples of co-stimulation include antigen nonspecific T cell co-stimulation following antigen specific signaling through the T cell receptor and antigen nonspecific B cell co-stimulation following signaling through the B cell receptor. Co-stimulation, e.g., T cell co-stimulation, and the factors involved are described in Chen & Flies (2013) Nat Rev Immunol 13(4):227-42. In some embodiments of the present disclosure, the CSD comprises one or more of members of the TNFR superfamily, CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40 or combinations thereof.

CARs useful in the practice of the methods of the present disclosure may optionally include one or more polypeptide spacers linking the domains of the CAR, in particular the linkage between the ABD to the transmembrane spanning domain of the CAR. Although not an essential element of the CAR structure, the inclusion of a spacer domain is generally considered desirable to facilitate antigen recognition by the ARD. As used in conjunction with the CAR-T cell technology described herein, the terms “linker”, “linker domain” and “linker region” refer to a polypeptide from about 1 to 100 amino acids in length. Linkers are typically be composed of amino acid residues which permit flexibility of the polypeptide (e.g. glycine and serine) so that the adjacent domains of the CAR are provided greater freedom of movement relative to one another. Although there is no particularly defined length or sequence of amino acids that is necessary for the spacer to achieve its function, but the typical properties of the spacer are flexibility to enable freedom of movement of the ABD to facilitate targeting antigen recognition. Similarly, it has been found that there is there is substantial leniency in spacer length while retaining CAR function. Jensen and Riddell (2014) Immunol. Review 257(1) 127-144. Sequences useful as spacers in the construction of CARs useful in the practice of the present invention include but are not limited to the hinge region of IgG1, the immunoglobulin 1 CH2-CH3 region, IgG4 hinge-CH2-CH3, IgG4 hinge-CH3, and the IgG4 hinge. The hinge and transmembrane domains may be derived from the same molecule such as the hinge and transmembrane domains of CD8-alpha. Imai, et al. (2004) Leukemia 18(4):676-684.

CARs are often referred to as first, second, third or fourth generation. The term first-generation CAR refers to a CAR wherein the cytoplasmic domain transmits the signal from antigen binding through only a single signaling domain, for example a signaling domain derived from the high-affinity receptor for IgE FcεR1γ or the CD3ζ chain. The domain contains one or three immunoreceptor tyrosine-based activating motif(s) [ITAM(s)] for antigen-dependent T-cell activation. The ITAM-based activating signal endows T-cells with the ability to lyse the target tumor cells and secret cytokines in response to antigen binding. Second-generation CARs include a co-stimulatory signal in addition to the CD3ζ signal. Coincidental delivery of the co-stimulatory signal enhances cytokine secretion and antitumor activity induced by CAR-transduced T-cells. The co-stimulatory domain is usually be membrane proximal relative to the CD3ζ domain. Third-generation CARs include a tripartite signaling domain, comprising for example a CD28, CD3ζ, OX40 or 4-1BB signaling region. In fourth generation, or “armored car” CAR T-cells are further modified to express or block molecules and/or receptors to enhance immune activity such as the expression of IL12, IL18, IL7, and/or IL10; 4-1BB ligand, CD-40 ligand. Examples of intracellular signaling domains comprising may be incorporated into the CAR of the present invention include (amino to carboxy): CD3ζ; CD28-41BB-CD3ζ; CD28-OX40-CD3ζ; CD28-41BB-CD3ζ; 41BB-CD-28-CD3ζ and 41BB-CD3ζ.

The term CAR includes CAR variants including but not limited split CARs, ON-switch CARS, bispecific or tandem CARs, inhibitory CARs (iCARs) and induced pluripotent stem (iPS) CAR-T cells. The term “Split CARs” refers to CARs wherein the extracellular portion, the ABD and the cytoplasmic signaling domain of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR molecules and derivatives thereof (i.e., CAR variants) are described, e.g., in PCT Application Nos. US2014/016527, US 1996/017060, US2013/063083; Fedorov et al. Sci Transi Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. The term “bispecific or tandem CARs” refers to CARs which include a secondary CAR binding domain that can either amplify or inhibit the activity of a primary CAR The term “inhibitory chimeric antigen receptors” or “iCARs” are used interchangeably herein to refer to a CAR where binding iCARs use the dual antigen targeting to shut down the activation of an active CAR through the engagement of a second suppressive receptor equipped with inhibitory signaling domains of a secondary CAR binding domain results in inhibition of primary CAR activation. Inhibitory CARs (iCARs) are designed to regulate CAR-T cells activity through inhibitory receptors signaling modules activation. This approach combines the activity of two CARS, one of which generates dominant negative signals limiting the responses of CAR-T cells activated by the activating receptor. iCARs can switch off the response of the counteracting activator CAR when bound to a specific antigen expressed only by normal tissues. In this way, iCARs-T cells can distinguish cancer cells from healthy ones, and reversibly block functionalities of transduced T cells in an antigen-selective fashion. CTLA-4 or PD-1 intracellular domains in iCARs trigger inhibitory signals on T lymphocytes, leading to less cytokine production, less efficient target cell lysis, and altered lymphocyte motility. The term “tandem CAR” or “TanCAR” refers to CARs which mediate bispecific activation of T cells through the engagement of two chimeric receptors designed to deliver stimulatory or costimulatory signals in response to an independent engagement of two different tumor associated antigens.

Typically, the chimeric antigen receptor T-cells (CAR-T cells) are T-cells which have been recombinantly modified by transduction with an expression vector encoding a CAR in substantial accordance with the teaching above.

In some embodiments, the engineered T cell is allogeneic with respect to the individual that is treated. Graham et al. (2018) Cell 7(10) E155. In some embodiments an allogeneic engineered T cell is fully HLA matched. However not all patients have a fully matched donor and a cellular product suitable for all patients independent of HLA type provides an alternative.

Because the cell product may consist of a subject's own T-cells, the population of the cells to be administered is to the subject is necessarily variable. Consequently, identifying the optimal concentration of the CAR-T cell will be optimized by the caregiver in accordance with the needs of the subject to be treated and monitored by conventional laboratory testing. Additionally, since the CAR-T cell agent is variable, the response to such agents can vary and thus involves the ongoing monitoring and management of therapy related toxicities which are managed with a course of pharmacologic immunosuppression or B cell depletion prior to the administration of the CAR-T cell treatment. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection. The engineered cells may be infused to the subject in any physiologically acceptable medium by any convenient route of administration, normally intravascularly, although they may also be introduced by other routes, where the cells may find an appropriate site for growth

If the T cells used in the practice of the present invention are allogeneic T cells, such cells may be modified to reduce graft versus host disease. For example, the engineered cells of the present invention may be TCRαβ receptor knock-outs achieved by gene editing techniques. TCRαβ is a heterodimer and both alpha and beta chains need to be present for it to be expressed. A single gene codes for the alpha chain (TRAC), whereas there are 2 genes coding for the beta chain, therefore TRAC loci KO has been deleted for this purpose. A number of different approaches have been used to accomplish this deletion, e.g. CRISPR/Cas9; meganuclease; engineered I-Crel homing endonuclease, etc. See, for example, Eyquem et al. (2017) Nature 543:113-117, in which the TRAC coding sequence is replaced by a CAR coding sequence; and Georgiadis et al. (2018) Mol. Ther. 26:1215-1227, which linked CAR expression with TRAC disruption by clustered regularly interspaced short palindromic repeats (CRISPR)/Cas9 without directly incorporating the CAR into the TRAC loci. An alternative strategy to prevent GVHD modifies T cells to express an inhibitor of TCRαβ signaling, for example using a truncated form of CD3ζ as a TCR inhibitory molecule.

### Chemokine and Cytokine Agents as Supplementary Agents:

In some embodiments the binding protein is administered in combination with additional cytokines including but not limited to IL2, IL7, IL12, IL15 (See U.S. Pat. No. 10,398,761 issued Sep. 13, 2019) and IL18 including analogs and variants of each thereof.

### Activation-induced Cell Death Inhibitors

In some embodiments the binding protein is administered in combination with one or more supplementary agents that inhibit Activation-Induced Cell Death (AICD). AICD is a form of programmed cell death resulting from the interaction of Fas receptors (e.g., Fas, CD95) with Fas ligands (e.g., FasL, CD95 ligand), helps to maintain peripheral immune tolerance. The AICD effector cell expresses FasL, and apoptosis is induced in the cell expressing the Fas receptor. Activation-induced cell death is a negative regulator of activated T lymphocytes resulting from repeated stimulation of their T-cell receptors. Examples of agents that inhibit AICD that may be used in combination with the binding proteins described herein include but are not limited to cyclosporin A (Shih, et al., (1989) Nature 339:625-626, IL16 and analogs (including rhIL16, Idziorek, et al., (1998) Clinical and Experimental Immunology 112:84-91), TGFb 1 (Genesteir, et al., (1999) J Exp Med 189(2): 231-239), and vitamin E (Li-Weber, et al., (2002) J Clin Investigation 110(5):681-690).

### Physical Methods

In some embodiments, the supplementary agent is an anti-neoplastic physical methods including but not limited to radiotherapy, cryotherapy, hyperthermic therapy, surgery, laser ablation, and proton therapy.

### Immune Diseases

The present disclosure further provides methods of treating a subject suffering from a disease disorder or condition by the administration of a therapeutically effective amount of an IL2R binding protein (or nucleic acid encoding an IL2R binding protein including recombinant viruses encoding the IL2R binding protein) of the present disclosure. Disorders amenable to treatment with IL2R binding proteins (including pharmaceutically acceptable formulations comprising IL2R binding proteins and/or the nucleic acid molecules that encode them including recombinant viruses encoding such IL2R binding proteins) of the present disclosure include inflammatory or autoimmune diseases including but not limited to, viral infections (e.g., AIDS, influenza, chronic HCV, chronic viral hepatitis B, C or D), heliobacter pylori infection, HTLV, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes including Type 1 or type 2 diabetes, inflammation, autoimmune disease, atopic diseases, paraneoplastic autoimmune diseases, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reitees Syndrome, SEA Syndrome (Seronegativity Enthesopathy Arthropathy Syndrome), juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reitees syndrome, SEA Syndrome(Seronegativity, Enthesopathy, Arthropathy Syndrome).

Other examples of proliferative and/or differentiative disorders amenable to treatment with IL2R binding proteins (including pharmaceutically acceptable formulations comprising IL2R binding proteins and/or the nucleic acid molecules that encode them including recombinant viruses encoding such IL2R binding proteins) of the present disclosure include, but are not limited to, skin disorders. The skin disorder may involve the aberrant activity of a cell or a group of cells or layers in the dermal, epidermal, or hypodermal layer, or an abnormality in the dermal-epidermal junction. For example, the skin disorder may involve aberrant activity of keratinocytes (e.g., hyperproliferative basal and immediately suprabasal keratinocytes), melanocytes, Langerhans cells, Merkel cells, immune cell, and other cells found in one or more of the epidermal layers, e.g., the stratum basale (stratum gemvnativum), stratum spinosum, stratum granulosum, stratum lucidum or stratum comeum. In other embodiments, the disorder may involve aberrant activity of a dermal cell, for example, a dermal endothelial, fibroblast, immune cell (e.g., mast cell or macrophage) found in a dermal layer, for example, the papillary layer or the reticular layer.

Examples of skin disorders include psoriasis, psoriatic arthritis, dermatitis (eczema), for example, exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitises such as seborrheic dermatitis and solar dermatitis; keratoses such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis. The skin disorder can be dermatitis, e.g., atopic dermatitis or allergic dermatitis, or psoriasis.

The compositions of the present disclosure (including pharmaceutically acceptable formulations comprising IL2R binding proteins and/or the nucleic acid molecules that encode them including recombinant viruses encoding such IL2R binding proteins) can also be administered to a patient who is suffering from (or may suffer from) psoriasis or psoriatic disorders. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis. Examples of psoriatic disorders include chronic stationary psoriasis, plaque psoriasis, moderate to severe plaque psoriasis, psoriasis vulgaris, eruptive psoriasis, psoriatic erythroderma, generalized pustular psoriasis, annular pustular psoriasis, or localized pustular psoriasis.

Combination Of IL2R Binding Proteins with Additional Therapeutic Agents for Autoimmune Disease:

The present disclosure provides the for the use of the IL2R binding proteins of the present disclosure in combination with one or more additional active agents (“supplementary agents”) in the treatment of autoimmune disease. As used herein, the term “supplementary agents” includes agents that can be administered or introduced separately, for example, formulated separately for separate administration (e.g., as may be provided in a kit) and/or therapies that can be administered or introduced in combination with the IL2R binding proteins.

As used herein, the term “in combination with” when used in reference to the administration of multiple agents to a subject refers to the administration of a first agent at least one additional (i.e., second, third, fourth, fifth, etc.) agent to a subject. For purposes of the present invention, one agent (e.g., IL2R binding protein) is considered to be administered in combination with a second agent (e.g. a therapeutic autoimmune antibody such as Humira®) if the biological effect resulting from the administration of the first agent persists in the subject at the time of administration of the second agent such that the therapeutic effects of the first agent and second agent overlap. For example, the therapeutic antibodies are sometimes administered by IV infusion every two weeks (e.g. adalimumab in the treatment of Crohn's disease) while the IL2R binding proteins of the present disclosure may be administered more frequently, e.g. daily, BID, or weekly. However, the administration of the first agent (e.g. entaercept) provides a therapeutic effect over an extended time and the administration of the second agent (e.g. an IL2R binding protein) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject. In certain embodiments, the IL2R binding protein and the supplementary agent(s) are administered or applied sequentially, e.g., where one agent is administered prior to one or more other agents. In other embodiments, the IL2R binding protein and the supplementary agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

In some embodiments, the supplementary agent is one or more agents selected from the group consisting of corticosteroids (including but not limited to prednisone, budesonide, prednilisone), Janus kinase inhibitors (including but not limited to tofacitinib (Xeljanz®), calcineurin inhibitors (including but not limited to cyclosporine and tacrolimus), mTor inhibitors (including but not limited to sirolimus and everolimus), IMDH inhibitors (including but not limited to azathioprine, leflunomide and mycophenolate), biologics such as abatcept (Orencia®) or etanercept (Enbrel®), and therapeutic antibodies. Examples of therapeutic antibodies that may be administered as supplementary agents in combination with the IL2R binding proteins of the present disclosure in the treatment of autoimmune disease include but are not limited to anti-CD25 antibodies (e.g. daclizumab and basiliximab), anti-VLA-4 antibodies (e.g. natalizumab), anti-CD52 antibodies (e.g. alemtuzumab), anti-CD20 antibodies (e.g. rituximab, ocrelizumab), anti-TNF antibodies (e.g. infliximab, and adalimumab), anti-IL6R antibodies (e.g. tocilizumab), anti-TNFα antibodies (e.g. adalimumab (Humira®), golimumab, and infliximab), anti-integrin-α4β7 antibodies (e.g. vedolizumab), anti-IL17a antibodies (e.g. brodalumab or secukinumab), anti-IL4Ra antibodies (e.g. dupilumab), anti-RANKL antibodies, IL6R antibodies, anti-IL1β antibodies (e.g. canakinumab), anti-CD h a antibodies (e.g. efalizumab), anti-CD3 antibodies (e.g. muramonab), anti-IL5 antibodies (e.g. mepolizumab, reslizumab), anti-bLyS antibodies (e.g. belimumab); and anti-IL12/IL23 antibodies (e.g ustekinumab).

Many therapeutic antibodies have been approved for clinical use against autoimmune disease. Examples of antibodies approved by the United States Food and Drug Administration (FDA) for use in the treatment of autoimmune diseases in a subject suffering therefrom that may be administered as supplementary agents in combination with the IL2R binding proteins of the present disclosure (and optionally additional supplementary agents) for the treatment of the indicated autoimmune disease are provided in Table 6.

TABLE 6
FDA Immune Disease Antibodies and Indications.

| Name | Target | Indication |
|---|---|---|
| belimumab | bLyS | Systemic lupus erythematosus |
| efalizumab | CD11a | Psoriasis |
| ocrelizumab | CD20 | Multiple sclerosis |
| rituximab | CD20 | Multiple sclerosis |
| basiliximab | CD25 | Transplantation rejection |
| daclizumab | CD25 | Transplantation rejection |
| muromonab | CD3 | Transplantation rejection |
| alemtuzumab | CD52 | Multiple sclerosis |
| omalizumab | IgE | Asthma |
| ustekinumab | IL12/IL23 | Plaque psoriasis |
| brodalumab | IL17a | Psoriasis, psoriatic arthritis, ankylosing spondylitis |
| secukinumab | IL17a | Psoriasis, psoriatic arthritis, ankylosing spondylitis |
| ixekizumab | IL17a | Psoriasis, psoriatic arthritis, ankylosing spondylitis |
| canakinumab | IL1ß | Cryopyrin-associated periodic syndrome, tumor necrosis factor |
| | | receptor associated periodic syndrome, hyperimmunoglobulin D |
| | | syndrome, mevalonate kinase deficiency, familial Mediterranean |
| | | fever, rheumatoid arthritis |
| dupilumab | IL4Rα | Asthma, dermatitis |
| mepolizumab | IL5 | Asthma |
| reslizumab | IL5 | Asthma |
| tocilizumab | IL6R | Rheumatoid arthritis |
| vedolizumab | Integrin-α4β7 | Ulcerative colitis, Crohn's disease |
| denosumab | RANKL | Osteoporosis |
| certolizumab | TNFa | Chron's disease, rheumatoid arthritis |
| golimumab | TNFa | Rheumatoid arthritis, psoriatic arthritis, ankylosing spondylitis |
| adalimumab | TNFα | Rheumatoid arthritis, juvenile idiopathic arthritis, psoriatic arthritis, |
| | | ankylosing spondylitis, Crohn's disease, plaque psoriasis |
| infliximab | TNFα | Crohn's disease, ulcerative colitis, rheumatoid arthritis, ankylosing |
| | | spondylitis, psoriatic arthritis, plaque psoriasis |
| ranibizumab | VEGF-A | Neovascular age-related macular degeneration, macular edema |
| natalizumab | VLA-4 | Multiple sclerosis, relapsing rultiple sclerosis, Crohn's disease |

The foregoing antibodies useful as supplementary agents in the practice of the methods of the present disclosure may be administered alone or in the form of any antibody drug conjugate (ADC) comprising the antibody, linker, and one or more drugs (e.g. 1, 2, 3, 4, 5, 6, 7, or 8 drugs) or in modified form (e.g. PEGylated).

In some embodiments the supplementary agent is a vaccine. The IL2R binding proteins of the present invention may be administered to a subject in combination with vaccines as an adjuvant to enhance the immune response to the vaccine in accordance with the teaching of Doyle, et al U.S. Pat. No. 5,800,819 issued Sep. 1, 1998. Examples of vaccines that may be combined with the IL2R binding proteins of the present invention include are HSV vaccines, Bordetella pertussis, Escherichia coli vaccines, pneumococcal vaccines including multivalent pneumococcal vaccines such as Prevnar® 13, diptheria, tetanus and pertussis vaccines (including combination vaccines such as Pediatrix®) and Pentacel®), varicella vaccines, Haemophilus influenzae type B vaccines, human papilloma virus vaccines such as Garasil®, polio vaccines, Leptospirosis vaccines, combination respiratory vaccine, Moraxella vaccines, and attenuated live or killed virus vaccine products such as bovine respiratory disease vaccine (RSV), multivalent human influenza vaccines such as Fluzone® and Quadravlent Fluzone®), feline leukemia vaccine, transmissible gastroenteritis vaccine, COVID-19 vaccine, and rabies vaccine.

### Selectivity

In some embodiments, provided herein are methods to selectively induce proliferation of a first cell type over a second cell type, comprising contacting a population of cells comprising both the first and second cell types with an IL2 binding protein described herein, thereby selectively inducing proliferation in one or more of the first cell type over one or more of the second cell type. In some embodiments, the first cell type is T cells and the second cell type is NK cells. In some embodiments, the first cell type is NK cells and the second cell type is T cells. In other embodiments, the number of cells of the first cell type is at least 1.2 (e.g., at least 1.4, 1.6, 1.8, 2, 2.2, 2.4, 2.6, 2.8, 3, 3.2, 3.4, 3.6, 3.8, 4, 6, 8, 10, 12, 14, 16, 18, or 20) fold more than the number of cells of the second cell type.

### Dosage

Dosage, toxicity and therapeutic efficacy of such binding proteins or nucleic acids compounds can be determined by standard pharmaceutical procedures in cell cultures or experimental animals. The data obtained from the cell culture assays and animal studies can be used in formulating a range of dosage for use in humans. The dosage of such compounds lies preferably within a range of circulating concentrations that include the ED50 with minimal acceptable toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 as determined in cell culture. Such information can be used to more accurately determine useful doses in humans. Levels in plasma may be measured, for example, by high performance liquid chromatography.

As defined herein, a therapeutically effective amount of a subject binding protein (i.e., an effective dosage) depends on the polypeptide selected. For instance, single dose amounts in the range of approximately 0.001 to 0.1 mg/kg of patient body weight can be administered; in some embodiments, about 0.005, 0.01, 0.05 mg/kg may be administered.

In some embodiments, the pharmaceutically acceptable forms of the binding proteins of the present disclosure are administered to a subject in accordance with a “low-dose” treatment protocol as described in Klatzman, et al. U.S. Pat. Nos. 9,669,071 and 10,293,028B2 the entire teachings of which are herein incorporated by reference. Additional low dose protocols are described in Smith, K. A. (1993) Blood 81(6):1414-1423, He, et al., (2016) Nature Medicine 22(9): 991-993

In some embodiments of the present disclosure provides methods and compositions for the treatment and/or prevention of neoplastic diseases, disorders or conditions in a subject by the administration to the subject a therapeutically effective amount of a binding protein of the present disclosure wherein the serum concentration of is maintained for a majority (i.e., greater than about 50% of the period of time, alternatively greater than about 60%, alternatively greater than about 70%, alternatively greater than about 80%, alternatively greater than about 90%) of a period of time (e.g. at least 24 hours, alternatively at least 48 hours, alternatively at least 72 hours, alternatively at least 96 hours, alternatively at least 120 hours, alternatively at least 144 hours, alternatively at least 7 days, alternatively at least 10 days, alternatively at least 12 days, alternatively at least 14 days, alternatively at least 28 days, alternatively at least 45 days, alternatively at least 60 days, or longer) at a serum concentration at or above the effective concentration of the binding protein sufficient to promote proliferation of CD3-activated primary human T-cells (e.g., at or above EC₁₀^{PRO}, alternatively at or above EC₂₀^{PRO}, alternatively at or above EC₃₀^{PRO}, alternatively at or above EC₄₀^{PRO}, at or above EC₅₀^{PRO}, alternatively at or above EC₆₀^{PRO}) with respect to such binding protein but at a serum concentration at or below of the effective concentration at a serum concentration of such binding protein sufficient to induce activation of T-cells (e.g., at or below EC₁₀₀^{PRO}, alternatively at or below EC₉₀^{PRO}, alternatively at or below EC₈₀^{PRO}, alternatively at or below EC₇₀^{PRO}, at or below EC₆₀^{PRO}, alternatively at or below EC₅₀^{PRO}) with respect to such binding protein.

In some embodiments of the present disclosure provides methods and compositions for the treatment and/or prevention of neoplastic diseases, disorders or conditions in a subject by the administration to the subject a therapeutically effective amount of a binding protein described herein sufficient to maintain a serum concentration of the binding protein for more than about 50%, alternatively greater than about 60%, alternatively greater than about 70%, alternatively greater than about 80%, alternatively greater than about 90%) of a period of time of at least 24 hours, alternatively at least 96 hours, alternatively at least 120 hours, alternatively at least 144 hours, alternatively at least 7 days, alternatively at least 10 days, alternatively at least 12 days, alternatively at least 14 days, alternatively at least 28 days, alternatively at least 45 days, alternatively at least 60 days, or longer.

In some embodiments of the present disclosure provides methods and compositions for the treatment and/or prevention of neoplastic diseases, disorders or conditions in a subject by the administration to the subject a therapeutically effective amount of a binding protein sufficient to maintain a serum concentration of the binding protein at or above the effective concentration for more than about 50%, alternatively greater than about 60%, alternatively greater than about 70%, alternatively greater than about 80%, alternatively greater than about 90%) of a period of time of at least 24 hours, alternatively at least 96 hours, alternatively at least 120 hours, alternatively at least 144 hours, alternatively at least 7 days, alternatively at least 10 days, alternatively at least 12 days, alternatively at least 14 days, alternatively at least 28 days, alternatively at least 45 days, alternatively at least 60 days, or longer.

In accordance with another aspect, there is provided a method for stimulating the immune system of an animal by administering the binding proteins of the present disclosure. The method is useful to treat disease states where the host immune response is deficient. In treating a subject, a therapeutically effective dose of compound (i.e., active ingredient) is administered. A therapeutically effective dose refers to that amount of the active ingredient that produces amelioration of symptoms or a prolongation of survival of a subject. An effective dose will vary with the characteristics of the binding protein to be administered, the physical characteristics of the subject to be treated, the nature of the disease or condition, and the like. A single administration can range from about 50,000 IU/kg to about 1,000,000 IU/kg or more, more typically about 600,000 IU/kg. This may be repeated several times a day (e.g., 2-3 times per day) for several days (e.g., about 3-5 consecutive days) and then may be repeated one or more times following a period of rest (e.g., about 7-14 days). Thus, an effective dose may comprise only a single administration or many administrations over a period of time (e.g., about 20-30 individual administrations of about 600,000 IU/kg each given over about a 10-20 day period).

The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the binding proteins can include a single treatment or, can include a series of treatments. In one embodiment, the compositions are administered every 8 hours for five days, followed by a rest period of 2 to 14 days, e.g., 9 days, followed by an additional five days of administration every 8 hours. In another embodiment, the compositions are administered every other day for a period of at least 6 days, optionally at least 10 days, optionally at least 14 days, optionally at least 30 days, optionally at least 60 days.

The pharmaceutical compositions can be included in a container, pack, or dispenser together with instructions for administration.

While compounds that exhibit toxic side effects may be used, care should be taken to design a delivery system that targets such compounds to the site of affected tissue in order to minimize potential damage to uninfected cells and, thereby, reduce side effects. Toxicity and therapeutic efficacy of a binding protein can be determined by standard pharmaceutical procedures in cell culture or experimental animals. Cell culture assays and animal studies can be used to determine the LD50 (the dose lethal to 50% of a population) and the ED₅₀ (the dose therapeutically effective in 50% of a population). The dose ratio between toxic and therapeutic effects is the therapeutic index, which can be expressed as the ratio LC₅₀/EC₅₀. Binding proteins that exhibit large therapeutic indices are preferred. The data obtained from these cell culture assays and animal studies can be used in formulating a range of dosages suitable for use in humans. The dosage of such mutants lies preferably within a range of circulating concentrations that include the ED₅₀ with little or no toxicity. The dosage may vary within this range depending upon a variety of factors, e.g., the dosage form employed, the route of administration utilized, the condition of the subject, and the like.

A therapeutically effective dose can be estimated initially from cell culture assays by determining an EC₅₀. A dose can then be formulated in animal models to achieve a circulating plasma concentration range that includes the EC₅₀ as determined in cell culture. Such information can be used to more accurately determine useful doses in humans. Levels in plasma may be measured, for example, by HPLC. The exact formulation, route of administration and dosage can be chosen by the individual physician in view of the patient's condition.

The attending physician for patients treated with binding proteins of the present disclosure would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

### EXAMPLES

### Example 1—V_{H}H Generation

Camels were acclimated at research facility for at least 7 days before immunization. Antigen was diluted with 1×PBS (antigen total about 1 mg). The quality of the antigen was assessed by SDS-PAGE to ensure purity (e.g., >80%). For the first time, 10 mL CFA (then followed 6 times using IFA) was added into mortar, then 10 mL antigen in 1×PBS was slowly added into the mortar with the pestle grinding. The antigen and CFA/IFA were ground until the component showed milky white color and appeared hard to disperse. Camels were injected with antigen emulsified in CFA subcutaneously at at least six sites on the body, injecting about 2 mL at each site (total of 10 mL per camel). A stronger immune response was generated by injecting more sites and in larger volumes. The immunization was conducted every week (7 days), for 7 times. The needle was inserted into the subcutaneous space for 10 to 15 seconds after each injection to avoid leakage of the emulsion. Alternatively, a light pull on the syringe plunger also prevented leakage. The blood sample was collected three days later after 7th immunization.

After immunization, the library was constructed. Briefly, RNA was extracted from blood and transcribed to cDNA. The V_{H}H regions were obtained via two-step PCR, which fragment about 400 bp. The PCR outcomes and the vector of pMECS phagemid were digested with Pst I and Not I, subsequently, ligated to pMECS/Nb recombinant. After ligation, the products were transformed into Escherichia coli (E. coli) TG1 cells by electroporation. Then, the transformants were enriched in growth medium and planted on plates. Finally, the library size was estimated by counting the number of colonies.

Library biopanning was conducted to screen candidates against the antigens after library construction. Phage display technology was applied in this procedure. Positive colonies were identified by PE-ELISA.

### Example 2—Recombinant Production and Purification

Codon optimized DNA inserts were cloned into modified pcDNA3.4 (Genscript) for small scale expression in HEK293 cells in 24 well plates. The binding molecules were purified in substantial accordance with the following procedure. Using a Hamilton Star automated system, 96×4 mL of supernatants in 4×24-well blocks were re-arrayed into 4×96-well, 1 mL blocks. PhyNexus micropipette tips (Biotage, San Jose CA) holding 80 μL of Ni-Excel IMAC resin (Cytiva) are equilibrated wash buffer. PBS pH 7.4, 30 mM imidazole. PhyNexus tips were dipped and cycled through 14 cycles of 1 mL pipetting across all 4×96-well blocks. PhyNexus tips were washed in 2×1 mL blocks holding wash buffer. PhyNexus tips were eluted in 3×0.36 mL blocks holding elution buffer: PBS pH 7.4, 400 mM imidazole. PhyNexus tips were regenerated in 3×1 mL blocks of 0.5 M sodium hydroxide.

The purified protein eluates were quantified using a Biacore® T200 as in substantial accordance with the following procedure. 10 μL of the first 96×0.36 mL eluates were transferred to a Biacore® 96-well microplate and diluted to 60 uL in HBS−EP+ buffer (10 mM Hepes pH 7.4, 150 mM NaCl, 1 mM EDTA, 0.05% Tween 20). Each of the 96 samples was injected on a CMS series S chip previously functionalized with anti-histidine capture antibody (Cytiva): injection is performed for 18 seconds at 5 μL/min. Capture levels were recorded 60 seconds after buffer wash. A standard curve of known VHH concentrations (270, 90, 30, 10, 3.3, 1.1 μg/mL) was acquired in each of the 4 Biacore chip flow cells to eliminate cell-to-cell surface variability. The 96 captures were interpolated against the standard curve using a non-linear model including specific and unspecific, one-site binding. Concentrations in the first elution block varied from 12 to 452 μg/mL corresponding to a 4-149 μg. SDS-PAGE analysis of 5 randomly picked samples was performed to ensure molecular weight of eluates corresponded to expected values (˜30 kDa).

The concentration of the proteins was normalized using the Hamilton Star automated system in substantial accordance with the following procedure. Concentration values are imported in an Excel spreadsheet where pipetting volumes were calculated to perform dilution to 50 μg/mL in 0.22 mL. The spreadsheet was imported in a Hamilton Star method dedicated to performing dilution pipetting using the first elution block and elution buffer as diluent. The final, normalized plate was sterile filtered using 0.22 μm filter plates (Corning).

### Example 3—ELISA Studies for IL2Rb VHH

The single domain antibodies of the present disclosure were obtained from camels by immunization with an extracellular domain of a IL2Rb receptor. IL2Rb VHH molecules of the present disclosure of the present disclosure were generated in substantial accordance with the teaching of the Examples. Briefly, a camel was sequentially immunized with the ECD of the human IL2Rb and mouse IL2Rb over a period several weeks of by the subcutaneous an adjuvanted composition containing a recombinantly produced fusion proteins comprising the extracellular domain of the IL2Rb, the human IgG1 hinge domain and the human IgG1 heavy chain Fc. Following immunization, RNAs extracted from a blood sample of appropriate size VHH-hinge-CH2-CH3 species were transcribed to generate DNA sequences, digested to identify the approximately 400 bp fragment comprising the nucleic acid sequence encoding the VHH domain was isolated. The isolated sequence was digested with restriction endonucleases to facilitate insertion into a phagemid vector for in frame with a sequence encoding a his-tag and transformed into E. coli to generate a phage library. Multiple rounds of biopanning of the phage library were conducted to identify VHHs that bound to the ECD of IL2Rb (human or mouse as appropriate). Individual phage clones were isolated for periplasmic extract ELISA (PE-ELISA) in a 96-well plate format and selective binding confirmed by colorimetric determination. The IL2Rb binding molecules that demonstrated specific binding to the IL2Rb antigen were isolated and sequenced and sequences analyzed to identify VHH sequences, CDRs and identify unique VHH clonotypes. As used herein, the term “clonotypes” refers a collection of binding molecules that originate from the same B-cell progenitor cell, in particular collection of antigen binding molecules that belong to the same germline family, have the same CDR3 lengths, and have 70% or greater homology in CDR3 sequence. The VHH molecules demonstrating specific binding to the hIL2Rb ECD antigen (anti-human IL2Rb VHHs) and the CDRs isolated from such VHHs are provided in Table 34. The VHH molecules demonstrating specific binding to the mIL2Rb ECD antigen (anti-mouse IL2Rb VHHs) and the CDRs isolated from such VHHs are provided in Table 35. Nucleic acid sequences encoding the VHHs of Table 34 and 35 are provided in Tables 38 and 39, respectively.

To more fully characterize the binding properties and evaluate binding affinity of the VHH molecules generated in accordance with the foregoing, representative examples of each of the human VHH clonotypes were subjected to analysis by surface plasmon resonance in substantial accordance with the teaching of Example 6 herein. The results of these SPR studies are summarized in Table 16 below.

### Example 4. Evaluation of Binding Affinity Via Surface Plasmon Resonance for IL2Rb VHH

A representative example from each hIL2Rb VHH clonotype generated as descried above was selected for evaluation of binding via SPR as follows. Evaluation of binding affinity of the hIL2Rb binding molecules shown in Table 16 was conducted using surface plasmon resonance (SPR) in substantial accordance with the following procedure. All experiments were conducted in 10 mM Hepes, 150 mM NaCl, 0.05% (v/v) Polysorbate 20 (PS20) and 3 mM EDTA (HBS−EP+ buffer) on a Biacore T200 instrument equipped with a Protein A derivatized sensor chip (Cytiva). Mono-Fc VHH ligands were flowed at 5 μl/min for variable time ranging from 18 to 300 seconds, reaching the capture loads listed in the tables below. Following ligand capture, injections of a 2-fold dilution series of the extracellular domain of the IL2Rb-receptor modified to incorporate a C-terminal poly-His sequence, typically comprising at least five concentrations between 1 μM and 1 nM, were performed in either high performance or single cycle kinetics mode. Surface regeneration was achieved by flowing 10 mM glycine-HCl, pH 1.5 (60 seconds, 50 μL/min). Buffer-subtracted sensograms were processed with Biacore T200 Evaluation Software and globally fit with a 1:1 Langmuir binding model (bulk shift set to zero) to extract kinetics and affinity constants (kₐ, k_{d}, K_{D}). R_{MAX}<100 RU indicates surface density compatible with kinetics analysis. Calculated Rₘₐₓ values were generated using the equation: Rmax=Load (RU)×valency of ligand×(Molecular weight of analyte/Molecular weight of ligand). Surface activity was defined as the ratio of experimental/calculated Rmax. The results of these binding affinity experiments are provided in Table 16.

TABLE 16
anti-hIL2Rb Mono-Fc VHHs binding to hIL2Rb-his

| | SEQ | k_{ON} | k_{OFF} | Affinity | Rmax | Load | Calc. Rmax | Surface |
|---|---|---|---|---|---|---|---|---|
| Ligand | ID NO | (1/Ms) | (1/s) | (nM) | (RU) | (RU) | (RU) | Activity |
| hIL2Rb VHH1 | 25 | 1.98E+07 | 1.99E−02 | 1 | 17.6 | 62.4 | 48 | 37% |
| hIL2Rb_VHH2 | 26 | 1.39E+05 | 2.24E−03 | 16 | 5.4 | 26.8 | 20 | 26% |
| hIL2Rb_VHH3 | 27 | 1.57E+05 | 6.99E−03 | 47 | 14.7 | 36.8 | 28 | 52% |
| hIL2Rb_VHH4 | 28 | 6.00E+05 | 2.05E−03 | 3.4 | 24.4 | 33.2 | 25 | 96% |
| hIL2Rb_VHH5 | 29 | 3.82E+06 | 1.54E−03 | 0.4 | 32.3 | 100 | 77 | 42% |
| hIL2Rb_VHH6 | 30 | 1.90E+07 | 2.29E−02 | 1.2 | 38.9 | 96.9 | 74 | 53% |
| hIL2Rb_VHH7 | 31 | 2.86E+06 | 3.17E−03 | 1.1 | 32.1 | 98.3 | 75 | 43% |
| hIL2Rb_VHH8 | 32 | ND | ND | ND | ~5 | 279 | 213 | <5% |

### Example 5—ELISA Studies for IL2R2g VHH

The single domain antibodies of the present disclosure were obtained from camels by immunization with an extracellular domain of a IL2Rg receptor (CD132). IL2Rg V_{H}H molecules of the present disclosure of the present disclosure were generated in substantial accordance with the teaching of the Examples. Briefly, a camel was sequentially immunized with the ECD of the human IL2Rg and mouse IL2Rg over a period several weeks of by the subcutaneous an adjuvanted composition containing a recombinantly produced fusion proteins comprising the extracellular domain of the IL2Rg, the human IgG1 hinge domain and the human IgG1 heavy chain Fc. Following immunization, RNAs extracted from a blood sample of appropriate size VHH-hinge-CH2-CH3 species were transcribed to generate DNA sequences, digested to identify the approximately 400 bp fragment comprising the nucleic acid sequence encoding the VHH domain was isolated. The isolated sequence was digested with restriction endonucleases to facilitate insertion into a phagemid vector for in frame with a sequence encoding a his-tag and transformed into E. coli to generate a phage library. Multiple rounds of biopanning of the phage library were conducted to identify VHHs that bound to the ECD of IL2Rg (human or mouse as appropriate). Individual phage clones were isolated for periplasmic extract ELISA (PE-ELISA) in a 96-well plate format and selective binding confirmed by colorimetric determination. The IL2Rg binding molecules that demonstrated specific binding to the IL2Rg antigen were isolated and sequenced and sequences analyzed to identify VHH sequences, CDRs and identify unique VHH clonotypes. As used herein, the term “clonotypes” refers a collection of binding molecules that originate from the same B-cell progenitor cell, in particular collection of antigen binding molecules that belong to the same germline family, have the same CDR3 lengths, and have 70% or greater homology in CDR3 sequence. The VHH molecules demonstrating specific binding to the hIL2Rg ECD antigen (anti-human IL2Rg VHHs) and the CDRs isolated from such VHHs are provided in Table 36. The VHH molecules demonstrating specific binding to the mIL2Rg ECD antigen (anti-mouse IL2Rg VHHs) and the CDRs isolated from such VHHs are provided in Table 37. Nucleic acid sequences encoding the VHHs of Table 36 and 37 are provided in Tables 40 and 41, respectively.

### Example 6. Evaluation of Binding Affinity Via Surface Plasmon Resonance for IL2Rg VHH

To more fully characterize the binding properties and evaluate binding affinity of the VHH molecules generated in accordance with the foregoing, representative examples of each of the human VHH clonotypes were subjected to analysis of by surface plasmon resonance in substantial accordance with the teaching of the examples herein. The results of these SPR studies are summarized in Table 17 below.

TABLE 17
anti-hIL2Rg Mono-Fc VHHs binding to hIL2Rg-his
(Antigen: Sino Biological, Catalog# 10555)

| | SEQ | k_{ON} | k_{OFF} | Affinity | Rmax | Load | Calc. Rmax | Surface |
|---|---|---|---|---|---|---|---|---|
| Ligand | ID NO | (1/Ms) | (1/s) | (nM) | (RU) | (RU) | (RU) | Activity |
| hIL-2Rg_VHH8 | 306 | 3.66E+05 | 8.12E−04 | 2.2 | 41.6 | 49.3 | 38 | 110% |
| hIL-2Rg_VHH15 | 313 | 9.68E+04 | 2.51E−03 | 26 | 47.1 | 84 | 64 | 73% |
| hIL-2Rg_VHH19 | 317 | 2.85E+06 | 6.93E−03 | 2.4 | 7 | 28 | 21 | 33% |
| hIL-2Rg_VHH20 | 318 | 1.92E+05 | 2.70E−03 | 14.1 | 227 | 103 | 475.4 | 48% |
| hIL-2Rg_VHH21 | 319 | 6.56E+04 | 1.13E−03 | 17.2 | 14.6 | 57.5 | 245.8 | 6% |
| hIL-2Rg_VHH22 | 320 | 5.54E+05 | 3.54E−03 | 6.4 | 39.6 | 47.8 | 37 | 108% |

As illustrated by the data presented in Table 17, the hIL2Rg binding molecules generated in accordance with the teaching of present disclosure exhibit specific binding and provided a range of affinities to the extracellular domain of hIL2Rg.

### Example 7 Evaluation of Binding Affinity Via Surface Plasmon Resonance for IL2Rb/IL2R2 VHH Murine Dimer Constructs

Additional experiments were conducted with murine dimer constructs. All experiments were conducted in 10 mM Hepes, 150 mM NaCl, 0.05% (v/v) Polysorbate 20 (PS20) and 3 mM EDTA (HBS−EP+ buffer) on a Biacore T200 instrument equipped with Protein A or CAP biotin chips (Cytiva). For experiments on Protein A chips, Fc-fused ligands were flowed at 5 μl/min for variable time ranging from 18 to 300 seconds, reaching the capture loads listed in the tables below.

Following ligand capture, injections of a 2-fold dilution series of analyte typically comprising at least five concentrations between 1 μM and 1 nM were performed in either high performance or single cycle kinetics mode. Surface regeneration was achieved by flowing 10 mM glycine-HCl, pH 1.5 (60 seconds, 50 μL/min). Buffer-subtracted sensograms were processed with Biacore T200 Evaluation Software and globally fit with a 1:1 Langmuir binding model (bulk shift set to zero) to extract kinetics and affinity constants (ka, kd, KD). RMAX<100 RU indicates surface density compatible with kinetics analysis.

Experiments on CAP chips were performed as described above with an additional capture step of Biotin CAPture reagent (10 seconds, 40 uL/min) performed prior to capture of biotinylated ligands.

Calculated Rmax were generated using the equation Rmax=Load (RU)×valency of ligand×(Molecular weight of analyte/Molecular weight of ligand. Surface activity was defined as the ratio experimental/calculated Rmax. See tables below for sample information and experimental results.

Results for Anti-mIL2Rb/mIL2Rg dual VHHs binding to mIL2Rb-Fc were as follows:

| | | k_{ON} | k_{OFF} | Affinity | Rmax | Load | Calc. Rmax | Surface |
|---|---|---|---|---|---|---|---|---|
| Analyte | Ligand | (1/Ms) | (1/s) | (nM) | (RU) | (RU) | (RU) | Activity |
| DR870-his | mIL2Rb-Fc | 4.0E+04 | 1.6E−03 | 39 | 13.9 | 181 | 169 | 8% |
| DR871-his | (SinoBiological | <5.2E+05 | 9.9E−04 | >1.9 | 18.4 | 175 | 164 | 11% |
| DR873-his | cat#50792) | 5.2E+04 | 1.6E−03 | 31 | 16.6 | 172 | 160 | 10% |

Results for Anti-mIL2Rb/mIL2Rg dual VHHs binding to mIL2Rg-Fc (Sino Biological, catalog #50087) were as follows:

| | | k_{ON} | k_{OFF} | Affinity | Rmax | Load | Calc. Rmax | Surface |
|---|---|---|---|---|---|---|---|---|
| Analyte | Ligand | (1/Ms) | (1/s) | (nM) | (RU) | (RU) | (RU) | Activity |
| DR870-his | mIL2Rg-Fc | 3.2E+06 | 1.6E−03 | 0.49 | 68.5 | 198 | 148 | 46% |
| DR871-his | (SinoBiological | 2.8E+06 | <1E−05 | <0.01 | 87.8 | 200 | 149 | 59% |
| DR873-his | cat#50087) | 6.1E+06 | 4.4E−04 | 0.072 | 80.9 | 195 | 146 | 56% |

### Example 8—Evaluation of STAT5 Activity of Anti-IL2Rβ/γ V_{H}H²s In NKL Cells

The anti-IL2Rβ/γ V_{H}H²s were evaluated for activity in NKL cells (as described in Robertson, et al (1996) Experimental Hematology 24(3):406-15). NKL is a human IL2 dependent Leukemic cell line with NK cell characteristics that expresses IL2Rβ and IL2Rγ chains and is able to phosphorylate STAT5 and proliferate in response to IL2 receptor signaling.

NKL cells were contacted with purified anti-IL2Rβ/γ V_{H}H²s to examine induction of STAT5 phosphorylation as follows as follows: Cells were cultured in medium consisting of RPMI 1640 (ThermoFisher), 10 percent fetal bovine serum (ThermoFisher), 1 percent penicillin/streptomycin (ThermoFisher), 1 percent glutamax (ThermoFisher) and 100 pM human IL2 (Synthekine P191029PL 1) at densities between 0.2-1 million cells per ml. Prior to the experiment NKL cells were harvested, centrifuged and washed in DPBS twice. Cells were resuspended at 2 million cells/mL in PBS. anti-IL2Rβ/γ V_{H}H²s were diluted into 96-well plates (Falcon) to 30 nM in 50 μl DPBS and 100 thousand NKL cells were added per well in 50 μl DPBS. Controls included wells without anti-IL2Rβ/γ V_{H}H²s or human IL2 (media) and wells with human IL2 at a final concentration of 100 pM. Plates were transferred to a humidified incubator (ThermoFisher) and incubated at 37 degrees centigrade, 5 percent carbon dioxide for 20 min.

Plates were removed from the incubator and cells were lysed by adding 100 μl per well of Tris Lysis buffer supplemented with Protease Inhibitor Solution, Phosphatase Inhibitor I and Phosphatase Inhibitor II from MSD Multispot Assay System Phospho-STAT Panel Kit (MSD K15202D) according to manufacturer's instructions. Plates were incubated on ice for 15 minutes and centrifuged at 600×g for 6 minutes. Cell lysates were transferred to a new 96 well plate.

Induction of STAT5 phosphorylation was measured in the cell lysates using the MSD Multispot Assay System Phospho-STAT Panel (MSD K15202D) according to manufacturer's instructions. Briefly, mAb precoated MSD Phospho-STAT panel assay plates were incubated with 150 μL per well Blocker A in Tris Wash Buffer at 30 mg/mL for 60 min on an orbital shaker (VWR Scientific) at 300 rpm at room temperature. Plates were washed 3 times with Tris Wash Buffer and 30 μL Cell lysates were added per well. Plates were incubated for 60 min on an orbital shaker (VWR Scientific) at 300 rpm at room temperature. Plates were washed 3 times with Tris Wash Buffer and 25 μL 1×detection antibody in 10 mg/mL Blocker A in Tris Wash Buffer was added to each well. Plates were incubated for 60 min on an orbital shaker (VWR Scientific) at 300 rpm at room temperature. Plates were washed 3 times with Tris Wash Buffer and 150 μL 1× Read Buffer T was added to each well and Luminescence signal was read on a Mesoscale Quickplex SQ 120 instrument

To compare the effect of each anti-IL2Rβ/γ V_{H}H² variant upon STAT5 phosphorylation, MSD luminescence signal values for cells treated with anti-IL2Rβ/γ V_{H}H² were compared to those obtained for control cells treated with growth medium alone to yield a fold induction and with control cells treated with human IL2 to yield a level of activity relative to the response to IL2 as a percentage. The data from these experiments is presented in Table 7.

In this assay anti-IL2Rβ/γ V_{H}H²s induced STAT5 phosphorylation at distinct levels, but not above levels observed with IL2 at 100 pM.

TABLE 7
pSTAT5 Induction by anti-IL2Rβ/γ V_{H}H² on NKL Cells

| | NKL | Relative | Relative |
|---|---|---|---|
| | pSTAT5 | pSTAT5 | pSTAT5 |
| | Induction | Induction | Induction |
| Anti-IL2Rβ/γ V_{H}H² | (MSD | (fold | (% hIL2 |
| Construct | Signal) | induction) | response) |
| DR632(DR229-DR214) | 595 | 0.5 | 7.9 |
| DR633(DR229-DR217) | 870 | 0.7 | 11.5 |
| DR634(DR229-DR583) | 1189 | 1.0 | 15.7 |
| DR635(DR229-DR584) | 6264 | 5.2 | 82.7 |
| DR636(DR229-DR585) | 912 | 0.8 | 12.0 |
| DR637(DR229-DR586) | 850 | 0.7 | 11.2 |
| DR638(DR229-DR587) | 4495 | 3.8 | 59.4 |
| DR639(DR229-DR587) | 3077 | 2.6 | 40.6 |
| DR640(DR229-DR589) | 1145 | 1.0 | 15.1 |
| DR641(DR229-DR590) | 697 | 0.6 | 9.2 |
| DR642(DR230-DR214) | 3212 | 2.7 | 42.4 |
| DR643(DR230-DR217) | 5300 | 4.4 | 70.0 |
| DR644(DR230-DR583) | 1034 | 0.9 | 13.7 |
| DR645(DR230-DR584) | 2968 | 2.5 | 39.2 |
| DR646(DR230-DR585) | 1095 | 0.9 | 14.5 |
| DR647(DR230-DR586) | 5234 | 4.4 | 69.1 |
| DR648(DR230-DR587) | 3966 | 3.3 | 52.4 |
| DR649(DR230-DR588) | 579 | 0.5 | 7.6 |
| DR650(DR230-DR589) | 861 | 0.7 | 11.4 |
| DR651(DR230-DR590) | 742 | 0.6 | 9.8 |
| DR652(DR231-DR214) | 2626 | 2.2 | 34.7 |
| DR653(DR231-DR217) | 475 | 0.4 | 6.3 |
| DR654(DR231-DR583) | 923 | 0.8 | 12.2 |
| DR655(DR231-DR584) | 968 | 0.8 | 12.8 |
| DR656(DR231-DR585) | 1366 | 1.1 | 18.0 |
| DR657(DR231-DR586) | 1041 | 0.9 | 13.7 |
| DR658(DR231-DR587) | 683 | 0.6 | 9.0 |
| DR659(DR231-DR588) | 1037 | 0.9 | 13.7 |
| DR660(DR231-DR589) | 781 | 0.7 | 10.3 |
| DR661(DR231-DR590) | 888 | 0.7 | 11.7 |
| DR662(DR232-DR214) | 861 | 0.7 | 11.4 |
| DR663(DR232-DR217) | 1055 | 0.9 | 13.9 |
| DR664(DR232-DR583) | 967 | 0.8 | 12.8 |
| DR665(DR232-DR584) | 1105 | 0.9 | 14.6 |
| DR666(DR232-DR585) | 1188 | 1.0 | 15.7 |
| DR667(DR232-DR586) | 1110 | 0.9 | 14.7 |
| DR668(DR232-DR587) | 944 | 0.8 | 12.5 |
| DR669(DR232-DR588) | 931 | 0.8 | 12.3 |
| DR670(DR232-DR589) | 1245 | 1.0 | 16.4 |
| DR671(DR232-DR590) | 1599 | 1.3 | 21.1 |
| DR672(DR233-DR214) | 1587 | 1.3 | 21.0 |
| DR673(DR233-DR217) | 1653 | 1.4 | 21.8 |
| DR674(DR233-DR583) | 3347 | 2.8 | 44.2 |
| DR675(DR233-DR584) | 3561 | 3.0 | 47.0 |
| DR676(DR233-DR584) | 1409 | 1.2 | 18.6 |
| DR677(DR233-DR586) | 845 | 0.7 | 11.2 |
| DR678(DR233-DR587) | 6138 | 5.1 | 81.1 |
| DR679(DR233-DR588) | 1810 | 1.5 | 23.9 |
| DR680(DR233-DR589) | 2073 | 1.7 | 27.4 |
| DR681(DR233-DR590) | 1374 | 1.2 | 18.1 |
| DR682(DR234-DR214) | 1055 | 0.9 | 13.9 |
| DR683(DR234-DR217) | 1006 | 0.8 | 13.3 |
| DR684(DR234-DR583) | 725 | 0.6 | 9.6 |
| DR685(DR234-DR584) | 1070 | 0.9 | 14.1 |
| DR686(DR234-DR585) | 868 | 0.7 | 11.5 |
| DR687(DR234-DR586) | 1173 | 1.0 | 15.5 |
| DR688(DR234-DR587) | 997 | 0.8 | 13.2 |
| DR689(DR234-DR588) | 1227 | 1.0 | 16.2 |
| DR690(DR234-DR589) | 1280 | 1.1 | 16.9 |
| DR691(DR234-DR590) | 917 | 0.8 | 12.1 |
| DR692(DR214-DR229) | 898 | 0.8 | 11.9 |
| DR693(DR214-DR230) | 917 | 0.8 | 12.1 |
| DR694(DR214-DR231) | 984 | 0.8 | 13.0 |
| DR695(DR214-DR232) | 1262 | 1.1 | 16.7 |
| DR696(DR214-DR233) | 1603 | 1.3 | 21.2 |
| DR697(DR214-DR234) | 758 | 0.6 | 10.0 |
| DR698(DR217-DR229) | 1051 | 0.9 | 13.9 |
| DR699(DR217-DR230) | 837 | 0.7 | 11.1 |
| DR700(DR217-DR231) | 881 | 0.7 | 11.6 |
| DR701(DR217-DR232) | 2092 | 1.8 | 27.6 |
| DR702(DR217-DR233) | 1078 | 0.9 | 14.2 |
| DR703(DR217-DR234) | 734 | 0.6 | 9.7 |
| DR704(DR583-DR229) | 722 | 0.6 | 9.5 |
| DR705(DR583-DR230) | 1150 | 1.0 | 15.2 |
| DR706(DR583-DR231) | 752 | 0.6 | 9.9 |
| DR707(DR583-DR232) | 1119 | 0.9 | 14.8 |
| DR708(DR583-DR233) | 772 | 0.6 | 10.2 |
| DR709(DR583-DR234) | 1070 | 0.9 | 14.1 |
| DR710(DR584-DR229) | 812 | 0.7 | 10.7 |
| DR711(DR584-DR230) | 1091 | 0.9 | 14.4 |
| DR712(DR584-DR231) | 992 | 0.8 | 13.1 |
| DR713(DR584-DR232) | 1044 | 0.9 | 13.8 |
| DR714(DR584-DR233) | 923 | 0.8 | 12.2 |
| DR715(DR584-DR234) | 1003 | 0.8 | 13.2 |
| DR716(DR585-DR229) | 2639 | 2.2 | 34.9 |
| DR717(DR585-DR230) | 2103 | 1.8 | 27.8 |
| DR718(DR585-DR231) | 6348 | 5.3 | 83.8 |
| DR719(DR585-DR232) | 1957 | 1.6 | 25.8 |
| DR720(DR585-DR233) | 1999 | 1.7 | 26.4 |
| DR721(DR585-DR234) | 2778 | 2.3 | 36.7 |
| DR722(DR586-DR229) | 4231 | 3.5 | 55.9 |
| DR723(DR586-DR230) | 881 | 0.7 | 11.6 |
| DR724(DR586-DR231) | 5638 | 4.7 | 74.5 |
| DR725(DR586-DR232) | 910 | 0.8 | 12.0 |
| DR726(DR586-DR233) | 1132 | 0.9 | 15.0 |
| DR727(DR586-DR234) | 701 | 0.6 | 9.3 |
| DR728(DR587-DR229) | 859 | 0.7 | 11.3 |
| DR729(DR587-DR230) | 948 | 0.8 | 12.5 |
| DR730(DR587-DR231) | 662 | 0.6 | 8.7 |
| DR731(DR587-DR232) | 1055 | 0.9 | 13.9 |
| DR732(DR587-DR233) | 862 | 0.7 | 11.4 |
| DR733(DR587-DR234) | 771 | 0.6 | 10.2 |
| DR734(DR588-DR229) | 800 | 0.7 | 10.6 |
| DR735(DR588-DR230) | 1090 | 0.9 | 14.4 |
| DR736(DR588-DR231) | 1406 | 1.2 | 18.6 |
| DR737(DR588-DR232) | 932 | 0.8 | 12.3 |
| DR738(DR588-DR233) | 847 | 0.7 | 11.2 |
| DR739(DR588-DR234) | 1020 | 0.9 | 13.5 |
| DR740(DR589-DR229) | 947 | 0.8 | 12.5 |
| DR741(DR589-DR230) | 808 | 0.7 | 10.7 |
| DR742(DR589-DR231) | 1013 | 0.8 | 13.4 |
| DR743(DR589-DR232) | 938 | 0.8 | 12.4 |
| DR744(DR589-DR233) | 807 | 0.7 | 10.7 |
| DR745(DR589-DR234) | 638 | 0.5 | 8.4 |
| DR746(DR590-DR229) | 1304 | 1.1 | 17.2 |
| DR747(DR590-DR230) | 1622 | 1.4 | 21.4 |
| DR748(DR590-DR231) | 890 | 0.7 | 11.8 |
| DR749(DR590-DR232) | 432 | 0.4 | 5.7 |
| DR750(DR590-DR233) | 1041 | 0.9 | 13.7 |
| DR751(DR590-DR234) | 605 | 0.5 | 8.0 |
| medium | 1194 | 1.0 | 15.8 |
| IL2 | 7571 | 6.3 | 100.0 |

### Example 9—Evaluation of Proliferative Activity of Anti-IL2Rβ/γ V_{H}H²s In NKL Cells

The anti-IL2Rβ/γ V_{H}H²s were evaluated for activity in NKL cells (Robertson, et al (1996) Experimental Hematology 24(3):406-15). NKL is a human IL2 dependent Leukemic cell line with NK cell characteristics that expresses IL2Rβ and IL2Rγ chains and is able to phosphorylate STAT5 and proliferate in response to IL2 receptor signaling.

NKL cells were contacted with purified anti-IL2Rβ/γ V_{H}H²s as follows: Cells were cultured in medium consisting of RPMI 1640 (ThermoFisher), 10 percent fetal bovine serum (ThermoFisher), 1 percent penicillin/streptomycin (ThermoFisher), 1 percent glutamax (ThermoFisher) and 100 pM human IL2 (Synthekine P191029PL 1) at densities between 0.2-1 million cells per ml. Prior to the experiment NKL cells were harvested, centrifuged, and washed in DPBS twice. NKL cells were resuspended at 1 million cells/mL in growth medium without IL2. Anti-IL2Rβ/γ V_{H}H²s were diluted into 96-well plates (Falcon) at 30 nM in 50 μl growth medium without IL2 and 50 thousand NKL cells in 50 μl DPBS were added per well. Controls included wells without anti-IL2Rβ/γ V_{H}H²s or human IL2 (media) and wells with human IL2 at a final concentration of 100 pM. Plates were transferred to a humidified incubator (ThermoFisher) and incubated at 37 degrees centigrade, 5 percent carbon dioxide for 72 hrs.

Plates were removed from the incubator and cells were lysed by adding 100 μl per well of Celltiterglo™ (CTG) (Promega) according to manufacturer's instructions. Cell lysates were mixed on an orbital shaker (VWR Scientific) for 10 minutes at 300 rpm. Luminescence was read as counts per second on an Envision 2103 Multilabel Plate Reader (Perkin Elmer) using the ATPLite protocol.

To compare the effect of each IL-2 VHH dimer upon pSTAT5 induction and NKL cell proliferation, luminescence values from pSTAT and celltiterglo measurements were compared to those obtained for control cells treated with growth medium alone and control cells treated with human IL-2 at 100 pM. IL-2 V_{H}H dimers were identified that induced higher luminescence signals for pSTAT5 induction and cell proliferation than media control but lower than IL-2 at the concentrations used. The data from these experiments is presented in Table 8.

In this assay 19/120 anti-IL2Rβ/γ V_{H}H²s induced NKL cell proliferation at distinct levels (>1 fold), but not above levels observed with IL2 at 100 pM.

TABLE 8
Induction of Proliferation by Anti-IL2Rβ/γ V_{H}H² on NKL Cells

| | NKL | Relative | Relative |
|---|---|---|---|
| | proliferation | pSTAT5 | pSTAT5 |
| | Induction | Induction | Induction |
| Anti-IL2Rβ/γ V_{H}H² | CTG | (fold | (% hIL2 |
| Construct | (cpm) | induction) | response) |
| DR632(DR229-DR214) | 116506 | 0.8 | 56.6 |
| DR633(DR229-DR217) | 138124 | 0.9 | 67.1 |
| DR634(DR229-DR583) | 136398 | 0.9 | 66.3 |
| DR635(DR229-DR584) | 178418 | 1.2 | 86.7 |
| DR636(DR229-DR585) | 115410 | 0.8 | 56.1 |
| DR637(DR229-DR586) | 122216 | 0.8 | 59.4 |
| DR638(DR229-DR587) | 148994 | 1.0 | 72.4 |
| DR639(DR229-DR587) | 177470 | 1.2 | 86.2 |
| DR640(DR229-DR589) | 137282 | 0.9 | 66.7 |
| DR641(DR229-DR590) | 132522 | 0.9 | 64.4 |
| DR642(DR230-DR214) | 147492 | 1.0 | 71.7 |
| DR643(DR230-DR217) | 177402 | 1.2 | 86.2 |
| DR644(DR230-DR583) | 132806 | 0.9 | 64.5 |
| DR645(DR230-DR584) | 152106 | 1.0 | 73.9 |
| DR646(DR230-DR585) | 124118 | 0.8 | 60.3 |
| DR647(DR230-DR586) | 176110 | 1.2 | 85.6 |
| DR648(DR230-DR587) | 154232 | 1.0 | 74.9 |
| DR649(DR230-DR588) | 135958 | 0.9 | 66.0 |
| DR650(DR230-DR589) | 138590 | 0.9 | 67.3 |
| DR651(DR230-DR590) | 140664 | 1.0 | 68.3 |
| DR652(DR231-DR214) | 168884 | 1.1 | 82.0 |
| DR653(DR231-DR217) | 127516 | 0.9 | 61.9 |
| DR654(DR231-DR583) | 144488 | 1.0 | 70.2 |
| DR655(DR231-DR584) | 126880 | 0.9 | 61.6 |
| DR656(DR231-DR585) | 121026 | 0.8 | 58.8 |
| DR657(DR231-DR586) | 146186 | 1.0 | 71.0 |
| DR658(DR231-DR587) | 105546 | 0.7 | 51.3 |
| DR659(DR231-DR588) | 152634 | 1.0 | 74.1 |
| DR660(DR231-DR589) | 126740 | 0.9 | 61.6 |
| DR661(DR231-DR590) | 136314 | 0.9 | 66.2 |
| DR662(DR232-DR214) | 112768 | 0.8 | 54.8 |
| DR663(DR232-DR217) | 139844 | 0.9 | 67.9 |
| DR664(DR232-DR583) | 119004 | 0.8 | 57.8 |
| DR665(DR232-DR584) | 143704 | 1.0 | 69.8 |
| DR666(DR232-DR585) | 130088 | 0.9 | 63.2 |
| DR667(DR232-DR586) | 136678 | 0.9 | 66.4 |
| DR668(DR232-DR587) | 103200 | 0.7 | 50.1 |
| DR669(DR232-DR588) | 147228 | 1.0 | 71.5 |
| DR670(DR232-DR589) | 122194 | 0.8 | 59.4 |
| DR671(DR232-DR590) | 163078 | 1.1 | 79.2 |
| DR672(DR233-DR214) | 115320 | 0.8 | 56.0 |
| DR673(DR233-DR217) | 150560 | 1.0 | 73.1 |
| DR674(DR233-DR583) | 137342 | 0.9 | 66.7 |
| DR675(DR233-DR584) | 167770 | 1.1 | 81.5 |
| DR676(DR233-DR584) | 169444 | 1.2 | 82.3 |
| DR677(DR233-DR586) | 135714 | 0.9 | 65.9 |
| DR678(DR233-DR587) | 184958 | 1.3 | 89.9 |
| DR679(DR233-DR588) | 156864 | 1.1 | 76.2 |
| DR680(DR233-DR589) | 122214 | 0.8 | 59.4 |
| DR681(DR233-DR590) | 153052 | 1.0 | 74.4 |
| DR682(DR234-DR214) | 110310 | 0.7 | 53.6 |
| DR683(DR234-DR217) | 140478 | 1.0 | 68.2 |
| DR684(DR234-DR583) | 107838 | 0.7 | 52.4 |
| DR685(DR234-DR584) | 139478 | 0.9 | 67.8 |
| DR686(DR234-DR585) | 120032 | 0.8 | 58.3 |
| DR687(DR234-DR586) | 128462 | 0.9 | 62.4 |
| DR688(DR234-DR587) | 136754 | 0.9 | 66.4 |
| DR689(DR234-DR588) | 149798 | 1.0 | 72.8 |
| DR690(DR234-DR589) | 126096 | 0.9 | 61.3 |
| DR691(DR234-DR590) | 139710 | 0.9 | 67.9 |
| DR692(DR214-DR229) | 108572 | 0.7 | 52.7 |
| DR693(DR214-DR230) | 130734 | 0.9 | 63.5 |
| DR694(DR214-DR231) | 113610 | 0.8 | 55.2 |
| DR695(DR214-DR232) | 137054 | 0.9 | 66.6 |
| DR696(DR214-DR233) | 96674 | 0.7 | 47.0 |
| DR697(DR214-DR234) | 132076 | 0.9 | 64.2 |
| DR698(DR217-DR229) | 124264 | 0.8 | 60.4 |
| DR699(DR217-DR230) | 141240 | 1.0 | 68.6 |
| DR700(DR217-DR231) | 174410 | 1.2 | 84.7 |
| DR701(DR217-DR232) | 163990 | 1.1 | 79.7 |
| DR702(DR217-DR233) | 136290 | 0.9 | 66.2 |
| DR703(DR217-DR234) | 140046 | 1.0 | 68.0 |
| DR704(DR583-DR229) | 118478 | 0.8 | 57.6 |
| DR705(DR583-DR230) | 136916 | 0.9 | 66.5 |
| DR706(DR583-DR231) | 113692 | 0.8 | 55.2 |
| DR707(DR583-DR232) | 130934 | 0.9 | 63.6 |
| DR708(DR583-DR233) | 100954 | 0.7 | 49.0 |
| DR709(DR583-DR234) | 138480 | 0.9 | 67.3 |
| DR710(DR584-DR229) | 113468 | 0.8 | 55.1 |
| DR711(DR584-DR230) | 134004 | 0.9 | 65.1 |
| DR712(DR584-DR231) | 133920 | 0.9 | 65.1 |
| DR713(DR584-DR232) | 149190 | 1.0 | 72.5 |
| DR714(DR584-DR233) | 116526 | 0.8 | 56.6 |
| DR715(DR584-DR234) | 136656 | 0.9 | 66.4 |
| DR716(DR585-DR229) | 156810 | 1.1 | 76.2 |
| DR717(DR585-DR230) | 158976 | 1.1 | 77.2 |
| DR718(DR585-DR231) | 167952 | 1.1 | 81.6 |
| DR719(DR585-DR232) | 158644 | 1.1 | 77.1 |
| DR720(DR585-DR233) | 145766 | 1.0 | 70.8 |
| DR721(DR585-DR234) | 168984 | 1.1 | 82.1 |
| DR722(DR586-DR229) | 151054 | 1.0 | 73.4 |
| DR723(DR586-DR230) | 125438 | 0.9 | 60.9 |
| DR724(DR586-DR231) | 180326 | 1.2 | 87.6 |
| DR725(DR586-DR232) | 126750 | 0.9 | 61.6 |
| DR726(DR586-DR233) | 125440 | 0.9 | 60.9 |
| DR727(DR586-DR234) | 127484 | 0.9 | 61.9 |
| DR728(DR587-DR229) | 104160 | 0.7 | 50.6 |
| DR729(DR587-DR230) | 124594 | 0.8 | 60.5 |
| DR730(DR587-DR231) | 102252 | 0.7 | 49.7 |
| DR731(DR587-DR232) | 127720 | 0.9 | 62.0 |
| DR732(DR587-DR233) | 108350 | 0.7 | 52.6 |
| DR733(DR587-DR234) | 133262 | 0.9 | 64.7 |
| DR734(DR588-DR229) | 119826 | 0.8 | 58.2 |
| DR735(DR588-DR230) | 141022 | 1.0 | 68.5 |
| DR736(DR588-DR231) | 145904 | 1.0 | 70.9 |
| DR737(DR588-DR232) | 136648 | 0.9 | 66.4 |
| DR738(DR588-DR233) | 104204 | 0.7 | 50.6 |
| DR739(DR588-DR234) | 137990 | 0.9 | 67.0 |
| DR740(DR589-DR229) | 131632 | 0.9 | 63.9 |
| DR741(DR589-DR230) | 144776 | 1.0 | 70.3 |
| DR742(DR589-DR231) | 135948 | 0.9 | 66.0 |
| DR743(DR589-DR232) | 138268 | 0.9 | 67.2 |
| DR744(DR589-DR233) | 136268 | 0.9 | 66.2 |
| DR745(DR589-DR234) | 132428 | 0.9 | 64.3 |
| DR746(DR590-DR229) | 132472 | 0.9 | 64.4 |
| DR747(DR590-DR230) | 157594 | 1.1 | 76.6 |
| DR748(DR590-DR231) | 132586 | 0.9 | 64.4 |
| DR749(DR590-DR232) | 117108 | 0.8 | 56.9 |
| DR750(DR590-DR233) | 142346 | 1.0 | 69.2 |
| DR751(DR590-DR234) | 114290 | 0.8 | 55.5 |
| medium | 147254 | 1.0 | 71.5 |
| IL2 | 205850 | 1.4 | 100.0 |

### Example 10—Evaluation of Activity of Anti-IL2Rβ/γ V_{H}H²s In Primary NK Cells

The anti-IL2Rβ/γ V_{H}H²s were evaluated for activity in primary NK cells isolated from human peripheral blood. Primary NK cells express IL2Rβ and IL2Rγ chains and are able to phosphorylate STAT5, proliferate and produce IFN-γ in response to IL2 receptor signaling.

NK cells were isolated form peripheral blood of healthy donors collected in Leukoreduction System (LRS) Chambers at the Stanford Blood Bank (Palo Alto). Briefly, PBMC were isolated from LRS Chambers using the human Buffy Coat/LRSC PBMC Isolation Kit (Miltenyi). LRS Chambers were harvested in separation buffer (DPBS, 0.5% BSA, 2 mM EDTA) and mixed with sedimentation buffer and Red Blood Cell (RBC) removal antibodies and EDTA at a final concentration of 5 mM in 50 mL Centrifuge tubes. Tubes were centrifuged at 50×g for 3 minutes at room temperature. Supernatant with cells was collected and transferred to a new 50 mL centrifuge tube and separation buffer was added to 50 mL. Tubes were centrifuged at 300×g for 5 minutes at room temperature and supernatant discarded. Cell pellets were resuspended in 4 mL separation buffer and 2 mL Erythrocyte Depletion Microbeads and 1 mL Granulocyte Depletion Microbeads were added. Cells were incubated for 10 minutes at 2-8 degrees centigrade and PBMC were isolated on an AutoMacs Pro instrument (Miltenyi) using protocol ‘Cult 5’. PBMC were counted on a Vi-Cell XR instrument (Beckman).

NK cells were isolated form PBMC by positive selection using CD56 Microbeads (Miltenyi). Briefly, 1 billion PBMC were incubated with 2 mL CD56 Microbeads and incubated for 15 minutes at 2-8 degrees centigrade. Cells were washed, resuspended in separation buffer and NK cells isolated on an AutoMacs Pro instrument (Miltenyi) using protocol ‘possel’. NK cells were counted on a Vi-Cell XR instrument (Beckman).

Prior to the experiment NK cells were centrifuged and washed in DPBS twice. Cells were resuspended at 2 million cells/mL in PBS. Anti-IL2Rβ/γ V_{H}H²s were diluted into 96-well plates (Falcon) to 30 nM in 50 μl DPBS and 100 thousand NK cells were added per well in 50 μl DPBS. Controls included wells without anti-IL2Rβ/γ V_{H}H²s or human IL2 (media) and wells with human IL2 at a final concentration of 100 pM. Plates were transferred to a humidified incubator (ThermoFisher) and incubated at 37 degrees centigrade, 5 percent carbon dioxide for 20 min.

Plates were removed from the incubator and cells were lysed by adding 100 μl per well of Tris Lysis buffer supplemented with Protease Inhibitor Solution, Phosphatase Inhibitor I and Phosphatase Inhibitor II from MSD Multispot Assay System Phospho-STAT Panel Kit (MSD K15202D) according to manufacturer's instructions. Plates were incubated on ice for 15 minutes and centrifuged at 600×g for 6 minutes. Cell lysates were transferred to a new 96 well plate.

Induction of STAT5 phosphorylation was measured in the cell lysates using the MSD Multispot Assay System Phospho-STAT Panel (MSD K15202D) according to manufacturer's instructions. Briefly, mAb precoated MSD Phospho-STAT panel assay plates were incubated with 150 μL per well Blocker A in Tris Wash Buffer at 30 mg/mL for 60 min on an orbital shaker (VWR Scientific) at 300 rpm at room temperature. Plates were washed 3 times with Tris Wash Buffer and 30 μL Cell lysates were added per well. Plates were incubated for 60 min on an orbital shaker (VWR Scientific) at 300 rpm at room temperature. Plates were washed 3 times with Tris Wash Buffer and 25 μL 1×detection antibody in 10 mg/mL Blocker A in Tris Wash Buffer was added to each well. Plates were incubated for 60 min on an orbital shaker (VWR Scientific) at 300 rpm at room temperature. Plates were washed 3 times with Tris Wash Buffer and 150 μL 1× Read Buffer T was added to each well and Luminescence signal was read on a Mesoscale Quickplex SQ 120 instrument

To compare the effect of each anti-IL2Rβ/γ V_{H}H²s variant upon STAT5 phosphorylation, MSD luminescence signal values for cells treated with anti-IL2Rβ/γ V_{H}H²s were compared to those obtained for control cells treated with growth medium alone to yield a fold induction and with control cells treated with human IL2 to yield an activity relative to the response to IL2 as a percentage. The data from these experiments is presented in Table 9.

In this assay 45/120 anti-IL2Rβ/γ V_{H}H²s induced STAT5 phosphorylation at distinct levels, but not above levels observed with IL2 at 100 pM.

TABLE 9
pSTAT5 Induction by Anti-IL2Rβ/γ V_{H}H²s on Primary NK Cells

| | | | Relative |
|---|---|---|---|
| | | Relative | pSTAT5 |
| | NK pSTAT5 | pSTAT5 | Induction |
| Anti-IL2Rβ/γ V_{H}H² | Induction | Induction | (% hIL2 |
| Construct | (MSD Signal) | (fold induction) | response) |
| DR632(DR229-DR214) | 574 | 0.8 | 1.1 |
| DR633(DR229-DR217) | 339 | 0.5 | 0.7 |
| DR634(DR229-DR583) | 2165 | 3.1 | 4.3 |
| DR635(DR229-DR584) | 15080 | 21.4 | 30.0 |
| DR636(DR229-DR585) | 3220 | 4.6 | 6.4 |
| DR637(DR229-DR586) | 280 | 0.4 | 0.6 |
| DR638(DR229-DR587) | 24366 | 34.6 | 48.5 |
| DR639(DR229-DR587) | 3561 | 5.1 | 7.1 |
| DR640(DR229-DR589) | 1756 | 2.5 | 3.5 |
| DR641(DR229-DR590) | 195 | 0.3 | 0.4 |
| DR642(DR230-DR214) | 13823 | 19.6 | 27.5 |
| DR643(DR230-DR217) | 24065 | 34.1 | 47.9 |
| DR644(DR230-DR583) | 556 | 0.8 | 1.1 |
| DR645(DR230-DR584) | 7741 | 11.0 | 15.4 |
| DR646(DR230-DR585) | 1789 | 2.5 | 3.6 |
| DR647(DR230-DR586) | 21564 | 30.6 | 42.9 |
| DR648(DR230-DR587) | 13437 | 19.1 | 26.7 |
| DR649(DR230-DR588) | 236 | 0.3 | 0.5 |
| DR650(DR230-DR589) | 349 | 0.5 | 0.7 |
| DR651(DR230-DR590) | 338 | 0.5 | 0.7 |
| DR652(DR231-DR214) | 12830 | 18.2 | 25.5 |
| DR653(DR231-DR217) | 223 | 0.3 | 0.4 |
| DR654(DR231-DR583) | 220 | 0.3 | 0.4 |
| DR655(DR231-DR584) | 2826 | 4.0 | 5.6 |
| DR656(DR231-DR585) | 6038 | 8.6 | 12.0 |
| DR657(DR231-DR586) | 213 | 0.3 | 0.4 |
| DR658(DR231-DR587) | 202 | 0.3 | 0.4 |
| DR659(DR231-DR588) | 214 | 0.3 | 0.4 |
| DR660(DR231-DR589) | 204 | 0.3 | 0.4 |
| DR661(DR231-DR590) | 192 | 0.3 | 0.4 |
| DR662(DR232-DR214) | 224 | 0.3 | 0.4 |
| DR663(DR232-DR217) | 546 | 0.8 | 1.1 |
| DR664(DR232-DR583) | 1126 | 1.6 | 2.2 |
| DR665(DR232-DR584) | 221 | 0.3 | 0.4 |
| DR666(DR232-DR585) | 255 | 0.4 | 0.5 |
| DR667(DR232-DR586) | 604 | 0.9 | 1.2 |
| DR668(DR232-DR587) | 277 | 0.4 | 0.6 |
| DR669(DR232-DR588) | 456 | 0.6 | 0.9 |
| DR670(DR232-DR589) | 400 | 0.6 | 0.8 |
| DR671(DR232-DR590) | 1875 | 2.7 | 3.7 |
| DR672(DR233-DR214) | 1358 | 1.9 | 2.7 |
| DR673(DR233-DR217) | 3071 | 4.4 | 6.1 |
| DR674(DR233-DR583) | 5420 | 7.7 | 10.8 |
| DR675(DR233-DR584) | 8803 | 12.5 | 17.5 |
| DR676(DR233-DR584) | 580 | 0.8 | 1.2 |
| DR677(DR233-DR586) | 836 | 1.2 | 1.7 |
| DR678(DR233-DR587) | 16689 | 23.7 | 33.2 |
| DR679(DR233-DR588) | 5952 | 8.4 | 11.8 |
| DR680(DR233-DR589) | 5639 | 8.0 | 11.2 |
| DR681(DR233-DR590) | 1228 | 1.7 | 2.4 |
| DR682(DR234-DR214) | 1175 | 1.7 | 2.3 |
| DR683(DR234-DR217) | 480 | 0.7 | 1.0 |
| DR684(DR234-DR583) | 214 | 0.3 | 0.4 |
| DR685(DR234-DR584) | 881 | 1.2 | 1.8 |
| DR686(DR234-DR585) | 246 | 0.3 | 0.5 |
| DR687(DR234-DR586) | 289 | 0.4 | 0.6 |
| DR688(DR234-DR587) | 2121 | 3.0 | 4.2 |
| DR689(DR234-DR588) | 3040 | 4.3 | 6.0 |
| DR690(DR234-DR589) | 1389 | 2.0 | 2.8 |
| DR691(DR234-DR590) | 262 | 0.4 | 0.5 |
| DR692(DR214-DR229) | 586 | 0.8 | 1.2 |
| DR693(DR214-DR230) | 336 | 0.5 | 0.7 |
| DR694(DR214-DR231) | 214 | 0.3 | 0.4 |
| DR695(DR214-DR232) | 467 | 0.7 | 0.9 |
| DR696(DR214-DR233) | 356 | 0.5 | 0.7 |
| DR697(DR214-DR234) | 515 | 0.7 | 1.0 |
| DR698(DR217-DR229) | 242 | 0.3 | 0.5 |
| DR699(DR217-DR230) | 307 | 0.4 | 0.6 |
| DR700(DR217-DR231) | 802 | 1.1 | 1.6 |
| DR701(DR217-DR232) | 10020 | 14.2 | 19.9 |
| DR702(DR217-DR233) | 905 | 1.3 | 1.8 |
| DR703(DR217-DR234) | 419 | 0.6 | 0.8 |
| DR704(DR583-DR229) | 242 | 0.3 | 0.5 |
| DR705(DR583-DR230) | 203 | 0.3 | 0.4 |
| DR706(DR583-DR231) | 211 | 0.3 | 0.4 |
| DR707(DR583-DR232) | 193 | 0.3 | 0.4 |
| DR708(DR583-DR233) | 212 | 0.3 | 0.4 |
| DR709(DR583-DR234) | 203 | 0.3 | 0.4 |
| DR710(DR584-DR229) | 195 | 0.3 | 0.4 |
| DR711(DR584-DR230) | 182 | 0.3 | 0.4 |
| DR712(DR584-DR231) | 206 | 0.3 | 0.4 |
| DR713(DR584-DR232) | 201 | 0.3 | 0.4 |
| DR714(DR584-DR233) | 203 | 0.3 | 0.4 |
| DR715(DR584-DR234) | 192 | 0.3 | 0.4 |
| DR716(DR585-DR229) | 3207 | 4.5 | 6.4 |
| DR717(DR585-DR230) | 2034 | 2.9 | 4.0 |
| DR718(DR585-DR231) | 12281 | 17.4 | 24.4 |
| DR719(DR585-DR232) | 5818 | 8.3 | 11.6 |
| DR720(DR585-DR233) | 2254 | 3.2 | 4.5 |
| DR721(DR585-DR234) | 10913 | 15.5 | 21.7 |
| DR722(DR586-DR229) | 7654 | 10.9 | 15.2 |
| DR723(DR586-DR230) | 293 | 0.4 | 0.6 |
| DR724(DR586-DR231) | 10577 | 15.0 | 21.0 |
| DR725(DR586-DR232) | 442 | 0.6 | 0.9 |
| DR726(DR586-DR233) | 184 | 0.3 | 0.4 |
| DR727(DR586-DR234) | 195 | 0.3 | 0.4 |
| DR728(DR587-DR229) | 205 | 0.3 | 0.4 |
| DR729(DR587-DR230) | 221 | 0.3 | 0.4 |
| DR730(DR587-DR231) | 210 | 0.3 | 0.4 |
| DR731(DR587-DR232) | 217 | 0.3 | 0.4 |
| DR732(DR587-DR233) | 233 | 0.3 | 0.5 |
| DR733(DR587-DR234) | 221 | 0.3 | 0.4 |
| DR734(DR588-DR229) | 594 | 0.8 | 1.2 |
| DR735(DR588-DR230) | 528 | 0.7 | 1.1 |
| DR736(DR588-DR231) | 1909 | 2.7 | 3.8 |
| DR737(DR588-DR232) | 360 | 0.5 | 0.7 |
| DR738(DR588-DR233) | 266 | 0.4 | 0.5 |
| DR739(DR588-DR234) | 580 | 0.8 | 1.2 |
| DR740(DR589-DR229) | 312 | 0.4 | 0.6 |
| DR741(DR589-DR230) | 647 | 0.9 | 1.3 |
| DR742(DR589-DR231) | 330 | 0.5 | 0.7 |
| DR743(DR589-DR232) | 318 | 0.5 | 0.6 |
| DR744(DR589-DR233) | 801 | 1.1 | 1.6 |
| DR745(DR589-DR234) | 206 | 0.3 | 0.4 |
| DR746(DR590-DR229) | 375 | 0.5 | 0.7 |
| DR747(DR590-DR230) | 809 | 1.1 | 1.6 |
| DR748(DR590-DR231) | 474 | 0.7 | 0.9 |
| DR749(DR590-DR232) | 231 | 0.3 | 0.5 |
| DR750(DR590-DR233) | 275 | 0.4 | 0.5 |
| DR751(DR590-DR234) | 189 | 0.3 | 0.4 |
| medium | 705 | 1.0 | 1.4 |
| IL2 | 50261 | 71.3 | 100.0 |

In addition, EC50 values for pSTAT5 phosphorylation were determined for 36 Anti-IL2Rβ/γ V_{H}H²s. Purified NK cells from 4 different donors were resuspended at 2 million cells/mL in PBS. Anti-IL2Rβ/γ V_{H}H²'s or human IL-2 were titrated into 96-well plates (Falcon) starting at 200 nM in 50 μl DPBS and 6 times 10-fold diluted. 100 thousand NK cells were added per well in 50 μl DPBS. Controls included wells without anti-IL2Rβ/γ V_{H}H²'s or human IL-2 (media). Plates were transferred to a humidified incubator (ThermoFisher) and incubated at 37 degrees centigrade, 5 percent carbon dioxide for 20 min. and pSTAT5 phosphorylation measured as above.

To determine the EC50 of each anti-IL2Rβ/γ V_{H}H²'s variant upon STAT5 phosphorylation, MSD luminescence signal values for cells treated with anti-IL2Rβ/γ V_{H}H²'s and human IL-2 were analyzed by 4 parameter Nonlinear Regression on log transformed concentration values using Prism GraphPad software. Emax values were compared to those obtained for control cells treated with growth medium alone to yield a fold induction. The data from these experiments is presented in Table 10 and Table 11.

In this assay anti-IL2Rβ/γ V_{H}H²'s induced STAT5 phosphorylation on human NK cells at distinct EC50 values and Emax values, and relative potency was conserved between donors.

TABLE 10
pSTAT5 Induction by anti-IL2Rβ/γ VHH2 on Primary NK Cells

| anti-IL2Rβ/γ V_{H}H² | EC50 pSTAT5 Induction (pM) | | | |
|---|---|---|---|---|
| Construct | Donor 55 | Donor 56 | Donor 96 | Donor 97 |
| DR634(DR229-DR583) | 490 | 1017 | 13 | 106 |
| DR635(DR229-DR584) | 1164 | 1180 | 295 | 129 |
| DR636(DR229-DR585) | 11876 | 37143 | 4522 | 4555 |
| DR638(DR229-DR587) | 153 | 132 | 16 | 54 |
| DR639(DR229-DR588) | 985 | 1212 | 1463 | 988 |
| DR642(DR230-DR214) | 3191 | 3155 | 1029 | 1038 |
| DR643(DR230-DR217) | 11845 | 11941 | 4648 | 26864 |
| DR644(DR230-DR583) | 665 | 271 | 73 | NA |
| DR645(DR230-DR584) | 1144 | 1108 | 240 | 515 |
| DR648(DR230-DR587) | 982 | 918 | 60 | 111 |
| DR652(DR231-DR214) | 1284 | 1289 | 959 | 1079 |
| DR655(DR231-DR584) | NA | NA | 267 | NA |
| DR656(DR231-DR585) | 707 | 8484 | 2774 | 17693 |
| DR671(DR232-DR590) | NA | NA | 353 | 920 |
| DR674(DR233-DR583) | 1077 | 1027 | 171 | 217 |
| DR675(DR233-DR584) | 4703 | 1308 | 993 | 1571 |
| DR676(DR233-DR584) | NA | NA | 25553 | 256142 |
| DR678(DR233-DR587) | 431 | 279 | 13 | 114 |
| DR679(DR233-DR588) | 5356 | 1148 | 741 | 3234 |
| DR680(DR233-DR589) | 344541 | 12102 | 14276 | 29176 |
| DR681(DR233-DR590) | NA | NA | 527 | NA |
| DR682(DR234-DR214) | 17325 | 2809 | 1488 | 1462 |
| DR688(DR234-DR587) | 351 | 278 | 127 | 113 |
| DR696(DR214-DR233) | NA | NA | 11489 | 112347 |
| DR714(DR584-DR233) | NA | NA | NA | 14998 |
| DR716(DR585-DR229) | 1163 | 1105 | 428 | 252 |
| DR717(DR585-DR230) | 1209 | 1125 | 359 | 314 |
| DR718(DR585-DR231) | 1088 | 1076 | 245 | 256 |
| DR719(DR585-DR232) | 5063 | 2745 | 956 | 1145 |
| DR720(DR585-DR233) | 9199 | 9884 | 14260 | 21597 |
| DR721(DR585-DR234) | 1206 | 1692 | 177 | 188 |
| DR736(DR588-DR231) | 1382 | 1133 | 211 | 256 |
| DR740(DR589-DR229) | NA | NA | NA | NA |
| DR741(DR589-DR230) | NA | NA | NA | 21577 |
| DR744(DR589-DR233) | NA | NA | NA | NA |
| DR747(DR590-DR230) | 7368 | 759 | 177 | 238 |
| IL-2 | 3685 | 9861 | 2480 | 6282 |

NA: Not Available.

TABLE 11
pSTAT5 Induction by anti-IL2Rβ/γ VHH2 on Primary NK Cells

| | Relative pSTAT5 Induction at Emax | | | |
|---|---|---|---|---|
| anti-IL2Rβ/γ V_{H}H² | (fold over medium control) | | | |
| Construct | Donor 55 | Donor 56 | Donor 96 | Donor 97 |
| DR634(DR229-DR583) | 3.1 | 3.1 | 3.8 | 7.9 |
| DR635(DR229-DR584) | 7.0 | 9.1 | 11.2 | 14.3 |
| DR636(DR229-DR585) | 1.9 | 1.4 | 1.6 | 1.7 |
| DR638(DR229-DR587) | 16.4 | 19.7 | 17.4 | 25.3 |
| DR639(DR229-DR588) | 6.9 | 9.0 | 14.9 | 16.4 |
| DR642(DR230-DR214) | 13.8 | 15.2 | 18.6 | 19.5 |
| DR643(DR230-DR217) | 4.8 | 6.1 | 6.6 | 12.8 |
| DR644(DR230-DR583) | 2.2 | 1.5 | 2.0 | 2.9 |
| DR645(DR230-DR584) | 8.2 | 10.1 | 11.9 | 17.2 |
| DR648(DR230-DR587) | 14.6 | 17.6 | 14.5 | 21.8 |
| DR652(DR231-DR214) | 11.5 | 14.0 | 16.6 | 23.7 |
| DR655(DR231-DR584) | 1.4 | 1.1 | 1.3 | 1.2 |
| DR656(DR231-DR585) | 5.9 | 7.0 | 10.7 | 18.2 |
| DR671(DR232-DR590) | 1.4 | 1.6 | 1.6 | 1.3 |
| DR674(DR233-DR583) | 2.6 | 1.9 | 4.8 | 3.9 |
| DR675(DR233-DR584) | 5.3 | 5.0 | 9.5 | 7.6 |
| DR676(DR233-DR584) | 1.9 | 1.7 | 5.1 | 4.7 |
| DR678(DR233-DR587) | 10.1 | 12.9 | 16.1 | 14.0 |
| DR679(DR233-DR588) | 5.7 | 5.3 | 10.4 | 7.9 |
| DR680(DR233-DR589) | 3.1 | 3.2 | 7.2 | 3.9 |
| DR681(DR233-DR590) | 1.0 | 1.0 | 0.8 | 0.6 |
| DR682(DR234-DR214) | 2.1 | 1.4 | 2.1 | 2.1 |
| DR688(DR234-DR587) | 4.7 | 3.1 | 6.6 | 6.8 |
| DR696(DR214-DR233) | 1.7 | 1.3 | 2.5 | 2.0 |
| DR714(DR584-DR233) | 2.0 | 1.6 | 3.8 | 2.7 |
| DR716(DR585-DR229) | 4.5 | 5.0 | 8.9 | 6.2 |
| DR717(DR585-DR230) | 3.1 | 2.7 | 5.9 | 4.0 |
| DR718(DR585-DR231) | 12.7 | 13.4 | 17.8 | 16.1 |
| DR719(DR585-DR232) | 8.7 | 9.3 | 15.1 | 11.4 |
| DR720(DR585-DR233) | 2.1 | 1.8 | 6.3 | 2.6 |
| DR721(DR585-DR234) | 6.9 | 6.1 | 8.8 | 10.6 |
| DR736(DR588-DR231) | 4.8 | 4.7 | 8.8 | 6.5 |
| DR740(DR589-DR229) | 3.0 | 1.0 | 1.5 | 4.7 |
| DR741(DR589-DR230) | 1.2 | 0.9 | 1.5 | 1.3 |
| DR744(DR589-DR233) | 1.6 | 0.8 | 1.5 | 2.1 |
| DR747(DR590-DR230) | 2.7 | 1.9 | 2.9 | 3.3 |
| IL-2 | 22.8 | 31.7 | 31.8 | 38.7 |

NA: Not Available.

### Example 11—Evaluation of Activity of Anti-IL2Rβ/γ V_{H}H2s In Primary NK Cells: Proliferation and IFNγ production

The anti-IL2Rβ/γ V_{H}H²s were evaluated for activity in primary NK cells isolated from human peripheral blood. Primary NK cells express IL2Rβ and IL2Rγ chains and are able to phosphorylate STAT5, proliferate and produce IFN-γ in response to IL2 receptor signaling.

PBMC were isolated from human Buffy Coats or Leucocyte Reduction System Chambers (LRSC) using the Custom Sedimentation Kit (Miltenyi, #130-126-357) and Custom Buffy Coat/LRSC PBMC Isolation kits (Miltenyi, 130-126-448) using protocol Cust5 on an autoMACS Pro Separator (Miltenyi) according to manufacturer's instructions. Purified PBMC were counted on a Vi-cell XR (Beckman Coulter) or Vi-cell Blue (Beckman Coulter) cell viability analyzer. NK cells were isolated from human PBMC using CD56 microbeads (Miltenyi, 130-050-401) on an autoMACS Pro Separator (Miltenyi) with protocol possel according to manufacturer's instructions. Purified NK cells were counted on a Vi-cell XR (Beckman Coulter) or Vi-cell Blue (Beckman Coulter) cell viability analyzer. NK cells were contacted with purified VHH dimers to examine induction of STAT5 phosphorylation as follows: Cells were seeded into 96-well plates (Falcon) at 100 thousand cells per well in 95 μl DPBS prewarmed at 37 degrees centigrade. Five μl of each of the 120 purified VHH dimers in DPBS at 300 nM was added to the cells and plates were transferred to a humidified incubator (ThermoFisher) and incubated at 37 degrees centigrade, 5 percent carbon dioxide for 20 minutes.

NK cells were isolated form peripheral blood of healthy donors collected in Leukoreduction System (LRS) Chambers at the Stanford Blood Bank (Palo Alto). Briefly, PBMC were isolated from LRS Chambers using the human Buffy Coat/LRSC PBMC Isolation Kit (Miltenyi). LRS Chambers were harvested in separation buffer (DPBS, 0.5% BSA, 2 mM EDTA) and mixed with sedimentation buffer and Red Blood Cell (RBC) removal antibodies and EDTA at a final concentration of 5 mM in 50 mL Centrifuge tubes. Tubes were centrifuged at 50×g for 3 minutes at room temperature. Supernatant with cells was collected and transferred to a new 50 mL centrifuge tube and separation buffer was added to 50 mL. Tubes were centrifuged at 300×g for 5 minutes at room temperature and supernatant discarded. Cell pellets were resuspended in 4 mL separation buffer and 2 mL Erythrocyte Depletion Microbeads and 1 mL Granulocyte Depletion Microbeads were added. Cells were incubated for 10 minutes at 2-8 degrees centigrade and PBMC were isolated on an AutoMacs Pro instrument (Miltenyi) using protocol ‘Cust 5’. PBMC were counted on a Vi-Cell XR instrument (Beckman).

NK cells were isolated form PBMC by positive selection using CD56 Microbeads (Miltenyi). Briefly, 1 billion PBMC were incubated with 2 mL CD56 Microbeads and incubated for 15 minutes at 2-8 degrees centigrade. Cells were washed, resuspended in separation buffer and NK cells isolated on an AutoMacs Pro instrument (Miltenyi) using protocol ‘possel’. Purified NK cells were counted on a Vi-cell XR (Beckman Coulter) or Vi-cell Blue (Beckman Coulter) cell viability analyzer.

Prior to the experiment NK cells were centrifuged and washed in DPBS twice. Cells were resuspended at 2 million cells/mL in growth medium consisting of Yssel's medium (Iscove's modified Dulbecco's Medium (ThermoFisher), 0.25% w/v percent human albumin (Sigma), 1 percent penicillin/streptomycin (ThermoFisher), 1 percent ITS-X Insulin, Transferrin, Selenium (Gibco), 30 mg/L Tansferrin (Roche), 2 mg/L Palmitic Acid (Sigma), 1 percent LA-OA-Albumin Linoleic Acid, Oleic Acid (Sigma), 1 percent human serum (Gemini) (Yssel et al (1984) J Immunol Methods 72: 219-227). Anti-IL2Rβ/γ V_{H}H²s were diluted into 96-well plates (Falcon) to 30 nM in 75 μl Yssel's medium and 100 thousand NK cells were added per well in 75 μl DPBS. Controls included wells without anti-IL2Rβ/γ V_{H}H²s or human IL2 (media) and wells with human IL2 at a final concentration of 100 pM. Plates were transferred to a humidified incubator (ThermoFisher) and incubated at 37 degrees centigrade, 5 percent carbon dioxide for 5 days.

In a separate 96 well plate eleven anti-IL2Rβ/γ V_{H}H²s were diluted into 96-well plates (Falcon) to 30 nM in 75 μl Yssel's medium and six 10 fold serial dilutions were made in Yssel's medium. 100 thousand NK cells were added per well in 75 μl DPBS. Controls included wells without anti-IL2Rβ/γ V_{H}H²s or human IL2 (media) and wells with six 10 fold serial dilutions of human IL2 at a starting final concentration of 100 pM. Plates were transferred to a humidified incubator (ThermoFisher) and incubated at 37 degrees centigrade, 5 percent carbon dioxide for 5 days.

Plates were removed from the incubator and 50 μl of culture supernatant was harvested in to a 96 well flat bottom plate (Costar). Cells were lysed by adding 100 μl per well of Celltiterglo (Promega) according to manufacturer's instructions. Cell lysates were mixed on an orbital shaker (VWR Scientific) for two minutes at 300 rpm then held at room temperature for 10 minutes. Luminescence for NK cell lysates was read as counts per second on an Envision 2103 Multilabel Plate Reader (Perkin Elmer).

Production of IFNγ in the culture supernatants was measured using the MSD IFNγ V-Plex kit (MSD K151QOD) according to manufacturer's instructions. Briefly, mAb precoated MSD IFNγ assay plates were washed 3 times with 150 μL Tris Wash Buffer and IFNγ standards were diluted in Diluent 2. Culture supernatants were diluted 1:20 with Diluent 2 and 50 μL of samples and standards were added to the IFNγ assay plates and incubated for 120 min on an orbital shaker (VWR Scientific) at 300 rpm at room temperature. Plates were washed 3 times with Tris Wash Buffer and 25 μL 1×detection antibody in Diluent 3 was added to each well. Plates were incubated for 60 min on an orbital shaker (VWR Scientific) at 300 rpm at room temperature. Plates were washed 3 times with Tris Wash Buffer and 150 μL 2×Read Buffer T was added to each well and Luminescence signal was read on a Mesoscale Quickplex SQ 120 instrument. Concentration of IFNγ in the supernatants were calculated based on the standard curve with MSD Discovery Workbench software.

To compare the effect of each anti-IL2Rβ/γ V_{H}H²s variant upon NK cell proliferation and IFNγ production, Celltiterglo values and IFNγ concentrations for cells treated with anti-IL2Rβ/γ V_{H}H²s were compared to those obtained for control cells treated with growth medium alone to yield a fold induction and with control cells treated with human IL2 to yield an activity relative to the response to IL2 as a percentage. The data from these experiments is presented in Table 12 and Table 13.

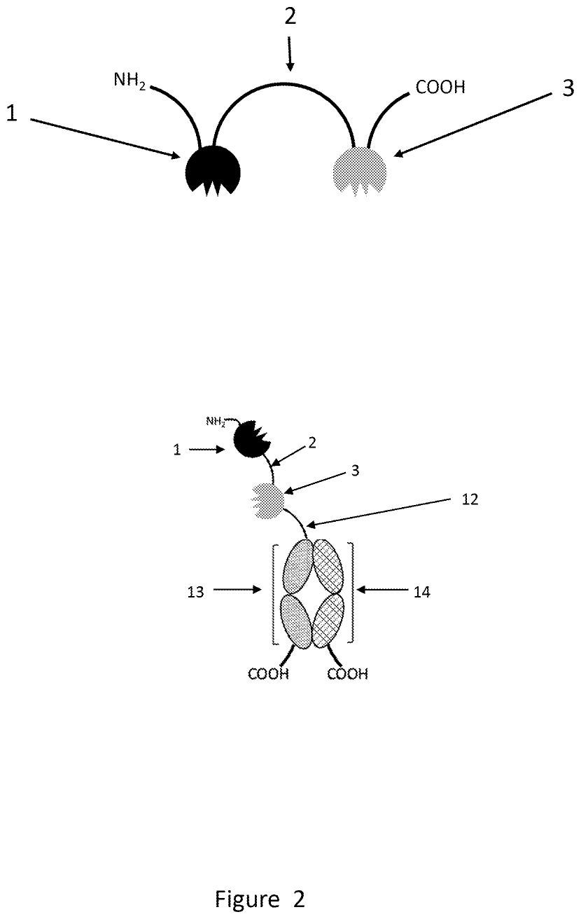

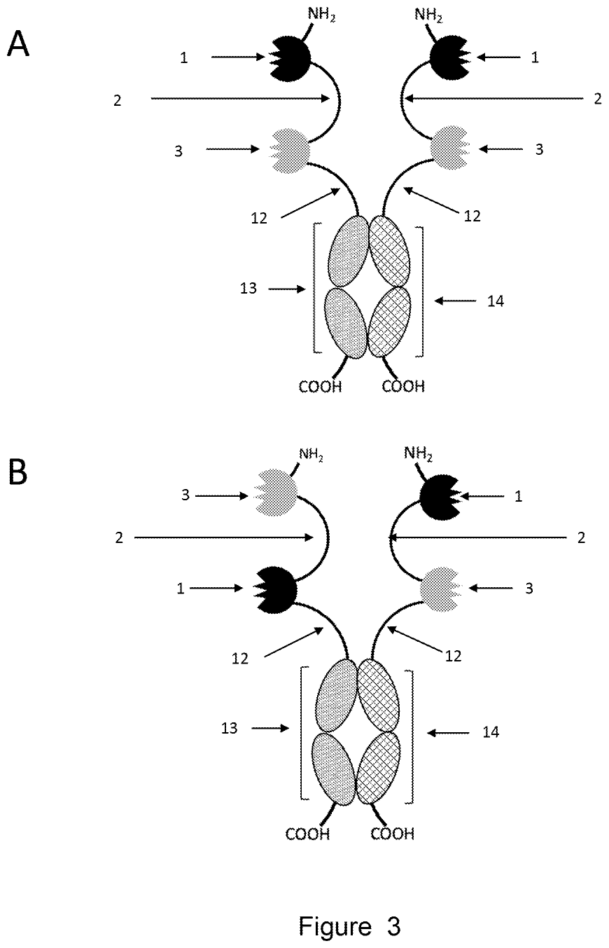

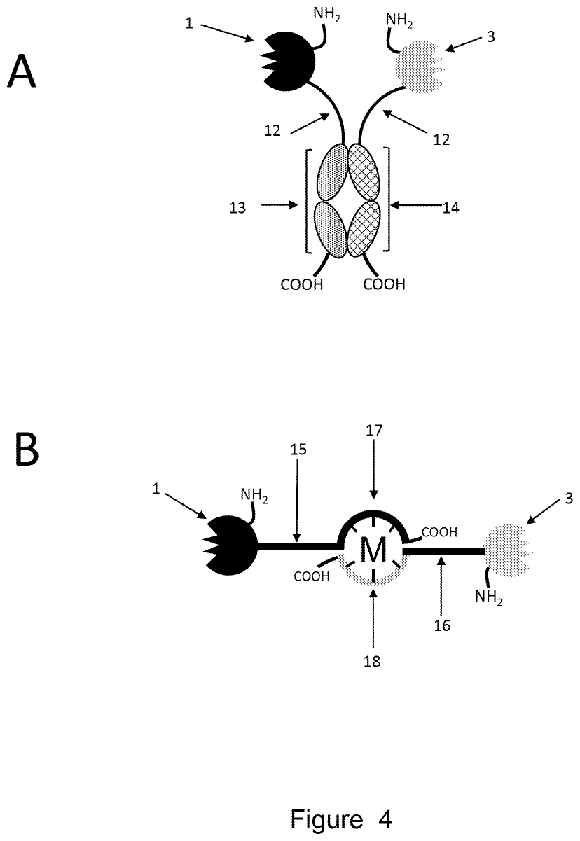

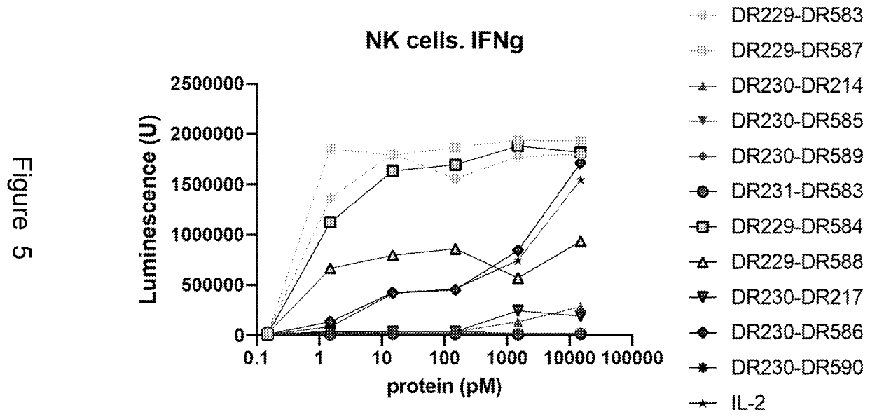

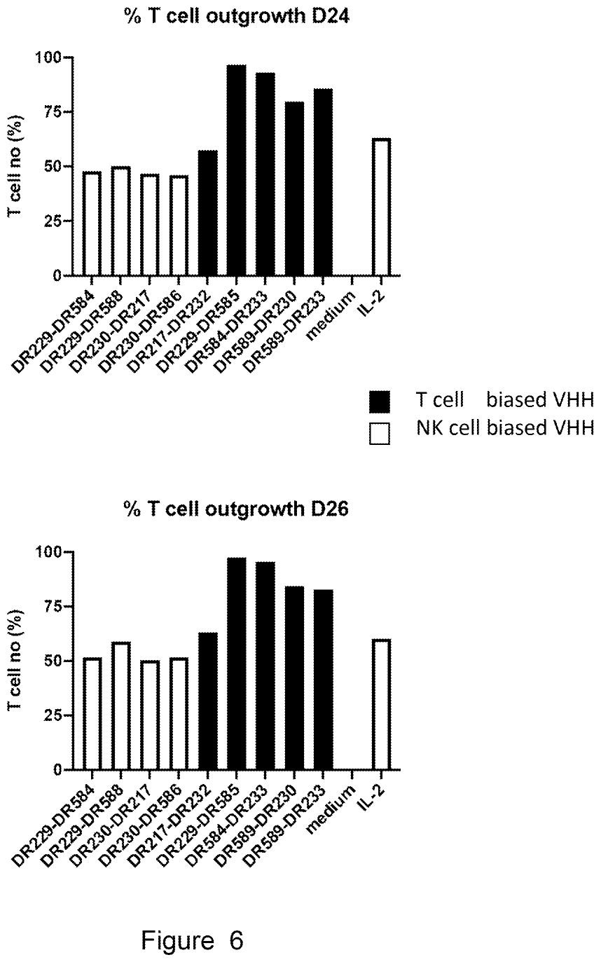

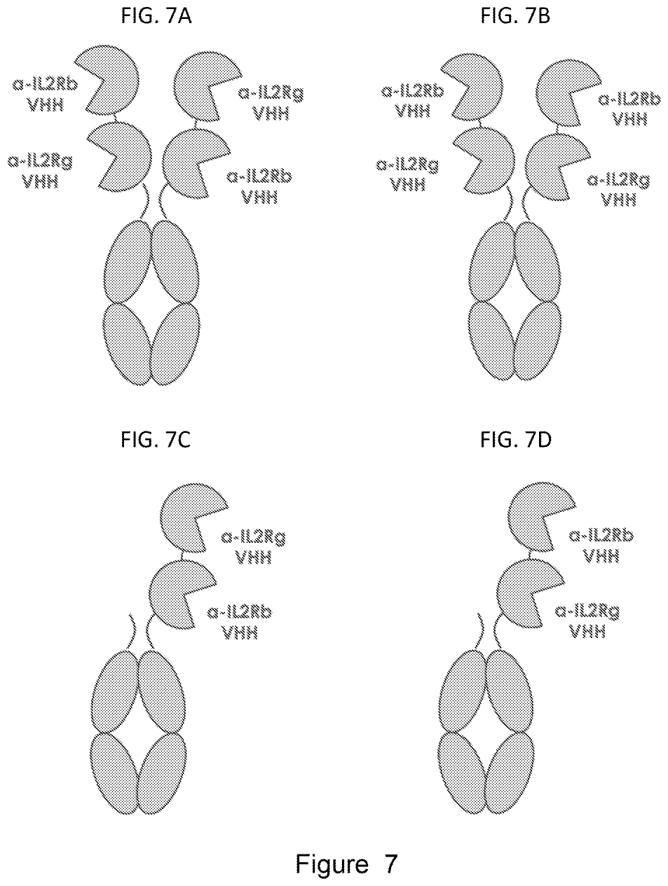

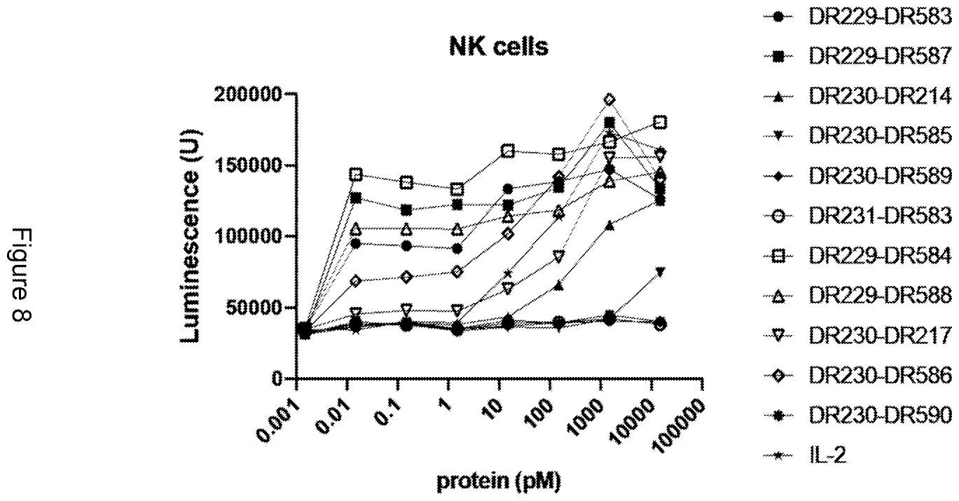

In this assay 111/120 anti-IL2Rβ/γ V_{H}H²s induced NK cell proliferation (>1Fold) at distinct levels, and 22/120 anti-IL2Rβ/γ V_{H}H²s above levels observed with IL2 at 100 pM. In this assay 109/120 anti-IL2Rβ/γ V_{H}H²s induced NK cell IFNγ production (>1Fold) at distinct levels, and 16/120 anti-IL2Rβ/γ V_{H}H²s above levels observed with IL2 at 100 pM. A dose-response titration of 11 anti-IL2Rβ/γ V_{H}H²s showed a concentration dependent induction of proliferation of 4 anti-IL2Rβ/γ V_{H}H²5 (DR230-DR586, DR230-DR217, DR230-DR214, DR230-DR585)) (FIG. 2). Three anti-IL2Rβ/γ V_{H}H²s (DR230-DR589, DR230-DR589, DR231-DR583) failed to induce proliferation of NK cells. Four anti-IL2Rβ/γ V_{H}H²s (DR229-DR584, DR229-DR587, DR229-DR588, DR229-DR583) induced strong proliferation of NK cells that was maintained even at the lowest concentration tested (15 fM).

TABLE 12
Proliferation induced by anti-IL2Rβ/γ V_{H}H² on Primary NK Cells

| | | Relative | Relative |
|---|---|---|---|
| | NK | Proliferation | Proliferation |
| | Proliferation | Induction | Induction |
| Anti-IL2Rβ/γ V_{H}H² | Induction | (fold | (% hIL2 |
| Construct | CTG (cpm) | induction) | response) |
| DR632(DR229-DR214) | 59374 | 1.6 | 44.7 |
| DR633(DR229-DR217) | 84476 | 2.2 | 63.6 |
| DR634(DR229-DR583) | 129450 | 3.4 | 97.5 |
| DR635(DR229-DR584) | 158792 | 4.2 | 119.5 |
| DR636(DR229-DR585) | 66196 | 1.8 | 49.8 |
| DR637(DR229-DR586) | 85292 | 2.3 | 64.2 |
| DR638(DR229-DR587) | 133116 | 3.5 | 100.2 |
| DR639(DR229-DR587) | 140702 | 3.7 | 105.9 |
| DR640(DR229-DR589) | 140980 | 3.7 | 106.1 |
| DR641(DR229-DR590) | 50668 | 1.3 | 38.1 |
| DR642(DR230-DR214) | 132720 | 3.5 | 99.9 |
| DR643(DR230-DR217) | 156030 | 4.1 | 117.5 |
| DR644(DR230-DR583) | 135388 | 3.6 | 101.9 |
| DR645(DR230-DR584) | 150146 | 4.0 | 113.0 |
| DR646(DR230-DR585) | 72526 | 1.9 | 54.6 |
| DR647(DR230-DR586) | 155762 | 4.1 | 117.3 |
| DR648(DR230-DR587) | 144822 | 3.8 | 109.0 |
| DR649(DR230-DR588) | 64048 | 1.7 | 48.2 |
| DR650(DR230-DR589) | 47462 | 1.3 | 35.7 |
| DR651(DR230-DR590) | 49600 | 1.3 | 37.3 |
| DR652(DR231-DR214) | 171010 | 4.5 | 128.7 |
| DR653(DR231-DR217) | 57866 | 1.5 | 43.6 |
| DR654(DR231-DR583) | 49196 | 1.3 | 37.0 |
| DR655(DR231-DR584) | 82024 | 2.2 | 61.8 |
| DR656(DR231-DR585) | 127580 | 3.4 | 96.0 |
| DR657(DR231-DR586) | 57212 | 1.5 | 43.1 |
| DR658(DR231-DR587) | 46432 | 1.2 | 35.0 |
| DR659(DR231-DR588) | 78720 | 2.1 | 59.3 |
| DR660(DR231-DR589) | 50996 | 1.4 | 38.4 |
| DR661(DR231-DR590) | 48910 | 1.3 | 36.8 |
| DR662(DR232-DR214) | 53038 | 1.4 | 39.9 |
| DR663(DR232-DR217) | 118242 | 3.1 | 89.0 |
| DR664(DR232-DR583) | 91608 | 2.4 | 69.0 |
| DR665(DR232-DR584) | 45064 | 1.2 | 33.9 |
| DR666(DR232-DR585) | 49560 | 1.3 | 37.3 |
| DR667(DR232-DR586) | 117570 | 3.1 | 88.5 |
| DR668(DR232-DR587) | 60456 | 1.6 | 45.5 |
| DR669(DR232-DR588) | 59204 | 1.6 | 44.6 |
| DR670(DR232-DR589) | 81900 | 2.2 | 61.7 |
| DR671(DR232-DR590) | 138056 | 3.7 | 103.9 |
| DR672(DR233-DR214) | 120064 | 3.2 | 90.4 |
| DR673(DR233-DR217) | 147252 | 3.9 | 110.9 |
| DR674(DR233-DR583) | 123220 | 3.3 | 92.8 |
| DR675(DR233-DR584) | 146160 | 3.9 | 110.0 |
| DR676(DR233-DR584) | 55708 | 1.5 | 41.9 |
| DR677(DR233-DR586) | 107368 | 2.8 | 80.8 |
| DR678(DR233-DR587) | 160706 | 4.3 | 121.0 |
| DR679(DR233-DR588) | 151432 | 4.0 | 114.0 |
| DR680(DR233-DR589) | 122932 | 3.3 | 92.6 |
| DR681(DR233-DR590) | 83802 | 2.2 | 63.1 |
| DR682(DR234-DR214) | 94996 | 2.5 | 71.5 |
| DR683(DR234-DR217) | 82018 | 2.2 | 61.7 |
| DR684(DR234-DR583) | 46514 | 1.2 | 35.0 |
| DR685(DR234-DR584) | 97470 | 2.6 | 73.4 |
| DR686(DR234-DR585) | 56082 | 1.5 | 42.2 |
| DR687(DR234-DR586) | 63300 | 1.7 | 47.7 |
| DR688(DR234-DR587) | 99810 | 2.6 | 75.1 |
| DR689(DR234-DR588) | 115836 | 3.1 | 87.2 |
| DR690(DR234-DR589) | 108534 | 2.9 | 81.7 |
| DR691(DR234-DR590) | 52358 | 1.4 | 39.4 |
| DR692(DR214-DR229) | 80168 | 2.1 | 60.4 |
| DR693(DR214-DR230) | 44468 | 1.2 | 33.5 |
| DR694(DR214-DR231) | 45548 | 1.2 | 34.3 |
| DR695(DR214-DR232) | 57448 | 1.5 | 43.3 |
| DR696(DR214-DR233) | 62564 | 1.7 | 47.1 |
| DR697(DR214-DR234) | 73146 | 1.9 | 55.1 |
| DR698(DR217-DR229) | 65260 | 1.7 | 49.1 |
| DR699(DR217-DR230) | 68008 | 1.8 | 51.2 |
| DR700(DR217-DR231) | 76818 | 2.0 | 57.8 |
| DR701(DR217-DR232) | 152808 | 4.1 | 115.0 |
| DR702(DR217-DR233) | 77978 | 2.1 | 58.7 |
| DR703(DR217-DR234) | 86778 | 2.3 | 65.3 |
| DR704(DR583-DR229) | 35080 | 0.9 | 26.4 |
| DR705(DR583-DR230) | 46406 | 1.2 | 34.9 |
| DR706(DR583-DR231) | 34682 | 0.9 | 26.1 |
| DR707(DR583-DR232) | 38418 | 1.0 | 28.9 |
| DR708(DR583-DR233) | 40002 | 1.1 | 30.1 |
| DR709(DR583-DR234) | 47088 | 1.2 | 35.5 |
| DR710(DR584-DR229) | 38422 | 1.0 | 28.9 |
| DR711(DR584-DR230) | 36094 | 1.0 | 27.2 |
| DR712(DR584-DR231) | 42562 | 1.1 | 32.0 |
| DR713(DR584-DR232) | 44564 | 1.2 | 33.6 |
| DR714(DR584-DR233) | 31896 | 0.8 | 24.0 |
| DR715(DR584-DR234) | 37140 | 1.0 | 28.0 |
| DR716(DR585-DR229) | 135888 | 3.6 | 102.3 |
| DR717(DR585-DR230) | 125778 | 3.3 | 94.7 |
| DR718(DR585-DR231) | 115728 | 3.1 | 87.1 |
| DR719(DR585-DR232) | 131414 | 3.5 | 98.9 |
| DR720(DR585-DR233) | 136050 | 3.6 | 102.4 |
| DR721(DR585-DR234) | 137718 | 3.7 | 103.7 |
| DR722(DR586-DR229) | 148710 | 3.9 | 112.0 |
| DR723(DR586-DR230) | 74564 | 2.0 | 56.1 |
| DR724(DR586-DR231) | 165338 | 4.4 | 124.5 |
| DR725(DR586-DR232) | 55684 | 1.5 | 41.9 |
| DR726(DR586-DR233) | 36126 | 1.0 | 27.2 |
| DR727(DR586-DR234) | 38650 | 1.0 | 29.1 |
| DR728(DR587-DR229) | 40442 | 1.1 | 30.4 |
| DR729(DR587-DR230) | 42958 | 1.1 | 32.3 |
| DR730(DR587-DR231) | 43068 | 1.1 | 32.4 |
| DR731(DR587-DR232) | 47702 | 1.3 | 35.9 |
| DR732(DR587-DR233) | 42812 | 1.1 | 32.2 |
| DR733(DR587-DR234) | 48304 | 1.3 | 36.4 |
| DR734(DR588-DR229) | 113408 | 3.0 | 85.4 |
| DR735(DR588-DR230) | 119102 | 3.2 | 89.7 |
| DR736(DR588-DR231) | 138570 | 3.7 | 104.3 |
| DR737(DR588-DR232) | 84578 | 2.2 | 63.7 |
| DR738(DR588-DR233) | 48734 | 1.3 | 36.7 |
| DR739(DR588-DR234) | 108850 | 2.9 | 81.9 |
| DR740(DR589-DR229) | 115378 | 3.1 | 86.9 |
| DR741(DR589-DR230) | 113032 | 3.0 | 85.1 |
| DR742(DR589-DR231) | 58200 | 1.5 | 43.8 |
| DR743(DR589-DR232) | 47630 | 1.3 | 35.9 |
| DR744(DR589-DR233) | 96038 | 2.5 | 72.3 |
| DR745(DR589-DR234) | 47518 | 1.3 | 35.8 |
| DR746(DR590-DR229) | 110814 | 2.9 | 83.4 |
| DR747(DR590-DR230) | 124594 | 3.3 | 93.8 |
| DR748(DR590-DR231) | 129464 | 3.4 | 97.5 |
| DR749(DR590-DR232) | 59380 | 1.6 | 44.7 |
| DR750(DR590-DR233) | 62470 | 1.7 | 47.0 |
| DR751(DR590-DR234) | 40034 | 1.1 | 30.1 |
| medium | 37693 | 1.0 | 28.4 |
| IL2 | 132827 | 3.5 | 100.0 |

TABLE 13
IFNγ production by anti-IL2Rβ/γ V_{H}H² on Primary NK Cells

| | | Relative | |
|---|---|---|---|
| | | IFNγ | Relative IFNγ |
| | NK Cell IFNγ | Induction | Induction |
| Anti-IL2Rβ/γ V_{H}H² | Production | (fold | (% hIL2 |
| Construct | (MSD Signal) | induction) | response) |
| DR632(DR229-DR214) | 144618 | 8.7 | 8.1 |
| DR633(DR229-DR217) | 80516 | 4.8 | 4.5 |
| DR634(DR229-DR583) | 1883810 | 113.0 | 105.0 |
| DR635(DR229-DR584) | 1911538 | 114.7 | 106.6 |
| DR636(DR229-DR585) | 36726 | 2.2 | 2.0 |
| DR637(DR229-DR586) | 142020 | 8.5 | 7.9 |
| DR638(DR229-DR587) | 1907171 | 114.4 | 106.3 |
| DR639(DR229-DR587) | 1128817 | 67.7 | 62.9 |
| DR640(DR229-DR589) | 1137944 | 68.3 | 63.4 |
| DR641(DR229-DR590) | 36793 | 2.2 | 2.1 |
| DR642(DR230-DR214) | 1849412 | 110.9 | 103.1 |
| DR643(DR230-DR217) | 1850211 | 111.0 | 103.2 |
| DR644(DR230-DR583) | 795501 | 47.7 | 44.4 |
| DR645(DR230-DR584) | 1960761 | 117.6 | 109.3 |
| DR646(DR230-DR585) | 129630 | 7.8 | 7.2 |
| DR647(DR230-DR586) | 1921088 | 115.2 | 107.1 |
| DR648(DR230-DR587) | 1845712 | 110.7 | 102.9 |
| DR649(DR230-DR588) | 61590 | 3.7 | 3.4 |
| DR650(DR230-DR589) | 23788 | 1.4 | 1.3 |
| DR651(DR230-DR590) | 13376 | 0.8 | 0.7 |
| DR652(DR231-DR214) | 1901886 | 114.1 | 106.0 |
| DR653(DR231-DR217) | 73604 | 4.4 | 4.1 |
| DR654(DR231-DR583) | 38744 | 2.3 | 2.2 |
| DR655(DR231-DR584) | 641257 | 38.5 | 35.8 |
| DR656(DR231-DR585) | 1901425 | 114.0 | 106.0 |
| DR657(DR231-DR586) | 133136 | 8.0 | 7.4 |
| DR658(DR231-DR587) | 83387 | 5.0 | 4.6 |
| DR659(DR231-DR588) | 218983 | 13.1 | 12.2 |
| DR660(DR231-DR589) | 31698 | 1.9 | 1.8 |
| DR661(DR231-DR590) | 12924 | 0.8 | 0.7 |
| DR662(DR232-DR214) | 127725 | 7.7 | 7.1 |
| DR663(DR232-DR217) | 787105 | 47.2 | 43.9 |
| DR664(DR232-DR583) | 431059 | 25.9 | 24.0 |
| DR665(DR232-DR584) | 17316 | 1.0 | 1.0 |
| DR666(DR232-DR585) | 55300 | 3.3 | 3.1 |
| DR667(DR232-DR586) | 658694 | 39.5 | 36.7 |
| DR668(DR232-DR587) | 57260 | 3.4 | 3.2 |
| DR669(DR232-DR588) | 23328 | 1.4 | 1.3 |
| DR670(DR232-DR589) | 215999 | 13.0 | 12.0 |
| DR671(DR232-DR590) | 1279164 | 76.7 | 71.3 |
| DR672(DR233-DR214) | 1690293 | 101.4 | 94.2 |
| DR673(DR233-DR217) | 1195243 | 71.7 | 66.6 |
| DR674(DR233-DR583) | 1520704 | 91.2 | 84.8 |
| DR675(DR233-DR584) | 1922798 | 115.3 | 107.2 |
| DR676(DR233-DR584) | 44387 | 2.7 | 2.5 |
| DR677(DR233-DR586) | 451720 | 27.1 | 25.2 |
| DR678(DR233-DR587) | 1877120 | 112.6 | 104.7 |
| DR679(DR233-DR588) | 1840663 | 110.4 | 102.6 |
| DR680(DR233-DR589) | 1574319 | 94.4 | 87.8 |
| DR681(DR233-DR590) | 79218 | 4.8 | 4.4 |
| DR682(DR234-DR214) | 266392 | 16.0 | 14.9 |
| DR683(DR234-DR217) | 66068 | 4.0 | 3.7 |
| DR684(DR234-DR583) | 9713 | 0.6 | 0.5 |
| DR685(DR234-DR584) | 451812 | 27.1 | 25.2 |
| DR686(DR234-DR585) | 79541 | 4.8 | 4.4 |
| DR687(DR234-DR586) | 122522 | 7.3 | 6.8 |
| DR688(DR234-DR587) | 1299512 | 77.9 | 72.5 |
| DR689(DR234-DR588) | 1546871 | 92.8 | 86.2 |
| DR690(DR234-DR589) | 851698 | 51.1 | 47.5 |
| DR691(DR234-DR590) | 33530 | 2.0 | 1.9 |
| DR692(DR214-DR229) | 341600 | 20.5 | 19.0 |
| DR693(DR214-DR230) | 32214 | 1.9 | 1.8 |
| DR694(DR214-DR231) | 13781 | 0.8 | 0.8 |
| DR695(DR214-DR232) | 50327 | 3.0 | 2.8 |
| DR696(DR214-DR233) | 20592 | 1.2 | 1.1 |
| DR697(DR214-DR234) | 128229 | 7.7 | 7.1 |
| DR698(DR217-DR229) | 373385 | 22.4 | 20.8 |
| DR699(DR217-DR230) | 108166 | 6.5 | 6.0 |
| DR700(DR217-DR231) | 68805 | 4.1 | 3.8 |
| DR701(DR217-DR232) | 1716480 | 103.0 | 95.7 |
| DR702(DR217-DR233) | 114876 | 6.9 | 6.4 |
| DR703(DR217-DR234) | 390416 | 23.4 | 21.8 |
| DR704(DR583-DR229) | 18481 | 1.1 | 1.0 |
| DR705(DR583-DR230) | 26339 | 1.6 | 1.5 |
| DR706(DR583-DR231) | 13676 | 0.8 | 0.8 |
| DR707(DR583-DR232) | 20801 | 1.2 | 1.2 |
| DR708(DR583-DR233) | 28936 | 1.7 | 1.6 |
| DR709(DR583-DR234) | 25068 | 1.5 | 1.4 |
| DR710(DR584-DR229) | 71711 | 4.3 | 4.0 |
| DR711(DR584-DR230) | 16215 | 1.0 | 0.9 |
| DR712(DR584-DR231) | 17139 | 1.0 | 1.0 |
| DR713(DR584-DR232) | 10805 | 0.6 | 0.6 |
| DR714(DR584-DR233) | 17361 | 1.0 | 1.0 |
| DR715(DR584-DR234) | 30424 | 1.8 | 1.7 |
| DR716(DR585-DR229) | 1415371 | 84.9 | 78.9 |
| DR717(DR585-DR230) | 1860610 | 111.6 | 103.7 |
| DR718(DR585-DR231) | 1665366 | 99.9 | 92.9 |
| DR719(DR585-DR232) | 1334193 | 80.0 | 74.4 |
| DR720(DR585-DR233) | 995884 | 59.7 | 55.5 |
| DR721(DR585-DR234) | 1937087 | 116.2 | 108.0 |
| DR722(DR586-DR229) | 1704185 | 102.2 | 95.0 |
| DR723(DR586-DR230) | 119540 | 7.2 | 6.7 |
| DR724(DR586-DR231) | 1928068 | 115.6 | 107.5 |
| DR725(DR586-DR232) | 38001 | 2.3 | 2.1 |
| DR726(DR586-DR233) | 54648 | 3.3 | 3.0 |
| DR727(DR586-DR234) | 15553 | 0.9 | 0.9 |
| DR728(DR587-DR229) | 60701 | 3.6 | 3.4 |
| DR729(DR587-DR230) | 54437 | 3.3 | 3.0 |
| DR730(DR587-DR231) | 30615 | 1.8 | 1.7 |
| DR731(DR587-DR232) | 49737 | 3.0 | 2.8 |
| DR732(DR587-DR233) | 596023 | 35.7 | 33.2 |
| DR733(DR587-DR234) | 562814 | 33.8 | 31.4 |
| DR734(DR588-DR229) | 641028 | 38.4 | 35.7 |
| DR735(DR588-DR230) | 757479 | 45.4 | 42.2 |
| DR736(DR588-DR231) | 1455626 | 87.3 | 81.2 |
| DR737(DR588-DR232) | 308300 | 18.5 | 17.2 |
| DR738(DR588-DR233) | 29645 | 1.8 | 1.7 |
| DR739(DR588-DR234) | 279562 | 16.8 | 15.6 |
| DR740(DR589-DR229) | 517465 | 31.0 | 28.9 |
| DR741(DR589-DR230) | 181177 | 10.9 | 10.1 |
| DR742(DR589-DR231) | 102522 | 6.1 | 5.7 |
| DR743(DR589-DR232) | 12119 | 0.7 | 0.7 |
| DR744(DR589-DR233) | 709867 | 42.6 | 39.6 |
| DR745(DR589-DR234) | 552283 | 33.1 | 30.8 |
| DR746(DR590-DR229) | 638377 | 38.3 | 35.6 |
| DR747(DR590-DR230) | 713175 | 42.8 | 39.8 |
| DR748(DR590-DR231) | 407832 | 24.5 | 22.7 |
| DR749(DR590-DR232) | 100488 | 6.0 | 5.6 |
| DR750(DR590-DR233) | 32777 | 2.0 | 1.8 |
| DR751(DR590-DR234) | 30496 | 1.8 | 1.7 |
| medium | 16672 | 1.0 | 0.9 |
| IL2 | 1793477 | 107.6 | 100.0 |

In addition, EC50 values for proliferation and IFNγ production were determined for 36 Anti-IL2Rβ/γ V_{H}H²'s. Purified NK cells from 4 different donors were resuspended at 2 million cells/mL in PBS. Anti-IL2Rβ/γ V_{H}H²'s or human IL-2 were titrated into 96-well plates (Falcon) starting at 200 nM in 75 μl DPBS and 6 times 10 fold diluted. 100 thousand NK cells were added per well in 75 μl DPBS. Controls included wells without anti-IL2Rβ/γ V_{H}H²'s or human IL-2 (media). Plates were transferred to a humidified incubator (ThermoFisher) and incubated at 37 degrees centigrade, 5 percent carbon dioxide for 5 days and proliferation and IFNγ production measured as above.

To determine the EC50 of each anti-IL2Rβ/γ V_{H}H²'s variant upon proliferation and IFNγ production, IFNγ MSD and Celltiterglo luminescence signal values for cells treated with anti-IL2Rβ/γ V_{H}H²'s and human IL-2 were analyzed by 4 parameter Nonlinear Regression on log transformed concentration values using Prism GraphPad software. Emax values were compared to those obtained for control cells treated with growth medium alone to yield a fold induction. The data from these experiments is presented in Table 14 and Table 15.

In this assay anti-IL2Rβ/γ V_{H}H²'s induced STAT5 proliferation and IFNγ production by human NK cells at distinct EC50 values and Emax values, and relative potency was conserved between donors.

TABLE 14
Proliferation and IFN γ production induced by anti-
IL2Rβ/γ VHH2 on Primary NK Cells

| | EC50 Proliferation | | EC50 IFNg Production | |
|---|---|---|---|---|
| anti-IL2Rβ/γ V_{H}H² | (pM) | | (pM) | |
| Construct | D56 | D97 | D56 | D97 |
| DR634(DR229-DR583) | 26 | 122 | 191 | 901 |
| DR635(DR229-DR584) | 52 | 148 | 1351 | 4558 |
| DR636(DR229-DR585) | 12164 | 25956 | 82659 | 522998 |
| DR638(DR229-DR587) | 10 | 19 | 223 | 1119 |
| DR639(DR229-DR588) | 62 | 185 | 20132 | 2235 |
| DR642(DR230-DR214) | 197 | 448 | 13151 | 11841 |
| DR643(DR230-DR217) | 28386 | 5100 | 156294 | 165620 |
| DR644(DR230-DR583) | 46 | 136 | 347 | 1009 |
| DR645(DR230-DR584) | 52 | 111 | 902 | 1734 |
| DR648(DR230-DR587) | 15 | 29 | 917 | 1442 |
| DR652(DR231-DR214) | 360 | 545 | 12857 | 34076 |
| DR655(DR231-DR584) | 234 | 1264 | 1034 | 2338 |
| DR656(DR231-DR585) | 16699 | 9265 | 301488 | 41214 |
| DR671(DR232-DR590) | 2667 | 13293 | 7982 | 36578 |
| DR674(DR233-DR583) | 257 | 186 | 5674 | 1986 |
| DR675(DR233-DR584) | 2661 | 844 | 22139 | 23915 |
| DR676(DR233-DR584) | 22073 | 11619 | 1286547 | 80838 |
| DR678(DR233-DR587) | 250 | 119 | 8234 | 7726 |
| DR679(DR233-DR588) | 5961 | 1254 | 40260 | 43815 |
| DR680(DR233-DR589) | 63518 | 17040 | 23035459 | 31157 |
| DR681(DR233-DR590) | NA | NA | 86809404 | 1.637 |
| DR682(DR234-DR214) | 3884 | 6421 | 12202 | 26022 |
| DR688(DR234-DR587) | 85 | 169 | 278 | 1842 |
| DR696(DR214-DR233) | 89543 | NA | 124132 | 21966 |
| DR714(DR584-DR233) | 70327 | 77963 | 311382317 | 660555722 |
| DR716(DR585-DR229) | 658 | 207 | 7300 | 3723 |
| DR717(DR585-DR230) | 621 | 550 | 3674 | 2560 |
| DR718(DR585-DR231) | 1712 | 461 | 16435 | 15917 |
| DR719(DR585-DR232) | 16556 | 2436 | 186469 | 40742 |
| DR720(DR585-DR233) | 24996 | 11540 | 177733 | 33994 |
| DR721(DR585-DR234) | 229 | 267 | 3129 | 8619 |
| DR736(DR588-DR231) | 886 | 712 | 12901 | 36398 |
| DR740(DR589-DR229) | 82708 | 23452 | 329845362 | 335393846 |
| DR741(DR589-DR230) | 40149 | 616219 | 131864 | 629271484 |
| DR744(DR589-DR233) | 175279 | 144690027 | 226140856 | 545550061 |
| DR747(DR590-DR230) | 304 | 316 | 1169 | 2414 |
| IL-2 | 350 | 275 | 122 | 9285 |

NA: Not Available. D56: Donor 56. D97: Donor 97.

TABLE 15
Proliferation and IFNγ production induced by anti-
IL2Rβ/γ VHH2 on Primary NK Cells

| | | | Emax IFNg | |
|---|---|---|---|---|
| | Emax Proliferation | | Production | |
| | (Fold over | | (Fold over | |
| anti-IL2Rβ/γ V_{H}H² | media control) | | media control) | |
| Construct | D56 | D97 | D56 | D97 |
| DR634(DR229-DR583) | 11.0 | 3.7 | 28 | 36 |
| DR635(DR229-DR584) | 9.7 | 3.2 | 73 | 106 |
| DR636(DR229-DR585) | 8.1 | 3.8 | 6 | 4 |
| DR638(DR229-DR587) | 8.5 | 3.5 | 105 | 142 |
| DR639(DR229-DR588) | 10.2 | 3.8 | 51 | 42 |
| DR642(DR230-DR214) | 10.5 | 3.2 | 71 | 70 |
| DR643(DR230-DR217) | 15.9 | 3.3 | 17 | 10 |
| DR644(DR230-DR583) | 8.5 | 4.2 | 9 | 7 |
| DR645(DR230-DR584) | 11.1 | 4.3 | 66 | 82 |
| DR648(DR230-DR587) | 10.9 | 4.1 | 98 | 142 |
| DR652(DR231-DR214) | 10.8 | 4.2 | 97 | 162 |
| DR655(DR231-DR584) | 4.9 | 2.6 | 3 | 2 |
| DR656(DR231-DR585) | 14.3 | 4.0 | 26 | 15 |
| DR671(DR232-DR590) | 4.5 | 2.3 | 2 | 3 |
| DR674(DR233-DR583) | 11.7 | 3.8 | 27 | 25 |
| DR675(DR233-DR584) | 15.4 | 5.1 | 67 | 81 |
| DR676(DR233-DR584) | 7.6 | 3.6 | 4 | 4 |
| DR678(DR233-DR587) | 10.6 | 4.5 | 73 | 126 |
| DR679(DR233-DR588) | 12.4 | 4.2 | 16 | 21 |
| DR680(DR233-DR589) | 10.2 | 3.4 | 3 | 3 |
| DR681(DR233-DR590) | 1.6 | 1.6 | 1 | 1 |
| DR682(DR234-DR214) | 9.6 | 3.9 | 7 | 5 |
| DR688(DR234-DR587) | 9.9 | 4.3 | 14 | 14 |
| DR696(DR214-DR233) | 5.2 | 2.3 | 1 | 1 |
| DR714(DR584-DR233) | 9.5 | 3.3 | 5 | 3 |
| DR716(DR585-DR229) | 12.2 | 5.1 | 28 | 29 |
| DR717(DR585-DR230) | 12.3 | 4.8 | 24 | 22 |
| DR718(DR585-DR231) | 14.9 | 5.4 | 66 | 88 |
| DR719(DR585-DR232) | 19.7 | 5.3 | 30 | 21 |
| DR720(DR585-DR233) | 9.7 | 3.4 | 5 | 4 |
| DR721(DR585-DR234) | 12.0 | 4.2 | 87 | 82 |
| DR736(DR588-DR231) | 12.5 | 4.5 | 35 | 31 |
| DR740(DR589-DR229) | 5.0 | 2.0 | 2 | 1 |
| DR741(DR589-DR230) | 7.0 | 2.9 | 3 | 2 |
| DR744(DR589-DR233) | 5.5 | 2.1 | 2 | 1 |
| DR747(DR590-DR230) | 10.3 | 4.6 | 16 | 13 |
| IL-2 | 9.9 | 4.1 | 128 | 175 |

NA: Not Available. D56: Donor 56. D97: Donor 97.

### Example 12—Evaluation of Activity of anti-IL2Rβ/γ V_{H}H²s In Primary Activated CD8 Positive T Cell Blasts: STAT5

The anti-IL2Rβ/γ V_{H}H²s were evaluated for activity in primary CD3/CD28 activated CD8 positive T cell blasts isolated and generated from human peripheral blood. Activated CD8 positive T Cell blasts express IL2Rβ and IL2Rγ chains and are able to phosphorylate STAT5, proliferate and produce IFN-γ in response to IL2 receptor signaling.

CD8 T cells were isolated form peripheral blood of healthy donors collected in Leukoreduction System (LRS) Chambers at the Stanford Blood Bank (Palo Alto). Briefly, PBMC were isolated from LRS Chambers using the human Buffy Coat/LRSC PBMC Isolation Kit (Miltenyi). LRS Chambers were harvested in separation buffer (DPBS, 0.5% BSA, 2 mM EDTA) and mixed with sedimentation buffer and Red Blood Cell (RBC) removal antibodies and EDTA at a final concentration of 5 mM in 50 mL Centrifuge tubes. Tubes were centrifuged at 50×g for 3 minutes at room temperature. Supernatant with cells was collected and transferred to a new 50 mL centrifuge tube and separation buffer was added to 50 mL. Tubes were centrifuged at 300×g for 5 minutes at room temperature and supernatant discarded. Cell pellets were resuspended in 4 mL separation buffer and 2 mL Erythrocyte Depletion Microbeads and 1 mL Granulocyte Depletion Microbeads were added. Cells were incubated for 10 minutes at 2-8 degrees centigrade and PBMC were isolated on an AutoMacs Pro instrument (Miltenyi) using protocol ‘Cult 5’. PBMC were counted on a Vi-Cell XR instrument (Beckman).

PBMC were cultured in growth medium consisting of Yssel's medium (Iscove's modified Dulbecco's Medium (ThermoFisher), 0.25% w/v percent human albumin (Sigma), 1 percent penicillin/streptomycin (ThermoFisher), 1 percent ITS-X Insulin, Transferrin, Selenium (Gibco), 30 mg/L Tansferrin (Roche), 2 mg/L Palmitic Acid (Sigma), 1 percent LA-OA-Albumin Linoleic Acid, Oleic Acid (Sigma), 1 percent human serum (Gemini) (Yssel et al (1984) J Immunol Methods 72: 219-227) at 1 million cells per ml with 1 μg/mL anti-CD3 mAb (OKT3 Biolegend) and 1 μg/mL anti-CD28 mAb (28.1 Biolegend) for 72 hrs.

Activated CD8 positive T cell blasts were isolated form PBMC by positive selection using CD8 Microbeads (Miltenyi). Briefly, 0.5 billion PBMC were incubated with 1 mL CD8 Microbeads and incubated for 15 minutes at 2-8 degrees centigrade. Cells were washed, resuspended in separation buffer and activated CD8 positive T cell blasts isolated on an AutoMacs Pro instrument (Miltenyi) using protocol ‘possel’. Activated CD8 positive T cell blasts were counted on a Vi-Cell XR instrument (Beckman).

Prior to the experiment activated CD8 positive T cell blasts were centrifuged and washed in DPBS twice. Cells were resuspended at 2 million cells/mL in PBS. Anti-IL2Rβ/γ V_{H}H²s were diluted into 96-well plates (Falcon) to 30 nM in 50 μl DPBS and 100 thousand activated CD8 positive T cell blasts were added per well in 50 μl DPBS. Controls included wells without anti-IL2Rβ/γ V_{H}H²s or human IL2 (media) and wells with human IL2 at a final concentration of 100 pM. Plates were transferred to a humidified incubator (ThermoFisher) and incubated at 37 degrees centigrade, 5 percent carbon dioxide for 20 min.

Plates were removed from the incubator and cells were lysed by adding 100 μl per well of Tris Lysis buffer supplemented with Protease Inhibitor Solution, Phosphatase Inhibitor I and Phosphatase Inhibitor II from MSD Multispot Assay System Phospho-STAT Panel Kit (MSD K15202D) according to manufacturer's instructions. Plates were incubated on ice for 15 minutes and centrifuged at 600×g for 6 minutes. Cell lysates were transferred to a new 96 well plate.

Induction of STAT5 phosphorylation was measured in the cell lysates using the MSD Multispot Assay System Phospho-STAT Panel (MSD K15202D) according to manufacturer's instructions. Briefly, mAb precoated MSD Phospho-STAT panel assay plates were incubated with 150 μL per well Blocker A in Tris Wash Buffer at 30 mg/mL for 60 min on an orbital shaker (VWR Scientific) at 300 rpm at room temperature. Plates were washed 3 times with Tris Wash Buffer and 30 μL Cell lysates were added per well. Plates were incubated for 60 min on an orbital shaker (VWR Scientific) at 300 rpm at room temperature. Plates were washed 3 times with Tris Wash Buffer and 25 μL 1×detection antibody in 10 mg/mL Blocker A in Tris Wash Buffer was added to each well. Plates were incubated for 60 min on an orbital shaker (VWR Scientific) at 300 rpm at room temperature. Plates were washed 3 times with Tris Wash Buffer and 150 μL 1× Read Buffer T was added to each well and Luminescence signal was read on a Mesoscale Quickplex SQ 120 instrument

To compare the effect of each anti-IL2Rβ/γ V_{H}H²s variant upon activated CD8 positive T cell blasts STAT5 phosphorylation, MSD luminescence signal values for cells treated with anti-IL2Rβ/γ V_{H}H²s were compared to those obtained for control cells treated with growth medium alone to yield a fold induction and with control cells treated with human IL2 to yield an activity relative to the response to IL2 as a percentage. The data from these experiments is presented in Table 18.

In this assay 48/120 anti-IL2Rβ/γ V_{H}H²s induced STAT5 phosphorylation in activated CD8 T cell blasts at distinct levels (Fold>1), but not above levels observed with IL2 at 100 pM.

TABLE 18
pSTAT5 Induction by anti-IL2Rβ/γ
V_{H}H² on activated CD8 T cell blasts

| | | Relative | Relative |
|---|---|---|---|
| | CD8 blasts | pSTAT5 | pSTAT5 |
| | pSTAT5 | Induction | Induction |
| Anti-IL2Rβ/γ V_{H}H² | Induction | (fold | (% hIL2 |
| Construct | (MSD Signal) | induction) | response) |
| DR632(DR229-DR214) | 1975 | 0.4 | 0.4 |
| DR633(DR229-DR217) | 2802 | 0.5 | 0.5 |
| DR634(DR229-DR583) | 7119 | 1.4 | 1.4 |
| DR635(DR229-DR584) | 69738 | 13.3 | 13.3 |
| DR636(DR229-DR585) | 4305 | 0.8 | 0.8 |
| DR637(DR229-DR586) | 3152 | 0.6 | 0.6 |
| DR638(DR229-DR587) | 67754 | 12.9 | 12.9 |
| DR639(DR229-DR587) | 29763 | 5.7 | 5.7 |
| DR640(DR229-DR589) | 15645 | 3.0 | 3.0 |
| DR641(DR229-DR590) | 2566 | 0.5 | 0.5 |
| DR642(DR230-DR214) | 37103 | 7.0 | 7.0 |
| DR643(DR230-DR217) | 83861 | 15.9 | 15.9 |
| DR644(DR230-DR583) | 5757 | 1.1 | 1.1 |
| DR645(DR230-DR584) | 46494 | 8.8 | 8.8 |
| DR646(DR230-DR585) | 11691 | 2.2 | 2.2 |
| DR647(DR230-DR586) | 88561 | 16.8 | 16.8 |
| DR648(DR230-DR587) | 55433 | 10.5 | 10.5 |
| DR649(DR230-DR588) | 2353 | 0.4 | 0.4 |
| DR650(DR230-DR589) | 4356 | 0.8 | 0.8 |
| DR651(DR230-DR590) | 4380 | 0.8 | 0.8 |
| DR652(DR231-DR214) | 62461 | 11.9 | 11.9 |
| DR653(DR231-DR217) | 2241 | 0.4 | 0.4 |
| DR654(DR231-DR583) | 3512 | 0.7 | 0.7 |
| DR655(DR231-DR584) | 10601 | 2.0 | 2.0 |
| DR656(DR231-DR585) | 31725 | 6.0 | 6.0 |
| DR657(DR231-DR586) | 5973 | 1.1 | 1.1 |
| DR658(DR231-DR587) | 2572 | 0.5 | 0.5 |
| DR659(DR231-DR588) | 2262 | 0.4 | 0.4 |
| DR660(DR231-DR589) | 4159 | 0.8 | 0.8 |
| DR661(DR231-DR590) | 4637 | 0.9 | 0.9 |
| DR662(DR232-DR214) | 3144 | 0.6 | 0.6 |
| DR663(DR232-DR217) | 3041 | 0.6 | 0.6 |
| DR664(DR232-DR583) | 10939 | 2.1 | 2.1 |
| DR665(DR232-DR584) | 4927 | 0.9 | 0.9 |
| DR666(DR232-DR585) | 2936 | 0.6 | 0.6 |
| DR667(DR232-DR586) | 3221 | 0.6 | 0.6 |
| DR668(DR232-DR587) | 4857 | 0.9 | 0.9 |
| DR669(DR232-DR588) | 7147 | 1.4 | 1.4 |
| DR670(DR232-DR589) | 3756 | 0.7 | 0.7 |
| DR671(DR232-DR590) | 6860 | 1.3 | 1.3 |
| DR672(DR233-DR214) | 19482 | 3.7 | 3.7 |
| DR673(DR233-DR217) | 15296 | 2.9 | 2.9 |
| DR674(DR233-DR583) | 29097 | 5.5 | 5.5 |
| DR675(DR233-DR584) | 16694 | 3.2 | 3.2 |
| DR676(DR233-DR584) | 6983 | 1.3 | 1.3 |
| DR677(DR233-DR586) | 5021 | 1.0 | 1.0 |
| DR678(DR233-DR587) | 71073 | 13.5 | 13.5 |
| DR679(DR233-DR588) | 9245 | 1.8 | 1.8 |
| DR680(DR233-DR589) | 35467 | 6.7 | 6.7 |
| DR681(DR233-DR590) | 14232 | 2.7 | 2.7 |
| DR682(DR234-DR214) | 6798 | 1.3 | 1.3 |
| DR683(DR234-DR217) | 6461 | 1.2 | 1.2 |
| DR684(DR234-DR583) | 3136 | 0.6 | 0.6 |
| DR685(DR234-DR584) | 11098 | 2.1 | 2.1 |
| DR686(DR234-DR585) | 5015 | 1.0 | 1.0 |
| DR687(DR234-DR586) | 5052 | 1.0 | 1.0 |
| DR688(DR234-DR587) | 20746 | 3.9 | 3.9 |
| DR689(DR234-DR588) | 29909 | 5.7 | 5.7 |
| DR690(DR234-DR589) | 8260 | 1.6 | 1.6 |
| DR691(DR234-DR590) | 3327 | 0.6 | 0.6 |
| DR692(DR214-DR229) | 7796 | 1.5 | 1.5 |
| DR693(DR214-DR230) | 5864 | 1.1 | 1.1 |
| DR694(DR214-DR231) | 4098 | 0.8 | 0.8 |
| DR695(DR214-DR232) | 4808 | 0.9 | 0.9 |
| DR696(DR214-DR233) | 4670 | 0.9 | 0.9 |
| DR697(DR214-DR234) | 4911 | 0.9 | 0.9 |
| DR698(DR217-DR229) | 4409 | 0.8 | 0.8 |
| DR699(DR217-DR230) | 3101 | 0.6 | 0.6 |
| DR700(DR217-DR231) | 6717 | 1.3 | 1.3 |
| DR701(DR217-DR232) | 40130 | 7.6 | 7.6 |
| DR702(DR217-DR233) | 6388 | 1.2 | 1.2 |
| DR703(DR217-DR234) | 2112 | 0.4 | 0.4 |
| DR704(DR583-DR229) | 3828 | 0.7 | 0.7 |
| DR705(DR583-DR230) | 3977 | 0.8 | 0.8 |
| DR706(DR583-DR231) | 2996 | 0.6 | 0.6 |
| DR707(DR583-DR232) | 4123 | 0.8 | 0.8 |
| DR708(DR583-DR233) | 4414 | 0.8 | 0.8 |
| DR709(DR583-DR234) | 3552 | 0.7 | 0.7 |
| DR710(DR584-DR229) | 4380 | 0.8 | 0.8 |
| DR711(DR584-DR230) | 3083 | 0.6 | 0.6 |
| DR712(DR584-DR231) | 4739 | 0.9 | 0.9 |
| DR713(DR584-DR232) | 4168 | 0.8 | 0.8 |
| DR714(DR584-DR233) | 3885 | 0.7 | 0.7 |
| DR715(DR584-DR234) | 3414 | 0.6 | 0.6 |
| DR716(DR585-DR229) | 47906 | 9.1 | 9.1 |
| DR717(DR585-DR230) | 19829 | 3.8 | 3.8 |
| DR718(DR585-DR231) | 99121 | 18.8 | 18.8 |
| DR719(DR585-DR232) | 41612 | 7.9 | 7.9 |
| DR720(DR585-DR233) | 29633 | 5.6 | 5.6 |
| DR721(DR585-DR234) | 48028 | 9.1 | 9.1 |
| DR722(DR586-DR229) | 68270 | 13.0 | 13.0 |
| DR723(DR586-DR230) | 2669 | 0.5 | 0.5 |
| DR724(DR586-DR231) | 78186 | 14.9 | 14.9 |
| DR725(DR586-DR232) | 2018 | 0.4 | 0.4 |
| DR726(DR586-DR233) | 3643 | 0.7 | 0.7 |
| DR727(DR586-DR234) | 555 | 0.1 | 0.1 |
| DR728(DR587-DR229) | 3292 | 0.6 | 0.6 |
| DR729(DR587-DR230) | 3154 | 0.6 | 0.6 |
| DR730(DR587-DR231) | 2908 | 0.6 | 0.6 |
| DR731(DR587-DR232) | 1983 | 0.4 | 0.4 |
| DR732(DR587-DR233) | 2854 | 0.5 | 0.5 |
| DR733(DR587-DR234) | 2417 | 0.5 | 0.5 |
| DR734(DR588-DR229) | 4609 | 0.9 | 0.9 |
| DR735(DR588-DR230) | 2445 | 0.5 | 0.5 |
| DR736(DR588-DR231) | 9338 | 1.8 | 1.8 |
| DR737(DR588-DR232) | 2946 | 0.6 | 0.6 |
| DR738(DR588-DR233) | 2876 | 0.5 | 0.5 |
| DR739(DR588-DR234) | 2011 | 0.4 | 0.4 |
| DR740(DR589-DR229) | 3880 | 0.7 | 0.7 |
| DR741(DR589-DR230) | 3493 | 0.7 | 0.7 |
| DR742(DR589-DR231) | 2713 | 0.5 | 0.5 |
| DR743(DR589-DR232) | 1579 | 0.3 | 0.3 |
| DR744(DR589-DR233) | 3969 | 0.8 | 0.8 |
| DR745(DR589-DR234) | 1741 | 0.3 | 0.3 |
| DR746(DR590-DR229) | 2869 | 0.5 | 0.5 |
| DR747(DR590-DR230) | 2890 | 0.5 | 0.5 |
| DR748(DR590-DR231) | 4161 | 0.8 | 0.8 |
| DR749(DR590-DR232) | 1252 | 0.2 | 0.2 |
| DR750(DR590-DR233) | 2182 | 0.4 | 0.4 |
| DR751(DR590-DR234) | 955 | 0.2 | 0.2 |
| medium | 5263 | 1.0 | 4.0 |
| IL2 | 130278 | 24.8 | 100.0 |

### Example 13—Evaluation of Activity of anti-IL2Rβ/γ V_{H}H²s In Primary Activated CD8 Positive T Cell Blasts: Proliferation and IFNγ Production

The anti-IL2Rβ/γ V_{H}H²s were evaluated for activity in primary CD3/CD28 activated CD8 positive T cell blasts isolated and generated from human peripheral blood. Activated CD8 positive T Cell blasts express IL2Rβ and IL2Rγ chains and are able to phosphorylate STAT5, proliferate and produce IFN-γ in response to IL2 receptor signaling.

CD8 T cells were isolated form peripheral blood of healthy donors collected in Leukoreduction System (LRS) Chambers at the Stanford Blood Bank (Palo Alto). Briefly, PBMC were isolated from LRS Chambers using the human Buffy Coat/LRSC PBMC Isolation Kit (Miltenyi). LRS Chambers were harvested in separation buffer (DPBS, 0.5% BSA, 2 mM EDTA) and mixed with sedimentation buffer and Red Blood Cell (RBC) removal antibodies and EDTA at a final concentration of 5 mM in 50 mL Centrifuge tubes. Tubes were centrifuged at 50×g for 3 minutes at room temperature. Supernatant with cells was collected and transferred to a new 50 mL centrifuge tube and separation buffer was added to 50 mL. Tubes were centrifuged at 300×g for 5 minutes at room temperature and supernatant discarded. Cell pellets were resuspended in 4 mL separation buffer and 2 mL Erythrocyte Depletion Microbeads and 1 mL Granulocyte Depletion Microbeads were added. Cells were incubated for 10 minutes at 2-8 degrees centigrade and PBMC were isolated on an AutoMacs Pro instrument (Miltenyi) using protocol ‘Cult 5’. PBMC were counted on a Vi-Cell XR instrument (Beckman).

PBMC were cultured in growth medium consisting of Yssel's medium (Iscove's modified Dulbecco's Medium (ThermoFisher), 0.25% w/v percent human albumin (Sigma), 1 percent penicillin/streptomycin (ThermoFisher), 1 percent ITS-X Insulin, Transferrin, Selenium (Gibco), 30 mg/L Tansferrin (Roche), 2 mg/L Palmitic Acid (Sigma), 1 percent LA-OA-Albumin Linoleic Acid, Oleic Acid (Sigma), 1 percent human serum (Gemini) (Yssel et al (1984) J Immunol Methods 72: 219-227) at 1 million cells per ml with 1 μg/mL anti-CD3 mAb (OKT3 Biolegend) and 1 μg/mL anti-CD28 mAb (28.1 Biolegend) for 72 hrs.

Activated CD8 positive T cell blasts were isolated form PBMC by positive selection using CD8 Microbeads (Miltenyi). Briefly, 0.5 billion PBMC were incubated with 1 mL CD8 Microbeads and incubated for 15 minutes at 2-8 degrees centigrade. Cells were washed, resuspended in separation buffer and activated CD8 positive T cell blasts isolated on an AutoMacs Pro instrument (Miltenyi) using protocol ‘possel’. Activated CD8 positive T cell blasts were counted on a Vi-Cell XR instrument (Beckman).

Prior to the experiment activated CD8 positive T cell blasts were centrifuged and washed in DPBS twice. Cells were resuspended at 1 million cells/mL in growth medium consisting of Yssel's medium (Iscove's modified Dulbecco's Medium (ThermoFisher), 0.25% w/v percent human albumin (Sigma), 1 percent penicillin/streptomycin (ThermoFisher), 1 percent ITS-X Insulin, Transferrin, Selenium (Gibco), 30 mg/L Tansferrin (Roche), 2 mg/L Palmitic Acid (Sigma), 1 percent LA-OA-Albumin Linoleic Acid, Oleic Acid (Sigma), 1 percent human serum (Gemini) (Yssel et al (1984) J Immunol Methods 72: 219-227). Anti-IL2Rβ/γ V_{H}H²s were diluted into 96-well plates (Falcon) to 30 nM in 75 μl Yssel's medium and 50 thousand activated CD8 positive T cell blasts were added per well in 75 μl DPBS. Controls included wells without anti-IL2Rβ/γ V_{H}H²s or human IL2 (media) and wells with human IL2 at a final concentration of 100 pM. Plates were transferred to a humidified incubator (ThermoFisher) and incubated at 37 degrees centigrade, 5 percent carbon dioxide for 5 days.

Plates were removed from the incubator and 50 μl of culture supernatant was harvested in to a 96 well flat bottom plate (Costar). Cells were lysed by adding 100 μl per well of Celltiterglo (Promega) according to manufacturer's instructions. Cell lysates were mixed on an orbital shaker (VWR Scientific) for two minutes at 300 rpm then held at room temperature for 10 minutes. Luminescence for activated CD8 positive T cell blasts lysates was read as counts per second on an Envision 2103 Multilabel Plate Reader (Perkin Elmer).

Production of IFNγ in the culture supernatants was measured using the MSD IFNγ V-Plex kit (MSD K151QOD) according to manufacturer's instructions. Briefly, mAb precoated MSD IFNγ assay plates were washed 3 times with 150 μL Tris Wash Buffer and IFNγ standards were diluted in Diluent 2. Culture supernatants were diluted 1:10 with Diluent 2 and 50 μL of samples and standards were added to the IFNγ assay plates and incubated for 120 min on an orbital shaker (VWR Scientific) at 300 rpm at room temperature. Plates were washed 3 times with Tris Wash Buffer and 25 μL 1×detection antibody in Diluent 3 was added to each well. Plates were incubated for 60 min on an orbital shaker (VWR Scientific) at 300 rpm at room temperature. Plates were washed 3 times with Tris Wash Buffer and 150 μL 2× Read Buffer T was added to each well and Luminescence signal was read on a Mesoscale Quickplex SQ 120 instrument. Concentration of IFNγ in the supernatants were calculated based on the standard curve with MSD Discovery Workbench software.

To compare the effect of each anti-IL2Rβ/γ V_{H}H²s variant upon activated CD8 positive T cell blasts proliferation and IFNγ production, Celltiterglo values and IFNγ concentrations for cells treated with anti-IL2Rβ/γ V_{H}H²s were compared to those obtained for control cells treated with growth medium alone to yield a fold induction and with control cells treated with human IL2 to yield an activity relative to the response to IL2 as a percentage. The data from these experiments is presented in Table 19 and Table 20.

In this assay 104/120 anti-IL2Rβ/γ V_{H}H²s induced activated CD8 positive T cell blasts proliferation (>1Fold) at distinct levels, and 28/120 anti-IL2Rβ/γ V_{H}H²s above levels observed with IL2 at 100 pM. In this assay 76/120 anti-IL2Rβ/γ V_{H}H²s induced activated CD8 positive T cell blasts IFNγ production (>1Fold) at distinct levels, but not above levels observed with IL2 at 100 pM.

TABLE 19
Proliferation Induced by anti-IL2Rβ/γ
V_{H}H² on activated CD8 T cell blasts

| | | Relative | Relative |
|---|---|---|---|
| | CD8 blasts | Proliferation | Proliferation |
| | Proliferation | Induction | Induction |
| Anti-IL2Rβ/γ V_{H}H² | Induction | (fold | (% hIL2 |
| Construct | CTG (cpm) | induction) | response) |
| DR632(DR229-DR214) | 74694 | 1.6 | 52.0 |
| DR633(DR229-DR217) | 97528 | 2.1 | 67.9 |
| DR634(DR229-DR583) | 169082 | 3.6 | 117.8 |
| DR635(DR229-DR584) | 199810 | 4.3 | 139.2 |
| DR636(DR229-DR585) | 96650 | 2.1 | 67.3 |
| DR637(DR229-DR586) | 96020 | 2.1 | 66.9 |
| DR638(DR229-DR587) | 196596 | 4.2 | 136.9 |
| DR639(DR229-DR587) | 186708 | 4.0 | 130.0 |
| DR640(DR229-DR589) | 166848 | 3.6 | 116.2 |
| DR641(DR229-DR590) | 59040 | 1.3 | 41.1 |
| DR642(DR230-DR214) | 169256 | 3.6 | 117.9 |
| DR643(DR230-DR217) | 199372 | 4.3 | 138.8 |
| DR644(DR230-DR583) | 150086 | 3.2 | 104.5 |
| DR645(DR230-DR584) | 234368 | 5.0 | 163.2 |
| DR646(DR230-DR585) | 100966 | 2.2 | 70.3 |
| DR647(DR230-DR586) | 207848 | 4.5 | 144.7 |
| DR648(DR230-DR587) | 193526 | 4.2 | 134.8 |
| DR649(DR230-DR588) | 89156 | 1.9 | 62.1 |
| DR650(DR230-DR589) | 59650 | 1.3 | 41.5 |
| DR651(DR230-DR590) | 60920 | 1.3 | 42.4 |
| DR652(DR231-DR214) | 214638 | 4.6 | 149.5 |
| DR653(DR231-DR217) | 68142 | 1.5 | 47.5 |
| DR654(DR231-DR583) | 64188 | 1.4 | 44.7 |
| DR655(DR231-DR584) | 96790 | 2.1 | 67.4 |
| DR656(DR231-DR585) | 149116 | 3.2 | 103.8 |
| DR657(DR231-DR586) | 68418 | 1.5 | 47.6 |
| DR658(DR231-DR587) | 50934 | 1.1 | 35.5 |
| DR659(DR231-DR588) | 78852 | 1.7 | 54.9 |
| DR660(DR231-DR589) | 70632 | 1.5 | 49.2 |
| DR661(DR231-DR590) | 61506 | 1.3 | 42.8 |
| DR662(DR232-DR214) | 58280 | 1.3 | 40.6 |
| DR663(DR232-DR217) | 127604 | 2.7 | 88.9 |
| DR664(DR232-DR583) | 99388 | 2.1 | 69.2 |
| DR665(DR232-DR584) | 60210 | 1.3 | 41.9 |
| DR666(DR232-DR585) | 57460 | 1.2 | 40.0 |
| DR667(DR232-DR586) | 123146 | 2.7 | 85.8 |
| DR668(DR232-DR587) | 77298 | 1.7 | 53.8 |
| DR669(DR232-DR588) | 78226 | 1.7 | 54.5 |
| DR670(DR232-DR589) | 90720 | 2.0 | 63.2 |
| DR671(DR232-DR590) | 142454 | 3.1 | 99.2 |
| DR672(DR233-DR214) | 170126 | 3.7 | 118.5 |
| DR673(DR233-DR217) | 188860 | 4.1 | 131.5 |
| DR674(DR233-DR583) | 175798 | 3.8 | 122.4 |
| DR675(DR233-DR584) | 182374 | 3.9 | 127.0 |
| DR676(DR233-DR584) | 102448 | 2.2 | 71.3 |
| DR677(DR233-DR586) | 112872 | 2.4 | 78.6 |
| DR678(DR233-DR587) | 196456 | 4.2 | 136.8 |
| DR679(DR233-DR588) | 172512 | 3.7 | 120.1 |
| DR680(DR233-DR589) | 140116 | 3.0 | 97.6 |
| DR681(DR233-DR590) | 110932 | 2.4 | 77.3 |
| DR682(DR234-DR214) | 113696 | 2.4 | 79.2 |
| DR683(DR234-DR217) | 87860 | 1.9 | 61.2 |
| DR684(DR234-DR583) | 48908 | 1.1 | 34.1 |
| DR685(DR234-DR584) | 112190 | 2.4 | 78.1 |
| DR686(DR234-DR585) | 68192 | 1.5 | 47.5 |
| DR687(DR234-DR586) | 76202 | 1.6 | 53.1 |
| DR688(DR234-DR587) | 113120 | 2.4 | 78.8 |
| DR689(DR234-DR588) | 153806 | 3.3 | 107.1 |
| DR690(DR234-DR589) | 106106 | 2.3 | 73.9 |
| DR691(DR234-DR590) | 63728 | 1.4 | 44.4 |
| DR692(DR214-DR229) | 81058 | 1.7 | 56.4 |
| DR693(DR214-DR230) | 74190 | 1.6 | 51.7 |
| DR694(DR214-DR231) | 53506 | 1.2 | 37.3 |
| DR695(DR214-DR232) | 73792 | 1.6 | 51.4 |
| DR696(DR214-DR233) | 75228 | 1.6 | 52.4 |
| DR697(DR214-DR234) | 76072 | 1.6 | 53.0 |
| DR698(DR217-DR229) | 59082 | 1.3 | 41.1 |
| DR699(DR217-DR230) | 67000 | 1.4 | 46.7 |
| DR700(DR217-DR231) | 72854 | 1.6 | 50.7 |
| DR701(DR217-DR232) | 151010 | 3.3 | 105.2 |
| DR702(DR217-DR233) | 83172 | 1.8 | 57.9 |
| DR703(DR217-DR234) | 73744 | 1.6 | 51.4 |
| DR704(DR583-DR229) | 47968 | 1.0 | 33.4 |
| DR705(DR583-DR230) | 53554 | 1.2 | 37.3 |
| DR706(DR583-DR231) | 43828 | 0.9 | 30.5 |
| DR707(DR583-DR232) | 51940 | 1.1 | 36.2 |
| DR708(DR583-DR233) | 50450 | 1.1 | 35.1 |
| DR709(DR583-DR234) | 52870 | 1.1 | 36.8 |
| DR710(DR584-DR229) | 41228 | 0.9 | 28.7 |
| DR711(DR584-DR230) | 45988 | 1.0 | 32.0 |
| DR712(DR584-DR231) | 47854 | 1.0 | 33.3 |
| DR713(DR584-DR232) | 50962 | 1.1 | 35.5 |
| DR714(DR584-DR233) | 44376 | 1.0 | 30.9 |
| DR715(DR584-DR234) | 49586 | 1.1 | 34.5 |
| DR716(DR585-DR229) | 161498 | 3.5 | 112.5 |
| DR717(DR585-DR230) | 163070 | 3.5 | 113.6 |
| DR718(DR585-DR231) | 176680 | 3.8 | 123.0 |
| DR719(DR585-DR232) | 145996 | 3.1 | 101.7 |
| DR720(DR585-DR233) | 144204 | 3.1 | 100.4 |
| DR721(DR585-DR234) | 167206 | 3.6 | 116.4 |
| DR722(DR586-DR229) | 171340 | 3.7 | 119.3 |
| DR723(DR586-DR230) | 75522 | 1.6 | 52.6 |
| DR724(DR586-DR231) | 211052 | 4.5 | 147.0 |
| DR725(DR586-DR232) | 61316 | 1.3 | 42.7 |
| DR726(DR586-DR233) | 49710 | 1.1 | 34.6 |
| DR727(DR586-DR234) | 40706 | 0.9 | 28.3 |
| DR728(DR587-DR229) | 34304 | 0.7 | 23.9 |
| DR729(DR587-DR230) | 43390 | 0.9 | 30.2 |
| DR730(DR587-DR231) | 44146 | 1.0 | 30.7 |
| DR731(DR587-DR232) | 46354 | 1.0 | 32.3 |
| DR732(DR587-DR233) | 51346 | 1.1 | 35.8 |
| DR733(DR587-DR234) | 53770 | 1.2 | 37.4 |
| DR734(DR588-DR229) | 91694 | 2.0 | 63.9 |
| DR735(DR588-DR230) | 81412 | 1.8 | 56.7 |
| DR736(DR588-DR231) | 109598 | 2.4 | 76.3 |
| DR737(DR588-DR232) | 70134 | 1.5 | 48.8 |
| DR738(DR588-DR233) | 55678 | 1.2 | 38.8 |
| DR739(DR588-DR234) | 94516 | 2.0 | 65.8 |
| DR740(DR589-DR229) | 81458 | 1.8 | 56.7 |
| DR741(DR589-DR230) | 88854 | 1.9 | 61.9 |
| DR742(DR589-DR231) | 56044 | 1.2 | 39.0 |
| DR743(DR589-DR232) | 48310 | 1.0 | 33.6 |
| DR744(DR589-DR233) | 75144 | 1.6 | 52.3 |
| DR745(DR589-DR234) | 46104 | 1.0 | 32.1 |
| DR746(DR590-DR229) | 91978 | 2.0 | 64.1 |
| DR747(DR590-DR230) | 128022 | 2.8 | 89.2 |
| DR748(DR590-DR231) | 94978 | 2.0 | 66.1 |
| DR749(DR590-DR232) | 47614 | 1.0 | 33.2 |
| DR750(DR590-DR233) | 65174 | 1.4 | 45.4 |
| DR751(DR590-DR234) | 41294 | 0.9 | 28.8 |
| medium | 46428 | 1.0 | 32.3 |
| IL2 | 143593 | 3.1 | 100.0 |

TABLE 20
IFNγ production by anti-IL2Rβ/γ
V_{H}H² on activated CD8 T cell blasts

| | CD8 | Relative | Relative |
|---|---|---|---|
| | Cell | IFNγ | IFNγ |
| | IFNγ | Induction | Induction |
| Anti-IL2Rβ/γ V_{H}H² | Production | (fold | (% hIL2 |
| Construct | (pg/mL) | induction) | response) |
| DR632(DR229-DR214) | 357 | 1.5 | 4.0 |
| DR633(DR229-DR217) | 400 | 1.7 | 4.5 |
| DR634(DR229-DR583) | 3050 | 13.1 | 34.1 |
| DR635(DR229-DR584) | 6050 | 26.1 | 67.6 |
| DR636(DR229-DR585) | 364 | 1.6 | 4.1 |
| DR637(DR229-DR586) | 407 | 1.8 | 4.5 |
| DR638(DR229-DR587) | 4429 | 19.1 | 49.4 |
| DR639(DR229-DR587) | 2653 | 11.4 | 29.6 |
| DR640(DR229-DR589) | 1413 | 6.1 | 15.8 |
| DR641(DR229-DR590) | 176 | 0.8 | 2.0 |
| DR642(DR230-DR214) | 2125 | 9.2 | 23.7 |
| DR643(DR230-DR217) | 5021 | 21.6 | 56.1 |
| DR644(DR230-DR583) | 1077 | 4.6 | 12.0 |
| DR645(DR230-DR584) | 5761 | 24.8 | 64.3 |
| DR646(DR230-DR585) | 305 | 1.3 | 3.4 |
| DR647(DR230-DR586) | 4615 | 19.9 | 51.5 |
| DR648(DR230-DR587) | 3168 | 13.7 | 35.4 |
| DR649(DR230-DR588) | 388 | 1.7 | 4.3 |
| DR650(DR230-DR589) | 146 | 0.6 | 1.6 |
| DR651(DR230-DR590) | 157 | 0.7 | 1.7 |
| DR652(DR231-DR214) | 5290 | 22.8 | 59.1 |
| DR653(DR231-DR217) | 356 | 1.5 | 4.0 |
| DR654(DR231-DR583) | 131 | 0.6 | 1.5 |
| DR655(DR231-DR584) | 480 | 2.1 | 5.4 |
| DR656(DR231-DR585) | 2033 | 8.8 | 22.7 |
| DR657(DR231-DR586) | 193 | 0.8 | 2.2 |
| DR658(DR231-DR587) | 232 | 1.0 | 2.6 |
| DR659(DR231-DR588) | 281 | 1.2 | 3.1 |
| DR660(DR231-DR589) | 202 | 0.9 | 2.3 |
| DR661(DR231-DR590) | 186 | 0.8 | 2.1 |
| DR662(DR232-DR214) | 243 | 1.0 | 2.7 |
| DR663(DR232-DR217) | 929 | 4.0 | 10.4 |
| DR664(DR232-DR583) | 378 | 1.6 | 4.2 |
| DR665(DR232-DR584) | 137 | 0.6 | 1.5 |
| DR666(DR232-DR585) | 161 | 0.7 | 1.8 |
| DR667(DR232-DR586) | 979 | 4.2 | 10.9 |
| DR668(DR232-DR587) | 196 | 0.8 | 2.2 |
| DR669(DR232-DR588) | 194 | 0.8 | 2.2 |
| DR670(DR232-DR589) | 349 | 1.5 | 3.9 |
| DR671(DR232-DR590) | 1306 | 5.6 | 14.6 |
| DR672(DR233-DR214) | 1188 | 5.1 | 13.3 |
| DR673(DR233-DR217) | 2153 | 9.3 | 24.0 |
| DR674(DR233-DR583) | 2296 | 9.9 | 25.6 |
| DR675(DR233-DR584) | 4191 | 18.1 | 46.8 |
| DR676(DR233-DR584) | 241 | 1.0 | 2.7 |
| DR677(DR233-DR586) | 686 | 3.0 | 7.7 |
| DR678(DR233-DR587) | 5977 | 25.8 | 66.7 |
| DR679(DR233-DR588) | 4357 | 18.8 | 48.6 |
| DR680(DR233-DR589) | 3008 | 13.0 | 33.6 |
| DR681(DR233-DR590) | 411 | 1.8 | 4.6 |
| DR682(DR234-DR214) | 840 | 3.6 | 9.4 |
| DR683(DR234-DR217) | 332 | 1.4 | 3.7 |
| DR684(DR234-DR583) | 166 | 0.7 | 1.9 |
| DR685(DR234-DR584) | 672 | 2.9 | 7.5 |
| DR686(DR234-DR585) | 277 | 1.2 | 3.1 |
| DR687(DR234-DR586) | 230 | 1.0 | 2.6 |
| DR688(DR234-DR587) | 1705 | 7.3 | 19.0 |
| DR689(DR234-DR588) | 933 | 4.0 | 10.4 |
| DR690(DR234-DR589) | 565 | 2.4 | 6.3 |
| DR691(DR234-DR590) | 159 | 0.7 | 1.8 |
| DR692(DR214-DR229) | 283 | 1.2 | 3.2 |
| DR693(DR214-DR230) | 202 | 0.9 | 2.3 |
| DR694(DR214-DR231) | 133 | 0.6 | 1.5 |
| DR695(DR214-DR232) | 184 | 0.8 | 2.1 |
| DR696(DR214-DR233) | 310 | 1.3 | 3.5 |
| DR697(DR214-DR234) | 217 | 0.9 | 2.4 |
| DR698(DR217-DR229) | 172 | 0.7 | 1.9 |
| DR699(DR217-DR230) | 232 | 1.0 | 2.6 |
| DR700(DR217-DR231) | 210 | 0.9 | 2.4 |
| DR701(DR217-DR232) | 2879 | 12.4 | 32.1 |
| DR702(DR217-DR233) | 208 | 0.9 | 2.3 |
| DR703(DR217-DR234) | 291 | 1.3 | 3.2 |
| DR704(DR583-DR229) | 192 | 0.8 | 2.1 |
| DR705(DR583-DR230) | 199 | 0.9 | 2.2 |
| DR706(DR583-DR231) | 265 | 1.1 | 3.0 |
| DR707(DR583-DR232) | 255 | 1.1 | 2.8 |
| DR708(DR583-DR233) | 166 | 0.7 | 1.9 |
| DR709(DR583-DR234) | 168 | 0.7 | 1.9 |
| DR710(DR584-DR229) | 231 | 1.0 | 2.6 |
| DR711(DR584-DR230) | 231 | 1.0 | 2.6 |
| DR712(DR584-DR231) | 160 | 0.7 | 1.8 |
| DR713(DR584-DR232) | 164 | 0.7 | 1.8 |
| DR714(DR584-DR233) | 154 | 0.7 | 1.7 |
| DR715(DR584-DR234) | 228 | 1.0 | 2.5 |
| DR716(DR585-DR229) | 2271 | 9.8 | 25.4 |
| DR717(DR585-DR230) | 2292 | 9.9 | 25.6 |
| DR718(DR585-DR231) | 7211 | 31.1 | 80.5 |
| DR719(DR585-DR232) | 1674 | 7.2 | 18.7 |
| DR720(DR585-DR233) | 1251 | 5.4 | 14.0 |
| DR721(DR585-DR234) | 4122 | 17.8 | 46.0 |
| DR722(DR586-DR229) | 4087 | 17.6 | 45.6 |
| DR723(DR586-DR230) | 468 | 2.0 | 5.2 |
| DR724(DR586-DR231) | 4012 | 17.3 | 44.8 |
| DR725(DR586-DR232) | 210 | 0.9 | 2.4 |
| DR726(DR586-DR233) | 227 | 1.0 | 2.5 |
| DR727(DR586-DR234) | 211 | 0.9 | 2.4 |
| DR728(DR587-DR229) | 251 | 1.1 | 2.8 |
| DR729(DR587-DR230) | 226 | 1.0 | 2.5 |
| DR730(DR587-DR231) | 203 | 0.9 | 2.3 |
| DR731(DR587-DR232) | 206 | 0.9 | 2.3 |
| DR732(DR587-DR233) | 390 | 1.7 | 4.3 |
| DR733(DR587-DR234) | 294 | 1.3 | 3.3 |
| DR734(DR588-DR229) | 676 | 2.9 | 7.5 |
| DR735(DR588-DR230) | 856 | 3.7 | 9.6 |
| DR736(DR588-DR231) | 1810 | 7.8 | 20.2 |
| DR737(DR588-DR232) | 545 | 2.3 | 6.1 |
| DR738(DR588-DR233) | 218 | 0.9 | 2.4 |
| DR739(DR588-DR234) | 710 | 3.1 | 7.9 |
| DR740(DR589-DR229) | 613 | 2.6 | 6.8 |
| DR741(DR589-DR230) | 593 | 2.6 | 6.6 |
| DR742(DR589-DR231) | 297 | 1.3 | 3.3 |
| DR743(DR589-DR232) | 166 | 0.7 | 1.9 |
| DR744(DR589-DR233) | 622 | 2.7 | 6.9 |
| DR745(DR589-DR234) | 283 | 1.2 | 3.2 |
| DR746(DR590-DR229) | 577 | 2.5 | 6.4 |
| DR747(DR590-DR230) | 1905 | 8.2 | 21.3 |
| DR748(DR590-DR231) | 962 | 4.1 | 10.7 |
| DR749(DR590-DR232) | 366 | 1.6 | 4.1 |
| DR750(DR590-DR233) | 275 | 1.2 | 3.1 |
| DR751(DR590-DR234) | 220 | 0.9 | 2.5 |
| medium | 232 | 1.0 | 2.6 |
| IL2 | 8956 | 38.6 | 100.0 |

In addition, EC50 values for proliferation and IFNγ production were determined for 36 Anti-IL2Rβ/γ V_{H}H²'s. Purified CD8 positive T cell blasts from 2 different donors were resuspended at 2 million cells/mL in PBS. Anti-IL2Rβ/γ V_{H}H²'s or human IL-2 were titrated into 96-well plates (Falcon) starting at 200 nM in 75 μl DPBS and 6 times 10 fold diluted. 100 thousand CD8 positive T cells were added per well in 75 μl DPBS. Controls included wells without anti-IL2Rβ/γ V_{H}H²'s or human IL-2 (media). Plates were transferred to a humidified incubator (ThermoFisher) and incubated at 37 degrees centigrade, 5 percent carbon dioxide for 72 hours and proliferation and IFNγ production measured as above.

To determine the EC50 of each anti-IL2Rβ/γ V_{H}H²'s variant upon proliferation and IFNγ production, IFNγ MSD and Celltiterglo luminescence signal values for cells treated with anti-IL2Rβ/γ V_{H}H²'s and human IL-2 were analyzed by 4 parameter Nonlinear Regression on log transformed concentration values using Prism GraphPad software. Emax values were compared to those obtained for control cells treated with growth medium alone to yield a fold induction. The data from these experiments is presented in Table 19 and Table 20.

In this assay anti-IL2Rβ/γ V_{H}H²'s induced STAT5 proliferation and IFNγ production by human CD8 positive T cell blasts at distinct EC50 values and Emax values, and relative potency was conserved between donors.

TABLE 19
Proliferation and IFNg production induced by anti-IL2Rβ/γ
V_{H}H² on Primary CD8 T cell Blasts

| | EC50 | | EC50 IFNg | |
|---|---|---|---|---|
| anti-IL2Rβ/γ V_{H}H² | Proliferation (pM) | | Production (pM) | |
| Construct | D12 | D16 | D12 | D16 |
| DR634(DR229-DR583) | 24 | 16 | 218 | 258 |
| DR635(DR229-DR584) | 39 | 73 | 1307 | 1239 |
| DR636(DR229-DR585) | 30299 | 13232 | 32580 | 176915 |
| DR638(DR229-DR587) | 15 | 24 | 232 | 365 |
| DR639(DR229-DR588) | 28 | 42 | 923 | 1143 |
| DR642(DR230-DR214) | 133 | 635 | 8004 | 13352 |
| DR643(DR230-DR217) | 8199 | 12706 | 197350 | 1142790 |
| DR644(DR230-DR583) | 12 | 494 | 110 | 1520 |
| DR645(DR230-DR584) | 41 | 154 | 503 | 1139 |
| DR648(DR230-DR587) | 23 | 49880 | 158 | 803794594 |
| DR652(DR231-DR214) | 657 | 388351 | 3137 | 636759161 |
| DR655(DR231-DR584) | NA | NA | 20241 | NA |
| DR656(DR231-DR585) | 9879 | 0 | 91914 | NA |
| DR671(DR232-DR590) | 20 | 2 | NA | NA |
| DR674(DR233-DR583) | 73 | 110 | 1680 | 764 |
| DR675(DR233-DR584) | 661 | 1027 | 6289 | 4111 |
| DR676(DR233-DR584) | 12748 | 28357 | 486445 | 55151 |
| DR678(DR233-DR587) | 425 | 1026 | 2424 | 1645 |
| DR679(DR233-DR588) | 191 | 1090 | 39053 | 15137 |
| DR680(DR233-DR589) | 49429 | NA | 2436507 | 534395 |
| DR681(DR233-DR590) | 5489 | 36 | NA | NA |
| DR682(DR234-DR214) | 4238 | 2181 | 14903 | 13002 |
| DR688(DR234-DR587) | 111 | 67 | 3157 | 197 |
| DR696(DR214-DR233) | 28968 | 33600 | 53221 | 43286 |
| DR714(DR584-DR233) | NA | 391738 | 767789054 | 704782797 |
| DR716(DR585-DR229) | 1216 | 6327 | 73363 | 22072 |
| DR717(DR585-DR230) | 146 | 156 | 1059 | 947 |
| DR718(DR585-DR231) | 900 | 881 | 2704 | 2907 |
| DR719(DR585-DR232) | 1091 | 2132 | 45349 | 49478 |
| DR720(DR585-DR233) | 10823 | 14194 | 116492 | 42782 |
| DR721(DR585-DR234) | 115 | 161 | 979 | 1708 |
| DR736(DR588-DR231) | 182 | 279 | 3935 | 9028 |
| DR740(DR589-DR229) | NA | 315755 | 791018218 | 500648091 |
| DR741(DR589-DR230) | NA | 48169 | 718953614 | 123207 |
| DR744(DR589-DR233) | NA | 75757 | 598388843 | 198121846 |
| DR747(DR590-DR230) | 12 | 141 | 466 | 679 |
| IL-2 | 130 | 142 | 447 | 357 |

NA: Not Available.
D12: Donor 12.
D16: Donor 16.

TABLE 20
Proliferation and IFNg production induced by anti-IL2Rβ/γ
V_{H}H² on Primary CD8 T cell Blasts

| | Emax Proliferation | | Emax IFNg Production | |
|---|---|---|---|---|
| anti-IL2Rβ/γ V_{H}H² | (Fold over media control) | | (Fold over media control) | |
| Construct | D12 | D16 | D12 | D16 |
| DR634(DR229-DR583) | 8.6 | 11.9 | 104 | 114 |
| DR635(DR229-DR584) | 10.6 | 19.5 | 187 | 318 |
| DR636(DR229-DR585) | 3.6 | 6.3 | 16 | 20 |
| DR638(DR229-DR587) | 13.3 | 26.7 | 275 | 441 |
| DR639(DR229-DR588) | 8.9 | 16.2 | 109 | 194 |
| DR642(DR230-DR214) | 9.6 | 22.8 | 186 | 336 |
| DR643(DR230-DR217) | 6.8 | 13.0 | 57 | 81 |
| DR644(DR230-DR583) | 4.1 | 10.0 | 17 | 22 |
| DR645(DR230-DR584) | 7.5 | 25.0 | 222 | 361 |
| DR648(DR230-DR587) | 13.5 | 29.0 | 256 | 237 |
| DR652(DR231-DR214) | 13.8 | 10.6 | 242 | 17 |
| DR655(DR231-DR584) | 1.0 | 1.1 | 1 | 1 |
| DR656(DR231-DR585) | 7.1 | 1.7 | 39 | 3 |
| DR671(DR232-DR590) | 1.9 | 0.9 | 2 | 1 |
| DR674(DR233-DR583) | 5.8 | 12.2 | 63 | 73 |
| DR675(DR233-DR584) | 5.7 | 19.8 | 162 | 243 |
| DR676(DR233-DR584) | 3.0 | 9.4 | 29 | 19 |
| DR678(DR233-DR587) | 9.4 | 26.1 | 250 | 285 |
| DR679(DR233-DR588) | 3.7 | 12.0 | 79 | 91 |
| DR680(DR233-DR589) | 3.8 | 9.7 | 15 | 21 |
| DR681(DR233-DR590) | 0.9 | 1.3 | 1 | 1 |
| DR682(DR234-DR214) | 3.5 | 7.3 | 15 | 19 |
| DR688(DR234-DR587) | 4.6 | 10.9 | 53 | 38 |
| DR696(DR214-DR233) | 2.0 | 3.6 | 5 | 4 |
| DR714(DR584-DR233) | 4.1 | 8.0 | 22 | 11 |
| DR716(DR585-DR229) | 3.8 | 12.7 | 99 | 104 |
| DR717(DR585-DR230) | 6.9 | 12.8 | 86 | 62 |
| DR718(DR585-DR231) | 8.5 | 18.3 | 213 | 255 |
| DR719(DR585-DR232) | 5.4 | 12.0 | 123 | 143 |
| DR720(DR585-DR233) | 4.8 | 7.7 | 40 | 28 |
| DR721(DR585-DR234) | 6.8 | 18.7 | 180 | 277 |
| DR736(DR588-DR231) | 3.6 | 10.6 | 106 | 106 |
| DR740(DR589-DR229) | 3.2 | 3.6 | 8 | 4 |
| DR741(DR589-DR230) | 3.8 | 5.3 | 20 | 9 |
| DR744(DR589-DR233) | 1.9 | 2.4 | 5 | 4 |
| DR747(DR590-DR230) | 4.9 | 10.3 | 43 | 39 |
| IL-2 | 12 | 27 | 315 | 472 |

NA: Not Available.
D12: Donor 12.
D16: Donor 16.

### Example 14—Evaluation of Activity of anti-IL2Rβ/γ V_{H}H²s On CD4 positive T cell Clone 3F8

The anti-IL2Rβ/γ V_{H}H²s were evaluated for activity in CD4 positive human T cell clone 3F8 cells. The CD4 positive T cell clone 3F8 was generated by activation of PBMC of a healthy donor with the EBV transformed B cell line JY in two successive rounds of Mixed Leukocyte Reactions followed by single cell cloning by limited dilution as described (Yssel and Spits (2002) Current Protocols in Immunology 7.19.1-7.19.12). The CD4 positive T cell clone 3F8 expresses CD25 and CD122 and proliferates in response to IL2.

3F8 cells were contacted with purified anti-IL2Rβ/γ V_{H}H²s as follows: Cells were grown in growth medium consisting of Yssel's medium (Iscove's modified Dulbecco's Medium (ThermoFisher), 0.25% w/v percent human albumin (Sigma), 1 percent penicillin/streptomycin (ThermoFisher), 1 percent ITS-X Insulin, Transferrin, Selenium (Gibco), 30 mg/L Tansferrin (Roche), 2 mg/L Palmitic Acid (Sigma), 1 percent LA-OA-Albumin Linoleic Acid, Oleic Acid (Sigma), 1 percent human serum (Gemini) (Yssel et al (1984) J Immunol Methods 72: 219-227) at 0.2 million cells per ml with 50 Gy irradiated JY cells at 0.1 million cells per well and 40 Gy irradiated allogeneic PBMC at 1 million cells per mL. After six days of culture and expansion with human IL2 at 100 pM, cells were washed and seeded into black, clear bottom 96 well plates (Costar) at 50 thousand cells per well in 50 μl growth medium. Anti-IL2Rβ/γ V_{H}H²s were diluted into 96-well plates (Falcon) at 30 nM in 50 μl growth medium without IL2 and added to the wells. Controls included wells without anti-IL2Rβ/γ V_{H}H²s or human IL2 (media) and wells with human IL2 at a final concentration of 100 pM. Plates were transferred to a humidified incubator (ThermoFisher) and incubated at 37 degrees centigrade, 5 percent carbon dioxide for 72 hrs.

Plates were removed from the incubator and cells were lysed by adding 100 μl per well of Celltiterglo™ (CTG) (Promega) according to manufacturer's instructions. Cell lysates were mixed on an orbital shaker (VWR Scientific) for 10 minutes at 300 rpm. Luminescence was read as counts per second on an Envision 2103 Multilabel Plate Reader (Perkin Elmer) using the ATPLite protocol.

To compare the effect of each anti-IL2Rβ/γ V_{H}H²s variant upon proliferation, CTG luminescence signal values for cells treated with anti-IL2Rβ/γ V_{H}H²s were compared to those obtained for control cells treated with growth medium alone to yield a fold induction and with control cells treated with human IL2 to yield an activity relative to the response to IL2 as a percentage. The data from these experiments is presented in Table 21.

In this assay 87/120 anti-IL2Rβ/γ V_{H}H²s induced 3F8 cell proliferation at distinct levels (>1 fold), but not above levels observed with IL2 at 100 pM.

TABLE 21
Induction of proliferation by anti-IL2Rβ/γ
V_{H}H² on 3F8 T Cell Clone

| | 3F8 | Relative | Relative |
|---|---|---|---|
| | proliferation | Proliferation | Proliferation |
| | Induction | Induction | Induction |
| Anti-IL2Rβ/γ V_{H}H² | CTG | (fold | (% hIL2 |
| Construct | (cpm) | induction) | response) |
| DR632(DR229-DR214) | 33888 | 0.8 | 36.9 |
| DR633(DR229-DR217) | 41194 | 1.0 | 44.8 |
| DR634(DR229-DR583) | 66462 | 1.6 | 72.4 |
| DR635(DR229-DR584) | 76938 | 1.9 | 83.8 |
| DR636(DR229-DR585) | 44314 | 1.1 | 48.2 |
| DR637(DR229-DR586) | 46870 | 1.1 | 51.0 |
| DR638(DR229-DR587) | 87848 | 2.1 | 95.6 |
| DR639(DR229-DR587) | 71832 | 1.7 | 78.2 |
| DR640(DR229-DR589) | 64522 | 1.6 | 70.2 |
| DR641(DR229-DR590) | 43324 | 1.0 | 47.2 |
| DR642(DR230-DR214) | 84204 | 2.0 | 91.7 |
| DR643(DR230-DR217) | 79866 | 1.9 | 86.9 |
| DR644(DR230-DR583) | 57066 | 1.4 | 62.1 |
| DR645(DR230-DR584) | 72772 | 1.8 | 79.2 |
| DR646(DR230-DR585) | 47376 | 1.1 | 51.6 |
| DR647(DR230-DR586) | 73828 | 1.8 | 80.4 |
| DR648(DR230-DR587) | 72194 | 1.7 | 78.6 |
| DR649(DR230-DR588) | 48562 | 1.2 | 52.9 |
| DR650(DR230-DR589) | 45092 | 1.1 | 49.1 |
| DR651(DR230-DR590) | 41826 | 1.0 | 45.5 |
| DR652(DR231-DR214) | 71060 | 1.7 | 77.4 |
| DR653(DR231-DR217) | 31888 | 0.8 | 34.7 |
| DR654(DR231-DR583) | 37502 | 0.9 | 40.8 |
| DR655(DR231-DR584) | 41534 | 1.0 | 45.2 |
| DR656(DR231-DR585) | 78076 | 1.9 | 85.0 |
| DR657(DR231-DR586) | 53584 | 1.3 | 58.3 |
| DR658(DR231-DR587) | 40568 | 1.0 | 44.2 |
| DR659(DR231-DR588) | 52996 | 1.3 | 57.7 |
| DR660(DR231-DR589) | 41636 | 1.0 | 45.3 |
| DR661(DR231-DR590) | 41330 | 1.0 | 45.0 |
| DR662(DR232-DR214) | 49936 | 1.2 | 54.4 |
| DR663(DR232-DR217) | 71038 | 1.7 | 77.3 |
| DR664(DR232-DR583) | 69286 | 1.7 | 75.4 |
| DR665(DR232-DR584) | 41762 | 1.0 | 45.5 |
| DR666(DR232-DR585) | 50210 | 1.2 | 54.7 |
| DR667(DR232-DR586) | 74854 | 1.8 | 81.5 |
| DR668(DR232-DR587) | 51260 | 1.2 | 55.8 |
| DR669(DR232-DR588) | 52574 | 1.3 | 57.2 |
| DR670(DR232-DR589) | 62090 | 1.5 | 67.6 |
| DR671(DR232-DR590) | 71850 | 1.7 | 78.2 |
| DR672(DR233-DR214) | 63810 | 1.5 | 69.5 |
| DR673(DR233-DR217) | 66648 | 1.6 | 72.6 |
| DR674(DR233-DR583) | 78250 | 1.9 | 85.2 |
| DR675(DR233-DR584) | 79716 | 1.9 | 86.8 |
| DR676(DR233-DR584) | 47408 | 1.1 | 51.6 |
| DR677(DR233-DR586) | 60714 | 1.5 | 66.1 |
| DR678(DR233-DR587) | 84816 | 2.1 | 92.3 |
| DR679(DR233-DR588) | 70776 | 1.7 | 77.1 |
| DR680(DR233-DR589) | 72106 | 1.7 | 78.5 |
| DR681(DR233-DR590) | 71270 | 1.7 | 77.6 |
| DR682(DR234-DR214) | 59464 | 1.4 | 64.7 |
| DR683(DR234-DR217) | 62038 | 1.5 | 67.5 |
| DR684(DR234-DR583) | 40188 | 1.0 | 43.8 |
| DR685(DR234-DR584) | 62736 | 1.5 | 68.3 |
| DR686(DR234-DR585) | 48414 | 1.2 | 52.7 |
| DR687(DR234-DR586) | 59830 | 1.4 | 65.1 |
| DR688(DR234-DR587) | 71834 | 1.7 | 78.2 |
| DR689(DR234-DR588) | 75204 | 1.8 | 81.9 |
| DR690(DR234-DR589) | 75612 | 1.8 | 82.3 |
| DR691(DR234-DR590) | 48538 | 1.2 | 52.8 |
| DR692(DR214-DR229) | 52982 | 1.3 | 57.7 |
| DR693(DR214-DR230) | 57408 | 1.4 | 62.5 |
| DR694(DR214-DR231) | 41318 | 1.0 | 45.0 |
| DR695(DR214-DR232) | 63280 | 1.5 | 68.9 |
| DR696(DR214-DR233) | 59748 | 1.4 | 65.0 |
| DR697(DR214-DR234) | 50344 | 1.2 | 54.8 |
| DR698(DR217-DR229) | 46812 | 1.1 | 51.0 |
| DR699(DR217-DR230) | 55404 | 1.3 | 60.3 |
| DR700(DR217-DR231) | 47242 | 1.1 | 51.4 |
| DR701(DR217-DR232) | 62282 | 1.5 | 67.8 |
| DR702(DR217-DR233) | 62942 | 1.5 | 68.5 |
| DR703(DR217-DR234) | 58558 | 1.4 | 63.7 |
| DR704(DR583-DR229) | 36402 | 0.9 | 39.6 |
| DR705(DR583-DR230) | 41940 | 1.0 | 45.7 |
| DR706(DR583-DR231) | 36666 | 0.9 | 39.9 |
| DR707(DR583-DR232) | 38206 | 0.9 | 41.6 |
| DR708(DR583-DR233) | 32604 | 0.8 | 35.5 |
| DR709(DR583-DR234) | 37410 | 0.9 | 40.7 |
| DR710(DR584-DR229) | 35512 | 0.9 | 38.7 |
| DR711(DR584-DR230) | 37716 | 0.9 | 41.1 |
| DR712(DR584-DR231) | 37102 | 0.9 | 40.4 |
| DR713(DR584-DR232) | 40972 | 1.0 | 44.6 |
| DR714(DR584-DR233) | 27114 | 0.7 | 29.5 |
| DR715(DR584-DR234) | 39740 | 1.0 | 43.3 |
| DR716(DR585-DR229) | 81372 | 2.0 | 88.6 |
| DR717(DR585-DR230) | 72588 | 1.8 | 79.0 |
| DR718(DR585-DR231) | 80658 | 2.0 | 87.8 |
| DR719(DR585-DR232) | 70762 | 1.7 | 77.0 |
| DR720(DR585-DR233) | 88944 | 2.2 | 96.8 |
| DR721(DR585-DR234) | 72802 | 1.8 | 79.3 |
| DR722(DR586-DR229) | 74538 | 1.8 | 81.1 |
| DR723(DR586-DR230) | 50868 | 1.2 | 55.4 |
| DR724(DR586-DR231) | 70232 | 1.7 | 76.5 |
| DR725(DR586-DR232) | 54800 | 1.3 | 59.7 |
| DR726(DR586-DR233) | 35400 | 0.9 | 38.5 |
| DR727(DR586-DR234) | 40006 | 1.0 | 43.6 |
| DR728(DR587-DR229) | 35520 | 0.9 | 38.7 |
| DR729(DR587-DR230) | 52430 | 1.3 | 57.1 |
| DR730(DR587-DR231) | 34202 | 0.8 | 37.2 |
| DR731(DR587-DR232) | 42902 | 1.0 | 46.7 |
| DR732(DR587-DR233) | 44550 | 1.1 | 48.5 |
| DR733(DR587-DR234) | 49704 | 1.2 | 54.1 |
| DR734(DR588-DR229) | 38080 | 0.9 | 41.5 |
| DR735(DR588-DR230) | 55892 | 1.4 | 60.8 |
| DR736(DR588-DR231) | 87242 | 2.1 | 95.0 |
| DR737(DR588-DR232) | 65336 | 1.6 | 71.1 |
| DR738(DR588-DR233) | 40950 | 1.0 | 44.6 |
| DR739(DR588-DR234) | 59136 | 1.4 | 64.4 |
| DR740(DR589-DR229) | 75534 | 1.8 | 82.2 |
| DR741(DR589-DR230) | 89468 | 2.2 | 97.4 |
| DR742(DR589-DR231) | 48714 | 1.2 | 53.0 |
| DR743(DR589-DR232) | 45886 | 1.1 | 50.0 |
| DR744(DR589-DR233) | 67692 | 1.6 | 73.7 |
| DR745(DR589-DR234) | 49226 | 1.2 | 53.6 |
| DR746(DR590-DR229) | 44046 | 1.1 | 48.0 |
| DR747(DR590-DR230) | 66262 | 1.6 | 72.1 |
| DR748(DR590-DR231) | 90162 | 2.2 | 98.2 |
| DR749(DR590-DR232) | 75762 | 1.8 | 82.5 |
| DR750(DR590-DR233) | 46084 | 1.1 | 50.2 |
| DR751(DR590-DR234) | 36334 | 0.9 | 39.6 |
| medium | 41308 | 1.0 | 45.0 |
| IL2 | 91857 | 2.2 | 100.0 |

### Example 15—Evaluation of Activity of anti-IL2Rβ/γ V_{H}H²s On CD8 positive T cell Clone 5B9

The anti-IL2Rβ/γ V_{H}H²s were evaluated for activity in CD8 positive human T cell clone 3F8 cells. The CD8 positive T cell clone 5B9 was generated by activation of PBMC of a healthy donor with the EBV transformed B cell line JY in two successive rounds of Mixed Leukocyte Reactions followed by single cell cloning by limited dilution as described (Yssel and Spits (2002) Current Protocols in Immunology 7.19.1-7.19.12). The CD8 positive T cell clone 5B9 expresses CD25 and CD122 and proliferates and phosphorylated STAT5 in response to IL2.

5B9 cells were contacted with purified anti-IL2Rβ/γ V_{H}H²s as follows: Cells were grown in growth medium consisting of Yssel's medium (Iscove's modified Dulbecco's Medium (ThermoFisher), 0.25% w/v percent human albumin (Sigma), 1 percent penicillin/streptomycin (ThermoFisher), 1 percent ITS-X Insulin, Transferrin, Selenium (Gibco), 30 mg/L Tansferrin (Roche), 2 mg/L Palmitic Acid (Sigma), 1 percent LA-OA-Albumin Linoleic Acid, Oleic Acid (Sigma), 1 percent human serum (Gemini) (Yssel et al (1984) J Immunol Methods 72: 219-227) at 0.2 million cells per ml with 50 Gy irradiated JY cells at 0.1 million cells per well and 40 Gy irradiated allogeneic PBMC at 1 million cells per mL. After twelve days of culture and expansion with human IL2 at 100 pM, 5B9 cells were centrifuged and washed in DPBS twice. Cells were resuspended at 2 million cells/mL in PBS. Anti-IL2Rβ/γ V_{H}H²s were diluted into 96-well plates (Falcon) to 30 nM in 50 μl DPBS and 100 thousand activated 5B9 cells were added per well in 50 μl DPBS. Controls included wells without anti-IL2Rβ/γ V_{H}H²s or human IL2 (media) and wells with human IL2 at a final concentration of 100 pM. Plates were transferred to a humidified incubator (ThermoFisher) and incubated at 37 degrees centigrade, 5 percent carbon dioxide for 20 min.

Plates were removed from the incubator and cells were lysed by adding 100 μl per well of Tris Lysis buffer supplemented with Protease Inhibitor Solution, Phosphatase Inhibitor I and Phosphatase Inhibitor II from MSD Multispot Assay System Phospho-STAT Panel Kit (MSD K15202D) according to manufacturer's instructions. Plates were incubated on ice for 15 minutes and centrifuged at 600×g for 6 minutes. Cell lysates were transferred to a new 96 well plate.

Induction of STAT5 phosphorylation was measured in the cell lysates using the MSD Multispot Assay System Phospho-STAT Panel (MSD K15202D) according to manufacturer's instructions. Briefly, mAb precoated MSD Phospho-STAT panel assay plates were incubated with 150 μL per well Blocker A in Tris Wash Buffer at 30 mg/mL for 60 min on an orbital shaker (VWR Scientific) at 300 rpm at room temperature. Plates were washed 3 times with Tris Wash Buffer and 30 μL Cell lysates were added per well. Plates were incubated for 60 min on an orbital shaker (VWR Scientific) at 300 rpm at room temperature. Plates were washed 3 times with Tris Wash Buffer and 25 μL 1×detection antibody in 10 mg/mL Blocker A in Tris Wash Buffer was added to each well. Plates were incubated for 60 min on an orbital shaker (VWR Scientific) at 300 rpm at room temperature. Plates were washed 3 times with Tris Wash Buffer and 150 μL 1× Read Buffer T was added to each well and Luminescence signal was read on a Mesoscale Quickplex SQ 120 instrument

To compare the effect of each anti-IL2Rβ/γ V_{H}H²s variant upon CD8 positive T cell clone 5B9 STAT5 phosphorylation, MSD luminescence signal values for cells treated with anti-IL2Rβ/γ V_{H}H²s were compared to those obtained for control cells treated with growth medium alone to yield a fold induction and with control cells treated with human IL2 to yield an activity relative to the response to IL2 as a percentage. The data from these experiments is presented in Table 22.

In this assay 9/120 anti-IL2Rβ/γ V_{H}H²s induced STAT5 phosphorylation in CD8 positive T cell clone 5B9 at distinct levels (Fold>1), but not above levels observed with IL2 at 100 pM.

TABLE 22
pSTAT5 Induction by anti-IL2Rβ/γ
V_{H}H² on CD8 T cell clone 5B9

| | 5B9 | Relative | Relative |
|---|---|---|---|
| | pSTAT5 | pSTAT5 | pSTAT5 |
| | Induction | Induction | Induction |
| Anti-IL2Rβ/γ V_{H}H² | (MSD | (fold | (% hIL2 |
| Construct | Signal) | induction) | response) |
| DR632(DR229-DR214) | 1269 | 0.4 | 13.9 |
| DR633(DR229-DR217) | 1218 | 0.4 | 13.3 |
| DR634(DR229-DR583) | 1752 | 0.6 | 19.1 |
| DR635(DR229-DR584) | 1897 | 0.6 | 20.7 |
| DR636(DR229-DR585) | 1304 | 0.4 | 14.2 |
| DR637(DR229-DR586) | 1136 | 0.4 | 12.4 |
| DR638(DR229-DR587) | 1633 | 0.5 | 17.8 |
| DR639(DR229-DR587) | 1780 | 0.6 | 19.4 |
| DR640(DR229-DR589) | 1227 | 0.4 | 13.4 |
| DR641(DR229-DR590) | 1089 | 0.4 | 11.9 |
| DR642(DR230-DR214) | 1785 | 0.6 | 19.5 |
| DR643(DR230-DR217) | 1647 | 0.5 | 18.0 |
| DR644(DR230-DR583) | 1137 | 0.4 | 12.4 |
| DR645(DR230-DR584) | 1244 | 0.4 | 13.6 |
| DR646(DR230-DR585) | 1580 | 0.5 | 17.3 |
| DR647(DR230-DR586) | 1601 | 0.5 | 17.5 |
| DR648(DR230-DR587) | 1280 | 0.4 | 14.0 |
| DR649(DR230-DR588) | 1161 | 0.4 | 12.7 |
| DR650(DR230-DR589) | 1546 | 0.5 | 16.9 |
| DR651(DR230-DR590) | 1343 | 0.4 | 14.7 |
| DR652(DR231-DR214) | 1238 | 0.4 | 13.5 |
| DR653(DR231-DR217) | 1192 | 0.4 | 13.0 |
| DR654(DR231-DR583) | 1534 | 0.5 | 16.8 |
| DR655(DR231-DR584) | 1543 | 0.5 | 16.9 |
| DR656(DR231-DR585) | 2108 | 0.7 | 23.0 |
| DR657(DR231-DR586) | 1958 | 0.6 | 21.4 |
| DR658(DR231-DR587) | 2853 | 0.9 | 31.2 |
| DR659(DR231-DR588) | 2750 | 0.9 | 30.0 |
| DR660(DR231-DR589) | 2149 | 0.7 | 23.5 |
| DR661(DR231-DR590) | 1964 | 0.6 | 21.4 |
| DR662(DR232-DR214) | 2965 | 1.0 | 32.4 |
| DR663(DR232-DR217) | 2800 | 0.9 | 30.6 |
| DR664(DR232-DR583) | 2176 | 0.7 | 23.8 |
| DR665(DR232-DR584) | 1956 | 0.6 | 21.4 |
| DR666(DR232-DR585) | 3292 | 1.1 | 36.0 |
| DR667(DR232-DR586) | 2493 | 0.8 | 27.2 |
| DR668(DR232-DR587) | 1963 | 0.6 | 21.4 |
| DR669(DR232-DR588) | 1630 | 0.5 | 17.8 |
| DR670(DR232-DR589) | 3099 | 1.0 | 33.8 |
| DR671(DR232-DR590) | 2864 | 0.9 | 31.3 |
| DR672(DR233-DR214) | 1851 | 0.6 | 20.2 |
| DR673(DR233-DR217) | 2285 | 0.7 | 25.0 |
| DR674(DR233-DR583) | 4006 | 1.3 | 43.7 |
| DR675(DR233-DR584) | 3299 | 1.1 | 36.0 |
| DR676(DR233-DR584) | 2050 | 0.7 | 22.4 |
| DR677(DR233-DR586) | 2085 | 0.7 | 22.8 |
| DR678(DR233-DR587) | 5875 | 1.9 | 64.2 |
| DR679(DR233-DR588) | 3141 | 1.0 | 34.3 |
| DR680(DR233-DR589) | 2088 | 0.7 | 22.8 |
| DR681(DR233-DR590) | 2923 | 1.0 | 31.9 |
| DR682(DR234-DR214) | 1090 | 0.4 | 11.9 |
| DR683(DR234-DR217) | 1064 | 0.3 | 11.6 |
| DR684(DR234-DR583) | 2148 | 0.7 | 23.5 |
| DR685(DR234-DR584) | 2720 | 0.9 | 29.7 |
| DR686(DR234-DR585) | 1114 | 0.4 | 12.2 |
| DR687(DR234-DR586) | 1138 | 0.4 | 12.4 |
| DR688(DR234-DR587) | 2837 | 0.9 | 31.0 |
| DR689(DR234-DR588) | 2317 | 0.8 | 25.3 |
| DR690(DR234-DR589) | 1118 | 0.4 | 12.2 |
| DR691(DR234-DR590) | 1087 | 0.4 | 11.9 |
| DR692(DR214-DR229) | 2156 | 0.7 | 23.5 |
| DR693(DR214-DR230) | 1988 | 0.6 | 21.7 |
| DR694(DR214-DR231) | 1060 | 0.3 | 11.6 |
| DR695(DR214-DR232) | 1162 | 0.4 | 12.7 |
| DR696(DR214-DR233) | 3354 | 1.1 | 36.6 |
| DR697(DR214-DR234) | 2683 | 0.9 | 29.3 |
| DR698(DR217-DR229) | 1114 | 0.4 | 12.2 |
| DR699(DR217-DR230) | 1092 | 0.4 | 11.9 |
| DR700(DR217-DR231) | 2454 | 0.8 | 26.8 |
| DR701(DR217-DR232) | 3240 | 1.1 | 35.4 |
| DR702(DR217-DR233) | 1074 | 0.4 | 11.7 |
| DR703(DR217-DR234) | 1257 | 0.4 | 13.7 |
| DR704(DR583-DR229) | 1258 | 0.4 | 13.7 |
| DR705(DR583-DR230) | 978 | 0.3 | 10.7 |
| DR706(DR583-DR231) | 1521 | 0.5 | 16.6 |
| DR707(DR583-DR232) | 1374 | 0.4 | 15.0 |
| DR708(DR583-DR233) | 1459 | 0.5 | 15.9 |
| DR709(DR583-DR234) | 947 | 0.3 | 10.3 |
| DR710(DR584-DR229) | 1506 | 0.5 | 16.4 |
| DR711(DR584-DR230) | 1287 | 0.4 | 14.1 |
| DR712(DR584-DR231) | 1136 | 0.4 | 12.4 |
| DR713(DR584-DR232) | 911 | 0.3 | 9.9 |
| DR714(DR584-DR233) | 1621 | 0.5 | 17.7 |
| DR715(DR584-DR234) | 1224 | 0.4 | 13.4 |
| DR716(DR585-DR229) | 1058 | 0.3 | 11.6 |
| DR717(DR585-DR230) | 831 | 0.3 | 9.1 |
| DR718(DR585-DR231) | 1529 | 0.5 | 16.7 |
| DR719(DR585-DR232) | 1097 | 0.4 | 12.0 |
| DR720(DR585-DR233) | 1228 | 0.4 | 13.4 |
| DR721(DR585-DR234) | 1002 | 0.3 | 10.9 |
| DR722(DR586-DR229) | 1765 | 0.6 | 19.3 |
| DR723(DR586-DR230) | 1034 | 0.3 | 11.3 |
| DR724(DR586-DR231) | 1044 | 0.3 | 11.4 |
| DR725(DR586-DR232) | 996 | 0.3 | 10.9 |
| DR726(DR586-DR233) | 1516 | 0.5 | 16.6 |
| DR727(DR586-DR234) | 1005 | 0.3 | 11.0 |
| DR728(DR587-DR229) | 2575 | 0.8 | 28.1 |
| DR729(DR587-DR230) | 3054 | 1.0 | 33.4 |
| DR730(DR587-DR231) | 3302 | 1.1 | 36.1 |
| DR731(DR587-DR232) | 5336 | 1.7 | 58.3 |
| DR732(DR587-DR233) | 2605 | 0.9 | 28.4 |
| DR733(DR587-DR234) | 2372 | 0.8 | 25.9 |
| DR734(DR588-DR229) | 3622 | 1.2 | 39.6 |
| DR735(DR588-DR230) | 3175 | 1.0 | 34.7 |
| DR736(DR588-DR231) | 3608 | 1.2 | 39.4 |
| DR737(DR588-DR232) | 2864 | 0.9 | 31.3 |
| DR738(DR588-DR233) | 3243 | 1.1 | 35.4 |
| DR739(DR588-DR234) | 3150 | 1.0 | 34.4 |
| DR740(DR589-DR229) | 2891 | 0.9 | 31.6 |
| DR741(DR589-DR230) | 2611 | 0.9 | 28.5 |
| DR742(DR589-DR231) | 3593 | 1.2 | 39.2 |
| DR743(DR589-DR232) | 3060 | 1.0 | 33.4 |
| DR744(DR589-DR233) | 2903 | 0.9 | 31.7 |
| DR745(DR589-DR234) | 2331 | 0.8 | 25.5 |
| DR746(DR590-DR229) | 2588 | 0.8 | 28.3 |
| DR747(DR590-DR230) | 2970 | 1.0 | 32.4 |
| DR748(DR590-DR231) | 2832 | 0.9 | 30.9 |
| DR749(DR590-DR232) | 2561 | 0.8 | 28.0 |
| DR750(DR590-DR233) | 3290 | 1.1 | 35.9 |
| DR751(DR590-DR234) | 2505 | 0.8 | 27.4 |
| medium | 3062 | 1.0 | 33.4 |
| IL2 | 9157 | 3.0 | 100.0 |

### Example 16—Evaluation of Proliferative Activity of Anti-IL2Rβ/γ V_{H}H²s On CTLL-2 Cells

The anti-IL2Rβ/γ V_{H}H²s were evaluated for activity in CTLL-2 cells (ATCC TIB-214). CTLL-2 is a murine IL2 dependent Leukemic cell line with T cell characteristics that expresses IL2Rβ and IL2Rγ chains and is able to proliferate in response to IL2 receptor signaling.

CTLL-2 cells were contacted with purified anti-IL2Rβ/γ V_{H}H²s as follows: Cells were cultured in medium consisting of RPM 1640 (ThermoFisher), 10 percent fetal bovine serum (ThermoFisher), 1 percent penicillin/streptomycin (ThermoFisher), 1 percent sodium pyruvate (ThermoFisher), 2-mercaptoethanol (Gibco) and 10% T-STIM (BD 354115) at densities between 0.2-1 million cells per ml. Prior to the experiment CTLL-2 cells were harvested, centrifuged and washed in DPBS twice. CTLL-2 cells were resuspended at 0.5 million cells/mL in growth medium without T-STIM. Anti-IL2Rβ/γ V_{H}H²s were diluted into 96-well plates (Falcon) at 30 nM in 50 μl growth medium without T-STIM and 25 thousand CTLL-2 cells in 50 μl growth medium without T-STIM were added per well. Controls included wells without anti-IL2Rβ/γ V_{H}H²s or human IL2 (media) and wells with human IL2 at a final concentration of 100 pM. Plates were transferred to a humidified incubator (ThermoFisher) and incubated at 37 degrees centigrade, 5 percent carbon dioxide for 48 hrs.

Plates were removed from the incubator and cells were lysed by adding 100 μl per well of Celltiterglo™ (CTG) (Promega) according to manufacturer's instructions. Cell lysates were mixed on an orbital shaker (VWR Scientific) for 10 minutes at 300 rpm. Luminescence was read as counts per second on an Envision 2103 Multilabel Plate Reader (Perkin Elmer) using the ATPLite protocol.

To compare the effect of each anti-IL2Rβ/γ V_{H}H²s variant upon proliferation, CTG luminescence signal values for cells treated with anti-IL2Rβ/γ V_{H}H²s were compared to those obtained for control cells treated with growth medium alone to yield a fold induction and with control cells treated with human IL2 to yield an activity relative to the response to IL2 as a percentage. The data from these experiments is presented in Table 23.

In this assay none of the anti-human-IL2Rβ/γ V_{H}H²s induced CTLL-2 cell proliferation indicating that anti-IL2Rβ/γ V_{H}H²s did not activate murine cells.

TABLE 23
Induction of Proliferation by Anti-IL2Rβ/γ V_{H}H² on CTLL-2 Cells

| | CTLL-2 | Relative | Relative |
|---|---|---|---|
| | proliferation | Proliferation | Proliferation |
| | Induction | Induction | Induction |
| Anti-IL2Rβ/γ V_{H}H² | CTG | (fold | (% hIL2 |
| Construct | (cpm) | induction) | response) |
| DR632(DR229-DR214) | 5004 | 1.0 | 4.1 |
| DR633(DR229-DR217) | 5500 | 1.1 | 4.5 |
| DR634(DR229-DR583) | 5402 | 1.1 | 4.5 |
| DR635(DR229-DR584) | 4134 | 0.8 | 3.4 |
| DR636(DR229-DR585) | 4908 | 1.0 | 4.1 |
| DR637(DR229-DR586) | 4572 | 0.9 | 3.8 |
| DR638(DR229-DR587) | 4576 | 0.9 | 3.8 |
| DR639(DR229-DR587) | 5242 | 1.1 | 4.3 |
| DR640(DR229-DR589) | 4794 | 1.0 | 4.0 |
| DR641(DR229-DR590) | 4670 | 0.9 | 3.9 |
| DR642(DR230-DR214) | 4160 | 0.8 | 3.4 |
| DR643(DR230-DR217) | 4622 | 0.9 | 3.8 |
| DR644(DR230-DR583) | 4656 | 0.9 | 3.9 |
| DR645(DR230-DR584) | 5964 | 1.2 | 4.9 |
| DR646(DR230-DR585) | 4132 | 0.8 | 3.4 |
| DR647(DR230-DR586) | 4892 | 1.0 | 4.0 |
| DR648(DR230-DR587) | 4224 | 0.9 | 3.5 |
| DR649(DR230-DR588) | 4712 | 1.0 | 3.9 |
| DR650(DR230-DR589) | 3930 | 0.8 | 3.3 |
| DR651(DR230-DR590) | 4888 | 1.0 | 4.0 |
| DR652(DR231-DR214) | 4470 | 0.9 | 3.7 |
| DR653(DR231-DR217) | 4136 | 0.8 | 3.4 |
| DR654(DR231-DR583) | 4534 | 0.9 | 3.7 |
| DR655(DR231-DR584) | 4856 | 1.0 | 4.0 |
| DR656(DR231-DR585) | 4780 | 1.0 | 4.0 |
| DR657(DR231-DR586) | 3848 | 0.8 | 3.2 |
| DR658(DR231-DR587) | 5052 | 1.0 | 4.2 |
| DR659(DR231-DR588) | 3596 | 0.7 | 3.0 |
| DR660(DR231-DR589) | 4004 | 0.8 | 3.3 |
| DR661(DR231-DR590) | 4644 | 0.9 | 3.8 |
| DR662(DR232-DR214) | 3868 | 0.8 | 3.2 |
| DR663(DR232-DR217) | 4032 | 0.8 | 3.3 |
| DR664(DR232-DR583) | 4272 | 0.9 | 3.5 |
| DR665(DR232-DR584) | 4096 | 0.8 | 3.4 |
| DR666(DR232-DR585) | 3702 | 0.8 | 3.1 |
| DR667(DR232-DR586) | 4084 | 0.8 | 3.4 |
| DR668(DR232-DR587) | 4176 | 0.8 | 3.5 |
| DR669(DR232-DR588) | 4330 | 0.9 | 3.6 |
| DR670(DR232-DR589) | 3540 | 0.7 | 2.9 |
| DR671(DR232-DR590) | 4458 | 0.9 | 3.7 |
| DR672(DR233-DR214) | 3636 | 0.7 | 3.0 |
| DR673(DR233-DR217) | 4986 | 1.0 | 4.1 |
| DR674(DR233-DR583) | 3960 | 0.8 | 3.3 |
| DR675(DR233-DR584) | 5088 | 1.0 | 4.2 |
| DR676(DR233-DR584) | 4692 | 1.0 | 3.9 |
| DR677(DR233-DR586) | 4190 | 0.9 | 3.5 |
| DR678(DR233-DR587) | 4488 | 0.9 | 3.7 |
| DR679(DR233-DR588) | 4550 | 0.9 | 3.8 |
| DR680(DR233-DR589) | 4064 | 0.8 | 3.4 |
| DR681(DR233-DR590) | 3826 | 0.8 | 3.2 |
| DR682(DR234-DR214) | 4498 | 0.9 | 3.7 |
| DR683(DR234-DR217) | 4198 | 0.9 | 3.5 |
| DR684(DR234-DR583) | 4024 | 0.8 | 3.3 |
| DR685(DR234-DR584) | 4090 | 0.8 | 3.4 |
| DR686(DR234-DR585) | 3738 | 0.8 | 3.1 |
| DR687(DR234-DR586) | 4344 | 0.9 | 3.6 |
| DR688(DR234-DR587) | 3936 | 0.8 | 3.3 |
| DR689(DR234-DR588) | 4376 | 0.9 | 3.6 |
| DR690(DR234-DR589) | 3916 | 0.8 | 3.2 |
| DR691(DR234-DR590) | 4522 | 0.9 | 3.7 |
| DR692(DR214-DR229) | 3442 | 0.7 | 2.8 |
| DR693(DR214-DR230) | 4450 | 0.9 | 3.7 |
| DR694(DR214-DR231) | 3786 | 0.8 | 3.1 |
| DR695(DR214-DR232) | 4152 | 0.8 | 3.4 |
| DR696(DR214-DR233) | 4728 | 1.0 | 3.9 |
| DR697(DR214-DR234) | 4504 | 0.9 | 3.7 |
| DR698(DR217-DR229) | 3678 | 0.7 | 3.0 |
| DR699(DR217-DR230) | 4686 | 1.0 | 3.9 |
| DR700(DR217-DR231) | 3620 | 0.7 | 3.0 |
| DR701(DR217-DR232) | 5122 | 1.0 | 4.2 |
| DR702(DR217-DR233) | 3906 | 0.8 | 3.2 |
| DR703(DR217-DR234) | 4974 | 1.0 | 4.1 |
| DR704(DR583-DR229) | 5820 | 1.2 | 4.8 |
| DR705(DR583-DR230) | 3818 | 0.8 | 3.2 |
| DR706(DR583-DR231) | 6230 | 1.3 | 5.2 |
| DR707(DR583-DR232) | 4102 | 0.8 | 3.4 |
| DR708(DR583-DR233) | 4226 | 0.9 | 3.5 |
| DR709(DR583-DR234) | 4088 | 0.8 | 3.4 |
| DR710(DR584-DR229) | 3950 | 0.8 | 3.3 |
| DR711(DR584-DR230) | 4766 | 1.0 | 3.9 |
| DR712(DR584-DR231) | 3792 | 0.8 | 3.1 |
| DR713(DR584-DR232) | 4372 | 0.9 | 3.6 |
| DR714(DR584-DR233) | 4544 | 0.9 | 3.8 |
| DR715(DR584-DR234) | 5128 | 1.0 | 4.2 |
| DR716(DR585-DR229) | 3756 | 0.8 | 3.1 |
| DR717(DR585-DR230) | 4592 | 0.9 | 3.8 |
| DR718(DR585-DR231) | 5416 | 1.1 | 4.5 |
| DR719(DR585-DR232) | 5422 | 1.1 | 4.5 |
| DR720(DR585-DR233) | 3590 | 0.7 | 3.0 |
| DR721(DR585-DR234) | 4936 | 1.0 | 4.1 |
| DR722(DR586-DR229) | 4006 | 0.8 | 3.3 |
| DR723(DR586-DR230) | 6542 | 1.3 | 5.4 |
| DR724(DR586-DR231) | 3444 | 0.7 | 2.8 |
| DR725(DR586-DR232) | 5026 | 1.0 | 4.2 |
| DR726(DR586-DR233) | 4974 | 1.0 | 4.1 |
| DR727(DR586-DR234) | 5820 | 1.2 | 4.8 |
| DR728(DR587-DR229) | 10826 | 2.2 | 9.0 |
| DR729(DR587-DR230) | 3782 | 0.8 | 3.1 |
| DR730(DR587-DR231) | 4658 | 0.9 | 3.9 |
| DR731(DR587-DR232) | 3740 | 0.8 | 3.1 |
| DR732(DR587-DR233) | 4268 | 0.9 | 3.5 |
| DR733(DR587-DR234) | 3770 | 0.8 | 3.1 |
| DR734(DR588-DR229) | 4510 | 0.9 | 3.7 |
| DR735(DR588-DR230) | 3648 | 0.7 | 3.0 |
| DR736(DR588-DR231) | 3610 | 0.7 | 3.0 |
| DR737(DR588-DR232) | 3492 | 0.7 | 2.9 |
| DR738(DR588-DR233) | 4114 | 0.8 | 3.4 |
| DR739(DR588-DR234) | 3380 | 0.7 | 2.8 |
| DR740(DR589-DR229) | 4142 | 0.8 | 3.4 |
| DR741(DR589-DR230) | 3336 | 0.7 | 2.8 |
| DR742(DR589-DR231) | 3804 | 0.8 | 3.1 |
| DR743(DR589-DR232) | 3622 | 0.7 | 3.0 |
| DR744(DR589-DR233) | 4364 | 0.9 | 3.6 |
| DR745(DR589-DR234) | 3950 | 0.8 | 3.3 |
| DR746(DR590-DR229) | 3910 | 0.8 | 3.2 |
| DR747(DR590-DR230) | 3730 | 0.8 | 3.1 |
| DR748(DR590-DR231) | 4022 | 0.8 | 3.3 |
| DR749(DR590-DR232) | 3822 | 0.8 | 3.2 |
| DR750(DR590-DR233) | 3642 | 0.7 | 3.0 |
| DR751(DR590-DR234) | 4052 | 0.8 | 3.4 |
| medium | 4917 | 1.0 | 4.1 |
| IL2 | 120918 | 24.6 | 100.0 |

### Example 17—Evaluation of Activity of Anti-IL2Rβ/γ V_{H}H²s On Total PBMC

The anti-IL2Rβ/γ V_{H}H²s were evaluated for activity in non-activated PBMC. T cells and NK cells express IL2Rβ and IL2Rγ chains and are able to proliferate and produce IFN-γ in response to IL2 receptor signaling.

PBMC were isolated from LRS Chambers using the human Buffy Coat/LRSC PBMC Isolation Kit (Miltenyi). LRS Chambers were harvested in separation buffer (DPBS, 0.5% BSA, 2 mM EDTA) and mixed with sedimentation buffer and Red Blood Cell (RBC) removal antibodies and EDTA at a final concentration of 5 mM in 50 mL Centrifuge tubes. Tubes were centrifuged at 50×g for 3 minutes at room temperature. Supernatant with cells was collected and transferred to a new 50 mL centrifuge tube and separation buffer was added to 50 mL. Tubes were centrifuged at 300×g for 5 minutes at room temperature and supernatant discarded. Cell pellets were resuspended in 4 mL separation buffer and 2 mL Erythrocyte Depletion Microbeads and 1 mL Granulocyte Depletion Microbeads were added. Cells were incubated for 10 minutes at 2-8 degrees centigrade and PBMC were isolated on an AutoMacs Pro instrument (Miltenyi) using protocol ‘Cust 5’. PBMC were counted on a Vi-Cell XR instrument (Beckman). PBMC were isolated from 2 independent donors designated D24 and D26.

PBMC were cultured in growth medium consisting of Yssel's medium (Iscove's modified Dulbecco's Medium (ThermoFisher), 0.25% w/v percent human albumin (Sigma), 1 percent penicillin/streptomycin (ThermoFisher), 1 percent ITS-X Insulin, Transferrin, Selenium (Gibco), 30 mg/L Tansferrin (Roche), 2 mg/L Palmitic Acid (Sigma), 1 percent LA-OA-Albumin Linoleic Acid, Oleic Acid (Sigma), 1 percent human serum (Gemini) (Yssel et al (1984) J Immunol Methods 72: 219-227). Cells were resuspended at 2 million cells/mL in Yssel's medium. Anti-IL2Rβ/γ V_{H}H²s were diluted into 96-well plates (Falcon) to 30 nM in 100 μl Yssel's medium and 200 thousand total PBMC were added per well in 100 μl Yssel's medium. Controls included wells without anti-IL2Rβ/γ V_{H}H²s or human IL2 (media) and wells with human IL2 at a final concentration of 100 pM. Plates were transferred to a humidified incubator (ThermoFisher) and incubated at 37 degrees centigrade, 5 percent carbon dioxide for 6 days.

Plates were removed from the incubator and 100 μl of culture supernatant was harvested in to a 96 well flat bottom plate (Costar). To examine proliferation in these cultures, cells were harvested from wells that still contained viable cells upon visual inspection and were phenotyped for expression of CD3, CD56, CD4, CD8, and CD25 by direct immunofluorescence using fluorochrome conjugated mAbs on a Cytometer. Briefly, cells were collected from tissue culture wells and transferred to a 96 well round v-bottom plate and washed twice with FACS buffer (PBS+0.5% BSA). Pellets were resuspended in 100 μL 1/1000 diluted Viabillity dye EF506 (Biolegend) and incubated for 30 min at room temperature. Cells were washed twice with FACS buffer and resuspended in 50 μL blocking solution (FACS buffer with 2% normal mouse serum (Jackson Labs) and 10% Human FcX Block (Biolegend)) and incubated for 30 min at 4 degrees centigrade. Anti-CD3-Fitc, anti-CD56-AF700, anti-CD4-BV786, anti-CD8-APC-Cy7 and anti-CD25-BV421 were added to the cells in FACS buffer at the manufacturer's recommended concentration and cells were incubated for 30 min at 4 degrees centigrade. Cells were washed twice with FACS buffer, fixed in DPBS with 1% paraformaldehyde (Sigma) and analyzed on an Aurora Cytek Cytometer. Expression of markers was analyzed on single live cells using SpectroFlo v2.2.0.4 software.

Production of IFNγ in the culture supernatants was measured using the MSD IFNγ V-Plex kit (MSD K151QOD) according to manufacturer's instructions. Briefly, mAb precoated MSD IFNγ assay plates were washed 3 times with 150 μL Tris Wash Buffer and IFNγ standards were diluted in Diluent 2. Culture supernatants were diluted 1:5 with Diluent 2 and 50 μL of samples and standards were added to the IFNγ assay plates and incubated for 120 min on an orbital shaker (VWR Scientific) at 300 rpm at room temperature. Plates were washed 3 times with Tris Wash Buffer and 25 μL 1×detection antibody in Diluent 3 was added to each well. Plates were incubated for 60 min on an orbital shaker (VWR Scientific) at 300 rpm at room temperature. Plates were washed 3 times with Tris Wash Buffer and 150 μL 2× Read Buffer T was added to each well and Luminescence signal was read on a Mesoscale Quickplex SQ 120 instrument. Concentration of IFNγ in the supernatants were calculated based on the standard curve with MSD Discovery Workbench software.

To compare the effect of each anti-IL2Rβ/γ V_{H}H²s variant upon total PBMC IFNγ production, IFNγ concentrations for cells treated with anti-IL2Rβ/γ V_{H}H²s were compared to those obtained for control cells treated with growth medium alone to yield a fold induction and with control cells treated with human IL2 to yield an activity relative to the response to IL2 as a percentage. The data from these experiments is presented in Table 18 and Table 19.

TABLE 24
Activity of VHH dimers on PBMC IFN-
γ production and phenotype Donor 1.

| Anti-IL2Rβ/γ V_{H}H² | IFN-γ | T cells | NK cells |
|---|---|---|---|
| Construct | Production (LU) | (% CD3+) | (% CD56+) |
| DR632(DR229-DR214) | 135 | | |
| DR633(DR229-DR217) | 104 | | |
| DR634(DR229-DR583) | 16798 | 48.49 | 35.09 |
| DR635(DR229-DR584) | 186103 | 47.77 | 50.35 |
| DR636(DR229-DR585) | 2396 | 96.58 | 2.03 |
| DR637(DR229-DR586) | 212 | | |
| DR638(DR229-DR587) | 399282 | 41.98 | 55.82 |
| DR639(DR229-DR587) | 5160 | 50.32 | 47.22 |
| DR640(DR229-DR589) | 786 | | |
| DR641(DR229-DR590) | 94 | | |
| DR642(DR230-DR214) | 9540 | 42.72 | 55.39 |
| DR643(DR230-DR217) | 43321 | 46.6 | 51.51 |
| DR644(DR230-DR583) | 14668 | 62.91 | 33.69 |
| DR645(DR230-DR584) | 1462066 | 36.3 | 60.92 |
| DR646(DR230-DR585) | 258 | | |
| DR647(DR230-DR586) | 50480 | 46 | 51.99 |
| DR648(DR230-DR587) | 70954 | 42.48 | 55.24 |
| DR649(DR230-DR588) | 301 | | |
| DR650(DR230-DR589) | 85 | | |
| DR651(DR230-DR590) | 76 | | |
| DR652(DR231-DR214) | 167926 | 44.52 | 53.45 |
| DR653(DR231-DR217) | 633 | | |
| DR654(DR231-DR583) | 275 | | |
| DR655(DR231-DR584) | 18389 | 74.95 | 21.18 |
| DR656(DR231-DR585) | 552204 | 38.8 | 59.01 |
| DR657(DR231-DR586) | 87 | | |
| DR658(DR231-DR587) | 97 | | |
| DR659(DR231-DR588) | 83 | | |
| DR660(DR231-DR589) | 87 | | |
| DR661(DR231-DR590) | 82 | | |
| DR662(DR232-DR214) | 94 | | |
| DR663(DR232-DR217) | 283 | | |
| DR664(DR232-DR583) | 113 | | |
| DR665(DR232-DR584) | 79 | | |
| DR666(DR232-DR585) | 161 | | |
| DR667(DR232-DR586) | 239 | | |
| DR668(DR232-DR587) | 81 | | |
| DR669(DR232-DR588) | 247 | | |
| DR670(DR232-DR589) | 90 | | |
| DR671(DR232-DR590) | 526 | | |
| DR672(DR233-DR214) | 1087 | | |
| DR673(DR233-DR217) | 8738 | 41.99 | 55.81 |
| DR674(DR233-DR583) | 2842 | 38.7 | 59.4 |
| DR675(DR233-DR584) | 32239 | 44.85 | 53.27 |
| DR676(DR233-DR584) | 83 | | |
| DR677(DR233-DR586) | 298 | | |
| DR678(DR233-DR587) | 32319 | 46.56 | 51.48 |
| DR679(DR233-DR588) | 121672 | 36.13 | 61.88 |
| DR680(DR233-DR589) | 11020 | 46.18 | 52.08 |
| DR681(DR233-DR590) | 159 | | |
| DR682(DR234-DR214) | 3571 | 59.92 | 36.44 |
| DR683(DR234-DR217) | 94 | | |
| DR684(DR234-DR583) | 82 | | |
| DR685(DR234-DR584) | 516 | | |
| DR686(DR234-DR585) | 168 | | |
| DR687(DR234-DR586) | 85 | | |
| DR688(DR234-DR587) | 2812 | 39.99 | 57.85 |
| DR689(DR234-DR588) | 890 | | |
| DR690(DR234-DR589) | 246 | | |
| DR691(DR234-DR590) | 85 | | |
| DR692(DR214-DR229) | 88 | | |
| DR693(DR214-DR230) | 74 | | |
| DR694(DR214-DR231) | 76 | | |
| DR695(DR214-DR232) | 828 | | |
| DR696(DR214-DR233) | 111 | | |
| DR697(DR214-DR234) | 132 | | |
| DR698(DR217-DR229) | 78 | | |
| DR699(DR217-DR230) | 104 | | |
| DR700(DR217-DR231) | 96 | | |
| DR701(DR217-DR232) | 2570 | 57.26 | 40.3 |
| DR702(DR217-DR233) | 90 | | |
| DR703(DR217-DR234) | 162 | | |
| DR704(DR583-DR229) | 85 | | |
| DR705(DR583-DR230) | 83 | | |
| DR706(DR583-DR231) | 81 | | |
| DR707(DR583-DR232) | 80 | | |
| DR708(DR583-DR233) | 77 | | |
| DR709(DR583-DR234) | 82 | | |
| DR710(DR584-DR229) | 67 | | |
| DR711(DR584-DR230) | 181 | | |
| DR712(DR584-DR231) | 94 | | |
| DR713(DR584-DR232) | 117 | | |
| DR714(DR584-DR233) | 1932 | 92.95 | 3.96 |
| DR715(DR584-DR234) | 76 | | |
| DR716(DR585-DR229) | 5178 | 36.89 | 61.04 |
| DR717(DR585-DR230) | 14665 | 48.32 | 49.33 |
| DR718(DR585-DR231) | 79834 | 40.28 | 58.21 |
| DR719(DR585-DR232) | 20846 | 38.28 | 59.66 |
| DR720(DR585-DR233) | 734 | | |
| DR721(DR585-DR234) | 68144 | 40.48 | 57.36 |
| DR722(DR586-DR229) | 5762 | 45.21 | 53 |
| DR723(DR586-DR230) | 120 | | |
| DR724(DR586-DR231) | 12094 | 46.7 | 51.26 |
| DR725(DR586-DR232) | 170 | | |
| DR726(DR586-DR233) | 79 | | |
| DR727(DR586-DR234) | 79 | | |
| DR728(DR587-DR229) | 92 | | |
| DR729(DR587-DR230) | 78 | | |
| DR730(DR587-DR231) | 93 | | |
| DR731(DR587-DR232) | 90 | | |
| DR732(DR587-DR233) | 503 | | |
| DR733(DR587-DR234) | 81 | | |
| DR734(DR588-DR229) | 878 | | |
| DR735(DR588-DR230) | 630 | | |
| DR736(DR588-DR231) | 136028 | 41.38 | 56.82 |
| DR737(DR588-DR232) | 567 | | |
| DR738(DR588-DR233) | 224 | | |
| DR739(DR588-DR234) | 1454 | | |
| DR740(DR589-DR229) | 1272 | 78.22 | 17.89 |
| DR741(DR589-DR230) | 2216 | 79.76 | 17 |
| DR742(DR589-DR231) | 296 | | |
| DR743(DR589-DR232) | 98 | | |
| DR744(DR589-DR233) | 1099 | 85.55 | 10.34 |
| DR745(DR589-DR234) | 91 | | |
| DR746(DR590-DR229) | 868 | | |
| DR747(DR590-DR230) | 3385 | 41.18 | 56.19 |
| DR748(DR590-DR231) | 834 | | |
| DR749(DR590-DR232) | 90 | | |
| DR750(DR590-DR233) | 115 | | |
| DR751(DR590-DR234) | 98 | | |
| medium | 235 | | |
| IL2 | 338863 | 63.24 | 34.52 |

TABLE 25
Activity of VHH dimers on PBMC IFN-
γ production and phenotype Donor 2.

| Anti-IL2Rβ/γ V_{H}H² | IFN-γ | T cells | NK cells |
|---|---|---|---|
| Construct | Production (LU) | (% CD+) | (% CD56+) |
| DR632(DR229-DR214) | 117 | | |
| DR633(DR229-DR217) | 163 | | |
| DR634(DR229-DR583) | 5188 | 62.22 | 35.73 |
| DR635(DR229-DR584) | 11871 | 51.63 | 46.16 |
| DR636(DR229-DR585) | 127 | 97.46 | 0.3 |
| DR637(DR229-DR586) | 126 | | |
| DR638(DR229-DR587) | 40748 | 50.69 | 47.15 |
| DR639(DR229-DR587) | 2237 | 58.85 | 39.54 |
| DR640(DR229-DR589) | 3128 | | |
| DR641(DR229-DR590) | 74 | | |
| DR642(DR230-DR214) | 1043 | 58.95 | 39.26 |
| DR643(DR230-DR217) | 12027 | 50.19 | 47.74 |
| DR644(DR230-DR583) | 1395 | 77.48 | 20 |
| DR645(DR230-DR584) | 147962 | 56.88 | 41.48 |
| DR646(DR230-DR585) | 105 | | |
| DR647(DR230-DR586) | 24426 | 51.6 | 46.43 |
| DR648(DR230-DR587) | 16661 | 53.06 | 45.12 |
| DR649(DR230-DR588) | 107 | | |
| DR650(DR230-DR589) | 86 | | |
| DR651(DR230-DR590) | 101 | | |
| DR652(DR231-DR214) | 21818 | 49.74 | 47.99 |
| DR653(DR231-DR217) | 265 | | |
| DR654(DR231-DR583) | 81 | | |
| DR655(DR231-DR584) | 8336 | 81.11 | 15.93 |
| DR656(DR231-DR585) | 165479 | 55.4 | 42.95 |
| DR657(DR231-DR586) | 81 | | |
| DR658(DR231-DR587) | 101 | | |
| DR659(DR231-DR588) | 85 | | |
| DR660(DR231-DR589) | 85 | | |
| DR661(DR231-DR590) | 77 | | |
| DR662(DR232-DR214) | 77 | | |
| DR663(DR232-DR217) | 232 | | |
| DR664(DR232-DR583) | 101 | | |
| DR665(DR232-DR584) | 82 | | |
| DR666(DR232-DR585) | 87 | | |
| DR667(DR232-DR586) | 180 | | |
| DR668(DR232-DR587) | 89 | | |
| DR669(DR232-DR588) | 82 | | |
| DR670(DR232-DR589) | 84 | | |
| DR671(DR232-DR590) | 250 | | |
| DR672(DR233-DR214) | 323 | | |
| DR673(DR233-DR217) | 1977 | 65.3 | 32.57 |
| DR674(DR233-DR583) | 2565 | 61.54 | 36.29 |
| DR675(DR233-DR584) | 41300 | 42.86 | 55.03 |
| DR676(DR233-DR584) | 82 | | |
| DR677(DR233-DR586) | 242 | | |
| DR678(DR233-DR587) | 18981 | 53.53 | 44.54 |
| DR679(DR233-DR588) | 5117 | 57.75 | 40.55 |
| DR680(DR233-DR589) | 2370 | 63.81 | 34.23 |
| DR681(DR233-DR590) | 154 | | |
| DR682(DR234-DR214) | 1462 | 68.15 | 29.23 |
| DR683(DR234-DR217) | 145 | | |
| DR684(DR234-DR583) | 109 | | |
| DR685(DR234-DR584) | 840 | | |
| DR686(DR234-DR585) | 89 | | |
| DR687(DR234-DR586) | 79 | | |
| DR688(DR234-DR587) | 653 | 58.97 | 38.6 |
| DR689(DR234-DR588) | 332 | | |
| DR690(DR234-DR589) | 132 | | |
| DR691(DR234-DR590) | 73 | | |
| DR692(DR214-DR229) | 101 | | |
| DR693(DR214-DR230) | 88 | | |
| DR694(DR214-DR231) | 76 | | |
| DR695(DR214-DR232) | 78 | | |
| DR696(DR214-DR233) | 92 | | |
| DR697(DR214-DR234) | 117 | | |
| DR698(DR217-DR229) | 82 | | |
| DR699(DR217-DR230) | 173 | | |
| DR700(DR217-DR231) | 100 | | |
| DR701(DR217-DR232) | 1227 | 63.05 | 35.35 |
| DR702(DR217-DR233) | 96 | | |
| DR703(DR217-DR234) | 1143 | | |
| DR704(DR583-DR229) | 185 | | |
| DR705(DR583-DR230) | 81 | | |
| DR706(DR583-DR231) | 84 | | |
| DR707(DR583-DR232) | 96 | | |
| DR708(DR583-DR233) | 83 | | |
| DR709(DR583-DR234) | 84 | | |
| DR710(DR584-DR229) | 72 | | |
| DR711(DR584-DR230) | 75 | | |
| DR712(DR584-DR231) | 75 | | |
| DR713(DR584-DR232) | 91 | | |
| DR714(DR584-DR233) | 63 | 95.35 | 2.33 |
| DR715(DR584-DR234) | 228 | | |
| DR716(DR585-DR229) | 1273 | 55.37 | 42.69 |
| DR717(DR585-DR230) | 26060 | 45.59 | 51.88 |
| DR718(DR585-DR231) | 38220 | 46.94 | 50.75 |
| DR719(DR585-DR232) | 1850 | 88.89 | 6.13 |
| DR720(DR585-DR233) | 766 | | |
| DR721(DR585-DR234) | 24693 | 48.32 | 49.32 |
| DR722(DR586-DR229) | 5851 | 52.03 | 46.36 |
| DR723(DR586-DR230) | 173 | | |
| DR724(DR586-DR231) | 3693 | 57.36 | 40.62 |
| DR725(DR586-DR232) | 93 | | |
| DR726(DR586-DR233) | 85 | | |
| DR727(DR586-DR234) | 91 | | |
| DR728(DR587-DR229) | 340 | | |
| DR729(DR587-DR230) | 95 | | |
| DR730(DR587-DR231) | 237 | | |
| DR731(DR587-DR232) | 99 | | |
| DR732(DR587-DR233) | 113 | | |
| DR733(DR587-DR234) | 142 | | |
| DR734(DR588-DR229) | 4151 | | |
| DR735(DR588-DR230) | 625 | | |
| DR736(DR588-DR231) | 2595 | 55.41 | 42.05 |
| DR737(DR588-DR232) | 923 | | |
| DR738(DR588-DR233) | 104 | | |
| DR739(DR588-DR234) | 830 | | |
| DR740(DR589-DR229) | 873 | 86.55 | 6.04 |
| DR741(DR589-DR230) | 629 | 84.17 | 9.79 |
| DR742(DR589-DR231) | 198 | | |
| DR743(DR589-DR232) | 82 | | |
| DR744(DR589-DR233) | 625 | 82.76 | 8.27 |
| DR745(DR589-DR234) | 81 | | |
| DR746(DR590-DR229) | 212 | | |
| DR747(DR590-DR230) | 2272 | 68.05 | 29.44 |
| DR748(DR590-DR231) | 1050 | | |
| DR749(DR590-DR232) | 386 | | |
| DR750(DR590-DR233) | 138 | | |
| DR751(DR590-DR234) | 88 | | |
| medium | 226 | | |
| IL2 | 174981 | 64.09 | 33.48 |

LU: Luminescence Units.
n/a: not applicable

In this assay 54/120 anti-IL2Rβ/γ V_{H}H²s induced PBMC IFNγ production in D24 (>1Fold) and 48/120 anti-IL2Rβ/γ V_{H}H²s induced PBMC IFNγ production in D26 at distinct levels. Anti-IL2Rβ/γ V_{H}H²s that induced IFNγ production generally did so in both donors. One Anti-IL2Rβ/γ V_{H}H² in D24 PBMC induced IFNγ production above levels observed with IL2 at 100 pM.

Thirty six of the PBMC cultures that expanded and showed induction of IFNγ production were phenotyped to determine which cell populations were affected by IL2Rβ/γ V_{H}H²s. Percentages of T cells (CD3 positive) and percentages NK cells (CD56 positive) in each culture of PBMC D24 and D26 are shown in Table 26. In this assay 7/36 anti-IL2Rβ/γ V_{H}H²s in D24 PBMC and 15/36 anti-IL2Rβ/γ V_{H}H²s in D26 PBMC cultures showed a T cell biased expansion as defined by a greater than 60% of the population CD3 positive with some cultures as high as >90%. Conversely, 26/36 anti-IL2Rβ/γ V_{H}H²s in D24 PBMC and 11/36 anti-IL2Rβ/γ V_{H}H²s in D26 PBMC cultures showed an NK cell bias as defined by a greater than 45% of the population CD56 positive. In either T cell biased cultures and NK cell biased cultures anti-IL2Rβ/γ V_{H}H²s did not induce a preferential outgrowth of CD4 or CD8 positive T cells with ratio's CD4:CD8 around 2:1 (Table 29).

TABLE 26
IFNγ Production by Anti-IL2Rβ/γ V_{H}H² on Total PBMC Donor24

| | PBMC | Relative | Relative |
|---|---|---|---|
| | Cell | IFNγ | IFNγ |
| | IFNγ | Induction | Induction |
| Anti-IL2Rβ/γ V_{H}H² | Production | (fold | (% hIL2 |
| Construct | (pg/mL) | induction) | response) |
| DR632(DR229-DR214) | 4 | 0.4 | 0.0 |
| DR633(DR229-DR217) | 2 | 0.2 | 0.0 |
| DR634(DR229-DR583) | 1429 | 119.1 | 2.3 |
| DR635(DR229-DR584) | 17452 | 1454.3 | 28.0 |
| DR636(DR229-DR585) | 184 | 15.3 | 0.3 |
| DR637(DR229-DR586) | 10 | 0.8 | 0.0 |
| DR638(DR229-DR587) | 38635 | 3219.6 | 62.1 |
| DR639(DR229-DR587) | 415 | 34.6 | 0.7 |
| DR640(DR229-DR589) | 54 | 4.5 | 0.1 |
| DR641(DR229-DR590) | 1 | 0.1 | 0.0 |
| DR642(DR230-DR214) | 791 | 65.9 | 1.3 |
| DR643(DR230-DR217) | 3832 | 319.3 | 6.2 |
| DR644(DR230-DR583) | 1240 | 103.4 | 2.0 |
| DR645(DR230-DR584) | 150112 | 12509.3 | 241.1 |
| DR646(DR230-DR585) | 13 | 1.1 | 0.0 |
| DR647(DR230-DR586) | 4493 | 374.4 | 7.2 |
| DR648(DR230-DR587) | 6402 | 533.5 | 10.3 |
| DR649(DR230-DR588) | 17 | 1.4 | 0.0 |
| DR650(DR230-DR589) | 1 | 0.1 | 0.0 |
| DR651(DR230-DR590) | 0 | 0.0 | 0.0 |
| DR652(DR231-DR214) | 15682 | 1306.9 | 25.2 |
| DR653(DR231-DR217) | 42 | 3.5 | 0.1 |
| DR654(DR231-DR583) | 15 | 1.2 | 0.0 |
| DR655(DR231-DR584) | 1570 | 130.8 | 2.5 |
| DR656(DR231-DR585) | 54176 | 4514.7 | 87.0 |
| DR657(DR231-DR586) | 1 | 0.1 | 0.0 |
| DR658(DR231-DR587) | 2 | 0.1 | 0.0 |
| DR659(DR231-DR588) | 1 | 0.1 | 0.0 |
| DR660(DR231-DR589) | 1 | 0.1 | 0.0 |
| DR661(DR231-DR590) | 1 | 0.1 | 0.0 |
| DR662(DR232-DR214) | 1 | 0.1 | 0.0 |
| DR663(DR232-DR217) | 15 | 1.3 | 0.0 |
| DR664(DR232-DR583) | 3 | 0.2 | 0.0 |
| DR665(DR232-DR584) | 0 | 0.0 | 0.0 |
| DR666(DR232-DR585) | 6 | 0.5 | 0.0 |
| DR667(DR232-DR586) | 12 | 1.0 | 0.0 |
| DR668(DR232-DR587) | 1 | 0.0 | 0.0 |
| DR669(DR232-DR588) | 13 | 1.0 | 0.0 |
| DR670(DR232-DR589) | 1 | 0.1 | 0.0 |
| DR671(DR232-DR590) | 34 | 2.8 | 0.1 |
| DR672(DR233-DR214) | 78 | 6.5 | 0.1 |
| DR673(DR233-DR217) | 722 | 60.1 | 1.2 |
| DR674(DR233-DR583) | 221 | 18.4 | 0.4 |
| DR675(DR233-DR584) | 2818 | 234.8 | 4.5 |
| DR676(DR233-DR584) | 1 | 0.1 | 0.0 |
| DR677(DR233-DR586) | 16 | 1.4 | 0.0 |
| DR678(DR233-DR587) | 2825 | 235.4 | 4.5 |
| DR679(DR233-DR588) | 11217 | 934.8 | 18.0 |
| DR680(DR233-DR589) | 920 | 76.7 | 1.5 |
| DR681(DR233-DR590) | 6 | 0.5 | 0.0 |
| DR682(DR234-DR214) | 281 | 23.5 | 0.5 |
| DR683(DR234-DR217) | 1 | 0.1 | 0.0 |
| DR684(DR234-DR583) | 1 | 0.1 | 0.0 |
| DR685(DR234-DR584) | 33 | 2.8 | 0.1 |
| DR686(DR234-DR585) | 7 | 0.6 | 0.0 |
| DR687(DR234-DR586) | 1 | 0.1 | 0.0 |
| DR688(DR234-DR587) | 218 | 18.2 | 0.4 |
| DR689(DR234-DR588) | 62 | 5.2 | 0.1 |
| DR690(DR234-DR589) | 13 | 1.0 | 0.0 |
| DR691(DR234-DR590) | 1 | 0.1 | 0.0 |
| DR692(DR214-DR229) | 1 | 0.1 | 0.0 |
| DR693(DR214-DR230) | 0 | 0.0 | 0.0 |
| DR694(DR214-DR231) | 0 | 0.0 | 0.0 |
| DR695(DR214-DR232) | 57 | 4.8 | 0.1 |
| DR696(DR214-DR233) | 3 | 0.2 | 0.0 |
| DR697(DR214-DR234) | 4 | 0.3 | 0.0 |
| DR698(DR217-DR229) | 0 | 0.0 | 0.0 |
| DR699(DR217-DR230) | 2 | 0.2 | 0.0 |
| DR700(DR217-DR231) | 2 | 0.1 | 0.0 |
| DR701(DR217-DR232) | 198 | 16.5 | 0.3 |
| DR702(DR217-DR233) | 1 | 0.1 | 0.0 |
| DR703(DR217-DR234) | 6 | 0.5 | 0.0 |
| DR704(DR583-DR229) | 1 | 0.1 | 0.0 |
| DR705(DR583-DR230) | 1 | 0.1 | 0.0 |
| DR706(DR583-DR231) | 1 | 0.1 | 0.0 |
| DR707(DR583-DR232) | 1 | 0.1 | 0.0 |
| DR708(DR583-DR233) | 0 | 0.0 | 0.0 |
| DR709(DR583-DR234) | 1 | 0.1 | 0.0 |
| DR710(DR584-DR229) | 1 | 0.1 | 0.0 |
| DR711(DR584-DR230) | 10 | 0.8 | 0.0 |
| DR712(DR584-DR231) | 1 | 0.1 | 0.0 |
| DR713(DR584-DR232) | 3 | 0.3 | 0.0 |
| DR714(DR584-DR233) | 207 | 17.2 | 0.3 |
| DR715(DR584-DR234) | 1 | 0.0 | 0.0 |
| DR716(DR585-DR229) | 417 | 34.7 | 0.7 |
| DR717(DR585-DR230) | 1240 | 103.3 | 2.0 |
| DR718(DR585-DR231) | 12598 | 1049.9 | 20.2 |
| DR719(DR585-DR232) | 2891 | 241.0 | 4.6 |
| DR720(DR585-DR233) | 50 | 4.2 | 0.1 |
| DR721(DR585-DR234) | 6138 | 511.5 | 9.9 |
| DR722(DR586-DR229) | 702 | 58.5 | 1.1 |
| DR723(DR586-DR230) | 4 | 0.3 | 0.0 |
| DR724(DR586-DR231) | 1014 | 84.5 | 1.6 |
| DR725(DR586-DR232) | 7 | 0.6 | 0.0 |
| DR726(DR586-DR233) | 1 | 0.1 | 0.0 |
| DR727(DR586-DR234) | 1 | 0.1 | 0.0 |
| DR728(DR587-DR229) | 2 | 0.1 | 0.0 |
| DR729(DR587-DR230) | 1 | 0.1 | 0.0 |
| DR730(DR587-DR231) | 2 | 0.2 | 0.0 |
| DR731(DR587-DR232) | 2 | 0.1 | 0.0 |
| DR732(DR587-DR233) | 42 | 3.5 | 0.1 |
| DR733(DR587-DR234) | 1 | 0.1 | 0.0 |
| DR734(DR588-DR229) | 83 | 6.9 | 0.1 |
| DR735(DR588-DR230) | 56 | 4.7 | 0.1 |
| DR736(DR588-DR231) | 22589 | 1882.4 | 36.3 |
| DR737(DR588-DR232) | 49 | 4.1 | 0.1 |
| DR738(DR588-DR233) | 14 | 1.1 | 0.0 |
| DR739(DR588-DR234) | 150 | 12.5 | 0.2 |
| DR740(DR589-DR229) | 128 | 10.7 | 0.2 |
| DR741(DR589-DR230) | 242 | 20.1 | 0.4 |
| DR742(DR589-DR231) | 21 | 1.7 | 0.0 |
| DR743(DR589-DR232) | 2 | 0.2 | 0.0 |
| DR744(DR589-DR233) | 108 | 9.0 | 0.2 |
| DR745(DR589-DR234) | 2 | 0.1 | 0.0 |
| DR746(DR590-DR229) | 82 | 6.8 | 0.1 |
| DR747(DR590-DR230) | 389 | 32.4 | 0.6 |
| DR748(DR590-DR231) | 78 | 6.5 | 0.1 |
| DR749(DR590-DR232) | 2 | 0.1 | 0.0 |
| DR750(DR590-DR233) | 4 | 0.3 | 0.0 |
| DR751(DR590-DR234) | 2 | 0.2 | 0.0 |
| medium | 12 | 1.0 | 0.0 |
| IL2 | 62250 | 5187.5 | 100.0 |

TABLE 27
IFNγ Production by Anti-IL2Rβ/γ V_{H}H² on Total PBMC Donor 26

| | PBMC | Relative | Relative |
|---|---|---|---|
| | Cell | IFNγ | IFNγ |
| | IFNγ | Induction | Induction |
| Anti-IL2Rβ/γ V_{H}H² | Production | (fold | (% hIL2 |
| Construct | (pg/mL) | induction) | response) |
| DR632(DR229-DR214) | 4 | 0.3 | 0.0 |
| DR633(DR229-DR217) | 7 | 0.5 | 0.0 |
| DR634(DR229-DR583) | 476 | 34.0 | 1.6 |
| DR635(DR229-DR584) | 1133 | 81.0 | 3.7 |
| DR636(DR229-DR585) | 4 | 0.3 | 0.0 |
| DR637(DR229-DR586) | 4 | 0.3 | 0.0 |
| DR638(DR229-DR587) | 4099 | 292.8 | 13.4 |
| DR639(DR229-DR587) | 195 | 13.9 | 0.6 |
| DR640(DR229-DR589) | 279 | 19.9 | 0.9 |
| DR641(DR229-DR590) | 0 | 0.0 | 0.0 |
| DR642(DR230-DR214) | 85 | 6.1 | 0.3 |
| DR643(DR230-DR217) | 1149 | 82.1 | 3.8 |
| DR644(DR230-DR583) | 117 | 8.3 | 0.4 |
| DR645(DR230-DR584) | 15674 | 1119.6 | 51.2 |
| DR646(DR230-DR585) | 3 | 0.2 | 0.0 |
| DR647(DR230-DR586) | 2406 | 171.8 | 7.9 |
| DR648(DR230-DR587) | 1615 | 115.3 | 5.3 |
| DR649(DR230-DR588) | 3 | 0.2 | 0.0 |
| DR650(DR230-DR589) | 1 | 0.1 | 0.0 |
| DR651(DR230-DR590) | 2 | 0.2 | 0.0 |
| DR652(DR231-DR214) | 2139 | 152.8 | 7.0 |
| DR653(DR231-DR217) | 16 | 1.1 | 0.1 |
| DR654(DR231-DR583) | 1 | 0.0 | 0.0 |
| DR655(DR231-DR584) | 783 | 55.9 | 2.6 |
| DR656(DR231-DR585) | 17608 | 1257.7 | 57.5 |
| DR657(DR231-DR586) | 1 | 0.0 | 0.0 |
| DR658(DR231-DR587) | 2 | 0.2 | 0.0 |
| DR659(DR231-DR588) | 1 | 0.1 | 0.0 |
| DR660(DR231-DR589) | 1 | 0.1 | 0.0 |
| DR661(DR231-DR590) | 0 | 0.0 | 0.0 |
| DR662(DR232-DR214) | 0 | 0.0 | 0.0 |
| DR663(DR232-DR217) | 13 | 0.9 | 0.0 |
| DR664(DR232-DR583) | 2 | 0.2 | 0.0 |
| DR665(DR232-DR584) | 1 | 0.1 | 0.0 |
| DR666(DR232-DR585) | 1 | 0.1 | 0.0 |
| DR667(DR232-DR586) | 9 | 0.6 | 0.0 |
| DR668(DR232-DR587) | 1 | 0.1 | 0.0 |
| DR669(DR232-DR588) | 1 | 0.1 | 0.0 |
| DR670(DR232-DR589) | 1 | 0.1 | 0.0 |
| DR671(DR232-DR590) | 15 | 1.0 | 0.0 |
| DR672(DR233-DR214) | 21 | 1.5 | 0.1 |
| DR673(DR233-DR217) | 171 | 12.2 | 0.6 |
| DR674(DR233-DR583) | 226 | 16.1 | 0.7 |
| DR675(DR233-DR584) | 4157 | 296.9 | 13.6 |
| DR676(DR233-DR584) | 1 | 0.1 | 0.0 |
| DR677(DR233-DR586) | 14 | 1.0 | 0.0 |
| DR678(DR233-DR587) | 1850 | 132.1 | 6.0 |
| DR679(DR233-DR588) | 469 | 33.5 | 1.5 |
| DR680(DR233-DR589) | 207 | 14.8 | 0.7 |
| DR681(DR233-DR590) | 7 | 0.5 | 0.0 |
| DR682(DR234-DR214) | 123 | 8.8 | 0.4 |
| DR683(DR234-DR217) | 6 | 0.4 | 0.0 |
| DR684(DR234-DR583) | 3 | 0.2 | 0.0 |
| DR685(DR234-DR584) | 66 | 4.7 | 0.2 |
| DR686(DR234-DR585) | 1 | 0.1 | 0.0 |
| DR687(DR234-DR586) | 1 | 0.0 | 0.0 |
| DR688(DR234-DR587) | 50 | 3.6 | 0.2 |
| DR689(DR234-DR588) | 22 | 1.5 | 0.1 |
| DR690(DR234-DR589) | 5 | 0.3 | 0.0 |
| DR691(DR234-DR590) | 0 | 0.0 | 0.0 |
| DR692(DR214-DR229) | 2 | 0.2 | 0.0 |
| DR693(DR214-DR230) | 1 | 0.1 | 0.0 |
| DR694(DR214-DR231) | 0 | 0.0 | 0.0 |
| DR695(DR214-DR232) | 0 | 0.0 | 0.0 |
| DR696(DR214-DR233) | 2 | 0.1 | 0.0 |
| DR697(DR214-DR234) | 4 | 0.3 | 0.0 |
| DR698(DR217-DR229) | 1 | 0.1 | 0.0 |
| DR699(DR217-DR230) | 8 | 0.6 | 0.0 |
| DR700(DR217-DR231) | 2 | 0.2 | 0.0 |
| DR701(DR217-DR232) | 101 | 7.2 | 0.3 |
| DR702(DR217-DR233) | 2 | 0.1 | 0.0 |
| DR703(DR217-DR234) | 94 | 6.7 | 0.3 |
| DR704(DR583-DR229) | 9 | 0.7 | 0.0 |
| DR705(DR583-DR230) | 1 | 0.0 | 0.0 |
| DR706(DR583-DR231) | 1 | 0.1 | 0.0 |
| DR707(DR583-DR232) | 2 | 0.2 | 0.0 |
| DR708(DR583-DR233) | 1 | 0.1 | 0.0 |
| DR709(DR583-DR234) | 1 | 0.1 | 0.0 |
| DR710(DR584-DR229) | 0 | 0.0 | 0.0 |
| DR711(DR584-DR230) | 0 | 0.0 | 0.0 |
| DR712(DR584-DR231) | 0 | 0.0 | 0.0 |
| DR713(DR584-DR232) | 1 | 0.1 | 0.0 |
| DR714(DR584-DR233) | 0 | 0.1 | 0.0 |
| DR715(DR584-DR234) | 14 | 1.0 | 0.0 |
| DR716(DR585-DR229) | 106 | 7.5 | 0.3 |
| DR717(DR585-DR230) | 2574 | 183.8 | 8.4 |
| DR718(DR585-DR231) | 5621 | 401.5 | 18.4 |
| DR719(DR585-DR232) | 197 | 14.1 | 0.6 |
| DR720(DR585-DR233) | 60 | 4.3 | 0.2 |
| DR721(DR585-DR234) | 2433 | 173.8 | 7.9 |
| DR722(DR586-DR229) | 714 | 51.0 | 2.3 |
| DR723(DR586-DR230) | 9 | 0.6 | 0.0 |
| DR724(DR586-DR231) | 332 | 23.7 | 1.1 |
| DR725(DR586-DR232) | 2 | 0.1 | 0.0 |
| DR726(DR586-DR233) | 1 | 0.1 | 0.0 |
| DR727(DR586-DR234) | 2 | 0.1 | 0.0 |
| DR728(DR587-DR229) | 25 | 1.8 | 0.1 |
| DR729(DR587-DR230) | 2 | 0.1 | 0.0 |
| DR730(DR587-DR231) | 15 | 1.1 | 0.0 |
| DR731(DR587-DR232) | 2 | 0.2 | 0.0 |
| DR732(DR587-DR233) | 4 | 0.3 | 0.0 |
| DR733(DR587-DR234) | 6 | 0.4 | 0.0 |
| DR734(DR588-DR229) | 488 | 34.8 | 1.6 |
| DR735(DR588-DR230) | 55 | 3.9 | 0.2 |
| DR736(DR588-DR231) | 289 | 20.6 | 0.9 |
| DR737(DR588-DR232) | 88 | 6.3 | 0.3 |
| DR738(DR588-DR233) | 3 | 0.2 | 0.0 |
| DR739(DR588-DR234) | 78 | 5.6 | 0.3 |
| DR740(DR589-DR229) | 83 | 5.9 | 0.3 |
| DR741(DR589-DR230) | 56 | 4.0 | 0.2 |
| DR742(DR589-DR231) | 11 | 0.8 | 0.0 |
| DR743(DR589-DR232) | 1 | 0.1 | 0.0 |
| DR744(DR589-DR233) | 55 | 3.9 | 0.2 |
| DR745(DR589-DR234) | 1 | 0.1 | 0.0 |
| DR746(DR590-DR229) | 13 | 0.9 | 0.0 |
| DR747(DR590-DR230) | 249 | 17.8 | 0.8 |
| DR748(DR590-DR231) | 103 | 7.3 | 0.3 |
| DR749(DR590-DR232) | 30 | 2.1 | 0.1 |
| DR750(DR590-DR233) | 6 | 0.4 | 0.0 |
| DR751(DR590-DR234) | 1 | 0.1 | 0.0 |
| medium | 14 | 1.0 | 0.0 |
| IL2 | 30616 | 2186.9 | 100.0 |

TABLE 28
Cell populations in total PBMC cultured for
6 days with anti-IL2Rβ/γ V_{H}H²s

| | |
|---|---|
| D24 | D26 |

| | % T | % NK | % T | % NK |
|---|---|---|---|---|
| Construct | cells | Cells | cells | Cells |
| DR634(DR229-DR583) | 48.5 | 35.1 | 62.2 | 35.7 |
| DR635(DR229-DR584) | 47.8 | 50.4 | 51.6 | 46.2 |
| DR636(DR229-DR585) | 96.6 | 2.0 | 97.5 | 0.3 |
| DR638(DR229-DR587) | 42.0 | 55.8 | 50.7 | 47.2 |
| DR639(DR229-DR588) | 50.3 | 47.2 | 58.9 | 39.5 |
| DR642(DR230-DR214) | 42.7 | 55.4 | 59.0 | 39.3 |
| DR643(DR230-DR217) | 46.6 | 51.5 | 50.2 | 47.7 |
| DR644(DR230-DR583) | 62.9 | 33.7 | 77.5 | 20.0 |
| DR645(DR230-DR584) | 36.3 | 60.9 | 56.9 | 41.5 |
| DR647(DR230-DR586) | 46.0 | 52.0 | 51.6 | 46.4 |
| DR648(DR230-DR587) | 42.5 | 55.2 | 53.1 | 45.1 |
| DR652(DR231-DR214) | 44.5 | 53.5 | 49.7 | 48.0 |
| DR655(DR231-DR584) | 75.0 | 21.2 | 81.1 | 15.9 |
| DR656(DR231-DR585) | 38.8 | 59.0 | 55.4 | 43.0 |
| DR673(DR233-DR217) | 42.0 | 55.8 | 65.3 | 32.6 |
| DR674(DR233-DR583) | 38.7 | 59.4 | 61.5 | 36.3 |
| DR675(DR233-DR584) | 44.9 | 53.3 | 42.9 | 55.0 |
| DR678(DR233-DR587) | 46.6 | 51.5 | 53.5 | 44.5 |
| DR679(DR233-DR588) | 36.1 | 61.9 | 57.8 | 40.6 |
| DR680(DR233-DR589) | 46.2 | 52.1 | 63.8 | 34.2 |
| DR682(DR234-DR214) | 59.9 | 36.4 | 68.2 | 29.2 |
| DR688(DR234-DR587) | 40.0 | 57.9 | 59.0 | 38.6 |
| DR701(DR217-DR232) | 57.3 | 40.3 | 63.1 | 35.4 |
| DR714(DR584-DR233) | 93.0 | 4.0 | 95.4 | 2.3 |
| DR716(DR585-DR229) | 36.9 | 61.0 | 55.4 | 42.7 |
| DR717(DR585-DR230) | 48.3 | 49.3 | 45.6 | 51.9 |
| DR718(DR585-DR231) | 40.3 | 58.2 | 46.9 | 50.8 |
| DR719(DR585-DR232) | 38.3 | 59.7 | 88.9 | 6.1 |
| DR721(DR585-DR234) | 40.5 | 57.4 | 48.3 | 49.3 |
| DR722(DR586-DR229) | 45.2 | 53.0 | 52.0 | 46.4 |
| DR724(DR586-DR231) | 46.7 | 51.3 | 57.4 | 40.6 |
| DR736(DR588-DR231) | 41.4 | 56.8 | 55.4 | 42.1 |
| DR740(DR589-DR229) | 78.2 | 17.9 | 86.6 | 6.0 |
| DR741(DR589-DR230) | 79.8 | 17.0 | 84.2 | 9.8 |
| DR744(DR589-DR233) | 85.6 | 10.3 | 82.8 | 8.3 |
| DR747(DR590-DR230) | 41.2 | 56.2 | 68.1 | 29.4 |
| IL2 | 63.2 | 34.5 | 64.1 | 33.5 |

TABLE 29
T Cell populations in total PBMC cultured for
6 days with anti-IL2Rβ/γ V_{H}H²s

| | D24 T Cells | | D26 T Cells | |
|---|---|---|---|---|
| Construct | % CD4 | % CD8 | % CD4 | % CD8 |
| DR634(DR229-DR583) | 19.8 | 45.5 | 51.6 | 24.5 |
| DR635(DR229-DR584) | 42.4 | 18.2 | 47.6 | 26.7 |
| DR636(DR229-DR585) | 16.0 | 7.0 | 53.6 | 23.6 |
| DR638(DR229-DR587) | 42.3 | 16.0 | 48.9 | 27.5 |
| DR639(DR229-DR588) | 46.5 | 19.6 | 52.4 | 25.1 |
| DR642(DR230-DR214) | 45.7 | 19.1 | 45.0 | 33.9 |
| DR643(DR230-DR217) | 52.0 | 17.6 | 50.1 | 25.4 |
| DR644(DR230-DR583) | 46.7 | 16.1 | 54.8 | 20.2 |
| DR645(DR230-DR584) | 42.4 | 24.4 | 42.6 | 34.0 |
| DR647(DR230-DR586) | 49.0 | 20.6 | 52.5 | 24.8 |
| DR648(DR230-DR587) | 45.6 | 23.7 | 45.2 | 31.4 |
| DR652(DR231-DR214) | 43.9 | 26.0 | 40.8 | 27.1 |
| DR655(DR231-DR584) | 46.1 | 16.2 | 50.7 | 20.1 |
| DR656(DR231-DR585) | 46.9 | 14.7 | 44.1 | 26.0 |
| DR673(DR233-DR217) | 46.3 | 17.5 | 52.8 | 22.7 |
| DR674(DR233-DR583) | 42.7 | 24.5 | 46.3 | 27.1 |
| DR675(DR233-DR584) | 43.9 | 19.5 | 46.6 | 32.1 |
| DR678(DR233-DR587) | 44.2 | 18.0 | 48.0 | 29.2 |
| DR679(DR233-DR588) | 43.3 | 18.1 | 42.8 | 28.1 |
| DR680(DR233-DR589) | 50.4 | 17.3 | 52.2 | 27.0 |
| DR682(DR234-DR214) | 40.0 | 22.3 | 50.1 | 27.1 |
| DR688(DR234-DR587) | 38.2 | 22.6 | 41.8 | 31.9 |
| DR701(DR217-DR232) | 49.5 | 19.7 | 49.1 | 27.8 |
| DR714(DR584-DR233) | 44.2 | 14.8 | 53.4 | 22.9 |
| DR716(DR585-DR229) | 41.5 | 19.5 | 46.2 | 25.0 |
| DR717(DR585-DR230) | 46.7 | 19.0 | 45.9 | 27.6 |
| DR718(DR585-DR231) | 45.7 | 24.1 | 47.1 | 31.2 |
| DR719(DR585-DR232) | 48.7 | 16.4 | 51.3 | 21.1 |
| DR721(DR585-DR234) | 46.1 | 21.4 | 40.4 | 30.0 |
| DR722(DR586-DR229) | 50.1 | 19.7 | 46.4 | 31.7 |
| DR724(DR586-DR231) | 42.5 | 21.6 | 44.2 | 31.0 |
| DR736(DR588-DR231) | 51.3 | 14.6 | 40.9 | 31.4 |
| DR740(DR589-DR229) | 35.1 | 20.3 | 37.5 | 28.7 |
| DR741(DR589-DR230) | 39.5 | 20.2 | 39.8 | 22.2 |
| DR744(DR589-DR233) | 32.3 | 20.7 | 39.7 | 25.1 |
| DR747(DR590-DR230) | 41.0 | 20.1 | 40.4 | 26.0 |
| IL2 | 41.1 | 14.4 | 41.7 | 13.1 |

### Example 18 Generation of PEGylated DR638 and DR736

DNA coding for DR638 and DR736 dual VHH fused to a 6x-histidine tag (SEQ ID NO: 127) was transfected into suspension Expi293 cells using Expi-Fectamine according to manufacturer's instructions (ThermoFisher). After six days of cell culture, supernatant was harvested and sterile-filtered. Purification was achieved via affinity chromatography on Ni-Excel resin (Cytiva) after addition of 5 mM imidazole in the protein supe. Wash and elution were carried out in PBS buffer supplemented with 30 and 250 mM imidazole, respectively.

pH of the eluates was reduced to ˜6.3 via addition of glacial acetate (100× dilution). PEGylation was then triggered by addition of 40 KDa, branched polyethylene glycol (PEG, Nof Corporation) in a 20× weight to weight ratio over protein, followed by addition of 10 mM Sodium Cyanoborohydride. The reaction was incubated for 20 h at room temperature. Isolation of mono-pegylated, dual VHH was achieved by cationic exchange chromatography over an SP FF, 20 mL column (Cytiva). The reaction was diluted three-fold in water to reduce the conductivity under 7.5 mS/cm and then loaded on the column. A 10-500 mM linear gradient of NaCl in 20 mM sodium acetate, pH 5, 2% sucrose, 1 mM EDTA was used to elute and separate protein conjugated to 2, 1 and 0 PEG molecules. Analytical SEC was used to determine the purity of the mono PEGylated final products: 88.2% DR638-PEG40K2a and 87.4% DR736-PEG40K2a. Integrity, identity, in vitro activity and thermo-stability of the molecules were validated via SDS-PAGE, LCMS, Biacore and Nanotemper Panta assays.

### Example 19. Evaluation of PEGylated and Non-PEGylated DR638 and DR736 By Surface Plasmon Resonance

The affinity of the non-PEGylated and the PEGylated forms of DR638 and DR736 prepared in substantial accordance with the foregoing were evaluated by surface plasmon resonance with the VHH dimer molecule in the mobile phase and an immobilized Fc fused hCD122 and hCD132 monovalent and bivalent hCD122/hCD132 Fc construct. DR638 exhibited an affinity for hCD122 of less than 0.5 nM and for CD132 of approximately 0.5 nM with an avidity for the hCD/122/CD132 dimeric receptor of less than 0.5 nM. When PEGylated with the 40 kD PEG molecule there was a reduction in affinity for the IL2 receptor subunits, resulting an affinity for hCD122 of approximately 1.1 nM and for hCD132 of approximately 16.3 nM. With respect to DR736, affinity for CD122 of approximately 21 nM and for CD132 of approximately 6.5 nM with an avidity for the hCD122/hCD132 dimeric receptor of approximately 0.2 nM. Similarly, with respect to DR 736, the 40 kD PEG molecule resulted in a reduction in affinity for the hIL2 receptor subunits that was not detectable under the conditions evaluated.

### Example 20. Evaluation of PEGylated and Non-PEGylated DR638 and DR736 On Mouse Splenocytes

The DR638 and DR736 constructs were evaluated for activity in both PEGylated and non-PEGylated forms on mouse splenocytes. Mouse splenocytes were cultured with mouse anti-CD3/CD28 beads and IL-2 for 3 days, stimulated for 48 hours, and the activation evaluated using the CellTiter-Glo assay as described above. The component VHHs of each dimeric construct were generated by immunization with the human receptor ECD resulting in a low activity of the DR638 molecule on mouse splenocytes relative to PEG-neoleukin and human IL2 with the PEGylated form exhibiting even lower activity. With respect to DR736 there was essentially no detectable cross reactivity in either the PEGylated or non-PEGylated forms. A three-day culture of activated C57/BH mouse splenocytes neither the PEGylated or non-PEGylated forms of DR638 or DR736 demonstrated no detectable proliferation nor significant STAT5 signaling or IFN-gamma production (evaluated in substantial accordance with the foregoing assay methodologies) confirming that neither DR638 nor DR736 cross-react to a significant degree with the mouse CD122/CD132 intermediate affinity receptor.

### Example 21. Evaluation of PEGylated and Non-PEGylated DR638 and DR736 In Vivo

Since neither DR638 nor DR736 cross react with the murine CD122, CD132 or intermediate affinity murine IL2 receptor to a significant degree, to evaluate the effects of DR638 and DR736 constructs in vivo, these molecules were evaluated in double transgenic mouse model that expressed the human CD122 and CD132 receptors. hIL2Rβ/hIL2Rγ double transgenic mice (dtg) were generated and obtained from Biocytogen corporation (Boston). The mice were generated by replacement of exons 2-8 of mouse CD122 gene that encode the extracellular domain of mCD122 with hCD122 exons 2-8 and exons 1-8 of mouse CD132 gene that encode the full-length protein mCD132 were replaced by hCD132 exons 2-8. Mice are hemizygous and express both human and mouse IL2Rβ and IL2Rγ. The individual mouse lines were crossed to generate chimeric CD122/CD132 double transgenic mice. Wildtype C57/Bl 6 mice expressing only mouse IL2Rβ and IL2Rγ were used as control. As a comparator, the neoleukin (“Neo-2/1”5) molecule was employed. Neo-2/15, as described in Silva, et al. (2019) Nature 565:186 is a synthetic IL2/IL15 molecule that specifically binds to the CD122 and CD132 receptors but has no binding site for CD25 such that it is a ligand only for the dimeric intermediate affinity IL2 receptor. The 40 kd PEGylated neoleukin molecule (neoleukin-PEG) was generated in substantial accordance with the foregoing PEGylation protocol. The IL2Rβ/γ VHH dimer DR638 has approximately 10-100 fold higher activity relative to the IL2Rβ/γ VHH dimer DR736 in the biological assays described in examples 1-10.

The 40Kd PEGylated forms of DR638 (DR638peg) DR 736 (DR736peg) were dosed in hIL2Rβ/hIL2Rγ dtg mice as follows. DR638peg was dosed subcutaneously once at 250 mg/mouse, 40 mg/mouse, and 10 mg/mouse, and twice per week for 2 weeks for a total of 4 doses at 1 mg/mouse and 0.1 mg/mouse. DR736peg was dosed subcutaneously twice per week for 2 weeks for a total of 4 doses at 200 mg/mouse. Neoleukin-peg was dosed once at 3 mg/mouse and once per week for 2 weeks for a total of 2 doses at 1 mg/mouse. Wildtype C57/B16 mice were dosed with anti-IL2Rβ/γ V_{H}H²'s DR638peg twice per week for 2 weeks for a total of 4 doses at 10 mg/mouse and once per week for 2 weeks for a total of 2 doses at 1 mg/mouse. Each dose group consisted of 4-5 mice per group. Mice were bled on days 7 and 14 and the surviving mice were sacrificed on day 14.

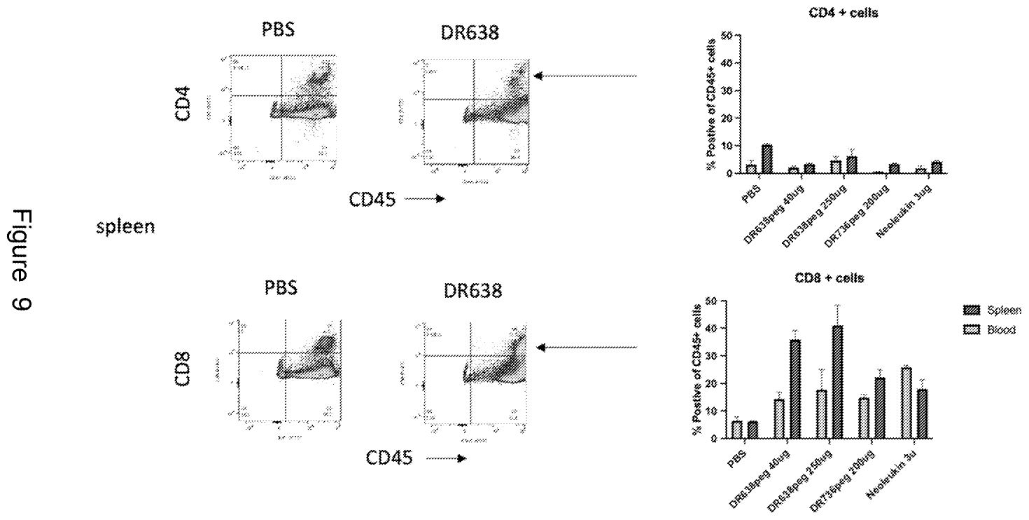

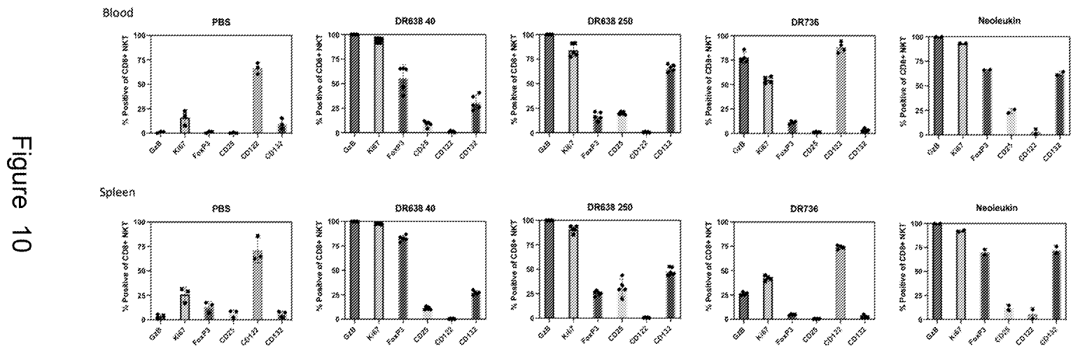

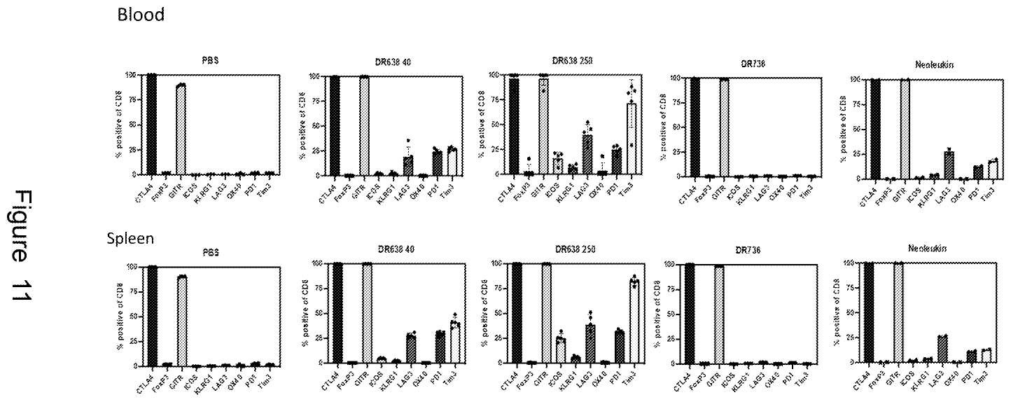

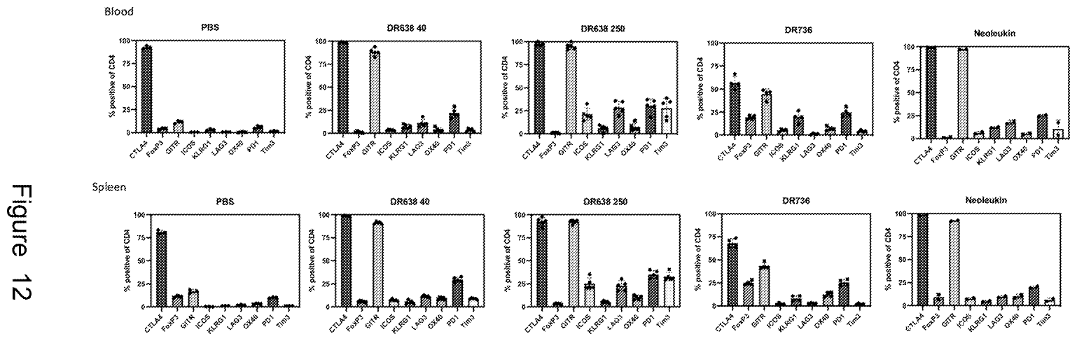

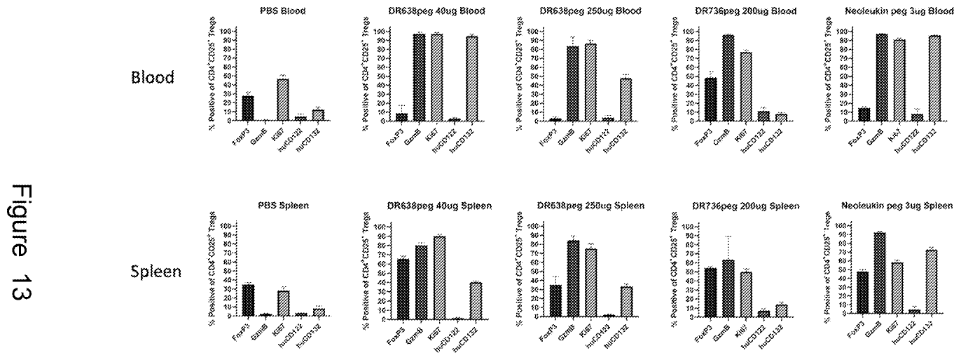

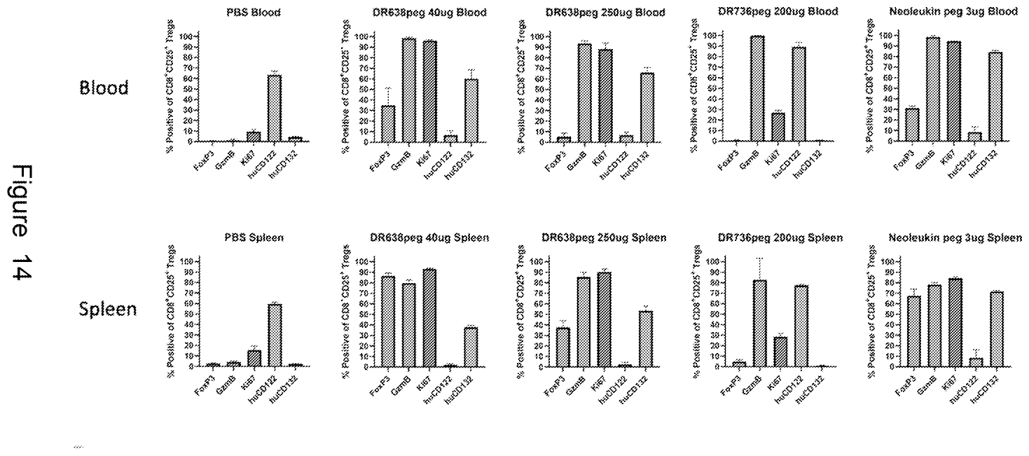

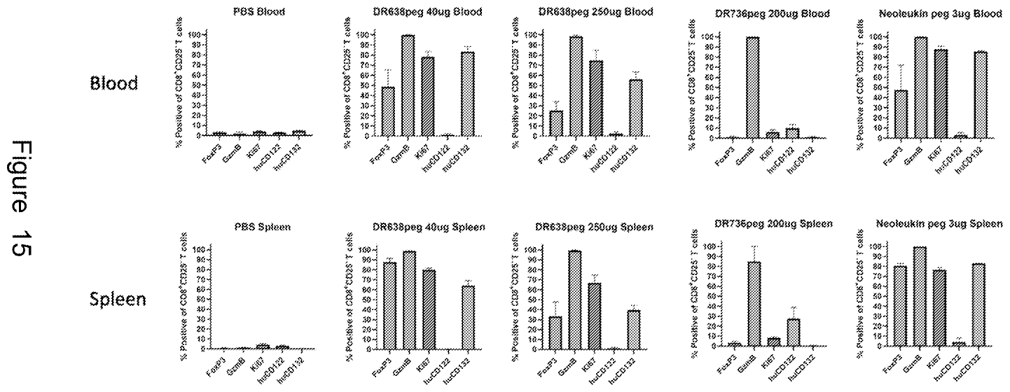

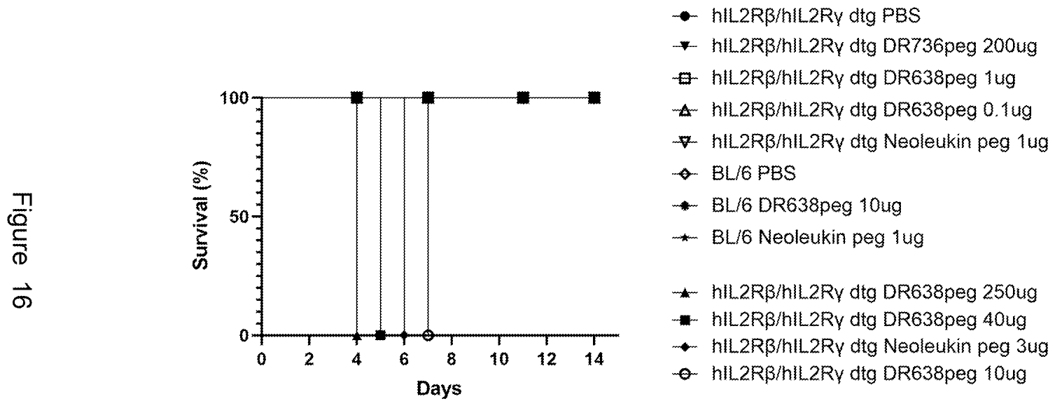

Blood and spleens were harvested from hIL2Rβ/hIL2Rγ dtg mice and wildtype C57/B16 mice when sacrificed either when moribund or after completion of treatment for FACS analyses. Blood and spleen cell suspensions were phenotyped for CD3, CD4, CD8a, CD11b, CD19, CD90.2, CD45, CD25, CD122, CD132, hCD122, hCD132, NK1.1, TCRgd, K167, FoxP3 and Granzyme B expression. Cells were washed in PBS and incubated in PBS with 1/3000 dilution of fix viability dye ZombiNIR for 15 min on ice and quenched in FACS buffer consisting of PBS, 2 mM EDTA, 0.5% BSA. Cells were washed and fixed with BD lyse fix buffer for 10 min, washed in FACS buffer twice and permeabilized with chilled BD Perm Buffer III for 30 minutes on ice. Cells were washed twice in FACS buffer, resuspended in 1:200 Fc-Block (#1) and stained with CD3 APC Fire 810, CD4 BV480, CD8a BV570, CD11b Pacific Blue, CD19 BV605, CD90.2 BV510, CD45 AF532, CD25 AF647, CD122 PE-Cy7, CD132 BV421, hCD122 PE, hCD132 APC, NK1.1BV711, TCRgd BV650, K167 BV786, FoxP3PerCP-eFluor 710 and Granzyme B FITC antibody-conjugates (All Biolegend) according to manufacturer's recommendation for 30 min on ice. Cells were washed, fixed with 0.1% paraformaldehyde and analyzed on an Aurora Flow Cytometer (Cytek) with SpectroFlo software. Immunohistochemistry evaluations of major orgrans (spleen, liver lung and intestine) was performed. The data generated in this study with respect to the various cell populations and functional markers from both blood and spleen are presented in FIGS. 9-15 of the attached drawings. The data from these experiments is presented in the following tables.

Cell populations in blood of hIL2Rβ/hIL2Rγ dtg and WT BL/6 mice
following administration of anti-IL2Rβ/γ V_{H}H² DR638peg, DR736peg or NeoleukinPEG.

| | | | | | | | Avg % | | Avg % | Avg % |
|---|---|---|---|---|---|---|---|---|---|---|
| | Mouse | Treatment | | Avg % | Avg % | Avg % | NK1.1+ | Avg % | CD4+ | CD8+ |
| Sex | Model | Group/Dose | Day | B cells | Mo/Gran | NK cells | T cells | T cells | T cells | T cells |
| M | dtg | PBS | 14 | 64.06 | 15.59 | 1.27 | 0.65 | 9.8 | 2.52 | 6.11 |
| M | dtg | DR638peg | 4 | 1.99 | 23.55 | 6.29 | 45.77 | 7.5 | 2.28 | 12.64 |
| | | 40 ug | | | | | | | | |
| M | dtg | DR638peg | 4 | 2.37 | 31.64 | 3.87 | 41.87 | 7.39 | 1.42 | 11.44 |
| | | 250 ug | | | | | | | | |
| M | dtg | DR736peg | 14 | 9.48 | 32.55 | 21.58 | 16.66 | 8.09 | 0.62 | 11.02 |
| | | 200 ug | | | | | | | | |
| M | dtg | Neoleukin | 4 | 3.15 | 29.72 | 4.26 | 36.8 | 7.45 | 2.33 | 16.29 |
| | | peg 3 ug | | | | | | | | |
| F | dtg | PBS | 14 | 48.78 | 6.25 | 1.2 | 11.25 | 18.11 | 6.95 | 7.35 |
| F | dtg | DR638peg | 7 | 7.2 | 23.7 | 24.76 | 35.82 | 4.51 | 1.79 | 1.92 |
| | | 10 ug | | | | | | | | |
| F | dtg | DR638peg | 14 | 29.69 | 13.69 | 3.14 | 22.75 | 14.86 | 7.51 | 8.29 |
| | | 1 ug | | | | | | | | |
| F | dtg | DR638peg | 14 | 47.1 | 6.32 | 1.65 | 8.71 | 19.14 | 8.81 | 8.16 |
| | | 0.1 ug | | | | | | | | |
| F | dtg | Neoleukin | 14 | 38.62 | 6.29 | 0.88 | 9.46 | 25.64 | 9.58 | 9.43 |
| | | peg 1 ug | | | | | | | | |
| F | WT | PBS | 14 | 47.65 | 5.13 | 1.07 | 8.03 | 22.67 | 8.02 | 7.25 |
| | BL/6 | | | | | | | | | |
| F | WT | DR638peg | 14 | 52.05 | 5.71 | 0.86 | 8.42 | 21.57 | 5.58 | 5.61 |
| | BL/6 | 10 ug | | | | | | | | |
| F | WT | Neoleukin | 14 | 42.47 | 9 | 1.66 | 14.43 | 19.3 | 5.7 | 7.27 |
| | BL/6 | peg 1 ug | | | | | | | | |

M = Male,
F = Female
Dtg = hIL2Rβ/hIL2Rγ double transgenic mouse;
WT BL/6 = wild type BL/6 mouse
Avg %: Average values of 4-5 individual mice per group.

Cell populations in spleen of hIL2Rβ/hIL2Rγ dtg and WT BL/6 mice
following administration of anti-IL2Rβ/γ V_{H}H² DR638peg, DR736peg, or Neoleukin.

| | | | | | | | Avg % | | Avg % | Avg % |
|---|---|---|---|---|---|---|---|---|---|---|
| | Mouse | Treatment | | Avg % | Avg % | Avg % | NK1.1+ | Avg % | CD4+ | CD8+ |
| Sex | Model | Group | Day | B cells | Mo/Gran | NK cells | T cells | T cells | T cells | T cells |
| M | dtg | PBS | 14 | 64.98 | 4.03 | 0.83 | 0.92 | 14.55 | 8.6 | 6.08 |
| M | dtg | DR638peg | 4 | 17.27 | 7.8 | 1.77 | 32.22 | 15.83 | 2.99 | 22.12 |
| | | 40 ug | | | | | | | | |
| M | dtg | DR638peg | 4 | 20.91 | 9.87 | 1.06 | 26.85 | 14.4 | 2.46 | 24.46 |
| | | 250 ug | | | | | | | | |
| M | dtg | DR736peg | 14 | 25.87 | 13.88 | 8.6 | 10.97 | 18.69 | 2.38 | 19.6 |
| | | 200 ug | | | | | | | | |
| M | dtg | Neoleukin | 4 | 51.55 | 0.36 | 0.7 | 13.34 | 14.63 | 4.33 | 15.09 |
| | | peg 3 ug | | | | | | | | |
| F | dtg | PBS | 14 | 49.26 | 2.99 | 1.68 | 10.64 | 21.94 | 8.37 | 4.91 |
| F | dtg | DR638peg | 7 | 18.2 | 7.11 | 7.9 | 59.94 | 2.56 | 1.67 | 0.81 |
| | | 10 ug | | | | | | | | |
| F | dtg | DR638peg | 14 | 45.16 | 5.23 | 1.73 | 14.65 | 18.66 | 8.69 | 5.67 |
| | | 1 ug | | | | | | | | |
| F | dtg | DR638peg | 14 | 52.05 | 2.51 | 1.46 | 9.28 | 19.03 | 9.85 | 5.59 |
| | | 0.1 ug | | | | | | | | |
| F | dtg | Neoleukin | 14 | 46.65 | 3.65 | 0.74 | 7.71 | 25.08 | 9.82 | 6.02 |
| | | peg 1 ug | | | | | | | | |
| F | WT | PBS | 14 | 31.67 | 2.1 | 3.63 | 35.94 | 14.91 | 6.7 | 4.68 |
| | BL/6 | | | | | | | | | |
| F | WT | DR638peg | 14 | 50.73 | 1.87 | 2.71 | 7.28 | 21.97 | 9.14 | 6.06 |
| | BL/6 | 10 ug | | | | | | | | |
| F | WT | Neoleukin | 14 | 52.07 | 2.63 | 1.17 | 7.59 | 21.33 | 8.75 | 6.16 |
| | BL/6 | peg 1 ug | | | | | | | | |

M = Male,
F = Female
Dtg = hIL2Rβ/hIL2Rγ double transgenic mouse;
WT BL/6 = wild type BL/6 mouse
Avg %: Average values of 4-5 individual mice per group.

hIL2Rβ/hIL2Rγ dtg mice dosed with DR638peg at 250 mg/mouse, 40 mg/mouse, and 10 mg/mouse were moribund on days 4, 5, and 7 respectively following dosing and had to be sacrificed. hIL2Rβ/hIL2Rγ dtg mice dosed with Neoleukin-peg at 3 mg/mouse were moribund on day 6 following dosing and had to be sacrificed. hIL2Rβ/hIL2Rγ mice and wildtype C57/B16 mice dosed with DR638peg at 1 mg/mouse, Neoleukin-peg at 1 mg/mouse and PBS controls as well as hIL2R43/hIL2Rγ mice dosed with DR638peg at 0.1 mg/mouse and DR736peg at 200 mg/mouse survived the dosing regimen. These results indicate that high doses of DR638peg affect physiology and induce lethality in hIL2Rβ/hIL2Rγ dtg mice similar to the hIL2Rβ/hIL2Rγ biased IL-2 agonist Neoleukin. The induced lethality of DR638peg is dose dependent, and specific for mice that express hIL2Rβ/hIL2Rγ as wildtype C57/B16 mice survived treatment.

Administration of DR638peg and DR736peg and Neoleukin induced significant changes in cell populations in blood and spleen in vivo in hIL2Rβ/hIL2Rγ dtg mice expressing hIL2Rβ and hIL2Rγ. The DR638peg induced a large population of NK1.1 positive T cells in blood and spleen in a dose dependent manner, similar to Neoleukin. Most of these cells were CD8 positive. The DR638peg and Neoleukin also increased the population of NK1.1 negative T cells. Concurrently the percentages of B cells were greatly reduced and there was a modest increase in myeloid/granulocyte populations. The DR736peg showed also modest increases in NK1.1 positive T cells, NK1.1 negative CD T cells and myeloid/granulocyte populations and also in NK cells in hIL2Rβ/hIL2Rγ dtg mice. The DR638peg had no effect on any of the subpopulations in wildtype C57Bl/6 mice that do not express hIL2Rβ and hIL2Rγ. Neoleukin, which can cross species did slightly increase the population of NK1.1 positive T cells in blood of wildtype C57Bl/6 mice. In addition, DR638peg and DR736 and Neoleukin induced proliferation and activation, of CD4 positive T cells, CD8 positive T cells and NK1.1 positive T cells as evidenced by increased expression of the proliferation market Ki67 and activation marker Granzyme B. In these experiments the anti-IL2Rβ/γ V_{H}H²'s DR638 and DR736 induced T cell activation, proliferation and changes in blood and spleen subpopulations in vivo in mice expressing hIL2Rβ and hIL2Rγ. Similar to Neoleukin, DR638 had a maximum tolerable dose.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference.

### TABLES

TABLE 30
anti-human IL2Rb sdAb CDRs

| | CDR 1 | | | | | |
|---|---|---|---|---|---|---|
| | (Chothia/ | | | | | |
| Name of VHH | Kabat) | SEQ ID NO: | CDR 2 | SEQ ID NO: | CDR 3 | SEQ ID NO: |
| hIL2Rb VHH- | YTYDTSDMS | 426 | DIDSGDWAA | 427 | SYWKWGKL | 428 |
| 1 | | | YADAVKG | | NNF | |
| hIL2Rb VHH- | FTFSNYWIF | 429 | TSNTGGDTT | 430 | GRCARSG | 431 |
| 2 | | | KYADSVKG | | | |
| hIL2Rb VHH- | FRFSNYGMS | 432 | YINGDGSRT | 433 | GLSRDGWSL | 434 |
| 3 | | | HYADSVKG | | SAAS | |
| hIL2Rb_VHH- | YTTYSFNYM | 435 | VIYTGGGSTL | 436 | DDQRFASPL | 437 |
| 4 | G | | YADSVKG | | YAYFGY | |
| hIL2Rb VHH- | DTKSIRCMG | 1438 | AIDREGFAT | 439 | QNMCRVVR | 440 |
| 5 | | | YADSVYD | | GAMTGVDY | |
| hIL2Rb VHH- | YTASRYCMA | 441 | AIHPGGGTT | 442 | GSLWVPFGD | 443 |
| 6 | | | YYADSVKG | | RCAANY | |
| hIL2Rb VHH- | YEYCRIHMT | 444 | SIGSDGRKTY | 445 | EYLYGLGCP | 446 |
| 7 | | | ANSVTG | | DGSAY | |
| hIL2Rb_VHH- | YTYSSYYCM | 447 | AIDSDGSTSY | 448 | SYEVVDCYP | 449 |
| 8 | G | | ADSVKG | | SGYGQDY | |

TABLE 31
murine IL2Rb sdAb CDRs

| | CDR1 | SEQ ID | | | | |
|---|---|---|---|---|---|---|
| Name | (Chothia/Kabat) | NO: | CDR2 | SEQ ID NO: | CDR3 | SEQ ID NO: |
| [symbol] | [symbol] | 450 | [symbol] | 451 | [symbol] | 452 |
| [symbol] | [symbol] | 453 | [symbol] | 454 | [symbol] | 455 |
| | | | | | [symbol] | |
| [symbol] | [symbol] | 456 | [symbol] | 457 | [symbol] | 458 |
| [symbol] | [symbol] | 459 | [symbol] | 460 | [symbol] | 461 |
| [symbol] | [symbol] | 462 | [symbol] | 463 | [symbol] | 464 |
| [symbol] | [symbol] | 465 | [symbol] | 466 | [symbol] | 467 |
| [symbol] | [symbol] | 468 | [symbol] | 469 | [symbol] | 470 |
| [symbol] | [symbol] | 471 | [symbol] | 472 | [symbol] | 473 |
| [symbol] | [symbol] | 474 | [symbol] | 475 | [symbol] | 476 |
| [symbol] | [symbol] | 477 | [symbol] | 478 | [symbol] | 479 |
| [symbol] | [symbol] | 480 | [symbol] | 481 | [symbol] | 482 |
| [symbol] | [symbol] | 483 | [symbol] | 484 | [symbol] | 485 |
| [symbol] | [symbol] | 486 | [symbol] | 487 | [symbol] | 488 |
| [symbol] | [symbol] | 489 | [symbol] | 490 | [symbol] | 491 |
| [symbol] | [symbol] | 492 | [symbol] | 493 | [symbol] | 494 |
| [symbol] | [symbol] | 495 | [symbol] | 496 | [symbol] | 497 |
| [symbol] | [symbol] | 498 | [symbol] | 499 | [symbol] | 500 |
| [symbol] | [symbol] | 501 | [symbol] | 502 | [symbol] | 503 |
| [symbol] | [symbol] | 504 | [symbol] | 505 | [symbol] | 506 |
| [symbol] | [symbol] | 507 | [symbol] | 508 | [symbol] | 509 |
| [symbol] | [symbol] | 510 | [symbol] | 511 | [symbol] | 512 |
| [symbol] | [symbol] | 513 | [symbol] | 514 | [symbol] | 515 |
| [symbol] | [symbol] | 516 | [symbol] | 517 | [symbol] | 518 |
| [symbol] | [symbol] | 519 | [symbol] | 520 | [symbol] | 521 |
| [symbol] | [symbol] | 522 | [symbol] | 523 | [symbol] | 524 |
| [symbol] | [symbol] | 525 | [symbol] | 526 | [symbol] | 527 |

[symbol]

indicates data missing or illegible when filed

TABLE 32
human IL2Rg sdAb CDRs

| | CDR1 | SEQ ID | | | | |
|---|---|---|---|---|---|---|
| Name | (Chothia/Kabat)) | NO: | CDR2 | SEQ ID NO: | CDR3 | SEQ ID NO: |
| hIL2Rg_VHH-1 | FTFDDSDMG | 528 | TISSDGSTYYADSVKG | 529 | DFMIAIQAPGAGC | 530 |
| hIL2Rg_VHH-2 | FSFSSYPMT | 531 | TIASDGGSTAYAASVE | 532 | GYGDGTPA | 533 |
| | | | G | | | |
| hIL2Rg_VHH-3 | FTFDDREMN | 534 | TISSDGSTYYADSVKG | 535 | DFMIAIQAPGAGC | 536 |
| hIL2Rg_VHH-4 | FTFDDSDMG | 537 | TISSDGNTYYTDSVKG | 538 | EPRGYYSNYGGRR | 539 |
| | | | | | ECNY | |
| hIL2Rg_VHH-5 | FSFSSYPMT | 540 | TIASDGGSTAYAASVE | 541 | GYGDGTPA | 542 |
| | | | G | | | |
| hIL2Rg_VHH-6 | FTFSNAHMS | 543 | SIYSGGSTWYADSVKG | 544 | NRLHYYSDDDSL | 545 |
| hIL2Rg_VHH-7 | FTFDDREMN | 546 | TISSDGSIYYADSVKG | 547 | DFMIAIQAPGAGC | 548 |
| hIL2Rg_VHH-8 | YTFSSYCMG | 549 | ALGGGSTYYADSVKG | 550 | AWVACLEFGGSWY | 551 |
| | | | | | DLARYKH | |
| hIL2Rg_VHH-9 | FTFDDSDMG | 552 | TISSDGSIYYADSVKG | 553 | EPRGYYSNYGGRR | 554 |
| | | | | | ECNY | |
| hIL2Rg_VHH- | SIYSSAYIG | 555 | GIYTRDGSTAYADSVK | 556 | GRRTKSYVYIFRPE | 557 |
| 10 | | | G | | EYNY | |
| hIL2Rg_VHH- | FTFSSAHMS | 558 | SIYSGGGTFYADSVKG | 559 | NRLHYYSDDDSL | 560 |
| 11 | | | | | | |
| hIL2Rg_VHH- | FTFSNAHMS | 561 | SIYSGGSTWYADSVKG | 562 | NRLHYYSDDDSL | 563 |
| 12 | | | | | | |
| hIL2Rg_VHH- | FIFDDSDMG | 564 | TISSDGSIYYADSVKG | 565 | EPRGYYSNYGGRR | 566 |
| 13 | | | | | ECNY | |
| hIL2Rg_VHH- | FTADDSDMG | 567 | TISSDGSTYYADSVKG | 568 | EPRGYYSNYGGRR | 569 |
| 14 | | | | | ECNY | |
| hIL2Rg_VHH- | FTFSSAHMS | 570 | SIYSGGGTFYADSVKG | 571 | NRLHYYSDDDSL | 572 |
| 15 | | | | | | |
| hIL2Rg_VHH- | FTFSNAHMS | 573 | SIYSGGSTWYADSVKG | 574 | NRLHYYSDDDSL | 575 |
| 16 | | | | | | |
| hIL2Rg_VHH- | FTFSNAHMS | 576 | SIYSGGSTWYADSVKG | 577 | NRLHYYSDDDSL | 578 |
| 17 | | | | | | |
| hIL2Rg_VHH- | FTFSSYPMT | 579 | TIASDGGSTAYAASVE | 580 | GYGDGTPA | 581 |
| 18 | | | G | | | |
| hIL2Rg_VHH- | FTFDDREMN | 582 | TISSDGSIYYADSVKG | 583 | DFMIAIQAPGAGC | 584 |
| 19 | | | | | | |
| hIL2Rg_VHH- | FTFDDSDMG | 585 | TISSDGSIYYADSVKG | 586 | EPRGYYSNYGGRR | 587 |
| 20 | | | | | ECNY | |
| hIL2Rg_VHH- | YTSCMG | 588 | TIYTRGRSIYYADSVK | 589 | GGYSWSAGCEFNY | 590 |
| 21 | | | G | | | |
| hIL2Rg_VHH- | FSFSSYPMT | 591 | TIASDGGSTAYAASVE | 592 | GYGDGTPA | 593 |
| 22 | | | G | | | |
| hIL2Rg_VHH- | FSFSSYPMT | 594 | TIASDGGSTAYAASVE | 595 | GYGDGTPA | 596 |
| 23 | | | G | | | |

TABLE 33
mouse IL2Rg sdAb CDRs

| | CDR 1 | SEQ ID | | SEQ ID | | |
|---|---|---|---|---|---|---|
| Name | (Chothia/Kabat) | NO: | CDR 2 | NO: | CDR 3 | SEQ ID NO: |
| DR604 | YGYNYIG | 597 | VIYTGGGDTYYAD | 598 | SVYACLRGGHDE | 599 |
| | | | SVKG | | YAH | |
| mIL2Rg_ | STYANYLMG | 600 | AIYSGGGSTYYAD | 601 | ASAVKGDKGDIV | 602 |
| VHH2 | | | SVKG | | VVVTGTQRMEYD | |
| | | | | | Y | |
| mIL2Rg_ | FTFDESVMS | 603 | IISSDDNTYYDDSV | 604 | RRRRPVYDSDYEL | 605 |
| VHH3 | | | KG | | RPRPLCGDFGV | |
| mIL2Rg_ | LPFDEDDMG | 606 | SISSDGTAYYADS | 607 | GVHRQFGGSSSCG | 608 |
| VHH4 | | | VKG | | DAFYGMDY | |
| mIL2Rg_ | DVYGRNSMA | 609 | VGYSVVTTTYYA | 610 | DGNLWRGLRPSE | 611 |
| VHH5 | | | DSVKG | | YTY | |
| mIL2Rg_ | FPYSRYCMG | 612 | AIEPDGSTSYADS | 613 | DERCFYLKDYDLR | 614 |
| VHH6 | | | VKG | | RPAQYRY | |
| mIL2Rg_ | FTFDESDMG | 615 | VITSDDNPYYDDS | 616 | RSRQPVYSRDYEL | 617 |
| VHH7 | | | VKG | | RPRPLCGDFGV | |
| mIL2Rg_ | FTFDDFDMG | 618 | TISDDGSTYYADS | 619 | EGALGSKTNCGW | 620 |
| VHH8 | | | VKG | | VGNFGY | |
| mIL2Rg_ | FTFDDFDMG | 621 | TISDDGSTYYADS | 622 | EGALGSKTNCGW | 623 |
| VHH9 | | | VKG | | VGNFGY | |
| mIL2Rg_ | FTFDDFDMG | 624 | TISDDGSTYYADS | 625 | EGALGSKTNCGW | 626 |
| VHH10 | | | VKG | | VGNFGY | |
| mIL2Rg_ | FTFSDRDMG | 627 | TISDDGSTYYADS | 628 | EGALGSKTNCGW | 629 |
| VHH11 | | | VKG | | VGNFGY | |
| mIL2Rg_ | YGYNYIG | 630 | VIYIGGGDTYYAD | 631 | RYCVGSVYACLR | 632 |
| VHH12 | | | SVKG | | GGHDEYAH | |
| mIL2Rg_ | YGYNYIG | 633 | VIYTGGGDTYYAD | 634 | RYCVGSVYACLR | 635 |
| VHH13 | | | SVKG | | GGHDEYAH | |
| mIL2Rg_ | FTFDDFDMG | 636 | TISDDGSTYYANS | 637 | EGALGSKTNCGW | 638 |
| VHH14 | | | VKG | | VGNFGY | |
| mIL2Rg_ | FTFDDFDMG | 639 | TISDDGSTYYADS | 640 | EGALGSKMNCGW | 641 |
| VHH15 | | | VKG | | VGNFGY | |

TABLE 34
human anti-IL2Rb VHH Amino Acid Sequences

| Name | VHH Sequence | SEQ ID NO: |
|---|---|---|
| hIL2Rb_ | QVQLQESGGGSVQAGGSLRLSC | 642 |
| VHH-1 | VGSGYTYDTSDMSWYRQAPGKEREFVS | |
| | DIDSGDWAAYADAVKGRFTISRDNAKK | |
| | TVYLQMNSLEPEDTAMYYCKASYWKW | |
| | GKLNNFWGPGTQVTVSS | |
| hIL2Rb_ | QVQLQESGGGLVQPGGSLRLSCV | 643 |
| VHH-2 | ASGFTFSNYWIFWVRQAAGKGLEWLST | |
| | SNTGGDTTKYADSVKGRFTISRDSAKNT | |
| | EYLQMNSLKPEDTAVYYCETGRCARSG | |
| | GYQGTQVTVSS | |
| hIL2Rb_ | QVQLQESGGGLVQPGGSLKLSCA | 644 |
| VHH-3 | ASGFRFSNYGMSWVRQAPGEGLEWVSY | |
| | INGDGSRTHYADSVKGRFTISRDNAKNT | |
| | LYLQLNSLKTEDTAMYYCEKGLSRDGW | |
| | SLSAASRGQGTQVTVSS | |
| hIL2Rb_ | QVQLQESGGGSVQTGGSLRLSCA | 645 |
| VHH-4 | VSGYTTYSFNYMGWFRQAPGKEREGVA | |
| | VIYTGGGSTLYADSVKGRFTISQDNAKN | |
| | TVYLQMNSLKPEDTAMYYCAADDQRF | |
| | ASPLYAYFGYWGQGTQVTVSS | |
| hIL2Rb_ | QVQLQESGGGSVQVGGSLRLSC | 646 |
| VHH-5 | ATSGDTKSIRCMGWFRQTPGKEREGIAA | |
| | IDREGFATYADSVYDRFTIAQDNAQNTL | |
| | YLEMNALKPEDTAMYYCAAQNMCRVV | |
| | RGAMTGVDYWGKGTQVTVSS | |
| hIL2Rb_ | QVQLQESGGGSVQAGGSLRLSC | 647 |
| VHH-6 | AASEYTASRYCMAWFRQAPGKEREGVA | |
| | AIHPGGGTTYYADSVKGRFSISQDSADN | |
| | TLYLQMNSLKPEDTAMYYCAAGSLWVP | |
| | FGDRCAANYWGQGTQVTVSS | |
| hIL2Rb_ | QVQLQESGGGSVQAGGSLRLSC | 648 |
| VHH-7 | AASGYEYCRIHMTWYRQGPGKEREFVS | |
| | SIGSDGRKTYANSVTGRFTISRDNANHT | |
| | VYLQMNSLSPEDTAMYYCKTEYLYGLG | |
| | CPDGSAYWGQGTQVTVSS | |
| hIL2Rb_ | QVQLQESGGGSVQVGGSLKLSC | 649 |
| VHH-8 | AASGYTYSSYYCMGWFRQAPGKEREGV | |
| | AAIDSDGSTSYADSVKGRFTISQDDAKN | |
| | TLYLQMNSLKPEDTAMYYCAASYEVVD | |
| | CYPSGYGQDYWGKGTQVTVSS | |

TABLE 35
murine anti-IL2Rb VHH Amino Acid Sequences

| | VHH AA Sequence | VHH |
|---|---|---|
| Name | (CDRs Underlined) | SEQ ID |
| DR857 | QVQLQESGGGLVQPGGSLRLSCAA | 650 |
| | SGFTFSLYDMSWVRQAPGKGLEWVSGINS | |
| | GGYSTYYAASAKGRFTISRDNAKNTLYLQ | |
| | LSSVKTEDTAMYYCAQRGLTSPYVIPNIRL | |
| | QGTQVTVSS | |
| DR1448 | EVQLVESGGRLVQAGDSLRLSCVA | 651 |
| | SGKSFSDYPLGWFRQAPGKAREYVAHISW | |
| | SGKLTYYRSTVKGRFTISRDNAENKLYLQ | |
| | MNALKPEDTAVYYCAAMKLFNYGGRYC | |
| | VLKPLTMYQQWSQGTQVTVSS | |
| DR1449 | EVQLVESGGGLVQAGGSLRLSCAA | 652 |
| | SGRSFSGYAIGWFRQAPGKEREFVAVVSW | |
| | RGSSTYYADSVKGRFTISRDNAKGTVYLQ | |
| | MNSLKPEDTAAYYCAAVPSGRSWYGRNR | |
| | YWGQGTQVTVSS | |
| DR1450 | EVQLVESGGGLVQAGGSLRLSCVI | 653 |
| | SGRSINYYRMGWFRQAPGNRRQFVAAIK | |
| | WGGDGVYADSVKGRFTISRDNTKNTVYL | |
| | QMDSLKPEDTGTYYCAKMPLSSWSRGGY | |
| | LEVWGQGTLVTVSS | |
| DR1451 | EVQLVESGGGLVQAGDSLRLSCAA | 654 |
| | SERFSWGNYAMYWFRQAPGKEREFVAAI | |
| | GRNSMATYYRDSAKGRFVISRDNAKNTL | |
| | YLEMNALKPEDTARYYCAAKFMVADGW | |
| | SRQYDYWGQGTLVTVSS | |
| DR1452 | EVQLVESGGGLVQAGGALRLSCA | 655 |
| | ASGRTFRRFMGWFRQAPGKEREFVAAIN | |
| | WPGGGTYYGDSVKGRFTISRDNAKNTVY | |
| | LQMNSLKPEDTANYYCAATRKYNLYKFA | |
| | DWGQGTQVTVSS | |
| DR1453 | EVQLVESGGRLVQAGDSLRLSCVA | 656 |
| | SGRIFNTYSMGWFRQVPGKERDFVAAIRW | |
| | SGGTTYYTDSVKGRFTISRDNAKNTVYLQ | |
| | MNSLKPEDTAVYYCWVRVRLSNTALLQR | |
| | YWGQGTLVTVSS | |
| DR1454 | EVQLVESGGGLVQAGGSLRLFCAS | 657 |
| | SERTFGDYPIGWFRQAPGKEREFVASISWG | |
| | GSRQYYTDSVKGRFTITRDNDKNTVYLQ | |
| | MNSLKPEDTAVYYCWVRVRLSNTALLQR | |
| | YWGQGTLVTVSS | |
| DR1455 | EVQLVESGGGLVQTGGSLRLSCAA | 658 |
| | SGRTFNSYAMGWFRQSPGKEREFVAVITW | |
| | NSGRTYYADSVKGRFTISRDNAKNTVYLQ | |
| | MNSLKPEDTAVYYCNSAPWAHNREWGQ | |
| | GTLVTVSS | |
| DR1456 | EVQLVESGGGLVQAGGSLRLSCAA | 659 |
| | SGLTFRTYYMSWFRQAPGKEREFVGVISW | |
| | IGSTTLYADSVKGRFSISRDNAKNTVYLQ | |
| | MNNLKPEDTAVYYCAANFLREGKREPRY | |
| | WGQGTQVTVSS | |
| DR1457 | EVQLVESGGRLVQAGDSLRLSCVA | 660 |
| | SGRIFNTYSMGWFRQVPGKERDFVAAIRW | |
| | SGGTTYYTDSVKGRFTISRDNAKNTVYLQ | |
| | MNSLKPEDTAVYYCYLRVFARRYWGQGT | |
| | QVTVSS | |
| DR1458 | EVQLVESGGGLVQAGGSLRLSCAA | 661 |
| | SGRTLSTYAMGWFRQAPGKEREFVAAIR | |
| | WASGRTYYGDSVKGRFTISRDSAKNTVYL | |
| | QMNSLKPEDTAVYYCAARSRPYLNYGDF | |
| | GYWGQGTQVTVSS | |
| DR1459 | EVQLVESGGGLVQAGGSLRLSCAA | 662 |
| | SGRTISTYAMVWFRQASGKEREFVGVISRS | |
| | GDRTYYADSVKGRFTISRDNLGNIVRLQL | |
| | NSLKPEDTAVYYCARGGYTGIETITARGR | |
| | GTLVTVSS | |
| DR1460 | EVQLVESGGGLVQTGDSLRLSCAA | 663 |
| | PESIFNNNAVYWYRQFPGKEREYVGLITIG | |
| | GRTGYADSVKGRFTISRDNANNVAFLQM | |
| | DSLKPEDTAVYYCATGLKFGFNFYSKTAY | |
| | DYWGQGTQVTVSS | |
| DR1461 | EVQLVESGGRLVQAGDSLRLSCVA | 664 |
| | SGRIFNTYSMGWFRQVPGKERDFVAAIRW | |
| | SGGTTYYTDSVKGRFTISRDNAKNTVYLQ | |
| | MKDLKPQDTAVYYCAAVPSGRSWYGRN | |
| | RYWGQGTLVTVSS | |
| DR1462 | EVQLVESGGGLVQAGGSLRLSCVS | 665 |
| | SGRTFGYVAMGWFRQAPGKEREFVASIN | |
| | WSGGSTAYADSVKGRFTISRDNAKNTVYL | |
| | QMNSLKPEDTAVYYCAGSTRFYIATMEQG | |
| | SYDYWGQGTQVTVSS | |
| DR1463 | EVQLVESGGSVVQPGDSLRLACTA | 666 |
| | SGRSFRSYAIGWFRQASGKERVFVAAISYD | |
| | GRRTYYGRSLKDRFTISRDNAKNTVYLQM | |
| | NSLKPEDTAVYYCATHRSGTMFARYGMD | |
| | YWGKGTLVTVSS | |
| DR1464 | EVQLVESGGGLVQAGGSLRLSCAA | 667 |
| | SGRTFSSYAMGWFRQAPGKEREFVTAISR | |
| | SGGYTSYADSVKGRFTISRDNAKNTVYLQ | |
| | MNSLKPEDTAVYYCAKLIAPFYYGMDYW | |
| | TKGTQVTVSS | |
| DR1465 | EVQLVESGGGLMQAGGALRLSCT | 668 |
| | ASGPTFTSYTMGWFRQSPGKRREFVAVIS | |
| | KGGRTYYADSVKGRFTISRDNAKNTFYLQ | |
| | MSSLKPEDTAVYYCAGQRVGATSKYEYD | |
| | YWGQGTQVTVSS | |
| DR1466 | EVQLVESGGGLVRAGGSLRLSCAA | 669 |
| | SGFTFSTDWMYWVRRAPGKGLEWVSLIN | |
| | TDGTSTSYTKSVKGRFTVSRDNAKNTLYL | |
| | QMNSLKPEDTALYYCARGRTYWFYAMD | |
| | YWGKGTQVTVSS | |
| DR1467 | EVQLVESGGGLVQAGDSLRLSCAA | 670 |
| | SGRISNYAMGWFRQAPGKEREFVAVITRS | |
| | GGSTYYADSVKGRFTISRDNGKNTIDLQM | |
| | NRLKPEDTAVYYCAVRRSQKLVTFGAEYP | |
| | WWGQGTLVTVSS | |
| DR1468 | EVQLVESGGGLVQAGGSLRLSCTT | 671 |
| | SGRTGTHYAMGWFRQAPGKEREFVSLIL | |
| | WNGEFTTYKDSVKGRFTISREKGENTVYL | |
| | QMNSLKPEDTAVYYCYLRVFARRYWGQG | |
| | TQVTVSS | |
| DR1469 | EVQLVESGGGLVQPGGSLRLSCEV | 672 |
| | SGFTFSNYWMYWIRQAPGKGLEWVSHIN | |
| | TNGGNTYYRHSVKGRFTISRDNAKNTLYL | |
| | QMNGLKSEDTAVYYCAKANSDVGLGYY | |
| | GMDYWGKGTQVTVSS | |
| DR1470 | EVQLVESGGGSVQPGGSLRLSCAA | 673 |
| | PESIFNNNAVYWYRQFPGKEREYVGLITIG | |
| | GRTGYADSVKGRFTISRDNANNVAFLQM | |
| | DNLKPEDTAVYYCAARPGYWSSSYDYWG | |
| | QGTQVTVSS | |
| DR1471 | EVQLVESGGGLVQAGGSLRLSCVF | 674 |
| | SGRAPASYAMAWFRQAVGNEREFVAAIN | |
| | WSGRRTYYADSVKGRFTISKDNAQNTAY | |
| | LQMTNLEPEDTATYYCNAYLSGTYYWGQ | |
| | GTQVTVSS | |
| DR1472 | EVQLVESGGGLVRAGDSLRLSCAV | 675 |
| | SGLASSSFFMTWFRQGQGKEREFVATISW | |
| | TGRTSYYAASVKGRFTVSRDNAKNTVYL | |
| | QMNSLNSEDTAVYFCAAYPRTLVRNREPI | |
| | HWGQGTQVTVSS | |

TABLE 36
human anti-IL2Rg VHH Amino Acid Sequences

| | VHH Sequence | SEQ ID |
|---|---|---|
| Name | (CDRs are underlined) | NO: |
| hIL2Rg_ | QVQLQESGGGSVQAGGSLRLSCAASGE | 676 |
| VHH-1 | TFDDSDMGWYRQAPGNECDLVSTISSDGSTY | |
| | YADSVKGRFTISQDNAKNTVYLQMDSVKPED | |
| | TAVYYCAADFMIAIQAPGAGCWGQGTQVTVS | |
| | S | |
| hIL2Rg_ | QVQLQESGGGSVPAGGSLKLSCAASGF | 677 |
| VHH-2 | SFSSYPMTWARQAPGKGLEWVSTIASDGGST | |
| | AYAASVEGRFTISRDNAKSTLYLQLNSLKTED | |
| | TAMYYCTKGYGDGTPAPGQGTQVTVSS | |
| hIL2Rg_ | QVQLQESGGGSVQTGGSLRLSCTASGF | 678 |
| VHH-3 | TFDDREMNWYRQAPGNECELVSTISSDGSTYY | |
| | ADSVKGRFTISQDNAKNTVYLQMDSVKPEDT | |
| | AVYYCAADFMIAIQAPGAGCWGQGTQVTVSS | |
| hIL2Rg_ | QVQLQESGGGSVQAGGSLRLSCTASGF | 679 |
| VHH-4 | TFDDSDMGWYRQAPGNECELVSTISSDGNTY | |
| | YTDSVKGRFTISQDNAKNTVYLQMNSLGPED | |
| | TAVYYCAAEPRGYYSNYGGRRECNYWGQGT | |
| | QVTVSS | |
| hIL2Rg_ | QVQLQESGGGSVQAGGSLRLSCAASGF | 680 |
| VHH-5 | SFSSYPMTWARQAPGKGLEWVSTIASDGGST | |
| | AYAASVEGRFTISRDNAKSTLYLQLNSLKTED | |
| | TAMYYCTKGYGDGTPAPGQGTQVTVSS | |
| hIL2Rg_ | QVQLQESGGGAVQAGGSLRLSCAASGF | 681 |
| VHH-6 | TFSNAHMSWVRQAPGKGREWISSIYSGGSTW | |
| | YADSVKGRFTISRDNSKNTLYLQLNSLKTEDT | |
| | AMYYCAENRLHYYSDDDSLRGQGTQVTVSS | |
| hIL2Rg_ | QVQLQESGGGLVQPGGSLRLSCAASGF | 682 |
| VHH-7 | TFDDREMNWYRQAPGNECELVSTISSDGSTYY | |
| | ADSVKGRFTISQDNAKNTVYLQMDSVKPEDT | |
| | AVYYCAADFMIAIQAPGAGCWGQGTQVTVSS | |
| hIL2Rg_ | QVQLQESGGGSVQAGGSLRLSCVASGY | 683 |
| VHH-8 | TFSSYCMGWFRQAPGKEREGVAALGGGSTYY | |
| | ADSVKGRFTISQDNAKNTLYLQMNSLKPEDT | |
| | AMYYCAAAWVACLEFGGSWYDLARYKHWG | |
| | QGTQVTVSS | |
| hIL2Rg_ | QVQLQESGGGSVQAGGSLRLSCTASGF | 684 |
| VHH-9 | TFDDSDMGWYRQAPGGECELVTISSDGSTYY | |
| | ADSVKGRFTISQDNAKNTVYLQMNSLKPEDT | |
| | AVYYCAAEPRGYYSNYGGRRECNYWGQGTQ | |
| | VTVSS | |
| hIL2Rg_ | QVQLQESGGGSVQAGGSLRLSCAASGS | 685 |
| VHH-10 | IYSSAYIGWFRQAPGKKREGVAGIYTRDGSTA | |
| | YADSVKGRFTISQDSAKKTVYLQMNSLKPEDT | |
| | AMYYCAAGRRTKSYVYIFRPEEYNYWGQGT | |
| | QVTVSS | |
| hIL2Rg_ | QVQLQESGGGSVQAGGSLRLSCAASGF | 686 |
| VHH-11 | TFSSAHMSWVRQAPGKGREWIASIYSGGGTFY | |
| | ADSVKGRFTISRDNAKNTLYLQLNSLKTEDTA | |
| | MYYCATNRLHYYSDDDSLRGQGTQVTVSS | |
| hIL2Rg_ | QVQLQESGGGSVQAGGSLRLSCAASGF | 687 |
| VHH-12 | TFSNAHMSWVRQAPGKGREWISSIYSGGSTW | |
| | YADSVKGRFTISRDNSKNTLYLQLNSLKTEDT | |
| | AMYYCAENRLHYYSDDDSLRGQGTQVTVSS | |
| hIL2Rg_ | QVQLQESGGGSVQAGGSLRLSCTASRFI | 688 |
| VHH-13 | FDDSDMGWYRQAPGNECELVSTISSDGSTYY | |
| | ADSVKGRFTISRDNAKNTVYLQMNSLKPEDT | |
| | AVYYCAAEPRGYYSNYGGRRECNYWGQGTQ | |
| | VTVSS | |
| hIL2Rg_ | QVQLQESGGGSVQAGGSLKLSCTVSGF | 689 |
| VHH-14 | TADDSDMGWYRQGPGNECELVTISSDGSTYY | |
| | ADSVKGRFTISQDNAKNTVYLQMNSLKPEDT | |
| | AVYYCAAEPRGYYSNYGGRRECNYWGQGTQ | |
| | VTVSS | |
| hIL2Rg_ | QVQLQESGGGLVQPGGSLRLSCAASGF | 690 |
| VHH-15 | TFSSAHMSWVRQAPGKGREWIASIYSGGGTFY | |
| | ADSVKGRFTISRDNAKNTLYLQLNSLKAEDTA | |
| | MYYCATNRLHYYSDDDSLRGQGTQVTVSS | |
| hIL2Rg_ | QVQLQESGGGLVQPGGSLRLSCVASGF | 691 |
| VHH-16 | TFSNAHMSWVRQAPGKGREWISSIYSGGSTW | |
| | YADSVKGRFTISRDNSKNTLYLQLNSLKTEDT | |
| | AMYYCAENRLHYYSDDDSLRGQGTQVTVSS | |
| hIL2Rg_ | QVQLQESGGGLVQPGGSLRLSCAASGF | 692 |
| VHH-17 | TFSNAHMSWVRQAPGKGREWISSIYSGGSTW | |
| | YADSVKGRFTISRDNSKNTLYLQLNSLKTEDT | |
| | AMYYCAENRLHYYSDDDSLRGQGTQVTVSS | |
| hIL2Rg_ | QVQLQESGGGLVQPGGSLRLSCAASGF | 693 |
| VHH-18 | TFSSYPMTWARQAPGKGLEWVSTIASDGGST | |
| | AYAASVEGRFTISRDNAKSTLYLQLNSLKTED | |
| | TAMYYCTKGYGDGTPAPGQGTQVTVSS | |
| hIL2Rg_ | QVQLQESGGGSVQAGGSLRLSCTASGF | 694 |
| VHH-19 | TFDDREMNWYRQAPGNECELVSTISSDGSTYY | |
| | ADSVKGRFTISQDNAKNTVYLQMDSVKPEDT | |
| | AVYYCAADFMIAIQAPGAGCWGQGTQVTVSS | |
| hIL2Rg_ | QVQLQESGGGSVQAGGSLRLSCTASGF | 695 |
| VHH-20 | TFDDSDMGWYRQAPGNECELVSTISSDGSTYY | |
| | ADSVKGRFTISQDNAKNTVYLQMNSLKPEDT | |
| | AVYYCAAEPRGYYSNYGGRRECNYWGQGTQ | |
| | VTVSS | |
| hIL2Rg_ | QVQLQESGGGSVQAGGSLRLSCVASGY | 696 |
| VHH-21 | TSCMGWFRQAPGKEREAVATIYTRGRSIYYA | |
| | DSVKGRFTISQDNAKNTLYLQMNSLKPEDIAM | |
| | YSCAAGGYSWSAGCEFNYWGQGTQVTVSS | |
| hIL2Rg_ | QVQLQESGGGLVQPGGSLRLSCTASGF | 697 |
| VHH-22 | SFSSYPMTWARQAPGKGLEWVSTIASDGGST | |
| | AYAASVEGRFTISRDNAKSTLYLQLNSLKTED | |
| | TAMYYCTKGYGDGTPAPGQGTQVTVSS | |
| hIL2Rg_ | QVQLQESGGGLVQPGGSLRLSCAASGF | 698 |
| VHH-23 | SFSSYPMTWARQAPGKGLEWVSTIASDGGST | |
| | AYAASVEGRFTISRDNAKSTLYLQLNSLKTED | |
| | TAMYYCTKGYGDGTPAPGQGTQVTVSS | |

TABLE 37
murine anti-IL2Rg VHH Amino Acid Sequences

| | VHH AA Sequence | SEQ ID |
|---|---|---|
| Name | (CDRs Underlined) | NO: |
| mIL2Rg_ | QVQLQESGGGSVLAGGSLRLSCVASGYGYNYIGWFRQTPG | 699 |
| VHH1 | KEREGVAVIYTGGGDTYYADSVKGRFTASRDNAKSTLYLQMN | |
| | SLEPEDTAMYYGVARYCVGSVYACLRGGHDEYAHWGQGTQVT | |
| | VSS | |
| mIL2Rg_ | QVQLQESGGGSVQPGGSLRLSCAASGSTYANYLMGWFRQAP | 700 |
| VHH2 | GKEREGVAAIYSGGGSTYYADSVKGRFTISQDNAKNTLYLQ | |
| | MNSLKPEDTAMYYCAAASAVKGDKGDIVVVVTGTQRMEYDY | |
| | WGHGTQVTVSS | |
| mIL2Rg_ | QVQLQESGGGSVQAGASLRLSCSVSGFTFDESVMSWLRQGPG | 701 |
| VHH3 | NECDAVAIISSDDNTYYDDSVKGRFTISEDNAKNMVYLQMNS | |
| | LKPEDTAVYYCAARRRRPVYDSDYELRPRPLCGDFGVWGQGT | |
| | QVTVSS | |
| mIL2Rg_ | QVQLQESGGGSVQAGGSLRLSCIGSGLPFDEDDMGWYRQAP | 702 |
| VHH4 | GNECELVSSISSDGTAYYADSVKGRFTISRDNAKNTVLLQMNS | |
| | LKPEDTAVYYCAAGVHRQFGGSSSCGDAFYGMDYWGKGTQVT | |
| | VSS | |
| mIL2Rg_ | QVQLQESGGGSVQAGGSLRLSCVASGDVYGRNSMAWFR | 703 |
| VHH5 | QAPGKEREGVAVGYSVVTTTYYADSVKGRFTISEDNDKNTVYLE | |
| | MNSLKPEDTAMYYCAADGNLWRGLRPSEYTYWGQGTQVTVSS | |
| mIL2Rg_ | QVQLQESGGGSVQAGGSLRLSCATSGFPYSRYCMGWFRQ | 704 |
| VHH6 | APGKEREGVAAIEPDGSTSYADSVKGRFTISQDNAVNTLYLQMN | |
| | NLKPEDTAMYYCAADERCFYLKDYDLRRPAQYRYWGQGTQVT | |
| | VSS | |
| mIL2Rg_ | QVQLQESGGGLVQPGGSLRLSCTVSGFTFDESDMGWLRQ | 705 |
| VHH7 | NPGNECGVVSVITSDDNPYYDDSVKGRFTISEDNAKNMVYLQM | |
| | NSLKPEDTGVYYCATRSRQPVYSRDYELRPRPLCGDFGVWGQGT | |
| | QVTVSS | |
| mIL2Rg_ | QVQLQESGGGSVQAGGSLRLSCTASGFTFDDFDMGWYRQAPG | 706 |
| VHH8 | NECELVSTISDDGSTYYADSVKGRSSISRDNAKNTVYLQMN | |
| | RLKPEDTGVYYCAAEGALGSKTNCGWVGNFGYWGQGTQVTVS | |
| | S | |
| mIL2Rg_ | QVQLQESGGGSVQAGGSLRLSCAASGFTFDDFDMGWYRQAP | 707 |
| VHH9 | GNECELVSTISDDGSTYYADSVKGRSSISRDNAKNTVYLQM | |
| | NSLKPEDTAVYYCAAEGALGSKTNCGWVGNFGYWGQGTQVT | |
| | VSS | |
| mIL2Rg_ | QVQLQESGGGLVQPGGSLRLSCAASGFTFDDFDMGWYRQAPG | 708 |
| VHH10 | NECELVSTISDDGSTYYADSVKGRSSISRDNAKSTVYLQMNR | |
| | LKPEDTGVYYCAAEGALGSKTNCGWVGNFGYWGQGTQVTVSS | |
| mIL2Rg_ | QVQLQESGGGLVQPGGSLKLSCAASGFTFSDRDMGWYRQ | 709 |
| VHH11 | APGNECERVSTISDDGSTYYADSVKGRSSISRDNAKNTVYLQMN | |
| | SLKPEDTAVYYCAAEGALGSKTNCGWVGNFGYWGQGTQVTVS | |
| | S | |
| mIL2Rg_ | QVQLQESGGGSVLAGGSLRLSCVASGYGYNYIGWFRQTP | 710 |
| VHH12 | GKEREGVAVIYIGGGDTYYADSVKGRFTASRDNAKSTLYLQMNS | |
| | LEPEDTAMYYCVARYCVGSVYACLRGGHDEYAHWGQGTQVTV | |
| | SS | |
| mIL2Rg_ | QVQLQESGGGSVLAGGSLRLSCVASGYGYNYIGWFRQTP | 711 |
| VHH13 | GKEREGVAVIYTGGGDTYYADSVKGRFTASRDNAKSTLYLQMN | |
| | SLEPEDTAMYYCVARYCVGSVYACLRGGHDEYAHWGQGTQVT | |
| | VSS | |
| mIL2Rg_ | QVQLQESGGGSVQAGGSLRLSCAASGFTFDDFDMGWYR | 712 |
| VHH14 | QAPGNECELVSTISDDGSTYYANSVKGRSSISRDNAKNMVYLQM | |
| | NSLKPEDTAVYYCAAEGALGSKTNCGWVGNFGYWGQGTQVTV | |
| | SS | |
| mIL2Rg_ | QVQLQESGGGSVQAGGSLRLSCTASGFTFDDFDMGWYRQ | 713 |
| VHH15 | APGNECELVSTISDDGSTYYADSVKGRSSISRDNAKNTVYLQMN | |
| | RLKPEDTGVYYCAAEGALGSKMNCGWVGNFGYWGQGTQVTVS | |
| | S | |

TABLE 38
IL2Rb sdAb VHH DNA SEQUENCE

| | | SEQ |
|---|---|---|
| | | ID |
| Name | Sequence | NO: |
| hIL2Rb_VHH | CAGGTCCAGTTGCAGGAGAGCGGTGGCGGT | 714 |
| -1 | AGCGTGCAGGCCGGTGGCAGTCTGCGCCTTTCCTG | |
| | CGTAGGCAGCGGTTACACCTACGACACCTCCGACA | |
| | TGAGCTGGTATAGGCAGGCCCCAGGCAAGGAGAGG | |
| | GAATTTGTCTCCGATATTGATTCCGGCGACTGGGC | |
| | TGCCTACGCTGATGCCGTGAAGGGCCGCTTCACAA | |
| | TCAGCCGTGACAACGCCAAAAAGACCGTGTATCTG | |
| | CAAATGAACAGTCTGGAACCTGAGGACACGGCAAT | |
| | GTACTATTGCAAAGCCTCTTATTGGAAGTGGGGCA | |
| | AGCTCAATAACTTCTGGGGTCCCGGCACACAGGTG | |
| | ACCGTGTCCTCT | |
| hIL2Rb_VHH | CAGGTGCAGCTCCAGGAAAGCGGCGGAGGC | 715 |
| -2 | CTGGTCCAGCCTGGCGGGAGCTTGCGTCTGTCCTG | |
| | CGTGGCAAGCGGATTCACGTTTAGTAATTACTGGA | |
| | TCTTTTGGGTACGGCAGGCAGCTGGCAAGGGGCTT | |
| | GAGTGGCTTTCAACATCCAACACAGGTGGCGATAC | |
| | TACAAAATACGCGGATTCTGTAAAAGGCCGGTTCA | |
| | CGATCAGTCGCGACTCCGCGAAGAACACCGAATAC | |
| | CTCCAGATGAACTCCTTGAAGCCTGAAGACACCGC | |
| | AGTCTACTATTGCGAAACCGGACGCTGCGCCAGGT | |
| | CTGGAGGTTACCAGGGCACGCAGGTGACCGTTTCC | |
| | TCC | |
| hIL2Rb_VHH | CAGGTGCAGCTCCAGGAGTCCGGCGGGGGA | 716 |
| -3 | CTGGTCCAGCCAGGAGGTTCTTTGAAGCTGAGTTG | |
| | CGCCGCTTCTGGTTTTAGATTCTCTAACTACGGCA | |
| | TGTCTTGGGTTCGCCAAGCGCCCGGAGAGGGCCTG | |
| | GAGTGGGTCAGTTACATTAACGGGGACGGCTCCCG | |
| | CACCCACTACGCTGACTCCGTCAAAGGGCGGTTCA | |
| | CCATCTCACGTGACAACGCTAAGAACACCCTGTAC | |
| | CTCCAGCTGAACAGCCTGAAGACAGAGGATACAGC | |
| | CATGTATTACTGTGAGAAGGGTCTGTCTCGCGACG | |
| | GTTGGTCCCTCAGCGCTGCCAGTCGCGGGCAGGGG | |
| | ACCCAAGTGACAGTCAGCTCT | |
| hIL2Rb_VHH | CAGGTCCAACTGCAAGAGAGCGGCGGGGGC | 717 |
| -4 | AGCGTGCAGACTGGAGGCTCCCTGCGTCTGTCCTG | |
| | TGCGGTGTCAGGGTATACAACCTATTCATTCAACT | |
| | ATATGGGATGGTTCCGCCAGGCTCCGGGCAAGGAG | |
| | CGCGAAGGCGTGGCGGTAATCTACACCGGCGGGGG | |
| | ATCTACCCTGTATGCTGATTCTGTTAAAGGGCGCT | |
| | TCACTATCTCCCAGGACAACGCCAAGAACACTGTG | |
| | TACCTCCAGATGAACTCCCTGAAACCCGAAGATAC | |
| | CGCGATGTATTACTGCGCTGCCGACGATCAGCGCT | |
| | TCGCCTCCCCGCTCTACGCCTACTTCGGTTACTGG | |
| | GGCCAGGGCACTCAGGTGACCGTGTCTAGC | |
| hIL2Rb_VHH | CAAGTGCAACTCCAGGAGAGCGGTGGAGGC | 718 |
| -5 | TCTGTGCAGGTGGGTGGCAGTCTGCGTCTCTCTTG | |
| | CGCTACCTCTGGTGACACCAAGAGCATCCGTTGTA | |
| | TGGGCTGGTTCCGTCAAACTCCTGGTAAGGAGCGC | |
| | GAAGGCATCGCCGCTATTGATCGCGAGGGTTTTGC | |
| | CACCTACGCTGATAGCGTGTATGATCGCTTCACCA | |
| | TCGCCCAGGATAACGCCCAGAATACCCTGTACCTG | |
| | GAGATGAATGCCCTGAAGCCTGAGGATACAGCAAT | |
| | GTATTACTGCGCTGCCCAGAATATGTGCCGCGTAG | |
| | TGAGAGGTGCCATGACGGGGGTGGACTATTGGGGC | |
| | AAGGGCACCCAAGTGACTGTGTCCAGC | |
| hIL2Rb_VHH | CAAGTTCAGCTGCAAGAGTCTGGGGGCGGT | 719 |
| -6 | AGCGTGCAGGCGGGTGGGTCCCTGCGCCTCTCTTG | |
| | CGCTGCCTCCGAGTACACAGCATCTCGGTACTGCA | |
| | TGGCCTGGTTTCGTCAGGCTCCGGGTAAGGAGCGG | |
| | GAGGGCGTTGCCGCTATTCATCCGGGCGGAGGTAC | |
| | GACCTACTATGCAGACTCCGTAAAGGGTCGCTTCT | |
| | CCATCAGCCAGGATTCTGCCGACAACACCTTGTAC | |
| | CTCCAGATGAACTCACTGAAACCTGAGGATACCGC | |
| | GATGTATTACTGCGCGGCTGGCTCTCTGTGGGTGC | |
| | CCTTCGGCGACCGCTGTGCTGCCAACTATTGGGGC | |
| | CAGGGAACCCAGGTTACAGTGTCTTCC | |
| hIL2Rb_VHH | CAGGTTCAGTTGCAGGAGTCCGGCGGTGGC | 720 |
| -7 | AGCGTACAGGCCGGGGGCTCCCTGAGACTTAGTTG | |
| | CGCAGCGTCCGGTTACGAGTACTGCCGTATTCACA | |
| | TGACTTGGTATAGGCAAGGCCCTGGTAAGGAACGC | |
| | GAGTTCGTTTCTTCCATCGGGAGTGATGGCCGTAA | |
| | AACCTACGCCAACAGCGTGACCGGACGTTTCACCA | |
| | TCAGTCGTGACAACGCTAACCACACGGTTTACTTG | |
| | CAGATGAACTCCCTCTCCCCTGAGGACACCGCCAT | |
| | GTACTATTGTAAGACCGAGTACCTGTATGGCCTCG | |
| | GCTGCCCAGATGGTAGCGCCTACTGGGGCCAGGGG | |
| | ACCCAGGTCACCGTTTCCAGT | |
| hIL2Rb_VHH | CAGGTCCAGTTGCAGGAGTCTGGAGGTGGA | 721 |
| -8 | TCAGTGCAGGTTGGGGGTTCACTGAAACTTAGCTG | |
| | TGCCGCTTCTGGGTATACATATTCTAGCTACTATT | |
| | GTATGGGCTGGTTTCGCCAGGCTCCTGGAAAGGAG | |
| | CGCGAAGGGGTGGCGGCCATCGACTCCGACGGCTC | |
| | CACATCCTACGCGGACTCCGTGAAGGGCCGCTTTA | |
| | CAATCAGTCAGGATGACGCTAAGAACACGCTGTAC | |
| | CTCCAGATGAATAGCCTGAAGCCCGAAGATACGGC | |
| | GATGTACTATTGCGCCGCGTCTTACGAAGTAGTGG | |
| | ACTGCTATCCGTCCGGCTATGGCCAAGATTACTGG | |
| | GGAAAAGGAACTCAAGTGACCGTGAGTTCC | |

TABLE 39
murine IL2Rb sdAb VHH DNA SEQUENCE

| Name | DNA Sequence | SEQ ID NO |
|---|---|---|
| DR857 DNA | CAGGTGCAGTTGCAGGAGAGCGGGGGCGGTCTG | 722 |
| | GTCCAGCCGGGCGGGTCACTGCGCCTGTCTTGTGCCGCT | |
| | TCAGGATTTACCTTTAGTTTGTACGACATGAGTTGGGTT | |
| | AGGCAAGCGCCTGGCAAGGGTCTGGAGTGGGTGTCTGG | |
| | CATCAACTCAGGAGGCTATAGCACCTATTACGCGGCCT | |
| | CCGCCAAGGGCCGCTTCACCATCTCTAGGGATAACGCA | |
| | AAGAACACTCTTTACCTCCAGCTCAGCTCTGTTAAGACT | |
| | GAGGATACTGCCATGTATTACTGTGCCCAGCGCGGCCT | |
| | CACCAGCCCGTATGTGATTCCGAACATTCGCTTGCAGG | |
| | GCACACAGGTGACTGTGTCCAGC | |
| DR1448 DNA | GAGGTCCAACTGGTGGAGAGCGGCGGAAGGCTG | 723 |
| | GTGCAGGCTGGCGACTCCCTGCGCTTGAGCTGTGTGGC | |
| | AAGCGGAAAGTCCTTTTCCGATTACCCTCTCGGTTGGTT | |
| | CCGTCAGGCTCCTGGAAAAGCTAGGGAGTATGTGGCCC | |
| | ACATCTCTTGGAGCGGCAAACTGACTTACTATCGCTCA | |
| | ACAGTGAAGGGCCGGTTTACTATCAGCCGCGATAACGC | |
| | TGAAAATAAACTGTACCTCCAGATGAACGCCCTGAAGC | |
| | CCGAGGATACTGCCGTGTATTACTGTGCTGCCATGAAG | |
| | TTGTTCAACTATGGCGGGCGTTACTGTGTTCTCAAGCCC | |
| | CTGACAATGTACCAACAGTGGAGCCAGGGTACTCAGGT | |
| | CACAGTTAGCTCC | |
| DR1449 DNA | GAGGTCCAGCTCGTTGAGAGCGGCGGGGGCCTG | 724 |
| | GTGCAGGCCGGTGGCAGCCTCCGTCTCTCCTGTGCCGCT | |
| | TCTGGCCGCAGTTTCTCCGGGTATGCTATCGGGTGGTTC | |
| | AGACAGGCACCAGGCAAGGAGCGCGAGTTTGTTGCTGT | |
| | CGTGAGCTGGCGGGGTTCTAGCACCTACTATGCCGACT | |
| | CAGTCAAGGGCCGCTTCACAATTAGCAGGGACAACGCC | |
| | AAGGGCACTGTATACCTCCAGATGAACTCCCTGAAGCC | |
| | AGAGGATACCGCCGCGTATTACTGCGCTGCCGTGCCAT | |
| | CTGGCCGCTCCTGGTACGGTAGGAACCGTTACTGGGGT | |
| | CAGGGAACTCAGGTCACCGTGTCCTCA | |
| DR1450 DNA | GAAGTGCAGCTCGTTGAAAGCGGCGGGGGCCTC | 725 |
| | GTGCAAGCTGGAGGCTCACTTCGCCTTTCTTGTGTCATC | |
| | AGTGGCCGCTCTATCAATTATTACCGGATGGGCTGGTTC | |
| | CGCCAGGCCCCTGGCAACCGCAGGCAATTCGTGGCGGC | |
| | TATCAAGTGGGGTGGCGACGGTGTGTACGCCGACTCCG | |
| | TGAAGGGGCGCTTTACCATTAGTCGGGACAACACCAAG | |
| | AACACCGTATACTTGCAGATGGACAGTCTGAAGCCCGA | |
| | AGACACCGGAACATATTACTGCGCCAAAATGCCTCTTT | |
| | CTAGCTGGTCCAGAGGTGGCTACCTTGAGGTGTGGGGT | |
| | CAAGGCACGCTGGTGACCGTGTCTTCT | |
| DR1451 DNA | GAAGTGCAACTCGTGGAAAGTGGAGGCGGTCTC | 726 |
| | GTCCAGGCGGGGGACAGCCTGCGTCTGTCTTGCGCCGC | |
| | ATCCGAGCGTTTTTCTTGGGGCAACTATGCTATGTATTG | |
| | GTTCAGGCAGGCCCCTGGCAAGGAACGCGAGTTCGTGG | |
| | CTGCCATTGGCCGCAACAGCATGGCCACGTATTACAGA | |
| | GATAGCGCCAAGGGCCGCTTCGTCATCAGCCGTGACAA | |
| | CGCTAAGAACACCCTGTACCTGGAAATGAACGCCTTGA | |
| | AGCCTGAAGATACTGCTAGGTACTATTGCGCCGCGAAG | |
| | TTCATGGTGGCCGACGGCTGGAGCAGACAGTATGACTA | |
| | CTGGGGCCAGGGCACTCTGGTAACGGTCTCCTCC | |
| DR1452 DNA | GAAGTTCAGCTTGTGGAAAGCGGCGGTGGGCTT | 727 |
| | GTCCAGGCTGGTGGAGCGCTGCGCCTCTCCTGCGCAGC | |
| | GAGTGGCAGGACCTTCCGCCGTTTCATGGGTTGGTTTCG | |
| | CCAGGCCCCAGGGAAGGAGCGCGAGTTTGTTGCTGCCA | |
| | TCAACTGGCCTGGAGGTGGCACCTACTATGGCGATAGC | |
| | GTGAAGGGCCGTTTCACAATCTCCAGGGACAACGCCAA | |
| | GAATACCGTCTACCTGCAAATGAACTCCCTGAAGCCGG | |
| | AGGACACCGCGAACTATTACTGCGCCGCAACCCGCAAG | |
| | TACAACCTGTATAAATTCGCGGACTGGGGCCAGGGCAC | |
| | CCAGGTGACAGTGTCATCT | |
| DR1453 DNA | GAGGTCCAGCTCGTCGAGTCCGGCGGGCGGCTG | 728 |
| | GTGCAGGCTGGCGACAGCCTTCGCCTGTCCTGTGTGGC | |
| | ATCCGGCAGAATCTTTAACACCTACTCAATGGGTTGGTT | |
| | TAGGCAGGTTCCCGGAAAGGAGAGGGATTTCGTGGCTG | |
| | CCATCAGATGGTCCGGTGGCACCACATATTACACTGAT | |
| | TCTGTCAAGGGGCGCTTCACCATTAGTCGCGATAACGC | |
| | AAAAAACACCGTGTACCTGCAAATGAATAGCCTGAAGC | |
| | CTGAGGACACCGCCGTATATTACTGTTGGGTGCGCGTTC | |
| | GCCTGAGCAACACAGCCCTGCTTCAGCGCTACTGGGGT | |
| | CAGGGAACCTTGGTTACCGTGTCAAGC | |
| DR1454 DNA | GAAGTCCAGCTCGTGGAGTCCGGGGGAGGTCTG | 729 |
| | GTTCAAGCTGGGGGTTCTTTGCGCCTCTTTTGCGCGTCC | |
| | AGCGAGCGTACTTTCGGAGATTACCCAATCGGATGGTT | |
| | CCGTCAAGCCCCAGGCAAGGAGCGCGAGTTTGTCGCGT | |
| | CCATCAGCTGGGGTGGCTCACGTCAGTACTATACTGAC | |
| | TCCGTTAAGGGCCGCTTCACGATTACAAGAGATAATGA | |
| | TAAGAACACCGTGTATCTCCAGATGAACTCCCTCAAGC | |
| | CCGAGGACACTGCTGTTTACTATTGCTGGGTGCGGGTG | |
| | CGTCTGTCAAACACGGCACTGCTTCAGCGCTATTGGGG | |
| | ACAGGGCACCCTGGTCACCGTCTCCTCA | |
| DR1455 DNA | GAAGTCCAGCTGGTCGAGTCAGGCGGGGGACTG | 730 |
| | GTGCAGACTGGGGGTAGTCTGCGCCTGAGCTGCGCAGC | |
| | TTCAGGAAGAACCTTCAACTCCTACGCTATGGGCTGGTT | |
| | CAGACAGAGCCCAGGCAAAGAGCGGGAGTTCGTGGCG | |
| | GTGATTACGTGGAACTCTGGCCGCACGTACTATGCTGA | |
| | CAGTGTCAAAGGCAGATTTACCATCAGTAGGGATAACG | |
| | CCAAGAACACAGTGTATCTCCAGATGAACTCTCTGAAG | |
| | CCCGAGGATACTGCTGTGTATTACTGTAACAGCGCCCC | |
| | CTGGGCTCACAATCGTGAGTGGGGGCAGGGGACCCTCG | |
| | TTACCGTCAGCAGC | |
| DR1456 DNA | GAGGTGCAGCTGGTGGAATCTGGTGGAGGGCTG | 731 |
| | GTGCAGGCTGGCGGTTCCCTCCGTCTGTCTTGTGCGGCC | |
| | TCAGGGCTGACCTTCAGGACCTACTATATGTCATGGTTC | |
| | CGCCAAGCGCCCGGCAAGGAACGCGAGTTCGTCGGAGT | |
| | GATCTCTTGGATCGGCTCCACTACCCTCTACGCCGATTC | |
| | TGTGAAAGGTAGGTTTTCCATCTCACGCGATAATGCTA | |
| | AGAACACCGTCTACCTCCAGATGAATAACTTGAAACCC | |
| | GAGGACACCGCCGTCTACTATTGCGCGGCCAACTTCCT | |
| | CAGAGAGGGAAAGCGCGAACCTCGGTATTGGGGACAA | |
| | GGGACCCAGGTGACCGTTTCCTCC | |
| DR1457 DNA | GAGGTGCAGTTGGTTGAGTCTGGCGGAAGGCTC | 732 |
| | GTTCAAGCTGGTGACAGCCTGCGGCTGTCTTGCGTCGCT | |
| | TCTGGACGCATCTTCAACACATATTCAATGGGCTGGTTC | |
| | AGACAGGTGCCTGGCAAGGAGCGCGACTTCGTGGCAGC | |
| | TATCCGTTGGAGCGGGGGCACTACGTATTACACCGATT | |
| | CTGTGAAGGGGCGCTTCACAATCTCCAGGGATAATGCA | |
| | AAGAACACCGTGTACCTTCAGATGAACAGCTTGAAGCC | |
| | TGAAGATACCGCAGTGTACTATTGTTATCTGAGGGTGTT | |
| | CGCTCGGCGCTATTGGGGCCAGGGCACACAGGTGACAG | |
| | TGTCCTCC | |
| DR1458 DNA | GAAGTGCAGCTGGTCGAGAGCGGGGGTGGACTT | 733 |
| | GTGCAGGCTGGTGGCTCCCTTAGGCTGAGCTGCGCCGC | |
| | TTCCGGCAGAACTCTCTCTACCTATGCTATGGGTTGGTT | |
| | CCGTCAGGCCCCCGGCAAGGAGCGCGAGTTCGTCGCGG | |
| | CCATCCGCTGGGCTTCTGGCCGTACTTATTACGGTGACA | |
| | GCGTGAAGGGTCGGTTCACCATCTCTCGTGACAGTGCG | |
| | AAAAATACCGTGTACCTCCAGATGAACTCCCTGAAGCC | |
| | GGAGGACACGGCGGTTTATTACTGCGCGGCCAGGAGCA | |
| | GGCCTTACCTGAACTACGGAGACTTTGGGTACTGGGGC | |
| | CAGGGGACCCAGGTCACCGTGTCATCC | |
| DR1459 DNA | GAAGTCCAGCTCGTGGAGTCTGGGGGTGGACTC | 734 |
| | GTACAAGCCGGGGGATCACTTCGCTTGTCCTGCGCGGC | |
| | TTCTGGCAGGACCATCTCAACTTACGCAATGGTTTGGTT | |
| | CAGGCAAGCCTCTGGTAAGGAGCGTGAGTTTGTGGGCG | |
| | TTATCTCCCGCAGTGGAGACCGCACTTACTATGCTGATT | |
| | CTGTGAAGGGCAGATTCACTATCAGTCGCGATAATCTG | |
| | GGCAACATTGTGCGTTTGCAGCTCAATTCACTTAAACCT | |
| | GAAGACACAGCCGTTTATTACTGCGCACGCGGCGGATA | |
| | TACCGGGATTGAGACAATTACGGCTCGGGGTCGCGGCA | |
| | CATTGGTCACCGTGTCCAGC | |
| DR1460 DNA | GAGGTTCAGCTCGTTGAGAGTGGTGGAGGCCTC | 735 |
| | GTGCAGACCGGGGATTCCCTTCGCCTTTCCTGTGCAGCT | |
| | CCAGAGTCCATCTTCAACAATAACGCCGTTTACTGGTAC | |
| | AGGCAGTTCCCCGGCAAGGAGAGGGAGTATGTTGGTCT | |
| | CATCACCATCGGTGGCAGGACCGGGTACGCGGACTCTG | |
| | TGAAAGGCCGCTTTACCATCTCCAGAGACAACGCCAAT | |
| | AACGTGGCCTTTTTGCAGATGGATTCCCTCAAGCCCGA | |
| | GGATACTGCTGTCTACTATTGTGCCACGGGCTTGAAGTT | |
| | CGGCTTCAACTTCTACAGTAAGACTGCCTACGACTACTG | |
| | GGGACAAGGGACCCAGGTGACCGTCAGCTCT | |
| DR1461 DNA | GAGGTGCAGCTCGTGGAGTCTGGAGGTCGCCTG | 736 |
| | GTGCAGGCTGGCGATTCCCTGCGCCTGTCCTGTGTGGCC | |
| | TCTGGTCGCATTTTCAACACTTATTCTATGGGTTGGTTC | |
| | AGACAGGTTCCTGGAAAGGAAAGAGACTTCGTGGCAGC | |
| | CATTCGGTGGAGTGGTGGCACCACTTATTACACAGACT | |
| | CCGTGAAGGGTCGCTTTACTATCTCTCGGGATAACGCC | |
| | AAAAACACTGTCTACCTCCAGATGAAAGACCTGAAGCC | |
| | CCAGGACACCGCCGTCTATTACTGTGCTGCCGTCCCCTC | |
| | TGGCCGCAGCTGGTACGGTCGCAACCGTTACTGGGGCC | |
| | AGGGCACTCTGGTGACCGTCAGCTCT | |
| DR1462 DNA | GAGGTGCAGTTGGTGGAGAGCGGCGGTGGCCTG | 737 |
| | GTCCAGGCGGGCGGGTCCCTCCGCCTGAGTTGTGTGTCT | |
| | TCAGGCCGGACCTTTGGATATGTCGCTATGGGTTGGTTC | |
| | CGTCAAGCCCCAGGTAAGGAACGCGAGTTCGTGGCGAG | |
| | CATTAACTGGAGCGGCGGGTCCACGGCCTATGCGGACT | |
| | CCGTAAAGGGCCGGTTCACTATCAGCCGCGACAACGCT | |
| | AAGAATACCGTGTACTTGCAGATGAACAGCCTGAAGCC | |
| | TGAGGATACAGCCGTGTATTACTGCGCTGGATCAACCC | |
| | GCTTCTATATCGCGACGATGGAACAGGGCTCCTACGAT | |
| | TACTGGGGCCAAGGTACTCAGGTGACCGTAAGCAGC | |
| DR1463 DNA | GAGGTGCAACTGGTGGAATCAGGAGGCTCCGTG | 738 |
| | GTCCAGCCAGGGGACAGCCTTCGTCTTGCCTGCACCGC | |
| | CTCTGGTCGCAGTTTCAGGTCTTACGCGATTGGCTGGTT | |
| | TAGGCAGGCATCCGGCAAGGAAAGGGTGTTTGTGGCTG | |
| | CCATCTCTTATGACGGTAGGCGCACCTACTATGGGCGTT | |
| | CATTGAAGGACCGTTTCACTATCTCTCGGGACAACGCT | |
| | AAGAACACAGTGTACTTGCAGATGAACTCCCTCAAGCC | |
| | CGAGGACACTGCCGTGTACTATTGCGCTACCCATCGCTC | |
| | CGGTACAATGTTCGCTCGGTATGGTATGGATTACTGGG | |
| | GTAAGGGTACTTTGGTTACCGTGTCCAGC | |
| DR1464 DNA | GAGGTGCAGCTGGTGGAGAGCGGCGGTGGCCTG | 739 |
| | GTGCAAGCAGGCGGATCTCTGCGTCTGTCTTGTGCTGCG | |
| | TCAGGCCGCACCTTCTCCTCTTATGCTATGGGGTGGTTT | |
| | AGACAAGCTCCTGGAAAGGAGAGGGAGTTTGTGACTGC | |
| | CATCTCCAGATCCGGTGGATACACTAGCTACGCCGATA | |
| | GTGTTAAGGGCCGGTTCACTATCTCTCGCGACAATGCC | |
| | AAGAACACCGTGTATCTTCAGATGAACTCCCTGAAACC | |
| | CGAGGACACCGCCGTCTACTATTGTGCGAAACTGATCG | |
| | CTCCATTCTATTACGGCATGGATTACTGGACCAAGGGG | |
| | ACCCAGGTGACAGTGTCTAGC | |
| DR1465 DNA | GAAGTGCAGCTGGTGGAAAGCGGCGGAGGTCTG | 740 |
| | ATGCAGGCAGGTGGAGCCCTTAGGCTCTCTTGTACCGC | |
| | CTCTGGGCCTACTTTTACCTCTTATACGATGGGCTGGTT | |
| | CCGCCAATCTCCTGGCAAGCGTCGCGAGTTTGTGGCCG | |
| | TCATCTCCAAAGGCGGGCGGACCTATTACGCCGACTCC | |
| | GTGAAGGGACGCTTCACTATTTCCCGCGACAACGCTAA | |
| | GAATACCTTCTATCTCCAGATGTCCTCTCTGAAGCCTGA | |
| | GGACACAGCAGTGTATTACTGCGCCGGGCAGCGTGTGG | |
| | GCGCGACTAGCAAGTATGAGTATGATTACTGGGGGCAG | |
| | GGCACCCAAGTGACCGTGTCATCC | |
| DR1466 DNA | GAAGTGCAACTGGTGGAGAGCGGAGGGGGTCTG | 741 |
| | GTACGCGCAGGTGGCTCCCTGAGGCTCTCCTGCGCTGC | |
| | GTCCGGCTTCACTTTTAGTACCGACTGGATGTACTGGGT | |
| | AAGACGCGCTCCAGGAAAGGGGCTGGAGTGGGTGTCCC | |
| | TTATCAACACTGACGGGACTTCTACCTCCTATACTAAGT | |
| | CTGTGAAGGGGCGCTTCACAGTCTCCCGCGATAATGCC | |
| | AAGAACACCTTGTACCTTCAGATGAACTCCCTCAAGCC | |
| | GGAGGACACAGCTCTGTATTACTGTGCACGCGGAAGAA | |
| | CCTACTGGTTTTACGCGATGGATTACTGGGGCAAGGGC | |
| | ACCCAGGTGACCGTCTCATCT | |
| DR1467 DNA | GAGGTCCAGTTGGTGGAATCTGGAGGCGGACTG | 742 |
| | GTGCAGGCTGGAGACAGTCTGAGATTGTCTTGTGCCGC | |
| | TTCTGGCCGGATCAGCAACTACGCAATGGGCTGGTTCC | |
| | GGCAGGCACCCGGTAAAGAAAGGGAGTTCGTCGCTGTC | |
| | ATCACCAGGAGCGGCGGAAGCACATACTATGCTGATAG | |
| | TGTTAAGGGCCGCTTCACCATTTCCAGAGATAACGGCA | |
| | AAAACACGATTGATCTTCAGATGAACAGACTGAAGCCT | |
| | GAAGACACAGCAGTGTACTATTGTGCCGTGAGGCGCAG | |
| | TCAAAAACTGGTAACCTTTGGCGCTGAGTATCCTTGGTG | |
| | GGGCCAGGGAACATTGGTGACTGTCAGCTCC | |
| DR1468 DNA | GAGGTGCAGTTGGTGGAGAGCGGCGGAGGCTTG | 743 |
| | GTTCAAGCTGGGGGCTCACTCAGGCTGTCTTGCACTACC | |
| | TCTGGGCGTACAGGCACCCATTATGCGATGGGTTGGTTT | |
| | AGGCAAGCGCCCGGCAAGGAACGCGAGTTCGTTAGTCT | |
| | CATCCTGTGGAACGGCGAGTTTACGACCTATAAAGATT | |
| | CTGTTAAGGGCCGCTTCACCATCTCCCGTGAGAAAGGC | |
| | GAAAACACGGTCTACTTGCAAATGAACTCTCTGAAACC | |
| | CGAGGATACTGCGGTGTATTACTGCTACCTGAGGGTGT | |
| | TTGCTAGGCGCTACTGGGGCCAGGGAACCCAGGTGACC | |
| | GTGTCCAGT | |
| DR1469 DNA | GAAGTGCAGCTGGTGGAAAGTGGAGGCGGACTG | 744 |
| | GTGCAGCCAGGGGGCAGCCTCCGCCTTTCTTGTGAGGT | |
| | GTCCGGCTTTACCTTCAGCAACTACTGGATGTACTGGAT | |
| | TCGCCAAGCCCCTGGGAAGGGACTGGAGTGGGTGTCCC | |
| | ACATTAACACCAACGGTGGCAACACTTATTACCGCCAT | |
| | AGTGTTAAAGGTAGATTCACTATCAGCAGGGATAACGC | |
| | TAAGAATACCCTGTACTTGCAGATGAACGGCCTGAAGT | |
| | CCGAGGACACCGCTGTGTATTACTGTGCCAAGGCTAAC | |
| | TCCGATGTCGGGTTGGGTTATTACGGCATGGATTACTGG | |
| | GGTAAGGGAACTCAGGTCACAGTGAGTTCT | |
| DR1470 DNA | GAGGTGCAGCTGGTGGAAAGTGGCGGGGGCTCT | 745 |
| | GTTCAGCCCGGCGGATCTCTGCGCCTGAGCTGTGCTGC | |
| | ACCAGAGTCCATCTTCAACAATAACGCTGTTTACTGGTA | |
| | TCGGCAATTTCCGGGCAAAGAAAGGGAGTACGTGGGCC | |
| | TCATCACGATTGGTGGGCGCACTGGATACGCCGACTCT | |
| | GTCAAGGGCCGCTTTACTATCAGTCGTGATAACGCCAA | |
| | CAATGTTGCTTTTCTCCAGATGGATAACCTGAAGCCGG | |
| | AAGATACTGCGGTATATTACTGTGCCGCTAGGCCTGGA | |
| | TATTGGTCCAGTTCCTACGATTATTGGGGTCAGGGAACC | |
| | CAAGTAACAGTGTCCTCT | |
| DR1471 DNA | GAAGTGCAGCTGGTGGAAAGCGGCGGTGGCCTC | 746 |
| | GTGCAGGCGGGCGGGTCCCTGAGACTGTCATGCGTCTT | |
| | CTCTGGCCGCGCCCCGGCTAGTTATGCAATGGCTTGGTT | |
| | TCGCCAGGCCGTGGGCAACGAGAGGGAGTTTGTCGCTG | |
| | CGATCAACTGGTCCGGCAGGCGCACTTACTATGCCGAC | |
| | TCAGTGAAGGGCCGCTTCACTATTTCCAAGGACAATGC | |
| | ACAGAACACCGCCTATCTCCAGATGACCAACTTGGAAC | |
| | CAGAGGATACTGCCACGTATTACTGTAATGCTTACTTGA | |
| | GCGGAACATATTACTGGGGCCAGGGCACCCAGGTGACC | |
| | GTCTCTAGC | |
| DR1472 DNA | GAGGTCCAGCTGGTCGAGTCTGGCGGTGGCTTG | 747 |
| | GTCCGCGCTGGGGACTCACTGCGCCTGAGTTGTGCTGT | |
| | GTCCGGCCTGGCCAGCTCCTCTTTCTTTATGACTTGGTT | |
| | CCGCCAAGGGCAGGGCAAGGAGCGGGAATTTGTGGCC | |
| | ACTATCAGTTGGACTGGCCGTACATCCTATTACGCTGCC | |
| | AGCGTGAAAGGCCGCTTTACCGTTAGTCGGGACAATGC | |
| | CAAGAATACCGTGTACCTTCAGATGAACTCTCTGAACT | |
| | CTGAGGATACAGCAGTCTACTTCTGTGCAGCCTACCCG | |
| | CGTACACTGGTGCGTAATCGCGAGCCGATCCATTGGGG | |
| | TCAGGGAACCCAGGTGACTGTGTCCTCC | |

TABLE 40
anti-IL2Rg VHH DNA sequences

| | | SEQ |
|---|---|---|
| | | ID |
| Name | Sequence | NO: |
| hIL2Rg_VHH-1 | CAGGTCCAGCTCCAGGAGAGC | 748 |
| | GGGGGCGGTTCTGTGCAAGCCGGAGG | |
| | CTCATTGAGACTCTCATGCGCTGCAAG | |
| | TGGTTTTACCTTCGATGACAGCGATAT | |
| | GGGATGGTATCGTCAGGCTCCGGGCAA | |
| | TGAGTGTGATCTGGTCTCCACTATCTCC | |
| | TCTGATGGTTCCACATACTATGCTGAC | |
| | TCTGTCAAGGGGCGCTTTACCATCTCC | |
| | CAAGATAATGCCAAGAACACCGTGTAC | |
| | CTTCAGATGGATTCAGTTAAGCCCGAG | |
| | GACACAGCCGTCTATTACTGCGCTGCG | |
| | GATTTTATGATTGCCATCCAAGCTCCC | |
| | GGAGCGGGATGCTGGGGCCAGGGAAC | |
| | CCAGGTCACTGTGAGCAGT | |
| hIL2Rg_VHH-2 | CAGGTGCAGTTGCAGGAGTCC | 749 |
| | GGCGGGGGTTCTGTGCCAGCGGGTGGG | |
| | AGCCTCAAGCTCTCCTGTGCCGCTTCC | |
| | GGCTTCTCATTCTCCTCTTACCCTATGA | |
| | CCTGGGCACGCCAAGCGCCCGGCAAG | |
| | GGACTGGAATGGGTGTCCACCATTGCT | |
| | TCCGATGGCGGTAGTACAGCCTACGCC | |
| | GCGTCAGTGGAGGGTCGGTTCACGATC | |
| | AGCCGGGACAACGCGAAGAGCACACT | |
| | CTACCTCCAGCTGAACTCTCTGAAGAC | |
| | CGAGGACACCGCCATGTACTATTGCAC | |
| | AAAGGGCTACGGCGACGGCACCCCGG | |
| | CACCCGGCCAGGGCACCCAGGTGACA | |
| | GTCTCTTCC | |
| hIL2Rg_VHH-3 | CAGGTGCAGTTGCAGGAAAGT | 750 |
| | GGTGGAGGGAGTGTGCAGACTGGGGG | |
| | CTCTCTCCGCCTCAGCTGCACAGCCTCT | |
| | GGATTTACCTTCGATGATCGCGAGATG | |
| | AACTGGTATCGCCAGGCTCCGGGAAAC | |
| | GAGTGCGAACTGGTGTCTACAATCAGT | |
| | TCTGACGGGTCCACCTATTACGCTGAT | |
| | AGTGTCAAGGGCCGCTTCACTATCTCT | |
| | CAGGACAACGCGAAGAACACCGTTTA | |
| | CTTGCAGATGGATAGCGTGAAGCCTGA | |
| | AGATACAGCGGTGTATTACTGCGCTGC | |
| | CGACTTTATGATTGCCATCCAGGCACC | |
| | GGGGGCGGGGTGTTGGGGACAGGGAA | |
| | CTCAGGTGACTGTGTCCTCC | |
| hIL2Rg_VHH-4 | CAGGTTCAACTCCAAGAGAGT | 751 |
| | GGTGGCGGAAGCGTGCAGGCGGGCGG | |
| | TTCTCTGCGTCTGAGTTGCACTGCCAG | |
| | CGGATTTACCTTCGACGATTCCGACAT | |
| | GGGATGGTACAGACAGGCCCCTGGTA | |
| | ACGAGTGCGAACTCGTGAGTACTATCA | |
| | GCTCCGACGGCAACACCTATTACACCG | |
| | ATTCTGTGAAGGGCAGGTTCACCATCT | |
| | CCCAGGACAACGCTAAGAACACTGTGT | |
| | ACCTGCAAATGAATAGCCTGGGACCCG | |
| | AGGACACAGCGGTCTATTACTGCGCGG | |
| | CAGAGCCGCGCGGCTATTACAGCAACT | |
| | ACGGCGGTAGACGCGAGTGCAACTACT | |
| | GGGGGCAGGGGACGCAAGTGACTGTC | |
| | TCCTCC | |
| hIL2Rg_VHH-5 | CAAGTGCAGCTTCAGGAGTCC | 752 |
| | GGGGGTGGCAGCGTCCAGGCTGGGGG | |
| | CAGCTTGCGCCTGTCTTGCGCTGCGTCT | |
| | GGGTTCAGCTTTAGCTCCTACCCTATG | |
| | ACCTGGGCTAGACAGGCCCCCGGCAA | |
| | GGGGCTGGAGTGGGTGAGTACAATCG | |
| | CCTCCGACGGAGGTAGTACGGCCTACG | |
| | CAGCGTCCGTCGAGGGTCGCTTCACCA | |
| | TCAGCCGGGATAACGCTAAGTCCACCC | |
| | TGTACCTTCAGCTCAATTCTCTCAAAA | |
| | CGGAGGATACCGCCATGTACTATTGCA | |
| | CCAAGGGATATGGCGACGGCACCCCA | |
| | GCTCCTGGACAGGGCACACAGGTCACC | |
| | GTTAGCTCC | |
| hIL2Rg_VHH-6 | CAGGTCCAGCTTCAGGAGTCTG | 753 |
| | GCGGGGGCGCAGTACAGGCAGGGGGT | |
| | TCTCTGCGTCTGTCCTGCGCCGCGTCCG | |
| | GCTTTACTTTCAGCAACGCACACATGA | |
| | GTTGGGTGCGCCAAGCGCCCGGCAAG | |
| | GGCCGGGAATGGATCAGTAGCATCTAC | |
| | AGTGGAGGCAGCACATGGTACGCCGA | |
| | CTCTGTTAAGGGTCGTTTTACGATCTCT | |
| | CGTGACAACTCCAAGAACACTTTGTAC | |
| | CTCCAGCTCAATTCTCTCAAGACCGAG | |
| | GACACCGCGATGTACTATTGTGCCGAG | |
| | AACAGGCTGCACTACTATTCCGACGAT | |
| | GACTCTCTCAGGGGCCAGGGAACTCAA | |
| | GTTACCGTGTCCAGC | |
| hIL2Rg_VHH-7 | CAAGTGCAGCTCCAAGAGAGT | 754 |
| | GGTGGCGGGCTGGTTCAGCCAGGGGG | |
| | CAGCTTGAGACTCTCCTGCGCAGCTTC | |
| | AGGCTTTACCTTCGATGACCGTGAGAT | |
| | GAACTGGTATCGTCAGGCCCCAGGCAA | |
| | CGAGTGTGAGCTGGTTAGCACGATTTC | |
| | TTCCGACGGTTCCACCTATTACGCCGA | |
| | CTCTGTGAAGGGACGTTTCACTATCTC | |
| | CCAGGACAATGCCAAGAACACCGTGT | |
| | ACCTCCAGATGGACAGCGTGAAGCCG | |
| | GAGGATACTGCTGTGTATTACTGCGCT | |
| | GCCGACTTTATGATCGCCATCCAGGCC | |
| | CCTGGCGCGGGTTGCTGGGGCCAGGGC | |
| | ACTCAGGTGACCGTGTCTTCC | |
| hIL2Rg_VHH-8 | CAAGTGCAACTGCAAGAGTCC | 755 |
| | GGCGGTGGATCTGTGCAGGCCGGAGG | |
| | CAGCCTGCGGCTGAGCTGTGTAGCTTC | |
| | CGGGTATACCTTTAGCTCATACTGTAT | |
| | GGGCTGGTTTCGTCAGGCCCCCGGTAA | |
| | GGAGCGCGAGGGCGTGGCCGCTCTTGG | |
| | TGGAGGCTCCACCTATTACGCCGATTC | |
| | CGTGAAGGGCAGGTTTACTATCTCCCA | |
| | GGACAACGCGAAGAATACGCTCTATCT | |
| | CCAGATGAATAGCCTGAAGCCCGAGG | |
| | ATACAGCTATGTATTACTGTGCTGCCG | |
| | CTTGGGTAGCCTGCCTGGAGTTCGGTG | |
| | GCTCCTGGTACGATCTGGCACGGTACA | |
| | AACATTGGGGGCAGGGCACCCAGGTC | |
| | ACCGTGTCTAGC | |
| hIL2Rg_VHH-9 | CAGGTCCAGTTGCAGGAATCTG | 756 |
| | GGGGCGGTTCCGTACAAGCAGGTGGCT | |
| | CCCTTCGGTTGAGCTGTACCGCATCCG | |
| | GCTTTACTTTCGACGATAGCGATATGG | |
| | GCTGGTATCGTCAGGCCCCAGGGGGCG | |
| | AGTGCGAGCTGGTTACAATCTCCTCTG | |
| | ACGGCAGTACCTATTACGCAGACTCCG | |
| | TCAAGGGCAGGTTCACTATCAGTCAGG | |
| | ACAATGCAAAGAACACTGTGTATCTCC | |
| | AGATGAACTCTCTGAAGCCAGAAGATA | |
| | CTGCCGTGTATTACTGCGCTGCGGAAC | |
| | CGAGAGGCTATTACTCTAATTATGGCG | |
| | GGCGTCGGGAGTGTAATTATTGGGGAC | |
| | AGGGAACCCAGGTGACCGTGTCCTCC | |
| hIL2Rg_VHH-10 | CAGGTGCAGCTCCAGGAGAGT | 757 |
| | GGCGGAGGCTCCGTGCAGGCTGGGGG | |
| | CTCTCTGCGTCTGAGCTGTGCCGCAAG | |
| | CGGTAGCATTTACAGCTCTGCCTACAT | |
| | CGGGTGGTTTCGTCAAGCGCCGGGCAA | |
| | AAAGCGCGAAGGCGTGGCCGGAATCT | |
| | ACACGCGCGATGGCTCCACCGCTTATG | |
| | CTGACAGCGTTAAGGGACGTTTTACGA | |
| | TCAGCCAGGACTCTGCCAAAAAGACTG | |
| | TGTATCTCCAGATGAACTCCCTGAAAC | |
| | CTGAGGACACAGCCATGTATTACTGCG | |
| | CCGCTGGCCGCCGTACAAAGAGCTATG | |
| | TTTACATCTTTCGCCCCGAAGAGTACA | |
| | ACTACTGGGGCCAGGGAACCCAAGTG | |
| | ACTGTGTCCAGT | |
| hIL2Rg_VHH-11 | CAGGTTCAGTTGCAGGAGTCCG | 758 |
| | GCGGAGGCAGCGTGCAGGCCGGAGGC | |
| | TCCTTGCGCTTGTCCTGTGCGGCTTCTG | |
| | GCTTCACCTTCTCATCTGCTCACATGAG | |
| | TTGGGTGCGTCAGGCCCCAGGGAAAG | |
| | GTCGCGAGTGGATTGCCTCCATCTACA | |
| | GCGGTGGGGGCACTTTTTATGCGGACA | |
| | GCGTGAAGGGCCGCTTTACCATCAGCC | |
| | GTGACAACGCTAAGAACACCCTGTATC | |
| | TCCAACTCAATTCCCTCAAGACCGAGG | |
| | ATACAGCGATGTACTATTGTGCAACCA | |
| | ACCGCCTTCACTATTACTCCGACGATG | |
| | ACAGCCTGCGCGGACAGGGGACCCAG | |
| | GTGACGGTGTCCAGC | |
| hIL2Rg_VHH-12 | CAGGTGCAACTCCAGGAAAGT | 759 |
| | GGCGGAGGCTCAGTGCAGGCAGGTGG | |
| | CTCTCTCCGCCTTTCCTGCGCTGCCAGC | |
| | GGATTCACCTTCTCTAACGCTCACATG | |
| | AGCTGGGTTCGTCAGGCTCCCGGCAAA | |
| | GGCCGTGAATGGATTAGCTCCATCTAT | |
| | AGTGGCGGAAGTACTTGGTACGCAGAT | |
| | AGCGTCAAGGGCCGCTTCACTATTAGT | |
| | CGGGATAACTCCAAGAACACTCTGTAC | |
| | CTCCAGCTGAACTCATTGAAAACCGAG | |
| | GACACGGCTATGTACTATTGTGCTGAG | |
| | AACAGGCTGCACTATTACTCCGACGAT | |
| | GACTCTCTGAGGGGTCAGGGCACCCAG | |
| | GTGACCGTCAGCTCC | |
| hIL2Rg_VHH-13 | CAGGTCCAACTCCAGGAGTCC | 760 |
| | GGCGGAGGCAGCGTGCAGGCTGGAGG | |
| | CTCTCTCCGCCTGAGCTGCACAGCTTC | |
| | CAGATTCATCTTCGATGACTCCGACAT | |
| | GGGCTGGTATCGCCAGGCTCCAGGGAA | |
| | CGAGTGCGAACTGGTGAGCACCATCTC | |
| | TTCAGACGGTAGCACCTATTACGCCGA | |
| | CAGTGTGAAGGGGCGCTTCACCATCTC | |
| | CCGCGACAATGCTAAAAATACGGTGTA | |
| | TCTCCAGATGAACTCCCTCAAACCGGA | |
| | GGACACAGCTGTATATTACTGTGCTGC | |
| | GGAACCACGGGGCTACTATAGCAACTA | |
| | TGGTGGAAGGCGCGAGTGCAACTACTG | |
| | GGGTCAGGGCACACAGGTGACGGTTTC | |
| | CTCC | |
| hIL2Rg_VHH-14 | CAGGTGCAGCTCCAGGAGAGC | 761 |
| | GGCGGTGGCTCCGTGCAGGCTGGTGGC | |
| | AGCCTGAAGCTGTCCTGCACCGTGAGT | |
| | GGCTTCACAGCCGACGATTCTGATATG | |
| | GGCTGGTATCGCCAAGGCCCCGGCAAT | |
| | GAGTGCGAGCTGGTAACCATTAGCTCA | |
| | GACGGCTCTACATACTATGCCGATTCT | |
| | GTTAAGGGCCGCTTTACTATCTCACAG | |
| | GATAATGCCAAGAACACAGTGTACTTG | |
| | CAGATGAACTCTCTGAAACCGGAAGAC | |
| | ACAGCTGTGTATTACTGTGCTGCGGAG | |
| | CCTAGAGGGTATTACAGCAATTACGGG | |
| | GGCCGGAGAGAGTGTAACTATTGGGG | |
| | GCAGGGCACCCAAGTGACCGTTTCCTC | |
| | C | |
| hIL2Rg_VHH-15 | CAGGTCCAGCTTCAGGAATCTG | 762 |
| | GGGGCGGTCTCGTGCAGCCCGGCGGGT | |
| | CCCTGCGTCTGTCTTGTGCTGCGAGCG | |
| | GCTTCACGTTCTCAAGTGCCCACATGA | |
| | GCTGGGTAAGGCAGGCACCGGGCAAG | |
| | GGGCGCGAGTGGATTGCAAGCATCTAT | |
| | TCAGGCGGGGGCACATTCTACGCCGAC | |
| | AGCGTGAAGGGACGTTTTACAATCTCC | |
| | AGAGATAACGCAAAGAACACTCTCTAC | |
| | CTCCAACTCAACTCCTTGAAGGCGGAA | |
| | GATACTGCAATGTATTACTGTGCTACT | |
| | AACCGTCTTCATTATTACTCTGACGAT | |
| | GACTCCCTGCGGGGGCAGGGTACACA | |
| | GGTGACAGTGAGTTCC | |
| hIL2Rg_VHH-16 | CAGGTGCAGCTGCAAGAATCT | 763 |
| | GGTGGAGGGCTGGTCCAGCCTGGGGG | |
| | CTCCCTGCGCCTCTCATGTGTCGCATCT | |
| | GGCTTCACCTTCAGCAACGCCCACATG | |
| | AGCTGGGTTCGCCAAGCCCCTGGGAAG | |
| | GGCCGGGAGTGGATCTCCAGTATCTAT | |
| | TCCGGCGGAAGCACTTGGTATGCAGAC | |
| | AGCGTCAAAGGACGGTTCACTATTTCT | |
| | CGTGATAATTCTAAGAACACCCTGTAC | |
| | CTTCAGCTGAACAGCCTGAAGACCGAG | |
| | GACACTGCTATGTACTATTGTGCTGAG | |
| | AATCGCCTGCATTACTATAGCGACGAT | |
| | GACAGTCTGCGCGGACAGGGGACCCA | |
| | GGTCACCGTGTCCTCT | |
| hIL2Rg_VHH-17 | CAGGTTCAGTTGCAGGAATCA | 764 |
| | GGAGGCGGTCTGGTGCAGCCTGGGGG | |
| | CTCTCTGCGTCTCTCCTGCGCCGCTTCC | |
| | GGCTTCACATTCTCCAACGCCCACATG | |
| | AGCTGGGTCCGCCAGGCCCCTGGGAAG | |
| | GGCCGCGAGTGGATCTCCAGTATCTAC | |
| | AGCGGGGGCTCCACTTGGTACGCAGAC | |
| | AGCGTCAAAGGGAGGTTTACCATTAGC | |
| | CGTGACAATTCTAAGAACACATTGTAT | |
| | TTGCAGCTGAACTCTCTTAAAACCGAG | |
| | GACACCGCCATGTACTATTGTGCTGAG | |
| | AACAGGCTCCACTATTACTCAGACGAT | |
| | GACTCACTTCGCGGGCAGGGAACCCAG | |
| | GTCACCGTCTCCTCT | |
| hIL2Rg_VHH-18 | CAAGTCCAGCTCCAGGAAAGC | 765 |
| | GGCGGTGGCCTGGTGCAACCTGGCGGG | |
| | TCTCTGCGCTTGTCATGCGCTGCCTCCG | |
| | GCTTCACCTTCTCATCTTACCCTATGAC | |
| | CTGGGCGCGTCAGGCTCCCGGCAAGGG | |
| | ATTGGAGTGGGTGTCTACTATTGCCTC | |
| | CGACGGTGGCAGCACGGCCTACGCAG | |
| | CGTCTGTAGAAGGACGCTTCACAATTA | |
| | GCAGAGACAACGCAAAATCTACTTTGT | |
| | ACCTTCAGCTCAACAGCCTGAAGACCG | |
| | AAGACACAGCTATGTATTACTGCACAA | |
| | AAGGCTACGGGGACGGCACGCCAGCG | |
| | CCTGGACAGGGGACACAGGTGACCGT | |
| | ATCTTCT | |
| hIL2Rg_VHH-19 | CAGGTGCAGTTGCAGGAATCA | 766 |
| | GGGGGTGGCTCTGTGCAGGCCGGGGG | |
| | CTCCCTGCGTCTGTCCTGTACTGCGAG | |
| | CGGCTTCACCTTTGATGACCGCGAGAT | |
| | GAACTGGTATCGCCAGGCTCCGGGGAA | |
| | CGAGTGCGAACTCGTGTCTACAATTAG | |
| | CTCCGATGGTTCAACATACTATGCTGA | |
| | TTCTGTCAAAGGTCGCTTTACCATCTCA | |
| | CAGGACAACGCCAAGAACACCGTCTA | |
| | CCTCCAGATGGACTCTGTGAAGCCTGA | |
| | AGATACCGCCGTATACTATTGCGCCGC | |
| | TGACTTTATGATTGCCATTCAGGCTCC | |
| | GGGTGCTGGATGCTGGGGTCAGGGGA | |
| | CTCAGGTGACCGTGTCTTCA | |
| hIL2Rg_VHH-20 | CAAGTGCAGTTGCAGGAAAGC | 767 |
| | GGCGGTGGGTCCGTGCAAGCCGGAGG | |
| | TTCTCTCCGCCTGTCTTGCACTGCCTCA | |
| | GGTTTTACCTTCGACGATTCCGATATG | |
| | GGCTGGTACAGGCAGGCTCCCGGCAAT | |
| | GAGTGCGAGCTGGTGTCTACGATCTCA | |
| | AGTGATGGCTCCACCTACTATGCCGAT | |
| | AGCGTAAAAGGAAGGTTTACTATTAGC | |
| | CAGGATAACGCGAAGAACACGGTGTA | |
| | CCTCCAGATGAACAGTCTCAAGCCGGA | |
| | GGATACTGCCGTGTATTACTGTGCTGC | |
| | CGAGCCGCGTGGCTATTACTCCAACTA | |
| | CGGTGGCAGACGTGAATGCAATTACTG | |
| | GGGACAGGGTACTCAGGTTACCGTGTC | |
| | CTCT | |
| hIL2Rg_VHH-21 | CAGGTTCAACTTCAGGAATCCG | 768 |
| | GGGGCGGTTCCGTGCAAGCCGGGGGT | |
| | AGCCTGCGTCTGTCTTGCGTGGCCAGC | |
| | GGCTATACCTCCTGTATGGGTTGGTTTC | |
| | GGCAGGCTCCTGGGAAGGAGCGCGAA | |
| | GCCGTGGCGACCATCTACACACGGGGC | |
| | CGCAGCATCTATTACGCTGACAGTGTG | |
| | AAGGGCCGCTTCACCATCTCCCAGGAT | |
| | AACGCCAAGAATACCCTGTATCTGCAA | |
| | ATGAACTCCCTGAAGCCTGAGGACATC | |
| | GCCATGTATTCCTGCGCAGCTGGAGGG | |
| | TACTCATGGTCCGCTGGGTGCGAGTTT | |
| | AATTATTGGGGCCAAGGAACCCAGGTG | |
| | ACCGTCTCCTCA | |
| hIL2Rg_VHH-22 | CAAGTGCAGCTCCAGGAGTCT | 769 |
| | GGCGGGGGCCTGGTTCAGCCTGGTGGG | |
| | TCCCTGCGCCTGTCTTGCACGGCTTCCG | |
| | GCTTTAGCTTCTCCTCATATCCAATGAC | |
| | CTGGGCACGCCAGGCTCCTGGTAAGGG | |
| | CCTGGAGTGGGTCTCCACCATCGCCTC | |
| | TGATGGTGGGTCAACTGCCTATGCTGC | |
| | CTCCGTCGAGGGTAGATTCACAATCAG | |
| | CAGAGACAACGCCAAATCCACGCTGTA | |
| | CCTGCAACTCAACTCCTTGAAGACCGA | |
| | GGACACAGCTATGTATTACTGTACCAA | |
| | AGGCTACGGCGACGGCACTCCTGCTCC | |
| | CGGACAGGGGACCCAGGTGACTGTGTC | |
| | TAGC | |
| hIL2Rg_VHH-23 | CAGGTCCAACTTCAGGAAAGC | 770 |
| | GGGGGTGGACTGGTACAGCCAGGGGG | |
| | CAGTCTGCGCCTGTCCTGTGCCGCAAG | |
| | CGGGTTTTCTTTCTCCAGTTACCCCATG | |
| | ACCTGGGCTCGCCAAGCACCTGGAAAG | |
| | GGACTGGAGTGGGTGTCTACTATTGCG | |
| | TCAGATGGTGGGAGTACGGCTTACGCC | |
| | GCGAGCGTGGAGGGTCGTTTTACGATC | |
| | AGTAGGGACAACGCCAAAAGCACTCT | |
| | GTACCTCCAGCTTAACAGCCTGAAGAC | |
| | CGAGGACACCGCCATGTATTACTGTAC | |
| | CAAGGGCTACGGAGACGGCACCCCTG | |
| | CGCCGGGGCAAGGCACCCAGGTGACC | |
| | GTAAGTTCA | |

TABLE 41
murine anti-IL2Rg VHH DNA sequences

| | | SEQ |
|---|---|---|
| | | ID |
| Name | DNA Sequence | NO: |
| mIL2Rg_ | CAGGTGCAACTCCAGGAGTCCGGCGGGGGCT | 771 |
| VHH1 | CCGTGCTGGCTGGCGGATCTTTGAGGCTGTCTTGCGT | |
| | GGCTTCTGGCTATGGCTATAATTACATCGGCTGGTTC | |
| | CGTCAGACACCCGGCAAGGAGCGCGAAGGGGTGGC | |
| | GGTCATTTACACAGGGGGTGGGGACACTTATTACGC | |
| | CGACTCCGTCAAGGGTAGGTTTACCGCTAGTCGCGAT | |
| | AATGCCAAAAGTACGCTGTACCTGCAAATGAACAGC | |
| | TTGGAGCCAGAGGACACCGCCATGTATTACGGAGTG | |
| | GCTCGCTACTGTGTGGGCAGTGTGTACGCTTGCCTGC | |
| | GCGGAGGCCACGACGAGTACGCACACTGGGGCCAGG | |
| | GAACCCAGGTGACAGTGTCTAGC | |
| mIL2Rg_ | CAGGTGCAGCTCCAGGAGTCTGGGGGTGGCA | 772 |
| VHH2 | GCGTCCAGCCAGGTGGCTCATTGAGACTGTCTTGTGC | |
| | TGCATCTGGCTCCACCTACGCTAATTACCTGATGGGA | |
| | TGGTTCAGGCAGGCCCCTGGTAAGGAGCGTGAGGGC | |
| | GTGGCCGCTATCTATTCTGGCGGTGGGTCCACCTACT | |
| | ATGCTGACTCCGTCAAGGGACGCTTCACTATTTCTCA | |
| | AGACAATGCCAAGAACACTTTGTACTTGCAAATGAA | |
| | CTCACTCAAACCTGAGGACACCGCGATGTACTATTGC | |
| | GCAGCGGCATCCGCAGTGAAGGGAGACAAAGGGGA | |
| | TATCGTGGTAGTTGTGACCGGCACCCAGCGTATGGA | |
| | GTACGACTACTGGGGACATGGCACCCAGGTGACAGT | |
| | TAGCTCC | |
| mIL2Rg_ | CAGGTACAGTTGCAGGAGAGTGGTGGGGGTT | 773 |
| VHH3 | CCGTCCAGGCCGGTGCCTCTCTTCGCCTCAGTTGTAG | |
| | CGTGAGCGGTTTCACGTTCGACGAGTCAGTGATGTCC | |
| | TGGTTGCGCCAGGGTCCCGGCAATGAGTGCGACGCG | |
| | GTCGCTATTATCAGCTCCGATGACAACACCTATTACG | |
| | ACGATAGCGTGAAAGGCCGCTTTACCATCTCCGAGG | |
| | ACAACGCCAAAAACATGGTGTATCTGCAAATGAACT | |
| | CACTGAAGCCGGAAGACACCGCAGTGTACTATTGCG | |
| | CCGCGCGTCGGCGCAGACCTGTGTACGATTCCGATTA | |
| | TGAACTCCGGCCACGTCCGCTGTGTGGCGATTTCGGC | |
| | GTGTGGGGCCAGGGGACCCAGGTGACGGTCTCCTCC | |
| mIL2Rg_ | CAGGTGCAGCTCCAGGAATCTGGCGGGGGCT | 774 |
| VHH4 | CTGTGCAGGCTGGTGGCTCCCTTCGCCTGTCCTGTAT | |
| | TGGCTCCGGTCTTCCTTTCGACGAGGATGACATGGGC | |
| | TGGTATCGCCAGGCCCCTGGGAATGAGTGTGAATTG | |
| | GTCAGCTCAATCTCCAGTGACGGCACCGCCTATTACG | |
| | CCGATTCCGTCAAGGGACGCTTCACTATCTCCAGAGA | |
| | CAACGCCAAGAACACTGTGCTGTTGCAGATGAACTC | |
| | CCTGAAGCCCGAGGATACCGCTGTCTATTACTGCGCA | |
| | GCCGGGGTCCACAGACAGTTCGGCGGTTCCAGTTCCT | |
| | GCGGCGACGCCTTCTACGGCATGGATTACTGGGGCA | |
| | AGGGAACTCAGGTCACAGTGTCTTCC | |
| mIL2Rg_ | CAGGTTCAGCTTCAGGAGTCCGGCGGGGGCT | 775 |
| VHH5 | CCGTACAGGCAGGGGGCTCACTGCGTCTTTCCTGTGT | |
| | GGCGAGTGGCGACGTGTATGGCCGTAACAGCATGGC | |
| | TTGGTTCCGGCAGGCACCTGGAAAGGAACGCGAGGG | |
| | CGTTGCAGTTGGGTATTCCGTAGTGACAACCACTTAC | |
| | TATGCCGACAGTGTGAAGGGCCGGTTTACGATCTCA | |
| | GAGGACAACGATAAAAACACAGTGTACCTGGAGATG | |
| | AACTCCCTGAAGCCGGAAGACACTGCTATGTATTACT | |
| | GCGCTGCCGATGGCAACCTGTGGCGCGGACTCAGGC | |
| | CCTCCGAGTACACTTATTGGGGTCAGGGCACCCAGG | |
| | TGACCGTTTCAAGT | |
| mIL2Rg_ | CAGGTCCAGCTTCAGGAGTCAGGTGGCGGTA | 776 |
| VHH6 | GTGTCCAGGCAGGCGGTAGCCTGCGCCTTAGCTGTG | |
| | CTACATCCGGCTTCCCTTACTCACGCTATTGTATGGG | |
| | CTGGTTCAGGCAAGCTCCCGGTAAAGAGCGCGAGGG | |
| | AGTGGCAGCCATCGAGCCTGACGGGAGCACATCTTA | |
| | TGCTGACTCTGTAAAGGGGCGTTTCACCATCTCTCAG | |
| | GACAACGCCGTTAATACACTGTACTTGCAAATGAAT | |
| | AACCTGAAGCCCGAGGACACAGCTATGTATTACTGC | |
| | GCAGCCGACGAGCGTTGCTTCTATTTGAAGGACTATG | |
| | ACCTCAGAAGGCCAGCCCAGTACCGCTACTGGGGGC | |
| | AGGGCACCCAGGTTACCGTGTCATCT | |
| mIL2Rg_ | CAGGTGCAGTTGCAGGAGAGTGGCGGTGGCC | 777 |
| VHH7 | TCGTGCAGCCTGGCGGAAGCCTCCGTCTGAGCTGCA | |
| | CTGTGTCCGGCTTCACTTTCGACGAGAGCGACATGGG | |
| | CTGGCTGAGGCAGAACCCTGGTAACGAGTGCGGCGT | |
| | TGTGAGTGTCATCACGTCTGATGACAACCCATACTAT | |
| | GATGACAGCGTCAAGGGCCGCTTTACTATCTCCGAG | |
| | GATAACGCCAAGAACATGGTGTACCTCCAGATGAAC | |
| | TCACTGAAGCCCGAGGATACCGGCGTTTATTACTGTG | |
| | CAACCAGGAGCCGTCAGCCTGTGTACTCACGCGATT | |
| | ACGAGCTGCGGCCCCGCCCCCTCTGTGGAGACTTTGG | |
| | TGTGTGGGGCCAGGGCACCCAGGTGACTGTTTCCAG | |
| | C | |
| mIL2Rg_ | CAGGTGCAGTTGCAGGAGAGTGGAGGGGGCT | 778 |
| VHH8 | CAGTGCAGGCTGGCGGGTCCTTGCGTCTGTCTTGCAC | |
| | CGCCTCTGGCTTCACCTTCGATGACTTCGATATGGGT | |
| | TGGTATCGCCAGGCTCCAGGGAACGAGTGCGAATTG | |
| | GTCAGCACTATCAGCGACGATGGCTCAACATATTAC | |
| | GCCGACTCTGTGAAGGGACGGTCTAGCATTAGCCGG | |
| | GACAACGCAAAGAACACCGTCTATCTCCAGATGAAC | |
| | CGCTTGAAGCCTGAGGATACCGGAGTCTATTACTGC | |
| | GCCGCTGAGGGCGCGTTGGGCTCCAAGACTAATTGT | |
| | GGCTGGGTGGGCAACTTCGGATATTGGGGCCAGGGA | |
| | ACACAGGTTACCGTTTCCAGC | |
| mIL2Rg_ | CAGGTGCAGTTGCAGGAGTCTGGAGGCGGTT | 779 |
| VHH9 | CCGTTCAGGCCGGGGGCTCTCTGCGCCTGTCCTGCGC | |
| | TGCCTCCGGGTTTACATTTGACGATTTCGATATGGGC | |
| | TGGTATCGCCAGGCCCCTGGCAACGAGTGCGAACTG | |
| | GTGTCTACTATCTCCGATGACGGCTCAACCTACTATG | |
| | CAGACTCCGTAAAGGGCAGATCCAGCATCTCCCGCG | |
| | ACAATGCCAAAAACACTGTGTACCTCCAGATGAACT | |
| | CCCTCAAGCCTGAGGATACGGCGGTGTACTATTGTGC | |
| | TGCCGAGGGTGCGCTCGGTAGCAAGACTAATTGCGG | |
| | CTGGGTGGGCAACTTCGGGTACTGGGGTCAGGGGAC | |
| | CCAGGTAACCGTGTCTTCT | |
| mIL2Rg_ | CAGGTGCAGTTGCAGGAAAGCGGTGGGGGCC | 780 |
| VHH10 | TGGTGCAGCCCGGAGGCAGCCTGCGCTTGAGCTGCG | |
| | CTGCCTCTGGCTTCACATTCGATGACTTCGATATGGG | |
| | CTGGTATCGTCAAGCACCCGGAAACGAGTGCGAGCT | |
| | GGTGAGTACAATCAGTGATGACGGATCTACCTACTA | |
| | TGCCGACAGCGTCAAGGGAAGATCCAGCATCAGTCG | |
| | CGACAACGCCAAGAGCACCGTTTACCTCCAGATGAA | |
| | CCGCCTCAAGCCTGAGGACACAGGAGTCTATTACTG | |
| | TGCTGCGGAGGGGGCCTTGGGCAGCAAGACTAACTG | |
| | TGGATGGGTGGGAAACTTCGGGTATTGGGGTCAGGG | |
| | TACACAGGTCACAGTGTCTTCA | |
| mIL2Rg_ | CAAGTTCAGCTTCAGGAAAGTGGGGGCGGGC | 781 |
| VHH11 | TGGTGCAGCCAGGGGGTTCCCTGAAGCTGAGCTGCG | |
| | CTGCCTCTGGGTTTACATTCTCTGATCGCGACATGGG | |
| | CTGGTATCGCCAAGCGCCGGGCAATGAATGCGAAAG | |
| | AGTGAGTACTATTTCTGACGATGGTTCTACTTACTAT | |
| | GCTGACTCCGTGAAGGGCCGTAGCTCCATTTCCAGG | |
| | GACAACGCGAAGAACACCGTATACCTCCAGATGAAC | |
| | TCTCTGAAGCCCGAGGACACCGCTGTGTATTACTGCG | |
| | CTGCCGAGGGGGCTCTCGGCTCAAAGACCAACTGCG | |
| | GATGGGTCGGTAACTTCGGCTACTGGGGCCAGGGCA | |
| | CCCAAGTGACAGTCTCCTCC | |
| mIL2Rg_ | CAGGTCCAGTTGCAGGAGAGCGGGGGTGGAA | 782 |
| VHH12 | GCGTCCTCGCCGGAGGGAGCCTCCGTTTGAGCTGCGT | |
| | CGCCTCAGGCTACGGCTACAATTACATCGGATGGTTC | |
| | AGACAGACGCCTGGTAAAGAGCGGGAAGGCGTCGCC | |
| | GTGATTTATATCGGTGGCGGAGACACCTATTACGCTG | |
| | ACTCAGTGAAGGGGCGTTTCACCGCAAGCCGGGACA | |
| | ACGCTAAGAGCACCCTGTACCTCCAGATGAACTCTCT | |
| | CGAACCTGAGGACACTGCAATGTATTACTGCGTGGC | |
| | TCGTTACTGCGTCGGGAGTGTCTACGCCTGCCTGAGG | |
| | GGCGGGCATGATGAGTATGCCCACTGGGGACAAGGA | |
| | ACACAGGTGACTGTCTCCAGT | |
| mIL2Rg_ | CAGGTTCAGCTCCAGGAGTCTGGTGGCGGTTC | 783 |
| VHH13 | CGTGCTGGCCGGGGGCTCTCTGCGCCTGTCTTGTGTC | |
| | GCCTCAGGGTACGGCTATAACTACATTGGCTGGTTCA | |
| | GACAGACCCCTGGGAAAGAGCGGGAGGGTGTGGCTG | |
| | TCATTTACACCGGCGGAGGCGACACCTACTATGCCG | |
| | ATTCAGTTAAGGGCAGGTTTACCGCGAGCCGTGACA | |
| | ACGCGAAGTCTACTCTGTACCTGCAAATGAACAGCC | |
| | TGGAACCTGAGGATACTGCGATGTACTATTGTGTGGC | |
| | CCGGTACTGCGTAGGCTCAGTGTATGCCTGCCTGCGC | |
| | GGGGGTCACGACGAGTACGCACACTGGGGACAGGG | |
| | AACTCAGGTCACCGTGTCTAGC | |
| mIL2Rg_ | CAGGTGCAACTCCAGGAGTCCGGCGGGGGCT | 784 |
| VHH14 | CCGTCCAAGCTGGTGGCTCACTGAGGCTTAGCTGTGC | |
| | TGCCTCCGGCTTTACTTTCGACGATTTCGACATGGGT | |
| | TGGTATCGCCAGGCTCCGGGCAATGAGTGCGAGCTG | |
| | GTCTCTACCATTTCCGATGACGGCTCTACCTACTATG | |
| | CCAACAGTGTTAAGGGTAGGTCTTCCATCTCCCGCGA | |
| | CAACGCTAAGAATATGGTGTACTTGCAGATGAACTC | |
| | TCTGAAGCCTGAGGACACTGCTGTCTACTATTGCGCT | |
| | GCCGAAGGTGCCCTGGGCTCAAAGACTAATTGCGGC | |
| | TGGGTCGGTAACTTTGGCTACTGGGGTCAGGGGACT | |
| | CAGGTGACCGTCAGCTCC | |
| mIL2Rg_ | CAGGTCCAGTTGCAGGAAAGCGGCGGGGGCT | 785 |
| VHH15 | CTGTTCAGGCAGGCGGAAGCCTTCGTCTGTCCTGTAC | |
| | TGCCAGTGGTTTCACCTTTGATGACTTTGACATGGGC | |
| | TGGTATCGGCAAGCCCCCGGAAACGAGTGCGAGCTG | |
| | GTATCCACCATTTCCGATGACGGGTCCACGTACTATG | |
| | CTGATAGCGTGAAGGGCAGGTCTTCCATCAGCCGGG | |
| | ACAACGCCAAGAACACAGTGTATTTGCAGATGAACC | |
| | GCCTCAAGCCAGAAGACACCGGGGTATATTACTGTG | |
| | CAGCGGAAGGTGCCCTGGGTAGCAAGATGAACTGCG | |
| | GATGGGTGGGTAATTTTGGATACTGGGGCCAGGGCA | |
| | CGCAGGTTACAGTGTCCAGC | |

1. An 112 receptor (IL2R) binding protein that specifically binds to IL2Rβ and IL2Rγ, comprising an anti-IL2Rβ VHH antibody and an anti-IL2Rγ VHH antibody.

2. The IL2R binding protein of claim 1, wherein the anti-IL2Rβ VHH antibody comprises a complementarity determining region 1 (CDR1) comprising an amino acid sequence of SEQ ID NO: 1 or SEQ ID NO: 410, a CDR2 comprising an amino acid sequence of SEQ ID NO: 2, and a CDR3 comprising an amino acid sequence of SEQ ID NO: 3; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
wherein each CDR independently comprises 0, 1, 2, or 3 amino acid changes relative to the sequence.

3. The IL2R binding protein of claim 1, wherein the anti-IL2Rβ VHH antibody comprises a complementarity determining region 1 (CDR1) comprising an amino acid sequence of SEQ ID NO: 5 or SEQ ID NO: 411, a CDR2 comprising an amino acid sequence of SEQ ID NO: 6, and a CDR3 comprising an amino acid sequence of SEQ ID NO: 7; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
wherein each CDR independently comprises 0, 1, 2, or 3 amino acid changes relative to the sequence.

4. The IL2R binding protein of claim 1, wherein the anti-IL2Rβ VHH antibody comprises a complementarity determining region 1 (CDR1) comprising an amino acid sequence of SEQ ID NO: 9 or SEQ ID NO: 412, a CDR2 comprising an amino acid sequence of SEQ ID NO: 10, and a CDR3 comprising an amino acid sequence of SEQ ID NO: 11; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
wherein each CDR independently comprises 0, 1, 2, or 3 amino acid changes relative to the sequence.

5. The IL2R binding protein of claim 1, wherein the anti-IL2Rβ VHH antibody comprises a complementarity determining region 1 (CDR1) comprising an amino acid sequence of SEQ ID NO: 13 or SEQ ID NO: 413, a CDR2 comprising an amino acid sequence of SEQ ID NO: 14, and a CDR3 comprising an amino acid sequence of SEQ ID NO: 15; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
wherein each CDR independently comprises 0, 1, 2, or 3 amino acid changes relative to the sequence.

6. The IL2R binding protein of claim 1, wherein the anti-IL2Rβ VHH antibody comprises a complementarity determining region 1 (CDR1) comprising an amino acid sequence of SEQ ID NO: 17 or SEQ ID NO: 414, a CDR2 comprising an amino acid sequence of SEQ ID NO: 18, and a CDR3 comprising an amino acid sequence of SEQ ID NO: 19; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
wherein each CDR independently comprises 0, 1, 2, or 3 amino acid changes relative to the sequence.

7. The IL2R binding protein of claim 1, wherein the anti-IL2Rβ VHH antibody comprises a complementarity determining region 1 (CDR1) comprising an amino acid sequence of SEQ ID NO: 21 or SEQ ID NO: 415, a CDR2 comprising an amino acid sequence of SEQ ID NO: 122, and a CDR3 comprising an amino acid sequence of SEQ ID NO: 23; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
wherein each CDR independently comprises 0, 1, 2, or 3 amino acid changes relative to the sequence.

8. The IL2R binding protein of claim 1, wherein the anti-IL2Rβ VHH antibody comprises a complementarity determining region 1 (CDR1) comprising an amino acid sequence of SEQ ID NO: 25 or SEQ ID NO: 416, a CDR2 comprising an amino acid sequence of SEQ ID NO: 26, and a CDR3 comprising an amino acid sequence of SEQ ID NO: 27; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
wherein each CDR independently comprises 0, 1, 2, or 3 amino acid changes relative to the sequence.

9. The IL2R binding protein of claim 1, wherein the anti-IL2Rβ VHH antibody comprises a complementarity determining region 1 (CDR1) comprising an amino acid sequence of SEQ ID NO: 29 or SEQ ID NO: 417, a CDR2 comprising an amino acid sequence of SEQ ID NO: 30, and a CDR3 comprising an amino acid sequence of SEQ ID NO: 31; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
wherein each CDR independently comprises 0, 1, 2, or 3 amino acid changes relative to the sequence.

10. The IL2R binding protein of claim 1, wherein the anti-IL2Rβ VHH antibody comprises a complementarity determining region 1 (CDR1) comprising an amino acid sequence of SEQ ID NO: 33 or SEQ ID NO: 418, a CDR2 comprising an amino acid sequence of SEQ ID NO: 34, and a CDR3 comprising an amino acid sequence of SEQ ID NO: 35; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
wherein each CDR independently comprises 0, 1, 2, or 3 amino acid changes relative to the sequence.

11. The IL2R binding protein of claim 1, wherein the anti-IL2Rβ VHH antibody comprises a complementarity determining region 1 (CDR1) comprising an amino acid sequence of SEQ ID NO: 37 or SEQ ID NO: 419, a CDR2 comprising an amino acid sequence of SEQ ID NO: 38, and a CDR3 comprising an amino acid sequence of SEQ ID NO: 39; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
wherein each CDR independently comprises 0, 1, 2, or 3 amino acid changes relative to the sequence.

12. (canceled)

13. (canceled)

14. The IL2R binding protein of claim 1, wherein the anti-IL2Rβ VHH antibody comprises a sequence having at least 90% identity to a sequence of any one of DR214 (SEQ ID NO: 4), DR217 (SEQ ID NO: 8), DR583 (SEQ ID NO: 12), DR584 (SEQ ID NO: 16), DR585 (SEQ ID NO: 20), DR586 (SEQ ID NO: 24), DR587 (SEQ ID NO: 28), DR588 (SEQ ID NO: 32), DR589 (SEQ ID NO: 36), and DR590 (SEQ ID NO: 40).

15. (canceled)

16. (canceled)

17. The IL2R binding protein of claim 1, wherein the anti-IL2Rγ VHH antibody comprises a sequence having at least 90% identity to a sequence of any one of DR229 (SEQ ID NO: 44), DR230 (SEQ ID NO: 48), DR231 (SEQ ID NO: 52), DR232 (SEQ ID NO: 56), DR233 (SEQ ID NO: 60), and DR234 (SEQ ID NO: 64).

18. The IL2R binding protein of claim 1, wherein the anti-IL2Rβ VHH antibody is at the N-terminus and the anti-IL2Rγ VHH antibody is at the C-terminus.

19. The IL2R binding protein of claim 18, wherein the binding protein comprises a sequence having at least 90% identity to a sequence of any one of SEQ ID NOS: 65-80.

20. The IL2R binding protein of claim 1, wherein the anti-IL2Rγ VHH antibody is at the N-terminus and the anti-IL2Rβ VHH antibody is at the C-terminus.

21. The IL2R binding protein of claim 20, wherein the binding protein comprises a sequence having at least 90% identity to a sequence of any one of SEQ ID NOS: 81-106.

22. The IL2R binding protein of claim 1, wherein the anti-IL2Rβ VHH antibody and the anti-IL2Rγ VHH antibody are joined by a peptide linker.

23. (canceled)

24. The IL2R binding protein of claim 1, wherein the binding protein comprises a sequence with at least 90% (e.g., 96%, identity to a sequence of any one of SEQ ID NOS: 65-80, SEQ ID NOS: 81-106, or SEQ ID NOS: 170-28.

25. The IL2R binding protein of claim 1, wherein the binding protein is conjugated to an Fc polypeptide or an Fc domain.

26. (canceled)

27. The IL2R binding protein of claim 1, wherein the binding protein is PEGylated.

28. (canceled)

29. A heterodimeric IL2Rβ binding protein/IL2Rγ binding protein pair, the heterodimeric IL2Rβ binding protein/IL2Rβ binding protein pair comprising a first polypeptide of the formula #1: 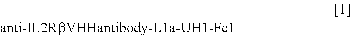
and a second polypeptide of the formula #2: 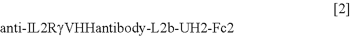
wherein:
L1 and L2 are GSA linkers and a and b are independently selected from 0 (absent) or 1 (present);
UH1 and UH2 are each an upper hinge domain of human immunoglobulin independently selected from the group consisting of the IgG1, IgG2, IgG3 and IgG4 upper hinge;
Fc1 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc2, and
Fc2 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc1, and
wherein the polypeptide of formula 1 and the polypeptide of formula 2 are linked by at least one interchain disulfide bond, and wherein
(A) the anti-IL2Rβ VHH antibody comprises a complementarity determining region 1 (CDR1) comprising an amino acid sequence of SEQ ID NO: 1 or SEQ ID NO: 410, a CDR2 comprising an amino acid sequence of SEQ ID NO: 2, and a CDR3 comprising an amino acid sequence of SEQ ID NO: 3; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
(B) the anti-IL2Rβ VHH antibody comprises a complementarity determining region 1 (CDR1) comprising an amino acid sequence of SEQ ID NO: 5 or SEQ ID NO: 411, a CDR2 comprising an amino acid sequence of SEQ ID NO: 6, and a CDR3 comprising an amino acid sequence of SEQ ID NO: 7; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
(C) the anti-IL2Rβ VHH antibody comprises a complementarity determining region 1 (CDR1) comprising an amino acid sequence of SEQ ID NO: 9 or SEQ ID NO: 412, a CDR2 comprising an amino acid sequence of SEQ ID NO: 10, and a CDR3 comprising an amino acid sequence of SEQ ID NO: 11; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
(D) the anti-IL2Rβ VHH antibody comprises a complementarity determining region 1 (CDR1) comprising an amino acid sequence of SEQ ID NO: 13 or SEQ ID NO: 413, a CDR2 comprising an amino acid sequence of SEQ ID NO: 14, and a CDR3 comprising an amino acid sequence of SEQ ID NO: 15; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
(E) the anti-IL2Rβ VHH antibody comprises a complementarity determining region 1 (CDR1) comprising an amino acid sequence of SEQ ID NO: 17 or SEQ ID NO: 414, a CDR2 comprising an amino acid sequence of SEQ ID NO: 18, and a CDR3 comprising an amino acid sequence of SEQ ID NO: 19; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
(F) the anti-IL2Rβ VHH antibody comprises a complementarity determining region 1 (CDR1) comprising an amino acid sequence of SEQ ID NO: 21 or SEQ ID NO: 415, a CDR2 comprising an amino acid sequence of SEQ ID NO: 122, and a CDR3 comprising an amino acid sequence of SEQ ID NO: 23; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
(G) the anti-IL2Rβ VHH antibody comprises a complementarity determining region 1 (CDR1) comprising an amino acid sequence of SEQ ID NO: 25 or SEQ ID NO: 416, a CDR2 comprising an amino acid sequence of SEQ ID NO: 26, and a CDR3 comprising an amino acid sequence of SEQ ID NO: 27; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
(H) the anti-IL2Rβ VHH antibody comprises a complementarity determining region 1 (CDR1) comprising an amino acid sequence of SEQ ID NO: 29 or SEQ ID NO: 417, a CDR2 comprising an amino acid sequence of SEQ ID NO: 30, and a CDR3 comprising an amino acid sequence of SEQ ID NO: 31; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
(I) the anti-IL2Rβ VHH antibody comprises a complementarity determining region 1 (CDR1) comprising an amino acid sequence of SEQ ID NO: 33 or SEQ ID NO: 418, a CDR2 comprising an amino acid sequence of SEQ ID NO: 34, and a CDR3 comprising an amino acid sequence of SEQ ID NO: 35; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63; or
(J) the anti-IL2Rβ VHH antibody comprises a complementarity determining region 1 (CDR1) comprising an amino acid sequence of SEQ ID NO: 37 or SEQ ID NO: 419, a CDR2 comprising an amino acid sequence of SEQ ID NO: 38, and a CDR3 comprising an amino acid sequence of SEQ ID NO: 39; and wherein the anti-IL2Rγ VHH antibody comprises:
i) a CDR1 comprising an amino acid sequence of SEQ ID NO: 41 or SEQ ID NO: 420, a CDR2 comprising an amino acid sequence of SEQ ID NO: 42, and a CDR3 comprising an amino acid sequence of SEQ ID NO: 43;
ii) a CDR1 comprising an amino acid sequence of SEQ ID NO: 45 or SEQ ID NO: 421, a CDR2 comprising an amino acid sequence of SEQ ID NO: 46, and a CDR3 comprising an amino acid sequence of SEQ ID NO: 47;
iii) a CDR1 comprising an amino acid sequence of SEQ ID NO: 49 or SEQ ID NO: 422, a CDR2 comprising an amino acid sequence of SEQ ID NO: 50, and a CDR3 comprising an amino acid sequence of SEQ ID NO: 51;
iv) a CDR1 comprising an amino acid sequence of SEQ ID NO: 53 or SEQ ID NO: 423, a CDR2 comprising an amino acid sequence of SEQ ID NO: 54, and a CDR3 comprising an amino acid sequence of SEQ ID NO: 55;
v) a CDR1 comprising an amino acid sequence of SEQ ID NO: 57 or SEQ ID NO: 424, a CDR2 comprising an amino acid sequence of SEQ ID NO: 58, and a CDR3 comprising an amino acid sequence of SEQ ID NO: 59;
vi) a CDR1 comprising an amino acid sequence of SEQ ID NO: 61 or SEQ ID NO: 425, a CDR2 comprising an amino acid sequence of SEQ ID NO: 62, and a CDR3 comprising an amino acid sequence of SEQ ID NO: 63;
wherein each CDR independently comprises 0, 1, 2, or 3 amino acid changes relative to the sequence, or
the anti-IL2Rβ VHH antibody comprises:
a CDR1 having at least 90% sequence identity, or having 0, 1, 2, or 3 amino acid changes, relative to the sequence of any CDR1 in a row of Table 30 or Table 31;
a CDR2 having at least 90% sequence identity, or having 0, 1, 2, or 3 amino acid changes, relative to the sequence of any CDR2 in a row of Table 30 or Table 31; and
a CDR3 having at least 90% sequence identity, or having 0, 1, 2, or 3 amino acid changes, relative to the sequence of any CDR3 listed in Table 30 or Table 31; and
the anti-IL2Rγ VHH antibody comprises:
a CDR1 having at least 90% sequence identity, or having 0, 1, 2, or 3 amino acid changes, relative to the sequence of any CDR1 listed in Table 32 or Table 33;
a CDR2 having at least 90% sequence identity, or having 0, 1, 2, or 3 amino acid changes, relative to the sequence of any CDR2 listed in Table 32 or Table 33; and
a CDR3 having at least 90% sequence identity, or having 0, 1, 2, or 3 amino acid changes, relative to the sequence of any CDR3 listed in Table 32 or Table 33.

30. An isolated nucleic acid encoding the IL2R binding protein of claim 1.

31. An expression vector comprising the nucleic acid of claim 30.

32. An isolated host cell comprising the vector of claim 31.

33. A pharmaceutical composition comprising the IL2R binding protein of claim 1.

34. A method of treating a neoplastic disease in a subject in need thereof, comprising administering to the subject a therapeutically effective amount of an IL2R binding protein of claim 1.

35-38. (canceled)

39. A method of treating an autoimmune or inflammatory disease, disorder, or condition or a viral infection in a subject in need thereof, comprising administering to the subject a therapeutically effective amount of an IL2R binding protein of claim 1.

40-42. (canceled)

43. A method to selectively induce proliferation of a first cell type over a second cell type, comprising contacting a population of cells comprising both the first and second cell types with an 112 binding protein of claim 1, thereby selectively inducing proliferation in one or more of the first cell type over one or more of the second cell type.

44-46. (canceled)
